(19) United States
(12) Patent Application Publication
(10) Pub. No.: US 2009/0018123 A1
(43) Pub. Date: Jan. 15, 2009

(54) Oxazolidinones Bearing Antimicrobial Activity Composition and Methods of Preparation

(76) Inventors: Milind D Sindkhedkar, Maharashtra (IN); Satish B. Bhavsar, Maharashtra (IN); Vijaykumar J. Patil, Maharashtra (IN); Prasad K. Deshpande, Maharashtra (IN); Mahesh V Patel, Maharashtra (IN)

Correspondence Address: BIO INTELLECTUAL PROPERTY SERVICES (BIO IPS) LLC, 8509 KERNON CT., LORTON, VA, 22079, US

(21) Appl. No.: 11/922,239

(22) PCT Filed: Jun. 19, 2006
(86) PCT No.: PCT/IN2006/000208

§ 371 Date: Aug. 8, 2008

(30) Foreign Application Priority Data
Jun. 20, 2005 (IN) 723/MUM/2005

(51) Int. Cl. A61K 31/541; C07D 417/14; C07D 413/14; A61K 31/496; C07D 491/113; A61K 31/438; A61K 31/4439; A61K 31/5377; A61P 31/00

(52) U.S. Cl. 514/227.8; 544/60; 544/369; 514/254.02; 546/16; 514/278; 514/326; 546/209; 544/130; 514/236.8

(57) ABSTRACT

The present invention concerns recombinant DNA's comprising cDNA of genomic RNA of a Salmonidae alphavirus preceded by a spacer sequence, under the control of a suitable promoter. Said recombinant DNA's are useful for obtaining expression vectors, producing recombinant Salmonidae alphavirus, and for obtaining vaccines.

### FIELD OF INVENTION

The present invention relates to the field of oxazolidinones having antimicrobial activity. The invention also relates to processes for preparation of the compounds, to pharmaceutical compositions containing the compounds and to methods of treating microbial infections with the compounds.

### BACKGROUND OF INVENTION

Oxazolidinones represent a novel chemical class of synthetic antimicrobial agents, with Linezolid, as a first representative, of this class^{1,2} This advance enabled the profiling of the unique properties of the members of this class, which is that they display activity against important Gram-positive human and veterinary pathogens including Methicillin-resistant Staphylococcus aureus (MRSA), vancomycin resistant enterococci (VRE) and β-lactam resistant Streptococcus pneumoniae (PRSP). The oxazolidinones also show activity against Gram-negative aerobic bacteria and Gram-positive and Gram-negative anaerobes³.

Some deficiencies of oxazolidinones have also surfaced. They are inactive against Enterobacteriaceae⁴. Moreover their potency for atypical respiratory pathogens such as Mycoplasma pneumoniae, M. hominis, Ureaplasma urealyticium and Chlamydia species is of a borderline range which could result into unacceptable clinical efficacy for the treatment of respiratory tract infections³.

Other limitations that have appeared through the clinical development studies and use of Linezolid and its potential successors in development are that the class has a propensity to induce myelosuppression with consequent thrombocytopenia⁵. Inhibition of monoamine oxidase by oxazolidinones has prompted a recommendation made to clinicians that clinical use of members of this class be done with caution during concomitant usage of adrenergic or serotonergic agents and selective serotonin reuptake inhibitors⁶.

Linezolid has been shown to have two targets in cells for its inhibitory effects. It binds to the 50S subunit within domain V of the 23S or RNA peptidyl transferase center near the interface with the 30S subunit, thereby blocking the formation of the tMet-tRNA-ribosome-mRNA ternary complex. In addition, linezolid associates with the nascent 50S particle and stops the assembly process⁷.

The present invention the identification of novel oxazolidinone compounds with antimicrobial activity, which is an embodiment of this invention.

The following publications may be referred to with respect to the statements made in the above-described background information.
¹ Slee A M, et al., Antimicrob. Agents Chemother (1987) 31:1791-1797;
² 2^{nd} European Congress of Chemotherapy and 7^{th} Biennial Conference on Antiinfective Agents and Chemotherapy (Final Program), (1998): 93;
³ Diekema D J et al., Lancet 2001; 358: 1975-82;
⁴ Zhanel G G et al., Canadian Journal of Infectious Diseases, 2001, 12: 379-390;
⁵ Kuter D J et al., Pharmacotherapy, 2001:21:1010-1030;
⁶ Ament P W et al., Am Fam Physician 2002, 65: 663-70;
⁷ Shinabarger D, Exp. Opin. Invest. Drugs (1999) 8:1195-1202; Champrey W S et al., Curr. Microb. 2002, 44: 350-356;

### Information Disclosure

There are several patents cited in the literature, which refer to oxazolidinones having antibacterial activity. Piperidinylphenyl moiety bearing oxazolidinones are described in following patents;

WO95/25106 discloses substituted piperidino phenyloxazolidinones. This corresponds to U.S. Pat. No. 5,668,286 and EP 0.750618.

WO 96/13502 discloses phenyloxazolidinones having a multisubstituted azetidinyl or pyrrolidinyl moiety.

U.S. application Ser. No. 10/475,735; 10/616,888 and 10/935,708 PCT application PCT/IN03/00237, PCT/IN03/00238 and PCT/IN04/00276 discloses piperidinyl phenyl oxazolidinones of antimicrobial use.

Pyrrolidinyl/piperidinyl phenyl oxazolidinone antibacterial agents are also described in Kim H Y et al., Bioorg. M Med. Chem. Lett., (2003), 13:2227-2230.

However in all above cited patents, substituted or unsubstituted heteroaryl bearing piperidinophenyl oxazolidinone compounds are not disclosed.

Similarly, following citations pertain to oxazolidinones having biaryl moiety attached to oxazolidinone ring.

WO 2004/048350 A2 describes pyridyl and pyrimidyl moiety as one of constituents of biaryl oxazolidinone compounds. However oxazolidinone moiety bearing methyl acetamide, or methyl thioacetamide are not described. This application does not disclose halogen substituted biphenyl oxazolidinone compounds with methyl acetamide moiety.

US 2004/147760 A1 and WO 2003/072553 A1 describe some biaryl oxazolidinone compounds but oxazolidinones with methyl acetamide are not described.

WO 0194342 A1 describes pyridinyl and pyrimidinyl moiety as one of constituents of biaryl oxazolidinone compounds. However oxazolidinone biaryl compounds bearing thiomorpholine are not described. This application does not disclose halogen substituted on both biphenyl rings of oxazolidinone compounds.

WO 04056819 A1 also describes pyridinyl and pyrimidinyl moiety as one of constituents of biaryl oxazolidinone compounds. However examples of biaryl oxazolidinone compounds bearing methyl acetamide are not disclosed. This application does not describe halogen substituted on both rings of biphenyl moiety.

WO 2005/058886 A1 describe heterocyclic ring attached to biaryl compounds. However examples of biaryl oxazolidinone compounds bearing morpholino derivatives are not disclosed.

WO 2005/012271 A2 describes a process for synthesis of biaryl compounds where one of the ring in biaryl moiety is either pyridine or desfluorophenyl ring however biaryl ring with both rings bearing fluorine atom are not described. WO 2005/012271 A2 does not describe thiomorpholine moiety as one of the substituent on biaryl moiety.

The present inventors have found that the novel piperidino substituted phenyloxazolidinones and biaryl oxazolidinones of the invention herein described have a favorable in vitro and in vivo efficacy advantages.

U.S. application Ser. No. 10/475,735; 10/616,888 and 10/935,708 and PCT application PCT/IN03/00237, PCT/IN03/00238 and PCT/IN04/00276 discloses a novel series of oxazolidinones which display increased potency.

### SUMMARY OF INVENTION

The present invention relates to novel substituted phenylpiperidino and biaryl oxazolidinone compounds of Formula I.

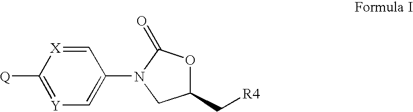

wherein Q is

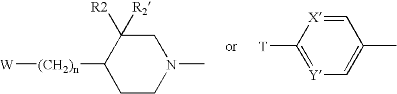

X and Y may be same or different, represent, CH, CF, N;
X′ and Y′ may be same or different, represent,
CH, CF, N, C—OCH₃, C—CH₂—Rₐ; wherein Rₐ is hydrogen, amino, halogen, hydroxyl, azido, carboxamido, NHCH₂CONH₂, N(CH₂CONH₂)₂;
R₂ and R_{2′} may be same or different, represent, hydrogen, methyl, hydroxyl, C₁-C₆ alkoxy or halogen;
R₄ is selected from the group comprising
a) O—R₁,
b) NR_{b}R_{c};
   wherein R_{b} is selected from hydrogen, C₁-C₆ alkylcarbonyl;
   R_{c} is selected from hydrogen, C(O)OCH₂OC(O)R_{d} or CH(CH₃)OC(O)Rₑ;
   wherein R_{d} is hydrogen, C₁-C₆ alkyl, substituted C₁-C₆ alkyl with substitutents selected from amino or C₁-C₆ alkylamido
   Rₑ is selected from hydrogen, C₁-C₆ alkyl, substituted C₁-C₆ alkyl;
c) formamide;
d) carbamate;
e) thiocarbamate;
f) urea;
g) C₁-C₆ alkylamido;
h) C₁-C₆ haloalkylamido;
i) C₁-C₆ thioalkylamido;
j) substituted C₁-C₆ thioalkylamido, wherein the alkyl group is substituted with halo;
k) unsubstituted or substituted heteroaryl, wherein substituents of the heteroaryl are selected from the group comprising C₁-C₆ alkyl, nitro, cyano, hydroxyl, halo, amino, C₁-C₆ alkylcarbonyl, C₁-C₆ alkoxycarbonyl, carboxy;
   wherein R₁ is selected from the group comprising hydrogen, C₁-C₆ alkylsulfonyl, C₁-C₆ alkylcarbonyl, —P(O)(OM)₂, wherein M is hydrogen, methyl, ethyl, t-butyl, phenyl or an alkali metal ion such as Li, Na, K;
   or R₁ is an amino acid residue derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, attached to the oxygen via carbonyl of the amino acid to form a ester linkage;

### When Q is

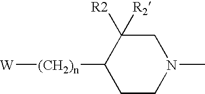

wherein n is 0, 1 or 2;
W represents 3-7 membered heterocyclyl bearing one or more heteroatom selected from N, O, S; optionally substituted with one or more substitutents selected from the group comprising C₁-C₆ alkyl, C₁-C₆ alkylcarbonyl, C₁-C₆ alkoxycarbonyl, carboxamide, cyano, hydroxyl, amino, heterocyclyl, substituted heterocyclyl, aryl, substituted aryl, heteroaryl, substituted heteroaryl
or
heteroaryl bearing one or more heteroatom selected from N, O S, optionally substituted with one or more substitutents selected from the group comprising
i. substituted or unsubstituted C₁-C₆ alkyl with substituents selected from the group hydroxyl, halo, nitro, cyano, aryl or heteroaryl;
ii. substituted or unsubstituted C₂-C₆ alkenyl with substituents selected from the group hydroxyl, halo, nitro, cyano, aryl or heteroaryl;
iii. C₁-C₆ alkylcarbonyl;
iv. C₁-C₆ alkoxycarbonyl;
v. C₁-C₆ alkoxycarbonylalkyl;
vi. C₁-C₆ alkylcarbonylalkyl;
vii. C₁-C₆ haloalkyl;
viii. formyl;
ix. carboxy;
x. carboxamide;
xi. cyano;
xii. amino;
xiii. nitro;
xiv. hydroxyl;
xv. halo;
xvi. morpholinocarbonylalkyl;
xvii. hydroxyiminoalkyl;
xviii. alkylcarbonylaminoalkyl;
xix. alkoxyiminoalkyl;
xx. aralkyloxyiminoalkyl;
xxi. hydroiminoaminoalkyl;
xxii. aryl, substituted aryl, with substituents selected from C₁-C₆ alkyl, nitro, cyano, hydroxyl, halo, amino, heterocyclyl;
xxiii. substituted or unsubstituted heteroaryl, with substituents selected from C₁-C₆ alkyl, nitro, cyano, hydroxyl, halo, amino, heterocyclyl;
xxiv. substituted or unsubstituted heterocyclyl, with substituents selected from C₁-C₆ alkyl, C₁-C₆ alkylcarbonyl, aralkyl, nitro, cyano, hydroxyl, halo, amino;
xxv. C₁-C₆ alkylthio;
xxvi. C₁-C₆ alkylsulfanylalkyl;
xxvii. C₁-C₆ aralkylsulfanyl;
xxviii. C₁-C₆ alkylsulfonylmethyl;
xxix. C₁-C₆ alkylsulfonyloxy;
xxx. NR_{b}R_{c};
xxxi. —(C₁-C₆ alkyl)-NR_{f}—(C₁-C₆ alkyl)-NR_{b}R_{c};
wherein R_{f} is hydrogen, C₁-C₆ alkyl, substituted C₁-C₆ alkyl with substitutents selected from amino or C₁-C₆ alkylamido;
   or
   W is heteroaryloxy group optionally substituted with one or more substitutents such as C₁-C₆ alkyl, C₂-C₆ alkenyl, C₁-C₆ alkylcarbonyl, C₁-C₆ alkoxycarbonyl, formyl, carboxy, carboxamide, cyano, amino, hydroxyiminoalkyl, alkoxyiminoalkyl, aralkyloxyimunoalkyl, hydroxyiminoaminoalkyl, aryl, substituted aryl, heteroaryl, substituted heteroaryl, heterocyclyl, substituted heterocyclyl;

### OR

when Q is

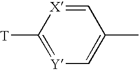

X′ and Y′ are as defined above;
T is hydrogen or

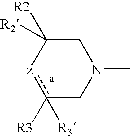

wherein
R₂ and R_{2′} are as defined above;
“a” is optional double bond;
R₃ and R_{3′} may be same or different, represent, hydrogen, methyl, hydroxyl, C₁-C₆ alkoxy or halogen;
with the proviso that when T is hydrogen, R₄ is not acetamido;
Z represents group selected from CH, NH, O, S, CH₂, C(R₆)R₆′, C═O, NR₇, C═C(R₈)R_{8′}, SO, SO₂, S═NH, S═NC(O)CH₃, S═NC(O)NHCH₃, S(O)═NH, S(O)═NCH₃, S(O)NC(O)CH₃, S(O)═NC(O)NHCH₃, S(O)═NC(O)NHCH₂CH₂Cl,
with proviso that when X is CH, Y is CF, one of X′ or Y′ or both X′, Y′ are N, then Z is not NH.
wherein R₆ and R₆′ may be same or different, represent, hydrogen, hydroxyl, amino, azido, C₁-C₆ alkoxy, C₁-C₆ alkylcarbonyloxy, C₁-C₆ alkylsulfonyloxy, heterocyclyl, substituted heterocyclyl, aryl, substituted aryl, heteroaryl, substituted heteroaryl, C₁-C₆ alkyl optionally substituted with one or more substituent selected from the group comprising azido, cyano, carboxamido, hydroxyl, C₁-C₆ alkyloxy, C₁-C₆ alkylcarbonyloxy, C₁-C₆ alkylcarbonyl or C₂-C₆ alkenyl optionally substituted with one or more azido, cyano, carboxamido or hydroxyl;
or
R₆ and R₆′ are combined together to provide 3-7 member heterocyclyl bearing one or more heteroatom selected from N, O, S optionally substituted with substituent selected from group comprising C₁-C₆ alkyl, C₂-C₆ alkenyl, C₁-C₆ alkylcarbonyl, C₁-C₆ alkoxycarbonyl, formyl, carboxy, carboxamide, cyano, amino, hydroxyiminoalkyl, alkoxyiminoalkyl, aralkyloxyiminoalkyl, hydroxyiminoaminoalkyl, aryl, substituted aryl, heteroaryl, substituted heteroaryl, heterocyclyl, substituted heterocycyl;
R₇ represents hydrogen, cyano, aralkyl, CO—OCH₂Ph, C₁-C₆-alkylcarbonyl, C₁-C₆ alkyl optionally substituted with one or more azido, cyano, heteroaryl, substituted heteroaryl, carboxamido or hydroxyl;
R₈ R_{8′} may be same or different, represent, hydrogen, C₁-C₆ alkyl, C₂-C₆ alkenyl, cyano, amino, C₁-C₆ alkylcarbonyl, C₁-C₆ alkoxycarbonyl, cyano, aryl, substituted aryl, heteroaryl, substituted heteroaryl, heterocyclyl, substituted heterocyclyl;
and isomers, polymorphs, N-oxides thereof or pharmaceutical acceptable salts thereof.

### DETAILED DESCRIPTION OF THE INVENTION

The present invention relates to novel substituted phenylpiperidino and biaryl oxazolidinone compounds of Formula I,

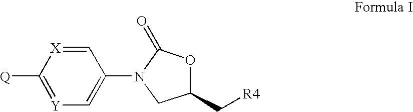

wherein Q is

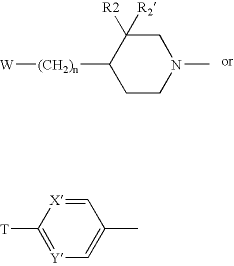

X and Y may be same or different, represent, CH, CF, N;
X′ and Y′ may be same or different, represent,
CH, CF, N, C—OCH₃, C—CH₂—Rₐ; wherein Rₐ is hydrogen, amino, halogen, hydroxyl, azido, carboxamido, NHCH₂CONH₂, N(CH₂CONH₂)₂;
R₂ and R_{2′} may be same or different, represent, hydrogen, methyl, hydroxyl, C₁-C₆ alkoxy or halogen;
R₄ is selected from the group comprising
a) O—R₁,
b) NR_{b}R_{c};
   wherein R_{b} is selected from hydrogen, C₁-C₆ alkylcarbonyl;
   R_{c} is selected from hydrogen, C(O)OCH₂OC(O)R_{d} or CH(CH₃)OC(O)Rₑ;
   wherein R_{d} is hydrogen, C₁-C₆ alkyl, substituted C₁-C₆ alkyl with substitutents selected from amino or C₁-C₆ alkylamido
   Rₑ is selected from hydrogen, C₁-C₆ alkyl, substituted C₁-C₆ alkyl;
c) formamide;
d) carbamate;
e) thiocarbamate;
f) urea;
g) C₁-C₆ alkylamido;
h) C₁-C₆ haloalkylamido;
i) C₁-C₆ thioalkylamido;
j) substituted C₁-C₆ thioalkylamido, wherein the alkyl group is substituted with halo;
k) unsubstituted or substituted heteroaryl, wherein substituents of the heteroaryl are selected from the group comprising C₁-C₆ alkyl, nitro, cyano, hydroxyl, halo, amino, C₁-C₆ alkylcarbonyl, C₁-C₆ alkoxycarbonyl, carboxy;
   wherein R₁ is selected from the group comprising hydrogen, C₁-C₆ alkylsulfonyl, C₁-C₆ alkylcarbonyl, —P(O)(OM)₂, wherein M is hydrogen, methyl, ethyl, t-butyl, phenyl or an alkali metal ion such as Li, Na, K;
   or R₁ is an amino acid residue derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, attached to the oxygen via carbonyl of the amino acid to form a ester linkage;

When Q is

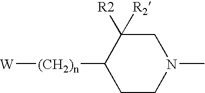

wherein n is 0, 1 or 2;
W represents 3-7 membered heterocyclyl bearing one or more heteroatom selected from N, O, S, optionally substituted with one or more substitutents selected from the group comprising C₁-C₆ alkyl, C₁-C₆ alkylcarbonyl, C₁-C₆ alkoxycarbonyl, carboxamide, cyano, hydroxyl, amino, heterocyclyl, substituted heterocyclyl, aryl, substituted aryl, heteroaryl, substituted heteroaryl
or
heteroaryl bearing one or more heteroatom selected from N, O S, optionally substituted with one or more substitutents-selected from the group comprising
i. substituted or unsubstituted C₁-C₆ alkyl with substituents selected from the group hydroxyl, halo, nitro, cyano, aryl or heteroaryl;
ii. substituted or unsubstituted C₂-C₆ alkenyl with substituents selected from the group hydroxyl, halo, nitro, cyano, aryl or heteroaryl;
iii. C₁-C₆ alkylcarbonyl;
iv. C₁-C₆ alkoxycarbonyl;
v. C₁-C₆ alkoxycarbonylalkyl;
vi. C₁-C₆ alkylcarbonylalkyl;
vii. C₁-C₆ haloalkyl;
viii. formyl;
ix. carboxy;
x. carboxamide;
xi. cyano;
xii. amino;
xiii nitro;
xiv. hydroxyl;
xv. halo;
xvi. morpholinocarbonylalkyl;
xvii. hydroxyiminoalkyl;
xviii. alkylcarbonylaminoalkyl;
xix. alkoxyiminoalkyl;
xx. aralkyloxyiminoalkyl;
xxi. hydroiminoaminoalkyl;
xxii. aryl, substituted aryl, with substituents selected from C₁-C₆ alkyl, nitro, cyano, hydroxyl, halo, amino, heterocyclyl;
xxiii. substituted or unsubstituted heteroaryl, with substituents selected from C₁-C₆ alkyl, nitro, cyano, hydroxyl, halo, amino, heterocyclyl;
xxiv. substituted or unsubstituted heterocyclyl, with substituents selected from C₁-C₆ alkyl, C₁-C₆ alkylcarbonyl, aralkyl, nitro, cyano, hydroxyl, halo, amino;
xxv. C₁-C₆ alkylthio;
xxvi. C₁-C₆ alkylsulfanylalkyl;
xxvii. C₁-C₆ aralkylsulfanyl;
xxviii. C₁-C₆ alkylsulfonylmethyl;
xxix. C₁-C₆ alkylsulfonyloxy;
xxx. NR_{b}R_{c}:
xxxi. —(C₁-C₆ alkyl)-NR_{f}—(C₁-C₆ alkyl)-NR_{b}R_{c}:
wherein R_{f} is hydrogen, C₁-C₆ alkyl, substituted C₁-C₆ alkyl with substitutents selected from amino or C₁-C₆ alkylamido;
   or
   W is heteroaryloxy group optionally substituted with one or more substitutents such as C₁-C₆ alkyl, C₂-C₆ alkenyl, C₁-C₆ alkylcarbonyl, C₁-C₆ alkoxycarbonyl, formyl, carboxy, carboxamide, cyano, amino, hydroxyiminoalkyl, alkoxyiminoalkyl, aralkyloxyiminoalkyl, hydroxyiminoaminoalkyl, aryl, substituted aryl, heteroaryl, substituted heteroaryl, heterocyclyl, substituted heterocyclyl;

### OR

when Q is

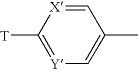

X′ and Y′ are as defined above;
T is hydrogen or

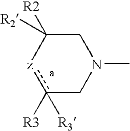

wherein
R₂ and R_{2′} are as defined above;
“a” is optional double bond;
R₃ and R_{3′} may be same or different, represent, hydrogen, methyl, hydroxyl, C₁-C₆ alkoxy or halogen;
with the proviso that when T is hydrogen, R₄ is not acetamido;
Z represents group selected from CH, NH, O, S, CH₂, C(R₆)R₆′, C═O, NR₇, C═C(R₈)R_{8′}, SO, SO₂, S═NH, S═NC(O)CH₃, S═NC(O)NHCH₃, S(O)═NH, S(O)═NCH₃, S(O)NC(O)CH₃, S(O)═NC(O)NHCH₃, S(O)═NC(O)NHCH₂CH₂Cl,
with proviso that when X is CH, Y is CF, one of X′ or Y′ or both X′, Y′ are N, then Z is not NH.
wherein R₆ and R₆′ may be same or different, represent, hydrogen, hydroxyl, amino, azido, C₁-C₆ alkoxy, C₁-C₆ alkylcarbonyloxy, C₁-C₆ alkylsulfonyloxy, heterocyclyl, substituted heterocyclyl, aryl, substituted aryl, heteroaryl, substituted heteroaryl, C₁-C₆ alkyl optionally substituted with one or more substituent selected from the group comprising azido, cyano, carboxamido, hydroxyl, C₁-C₆ alkyloxy, C₁-C₆ alkylcarbonyloxy, C₁-C₆ alkylcarbonyl or C₂-C₆ alkenyl optionally substituted with one or more azido, cyano, carboxamido or hydroxyl;
or
R₆ and R₆′ are combined together to provide 3-7 member heterocyclyl bearing one or more heteroatom selected from N, O, S optionally substituted with substituent selected from group comprising C₁-C₆ alkyl, C₂-C₆ alkenyl, C₁-C₆ alkylcarbonyl, C₁-C₆ alkoxycarbonyl, formyl, carboxy, carboxamide, cyano, amino, hydroxyiminoalkyl, alkoxyiminoalkyl, aralkyloxyiminoalkyl, hydroxyiminoaminoalkyl, aryl, substituted aryl, heteroaryl, substituted heteroaryl, heterocyclyl, substituted heterocyclyl;
R₇ represents hydrogen, cyano, aralkyl, CO—OCH₂Ph, C₁-C₆-alkylcarbonyl, C₁-C₆ alkyl optionally substituted with one or more azido, cyano, heteroaryl, substituted heteroaryl, carboxamido or hydroxyl;
R₈, R_{8′} may be same or different, represent, hydrogen, C₁-C₆ alkyl, C₂-C₆ alkenyl, cyano, amino, C₁-C₆ alkylcarbonyl, C₁-C₆ alkoxycarbonyl, cyano, aryl, substituted aryl, heteroaryl, substituted heteroaryl, heterocyclyl, substituted heterocyclyl;
and isomers, polymorphs, N-oxides thereof or pharmaceutical acceptable salts thereof.
The preferred compounds of the invention are
a) The compound of formula Ia,

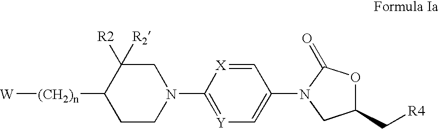

wherein, n is 0 or 1;
R₂ and R_{2′} are each independently hydrogen, methyl, hydroxyl, halogen;
X and Y each independently CH or CF;
W is heteroaryl or substituted heteroaryl, wherein heteroaryl is [1,2,3]-triazol, [1,2,4]triazol, pyrrol, pyrazol, tetrazol, imidazol, [1,3,4]-oxadiazol, [1,2,4]-thiadiazol, [1,3,4]-thiadiazol, oxazol, isoxazol, thiazol, benzotriazol;
the substituents on heteroaryl are selected from group comprising methyl, cyano, amino, fluoro, difluoromethyl, formyl, hydroxyl methyl, carboxamide, acetyl, 1-methoxyimino-methyl, 4-pyridinyl, 3-pyridinyl, diethylamino, methylsulfonylmethyl;
R₄ is C₁-C₆ alkylamido, acetamide, difluoroacetamide, thioacetamide, difluoroacetamide, carbamate, thiocarbamate, urea, [1,2,3]-triazol, [1,2,4]-triazol.
b) The compound of formula Ib

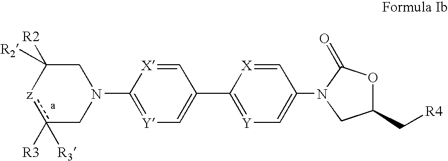

wherein,
a is optional double bond;
X and Y each independently CH, CF or N;
X′ and Y′ are each independently CH, CF, N, C—CH₃;
R₂, R_{2′}, R₃ and R_{3′} are each independently hydrogen, methyl, hydroxyl, halogen;

### Z is S, SO, SO₂;

R₄ is acetamido, [1,2,3]-triazol, methyl carbamate, t-butyl carbamate, OR₁ wherein R₁ is hydrogen, P(O)(OM)₂, wherein M is hydrogen, Na, methyl, ethyl, t-butyl, phenyl; or R₁ is an amino acid residue derived from one of the 20 naturally occurring amino acids viz. alanine, arginine, asparagine, aspartic acid, cysteine, glutamine, glutamic acid, glycine, histidine, isoleucine, leucine, lysine, Methionine, phenylalanine, proline, serine, threonine, tryptophan, tyrosine and valine, attached to the oxygen via carbonyl of amino acid to form an ester linkage.

### DESCRIPTION OF TERMS

The following definitions are used, unless otherwise described.

The term “C₁-C₆ alkyl” refers to saturated, straight or branched chain hydrocarbon having C—C₆ number of carbon atoms such as methyl, ethyl, propyl, isopropyl and so on.

“substituted C₁-C₆ alkyl” refers to one or more hydrogen atom of the alkyl group substituted with halogen, amino, hydroxy, carboaxldehyde, mercapto, nitro, carboxy, alkoxycarbonyl, carboxamide, aryl, heteroaryl, substituted aryl, substituted heteroaryl.

“C₂-C₆ alkenyl’ means straight or branched chain hydrocarbon comprising C₁-C₆ carbon atom containing one or more carbon-carbon double bonds for examples ethenyl, propenyl, butenyl, pentenyl, hexenyl.

“substituted C₁-C₆ alkenyl” refers to one or more hydrogen atom of the alkenyl group substituted with halogen, amino, hydroxy, carboaxldehyde, mercapto, nitro, carboxy, alkoxycarbonyl, carboxamide, aryl, heteroaryl, substituted aryl, substituted heteroaryl.

“C₂-C₆ alkynyl’ means straight or branched chain hydrocarbon comprising C₁-C₆ carbon atom containing one or more carbon-carbon triple bonds for examples ethynyl, propynyl, butynyl, pentynyl, hexynyl.

“C₁-C₆ alkylsulfonyloxy” means groups such as methanesulfonyloxy, ethanesulfonyloxy, propylsulfonyloxy and so on.

“C₁-C₆-alkoxycarbonyl” means group such as methoxycarbonyl (CH₃O—CO), ethoxycabonyl (C₂H₅O—CO), propoxycarbonyl (C₃H₇O—CO) and so on.

“halogen” or “halo” means atom selected from atom such as fluorine, chlorine, bromine.

“C₁-C₆ alkylcarbonyl” means groups such as acetyl, ethylcarbonyl, propylcarbonyl.

“C₁-C₆ alkylthio” means groups such as methylthio, ethylthio, propylthio.

“aryl” refers to a mono, fused bicyclic or fused tricyclic carbocyclic ring system having one or more aromatic rings including but not limited to phenyl, napthyl, indanyl, indenyl and so on.

“substituted aryl” refers to an aryl group as defined herein substituted by independent replacement of one or more hydrogen atoms thereon with Cl, Br, F, I, OH, CN, C₁-C₆ alkyl, C₁-C₆ alkoxy, haloalkyl, amino, alkylamino, dialkylamino, mercapto, nitro, carboxaldehyde, carboxy, alkoxycarbonyl, carboxamide.

“heteroaryl” refers to mono, fused bicyclic or tricyclic aromatic radical having 5-10 ring atoms of which one or more carbon atom of the ring is replaced by an atom selected from N, O, S, for example pyrrolyl, pyrazolyl, imidazolyl, [1,2,3]-triazolyl, [1,2,4]-triazolyl, tetrazolyl, [1,2,4]-oxadiazolyl, [1,3,4]-oxadiazolyl, furanyl, thiophenyl, [1,2,4]-thiadiazolyl, [1,3,4]-thiadiazolyl, thiazolyl, oxazolyl, isoxazolyl, pyridyl, pyrimidinyl, benzotriazolyl, quinolinyl, isoquinolinyl, and the like.

“substituted heteroaryl” refers to a heteroaryl group as defined herein substituted by independent replacement of one or more hydrogen atoms thereon with Cl, Br, F, 1, OH, CN, C₁-C₆ alkyl, C₁-C₆ alkoxy, haloalkyl, amino, alkylamino, dialkylamino, mercapto, nitro, carboxaldehyde, carboxy, alkoxycarbonyl, carboxamide.

“heterocyclyl” means mono-, bi- or tri-cyclic ring systems which may be partially or fully saturated having 3-10 ring atoms. The individual rings may be 3-7 member bearing one or more heteroatom selected from N, O, S. This includes aryl and heteroaryl ring stems fused to non aromatic ring. For example aziridinyl, oxiranyl, piperidinyl, piperazinyl, morpholinyl, thiomorpholinyl, oxadiazolonyl, oxazolidinonyl, thiadiazolonyl, 5-thioxo-4,5-dihydro-[1,2,4]triazol-1-yl and so on.

“substituted heterocyclyl” refers to a heterocyclyl group as defined above substituted by independent replacement of one or more hydrogen atoms thereon with Cl, Br, F, I, OH, CN, C₁-C₆ alkyl, C₁-C₆ alkoxy, haloalkyl, amino, alkylamino, dialkylamino, mercapto, nitro, carboxaldehyde, carboxy, alkoxycarbonyl, carboxamide.

“aralkyl” means groups like benzyl, benhydryl, trityl and so on.

“haloalkyl” refers to C₁-C₆ alkyl group substituted with one or more halogen for example chloromethyl, bromoethyl and the like.

“heteroaryloxy group’ means heteroaryl linked via ether linkage for example group such as isooxazolyloxy, thiophenyloxy, pyridinyloxy.

“C₁-C₆ alkoxy” refers for the C₁-C₆ alkyl group linked via ether linkage for example methoxy, ethoxy and so on.

“acetamido” stands for NHC(O)CH₃. “formamide” stands for NH—CHO.

“C₁-C₆ alkylamido” means an alkyl group attached to the carbonyl of the amide. Examples of alkyamido groups include acetamido, —NHC(O)—C₂H₅, —NHC(O)—C₃H₇ and so on.

“C₁-C₆ haloalkylamido” means alkyl group substituted with halogen, for example —NHC(O)—CHCl₂, —NHC(O)—CHF₂, —NHC(O)—CH₂CHCl₂ and so on.

“thioacetamido”, stands for NHC(S)CH₃.

“C₁-C₆ thioalkylamido” refers to alkylamido group wherein carbonyl group is replaced by C═S group; examples of alkyamido groups include thioacetamido, —NHC(S)—C₂H₅, —NHC(S)—C₃H₇ and so on.

“substituted C₁-C₆ thioalkylamido” refers to thioalkylamido group wherein alkyl group is substituted with halogen; for example —NHC(S)—CHCl₂, —NHC(S)—CHF₂, —NHC(S)—CH₂CHCl₂ and so on.

The term “carboxamide”, refers to a group of the formula —CONH₂, —C(O)NH(C₁-C₆alkyl) or —C(O)N(C₁-C₆alkyl)(C₁-C₆alkyl).

“formyl”, refers to the group —CHO. “cyano” refers to the group —CN.

The term “amino” refers to NH₂. “hydroxyl” refers to OH. “nitro” stands for NO₂. “azido” stands for N₃. The term “urea” stands for NH—CO—NH₂.

“substituted amino” refers to one or both hydrogen of amino substituted with optionally substituted C₁-C₆ alkyl or optionally substituted C₁-C₆ alkenyl.

“carboxy”, as used herein refers to a group of the formula —COOH.

“hydroxyiminoalkyl”, refers to —C═N(OH)(C₁-C₆ alkyl).

“alkoxyiminoalkyl”, stands for —C═N(O—(C₁-C₆ alkyl))(C₁-C₆ alkyl).

“aralkyloxyiminoalkyl”, stands for —C═N(O-(aralkyl)(C₁-C₆ alkyl) for example benzyloxyiminomethyl.

“hydroxyiminoaminoalkyl” refers to —C═N(OH)NH₂.

“C₁-C₆ alkoxycarbonylalkyl” refers to (C₁-C₆ alkyl)-O—CO—(C₁-C₆ alkyl) for example CH₃OCOCH₂—

“C₁-C₆ alkylcarbonylalkyl” refers to (C₁-C₆ alkyl)-CO—(C₁-C₆ alkyl) for example CH₃COCH₂—

“morpholinocarbonylalkyl” refers to morpholinocarbonyl-(C₁-C₆ alkyl) for example morpholinocarbonylmethyl.

“alkylcarbonylaminoalkyl” refers to (C₁-C₆ alkyl)-CO—NH₂—(C₁-C₆ alkyl) for example CH₃CONH₂CH₂—.

“C₁-C₆ alkylsulfanylalkyl” refers to (C₁-C₆ alkyl)-S—(C₁-C₆ alkyl) for example CH₃SCH₂.

“C₁-C₆ alkylsulfonylmethyl” refers to (C₁-C₆ alkyl)-SO₂—(C₁-C₆ alkyl) for example CH₃SO₂CH₂—.

“C₁-C₆ aralkylsulfanyl” refers to aralkyl group attached to sulfur for example PhCH₂S—.

“carbamate” refers to NH-CO—O-alkyl, for example NH—CO—O—CH₃ (methyl carbamate), NHCO₂C₂H₅ (ethyl carbamate)

“thiocarbamate” refers to NH—CS—O-alkyl, for example NH—CS—O—CH₃ (methyl thiocarbamate), NHCS—OC₂H₅ (ethyl thiocarbamate) and the like.

“Mammal” refers to human or animals including livestock and companion animals.

A “therapeutically effective amount” is an amount of a compound of the present invention that, when administered to a patient, provides the desired effect; i.e., lessening in the severity of the symptoms associated with a bacterial infection.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

Certain compounds of the invention are also useful as intermediates for preparing other compounds of the invention, a conversion which can occur both in vitro and in vivo.

Some of the compounds of the invention are capable of further forming pharmaceutically acceptable acid-addition and/or base salts. All of these forms are within the scope of the present invention. Thus, pharmaceutically acceptable acid addition salts of the compounds of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzensoulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, S. M. et. al., “Pharmaceutical Salts,” Journal of Pharmaceutical Science, 1977; 66:1-19).

The acid addition salt of said basic compounds are prepared by contacting the free base form with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

Preferred salts are those of hydrochloride, hydrobromide, hydroiodide, sulphate, phosphate and salts of organic acids such as acetate, lactate, succinate, oxalate, maleate, fumarate, malate, tartrate, citrate, ascorbate, cinnamate, gluconate, benzoate, methane sulfonate and p-toluene sulfonate; lithium, sodium, magnesium, calcium and potassium salts, and amino acids salts such as alanine, arginine, asparagine, aspartic acid, cysteine, glutamine, glutamic acid, glycine, histidine, isoleucine, leucine, lysine, methionine, phenylalanine, proline, serine, threonine, tryptophan tyrosine or valine.

The base addition salts of said acidic compounds are prepared by contacting the free acid form with a sufficient amount of the desired base to produce the salt in the conventional manner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

A “prodrug” is an inactive derivative of a drug molecule that requires a chemical or an enzymatic biotransformation in order to release the active parent drug in the body. Some of the representative examples of oxazolidinone derivatives of the present invention represented by the general formula I are as follows:
1. (S)—N-{3-[4-(4-(2-Cyanoaziridin-1-yl)-piperidin-1-yl)-3-fluorophenyl]-2-oxo-oxazolidin-5-ylmethyl}-acetamide;
2. (S)—N-{3-[4-(4-pyrrol-1-yl-piperidin-1-yl)-3-fluorophenyl]-2-oxo-oxazolidin-5-ylmethyl}-acetamide;
3. (S)—N-{3-[3,5-Difluoro-4-(4-pyrrol-1-yl-piperidin-1-yl)-phenyl]-2-oxo-oxazolidin-5-ylmethyl}-acetamide;
4. (S)—N-{3-[4-(4-(1H-imidazol-1-yl)-piperidin-1-yl)-3-fluorophenyl]-2-oxo-oxazolidin-5-ylmethyl}-acetamide;
5. (S)—N-{3-[4-(4-(1H-[1,2,3]-triazol-1-yl)-piperidin-1-yl)-3-fluorophenyl]-2-oxo-oxazolidin-5-ylmethyl}-acetamide;
6. (S)—N-{3-[4-(4-(2H-[1,2,3]-triazol-2-yl)-piperidin-1-yl)-3-fluorophenyl]-2-oxo-oxazolidin-5-ylmethyl}-acetamide;
7. (S)—N-{3-[4-(4-(1H-[1,2,3]-triazol-1-yl)-piperidin-1-yl)-3,5-difluorophenyl]-2-oxo-oxazolidin-5-ylmethyl}-acetamide;
8. (S)—N-{3-[4-(4-(2H-[1,2,3]-triazol-2-yl)-piperidin-1-yl)-3,5-difluorophenyl]-2-oxo-oxazolidin-5-ylmethyl}-acetamide;
9. (S)—N-{3-[4-(4-(1H-[1,2,3]-triazol-1-yl)-3-fluoropiperidin-1-yl)-3,5-difluorophenyl]-2-oxo-oxazolidin-5-ylmethyl}-acetamide;
10. (S)—N-{3-[4-(4-(2H-[1,2,3]-triazol-2-yl)-3-fluoropiperidin-1-yl)-3,5-difluorophenyl]-2-oxo-oxazolidin-5-ylmethyl}-acetamide;
11. (S)—N-{3-[4-(4-(1H-[1,2,3]-triazol-1-yl)-3-fluoropiperidin-1-yl)-3-fluorophenyl]-2-oxo-oxazolidin-5-ylmethyl}-acetamide;
12. (S)—N-{3-[4-(4-(2H-[1,2,3]-triazol-2-yl)-3-fluoropiperidin-1-yl)-3-fluorophenyl]-2-oxo-oxazolidin-5-ylmethyl}-acetamide;
13. (S)—N-{3-[4-(4-(1H-[1,2,4]-triazol-1-yl)-piperidin-1-yl)-3-fluorophenyl]-2-oxo-oxazolidin-5-ylmethyl}-acetamide;
14. (S)—N-{3-[4-(4-(tetrazol-5-yl)-piperidin-1-yl)-3-fluorophenyl]-2-oxo-oxazolidin-5-ylmethyl}-acetamide;
15. (S)—N-{3-[4-(4-(tetrazol-5-yl)-piperidin-1-yl)-3,5-difluorophenyl]-2-oxo-oxazolidin-5-ylmethyl}-acetamide;
16. (S)—N-{3-[4-(4-(1H-tetrazol-1-yl)-piperidin-1-yl)-3-fluorophenyl]-2-oxo-oxazolidin-5-ylmethyl}-acetamide;
17. (S)—N-{3-[4-(4-(2H-tetrazol-2-yl)-piperidin-1-yl)-3-fluorophenyl]-2-oxo-oxazolidin-5-ylmethyl}-acetamide;
8. (S)—N-{3-[4-(4-(1H-tetrazol-1-yl)-piperidin-1-yl)-3,5-difluorophenyl]-2-oxo-oxazolidin-5-ylmethyl}-acetamide;
19. (S)—N-{3-[4-(4-(2H-tetrazol-2-yl)-piperidin-1-yl)-3,5-difluorophenyl]-2-oxo-oxazolidin-5-ylmethyl}-acetamide;
20. (S)—N-{3-[4-(4-(1H-tetrazol-1-yl)-3-hydroxypiperidin-1-yl)-3-fluorophenyl]-2-oxo-oxazolidin-5-ylmethyl}-acetamide and isomer thereof;
21. (S)—N-{3-[4-(4-(1H-tetrazol-1-yl)-3-hydroxypiperidin-1-yl)-3,5-difluorophenyl]-2-oxo-oxazolidin-5-ylmethyl}-acetamide;
22. (S)—N-{3-[4-(4-(1H-tetrazol-1-yl)-3-fluoropiperidin-1-yl)-3,5-difluorophenyl]-2-oxo-oxazolidin-5-ylmethyl}-acetamide;
23. (S)—N-{3-[4-(4-(4-formyl-1H-[1,2,3]-triazol-1-yl)-piperidin-1-yl)-3-fluorophenyl]-2-oxo-oxazolidin-5-ylmethyl}-acetamide;
24. (S)—N-{3-[4-(4-(4-formyl-1H-[1,2,3]-triazol-1-yl)-piperidin-1-yl)-3,5-difluorophenyl]-2-oxo-oxazolidin-5-ylmethyl}-acetamide;
25. (S)—N-{3-[4-(4-(4-formyl-1H-[1,2,3]-triazol-1-yl)-3-fluoropiperidin-1-yl)-3-fluorophenyl]-2-oxo-oxazolidin-5-ylmethyl}-acetamide;
26. (R)-{3-[4-(4-(4-formyl-1H-[1,2,3]-triazol-1-yl)-piperidin-1-yl)-3-fluorophenyl]-5-(1H-[1,2,3]-triazol-1-ylmethyl)}-oxazolidin-2-one;
27. (S)—N-{3-[4-(4-(4-difluoromethyl-1H-[1,2,3]-triazol-1-yl)-piperidin-1-yl)-3-fluorophenyl]-2-oxo-oxazolidin-5-ylmethyl}-acetamide;
28. (S)—N-{3-[4-(4-(4-difluoromethyl-1H-[1,2,3]-triazol-1-yl)-piperidin-1-yl)-3,5-difluorophenyl]-2-oxo-oxazolidin-5-ylmethyl}-acetamide;
29. (S)—N-{3-[4-(4-(4-[1,3]-Dioxolan-2-yl-1H-[1,2,3]-triazol-1-yl)-piperidin-1-yl)-3-fluorophenyl]-5-(1H-[1,2,3]-triazol-1-ylmethyl)}-oxazolidin-2-one;
30. (S)—N-{3-[4-(4-(4-ethoxycarbonyl-1H-[1,2,3]-triazol-1-yl)-methyl-piperidin-1-yl)-3-fluoro-phenyl]-2-oxo-oxazolidin-5-ylmethyl}-acetamide;
31. (S)—N-{3-[4-(4-(5-ethoxycarbonyl-1H-[1,2,3]-triazol-1-yl)-methyl-piperidin-1-yl)-3-fluoro-phenyl]-2-oxo-oxazolidin-5-ylmethyl}-acetamide;
32. (S)—N-{3-[4-(4-(4-ethoxycarbonyl-1H-[1,2,3]-triazol-1-yl)-3-fluoropiperidin-1-yl)-3-fluorophenyl]-2-oxo-oxazolidin-5-ylmethyl}-acetamide;
33. (S)—N-{3-[4-(4-(4-ethoxycarbonyl-1H-[1,2,3]-triazol-1-yl)-piperidin-1-yl)-3,5-difluorophenyl]-2-oxo-oxazolidin-5-ylmethyl}-acetamide;
34. (R)-{3-[4-(4-(4-ethoxycarbonyl-1H-[1,2,3]-triazol-1-yl)-piperidin-1-yl)-3-fluorophenyl]-5-(1H-[1,2,3]-triazol-1-ylmethyl)}-oxazolidin-2-one;
35. (R)-{3-[4-(4-(4-ethoxycarbonyl-1H-[1,2,3]-triazol-1-yl)-piperidin-1-yl)-3,5-difluorophenyl]-5-(1H-[1,2,3]-triazol-1-ylmethyl)}-oxazolidin-2-one;
36. (S)—N-{3-[4-(4-(4-hydroxymethyl-1H-[1,2,3]-triazol-1-yl)-piperidin-1-yl)-3-fluorophenyl]-2-oxo-oxazolidin-5-ylmethyl}-acetamide;
37. (S)—N-{3-[4-(4-(5-hydroxymethyl-1H-[1,2,3]-triazol-1-yl)-piperidin-1-yl)-3-fluorophenyl]-2-oxo-oxazolidin-5-ylmethyl}-acetamide;
38. (S)—N-{3-[4-(4-(4-hydroxymethyl-1H-[1,2,3]-triazol-1-yl)-3-fluoropiperidin-1-yl)-3-fluorophenyl]-2-oxo-oxazolidin-5-ylmethyl}-acetamide;
39. (S)—N-{3-[4-(4-(4-hydroxymethyl-1H-[1,2,3]-triazol-1-yl)-piperidin-1-yl)-3,5-difluorophenyl]-2-oxo-oxazolidin-5-ylmethyl}-acetamide;
40. (R)-{3-[4-(4-(4-hydroxymethyl-1H-[1,2,3]-triazol-1-yl)-piperidin-1-yl)-3-fluorophenyl]-5-(1H-[1,2,3]-triazol-1-ylmethyl)}-oxazolidin-2-one;
41. (R)-{3-[4-(4-(4-hydroxymethyl-1H-[1,2,3]-triazol-1-yl)-piperidin-1-yl)-3,5-difluorophenyl]-5-(1H-[1,2,3]-triazol-1-ylmethyl)}-oxazolidin-2-one;
42. (S)—N-{3-[4-(4-((4-hydroxymethyl)-[1,2,3]-triazol-1-yl-methyl)-piperidin-1-yl)-3-fluoro-phenyl]-2-oxo-oxazolidin-5-ylmethyl}-acetamide;
43. (S)—N-{3-[4-(4-(4-cyano-1H-[1,2,3]-triazol-1-yl)-piperidin-1-yl)-3-fluorophenyl]-2-oxo-oxazolidin-5-ylmethyl}-acetamide;
44. (S)—N-{3-[4-(4-(4-carboxamido-1H-[1,2,3]-triazol-1-yl)-piperidin-1-yl)-3-fluorophenyl]-2-oxo-oxazolidin-5-ylmethyl}-acetamide;
45. (R)-{3-[4-(4-(4-carboxamido-1H-[1,2,3]-triazol-1-yl)-piperidin-1-yl)-3-fluorophenyl]-5-(1H-[1,2,3]-triazol-1-ylmethyl)}-oxazolidin-2-one;
46. (S)—N-{3-[4-(4-(4-carboxamido-1H-[1,2,3]-triazol-1-yl)-methyl-piperidin-1-yl)-3-fluoro-phenyl]-2-oxo-oxazolidin-5-ylmethyl}-acetamide;
47. (S)—N-{3-[4-(4-(5-carboxamido-1H-[1,2,3],-triazol-1-yl)-methyl-piperidin-1-yl)-3-fluoro-phenyl]-2-oxo-oxazolidin-5-ylmethyl}-acetamide;
48. (S)—N-{3-[4-(4-(4-hydroxycarbonyl-1H-[1,2,3]-triazol-1-yl)-methyl-piperidin-1-yl)-3-fluoro-phenyl]-2-oxo-oxazolidin-5-ylmethyl}-acetamide;
49. (S)—N-{3-[4-(4-(5-hydroxycarbonyl-1H-[1,2,3]-triazol-1-yl)-methyl-piperidin-1-yl)-3-fluoro-phenyl]-2-oxo-oxazolidin-5-ylmethyl}-acetamide;
50. (S)—N-{3-[4-(4-(4-acetyl-1H-[1,2,3]-triazol-1-yl)-piperidin-1-yl)-3-fluorophenyl]-2-oxo-oxazolidin-5-ylmethyl}-acetamide;
51. (S)—N-{3-[4-(4-(4-acetyl-1H-[1,2,3]-triazol-1-yl)-piperidin-1-yl)-3,5-difluorophenyl]-2-oxo-oxazolidin-5-ylmethyl}-acetamide;
52. (R)-{3-[4-(4-(4-acetyl-1H-[1,2,3]-triazol-1-yl)-piperidin-1-yl)-3-fluorophenyl]-5-(1H-[1,2,3]-triazol-1-ylmethyl)}-oxazolidin-2-one;
53. (S)—N-{3-[4-(4-(4-acetyl-1H-[1,2,3]-trizol-1-ylmethyl)-1H-[1,2,3]-triazol-1-yl)-piperidin-1-yl)-3-fluorophenyl]-2-oxo-oxazolidin-5-ylmethyl}-acetamide;
54. (S)—N-{3-[4-(4-(4-(1-hydroxyethyl)-1H-[1,2,3]-triazol-1-yl)-piperidin-1-yl)-3-fluorophenyl]-2-oxo-oxazolidin-5-ylmethyl}-acetamide;
55. (S)—N-{3-[4-(4-(4-(1-hydroxyethyl)-1H-[1,2,3]-triazol-1-yl)-piperidin-1-yl)-3,5-difluorophenyl]-2-oxo-oxazolidin-5-ylmethyl}-acetamide;
56. (R)-{3-[4-(4-(4-(2-hydroxyethyl)-1H-[1,2,3]-triazol-1-yl)-piperidin-1-yl)-3-fluorophenyl]-5-(1H-[1,2,3]-triazol-1-ylmethyl)}-oxazolidin-2-one;
57. (S)—N-{3-[4-(4-(4-(2-methyl-oxiranyl)-1H-[1,2,3]-triazol-1-yl)-piperidin-1-yl)-3-fluorophenyl]-2-oxo-oxazolidin-5-ylmethyl}-acetamide;
58. (R)-{3-[4-(4-(4-(2-methyl-oxiranyl)-1H-[1,2,3]-triazol-1-yl)-piperidin-1-yl)-3-fluorophenyl]-5-(1H-[1,2,3]-triazol-1-ylmethyl)}-oxazolidin-2-one;
59. (S)—N-{3-[4-(4-((4-((E/Z)-2-cyano-vinyl)-1H-[1,2,3]-triazol-1-yl)-piperidin-1-yl)-3-fluorophenyl]-2-oxo-oxazolidin-5-ylmethyl}-acetamide;
60. (S)—N-{3-[4-(4-(4-hydroxyimino-methyl)-1H-[1,2,3]-triazol-1-yl)-piperidin-1-yl)-3-fluorophenyl]-2-oxo-oxazolidin-5-ylmethyl}-acetamide;
61. (S)—N-{3-[4-(4-(1-methoxyimino-ethyl)-1H-[1,2,3]-triazol-1-yl)-piperidin-1-yl)-3,5-difluorophenyl]-2-oxo-oxazolidin-5-ylmethyl}-acetamide;
62. (S)—N-{3-[4-(4-(4-(1-methoxyimino-ethyl)-1H-[1,2,3]-triazol-1-yl)-piperidin-1-yl)-3-fluorophenyl]-2-oxo-oxazolidin-5-ylmethyl}-acetamide;
63. (R)-{3-[4-(4-(4-(1-methoxyimino-ethyl)-[1,2,3]-triazol-1-yl)-piperidin-1-yl)-3-fluorophenyl]-5-(1H-[1,2,3]-triazol-1-ylmethyl)}-oxazolidin-2-one;
64. (R)-{3-[4-(4-(4-(1-methoxyimino-ethyl)-[1,2,3]-triazol-1-yl)-piperidin-1-yl)-3,5-difluorophenyl]-5-(1H-[1,2,3]-triazol-1-ylmethyl)}-oxazolidin-2-one;
65. (S)—N-{3-[4-(4-(1-methoxyimino-methyl)-1H-[1,2,3]-triazol-1-yl)-piperidin-1-yl)-3-fluorophenyl]-2-oxo-oxazolidin-5-ylmethyl}-acetamide;
66. (S)—N-{3-[4-(4-(1-methoxyimino-methyl)-1H-[1,2,3]-triazol-1-yl)-piperidin-1-yl)-3,5-difluorophenyl]-2-oxo-oxazolidin-5-ylmethyl}-acetamide;
67. (S)—N-{3-[4-(4-(1-benzyloxyimino-methyl)-1H-[1,2,3]-triazol-1-yl)-piperidin-1-yl)-3-fluorophenyl]-2-oxo-oxazolidin-5-ylmethyl}-acetamide;
68. (S)—N-{3-[4-(4-(4-hydroxyiminoamino-methyl)-[1,2,3]-triazol-1-yl)-piperidin-1-yl)-3-fluorophenyl]-2-oxo-oxazolidin-5-ylmethyl}-acetamide;
69. (S)—N-{3-[4-(4-(4-N,N-dimethylaminoethylaminomethyl)-1H-[1,2,3]-triazol-1-yl)-piperidin-1-yl)-3-fluorophenyl]-2-oxo-oxazolidin-5-ylmethyl}-acetamide;
70. (S)—N-{3-[4-(4-(2H-benzotriazol-2-yl)-piperidin-1-yl)-3,5-difluorophenyl]-2-oxo-oxazolidin-5-ylmethyl}-acetamide;
71. (S)—N-{3-[4-(4-(1H-benzotriazol-1-yl)-piperidin-1-yl)-3,5-difluorophenyl]-2-oxo-oxazolidin-5-ylmethyl}-acetamide;
72. (S)—N-{3-[4-(4-Benzotriazol-2-yl-piperidin-1-yl)-3-fluorophenyl]-2-oxo-oxazolidin-5-ylmethyl}-acetamide;
73. (S)—N-{3-[4-(4-Benzotriazol-1-yl-piperidin-1-yl)-3-fluorophenyl]-2-oxo-oxazolidin-5-ylmethyl}-acetamide;
74. (S)—N-{3-[4-(4-(4-(1H-tetrazol-1-ylmethyl)-1H-[1,2,3]-triazol-1-yl)-piperidin-1-yl)-3-fluorophenyl]-2-oxo-oxazolidin-5-ylmethyl}-acetamide;
75. (S)—N-{3-[4-(4-(2H-tetrazol-2-ylmethyl)-[1,2,3]-triazol-1-yl)-piperidin-1-yl)-3-fluorophenyl]-2-oxo-oxazolidin-5-ylmethyl}-acetamide;
76. (S)—N-{3-[4-(4-(1H-imidazol-1-ylmethyl)-[1,2,3]-triazol-1-yl)-piperidin-1-yl)-3-fluorophenyl]-2-oxo-oxazolidin-5-ylmethyl}-acetamide;
77. (R)-{3-[4-(4-(1H-[1,2,3]-triazol-1-yl)-3-fluoropiperidin-1-yl)-3-fluorophenyl]-5-(1H-[1,2,3]-triazol-1-ylmethyl)}-oxazolidin-2-one;
78. (R)-{3-[4-(4-(1H-[1,2,3]-triazol-1-yl)-piperidin-1-yl)-3-fluorophenyl]-5-(1H-[1,2,3]-triazol-1-ylmethyl)}-oxazolidin-2-one;
79. (R)-{3-[4-(4-(1H-[1,2,3]-triazol-1-yl)-piperidin-1-yl)-3,5-difluorophenyl]-5-(1H-[1,2,3]-triazol-1-ylmethyl)}-oxazolidin-2-one;
80. (R)-{3-[4-(4-(1H-[1,2,3]-triazol-1-yl)-3-fluoropiperidin-1-yl)-3,5-difluorophenyl]-5-(1H-[1,2,3]-triazol-1-ylmethyl)}-oxazolidin-2-one;
81. (S)—N-{3-[4-(4-([1,2,3]-triazol-1-yl)-piperidin-1-yl)-3-fluorophenyl]-2-oxo-oxazolidin-5-ylmethyl}-thioacetamide;
82. (S)—N-{3-[4-(4-(5-methyl-2H-tetrazol-2-yl)-piperidin-1-yl)-3-fluorophenyl]-2-oxo-oxazolidin-5-ylmethyl}-acetamide;
83. (S)—N-{3-[4-(4-(5-methyl-1H-tetrazol-1-yl)-piperidin-1-yl)-3,5-difluorophenyl]-2-oxo-oxazolidin-5-ylmethyl}-acetamide;
84. (S)—N-{3-[4-(4-(5-methyl-1H-tetrazol-1-yl)-piperidin-1-yl)-3,5-difluorophenyl]-2-oxo-oxazolidin-5-ylmethyl}-acetamide;
85. (S)—N-{3-[4-(4-(5-methyl-2H-tetrazol-2-yl)-piperidin-1-yl)-3,5-difluorophenyl]-2-oxo-oxazolidin-5-ylmethyl}-acetamide;
86. (S)—N-{3-[4-(4-(2-methyl-2H-tetrazol-5-yl)-piperidin-1-yl)-3-fluorophenyl]-2-oxo-oxazolidin-5-ylmethyl}-acetamide;
87. (S)—N-{3-[4-(4-(1-methyl-1H-tetrazol-5-yl)-piperidin-1-yl)-3-fluorophenyl]-2-oxo-oxazolidin-5-ylmethyl}-acetamide;
88. (S)—N-{3-[4-(4-(2-methyl-2H-tetrazol-5-yl)-piperidin-1-yl)-3,5-difluorophenyl]-2-oxo-oxazolidin-5-ylmethyl}-acetamide;
89. (S)—N-{3-[4-(4-(1-methyl-1H-tetrazol-5-yl)-piperidin-1-yl)-3,5-difluorophenyl]-2-oxo-oxazolidin-5-ylmethyl}-acetamide;
90. (R)-{3-[4-(4-(2-methyl-2H-tetrazol-5-yl)-piperidin-1-yl)-3-fluorophenyl]-5-(1H-[1,2,3]-triazol-lylmethyl)}-oxazolidin-2-one;
91. (R)-{3-[4-(4-(1-methyl-1H-tetrazol-5-yl)-piperidin-1-yl)-3-fluorophenyl]-5-(1H-[1,2,3]-triazol-1-ylmethyl)})-oxazolidin-2-one;
92. (S)—N-{3-[4-(4-(1-methyl)-1H-tetrazol-5-ylmethyl-piperidin-1-yl)-3,5-difluoro-phenyl]-2-oxo-oxazolidin-5-ylmethyl}-acetamide;
93. (S)—N-{3-[4-(4-(2-methyl)-2H-tetrazol-5-ylmethyl-piperidin-1-yl)-3,5-difluoro-phenyl]-2-oxo-oxazolidin-5-ylmethyl}-acetamide;
94. (S)—N-{3-[4-(4-(1-methyl-1H-tetrazol-5-ylmethyl)-piperidin-1-yl)-3-fluoro-phenyl]-2-oxo-oxazolidin-5-ylmethyl}-acetamide;
95. (S)—N-{3-[4-(4-(2-methyl-2H-tetrazol-5-ylmethyl)-piperidin-1-yl)-3-fluoro-phenyl]-2-oxo-oxazolidin-5-ylmethyl}-acetamide;
96. (S)—N-{3-[4-(4-(5-methylsulfanylmethyl-2H-tetrazol-2-yl)-piperidin-1-yl)-3-fluorophenyl]-2-oxo-oxazolidin-5-ylmethyl}-acetamide;
97. (S)—N-{3-[4-(4-(5-methylsulfanylmethyl-1H-tetrazol-1-yl)-piperidin-1-yl)-3-fluorophenyl]-2-oxo-oxazolidin-5-ylmethyl}-acetamide;
98. (S)—N-{3-[4-(4-(5-methylsulfanylmethyl-2H-tetrazol-2-yl)-piperidin-1-yl)-3,5-difluorophenyl]-2-oxo-oxazolidin-5-ylmethyl}-acetamide;
99. (S)—N-{3-[4-(4-(5-methylsulfanylmethyl-1H-tetrazol-1-yl)-piperidin-1-yl)-3,5-fluorophenyl]-2-oxo-oxazolidin-5-ylmethyl}-acetamide;
100. (S)—N-{3-[4-(4-(5-benzylsulfanyl-1H-tetrazol-1-yl)-piperidin-1-yl)-3,5-difluorophenyl]-2-oxo-oxazolidin-5-ylmethyl}-acetamide;
101. (S)—N-{3-[4-(4-(5-(benzylsulfanyl)-2H-tetrazol-2-yl)-piperidin-1-yl)-3,5-difluorophenyl]-2-oxo-oxazolidin-5-ylmethyl}-acetamide;
102. (S)—N-{3-[4-(4-(5-(benzylsulfanyl)-2H-tetrazol-2-yl)-piperidin-1-yl)-3-fluorophenyl]-2-oxo-oxazolidin-5-ylmethyl}-acetamide;
103. (S)—N-{3-[4-(4-(5-(benzylsulfanyl)-2H-tetrazol-2-yl)-piperidin-1-yl)-3-fluorophenyl]-2-oxo-oxazolidin-5-ylmethyl}-acetamide;
104. (S)—N-{3-[4-(4-(5-methylsulfonylmethyl-1H-tetrazol-1-yl)-piperidin-1-yl)-3,5-difluorophenyl]-2-oxo-oxazolidin-5-ylmethyl}-acetamide;
105. (S)—N-{3-[4-(4-(5-methylsulfonylmethyl-1H-tetrazol-1-yl)-piperidin-1-yl)-3-fluorophenyl]-2-oxo-oxazolidin-5-ylmethyl}-acetamide;
106. (S)—N-{3-[4-(4-(5-(thiophen-2-yl-methyl)-1H-tetrazol-1-yl)-piperidin-1-yl)-3-fluorophenyl]-2-oxo-oxazolidin-5-ylmethyl}-acetamide;
107. (S)—N-{3-[4-(4-(5-(thiophen-2-yl-methyl)-1H-tetrazol-1-yl)-piperidin-1-yl)-3-fluorophenyl]-2-oxo-oxazolidin-5-ylmethyl}-acetamide;
108. (S)—N-{3-[4-(4-(5-(thiophen-2-yl-methyl)-1H-tetrazol-1-yl)-piperidin-1-yl)-3,5-difluorophenyl]-2-oxo-oxazolidin-5-ylmethyl}-acetamide;
109. (S)—N-{3-[4-(4-(5-(thiophen-2-yl-methyl)-2H-tetrazol-2-yl)-piperidin-1-yl)-3,5-difluorophenyl]-2-oxo-oxazolidin-5-ylmethyl}-acetamide;
110. (S)—N-{3-[4-(4-(5-(morpholinocarbonylmethyl)-1H-tetrazol-1-yl)-piperidin-1-yl)-3-fluorophenyl]-2-oxo-oxazolidin-5-ylmethyl}-acetamide;
111. (S)—N-{3-[4-(4-(5-(morpholinocarbonylmethyl)-1H-tetrazol-1-yl)-piperidin-1-yl)-3,5-difluorophenyl]-2-oxo-oxazolidin-5-ylmethyl}-acetamide;
112. (S)—N-{3-[4-(4-(5-(morpholin-4-yl)-2H-tetrazol-2-yl)-piperidin-1-yl)-3,5-difluorophenyl]-2-oxo-oxazolidin-5-ylmethyl}-acetamide;
113. (S)—N-{3-[4-(4-(5-(morpholin-4-yl)-2H-tetrazol-2-yl)-piperidin-1-yl)-3-fluorophenyl]-2-oxo-oxazolidin-5-ylmethyl}-acetamide;
114. (S)—N-{3-[4-(4-(5-phenyl-2H-tetrazol-2-yl)-piperidin-1-yl)-3,5-difluorophenyl]-2-oxo-oxazolidin-5-ylmethyl}-acetamide;
115. (S)—N-{3-[4-(4-(5-phenyl-2H-tetrazol-2-yl)-piperidin-1-yl)-3-fluorophenyl]-2-oxo-oxazolidin-5-ylmethyl}-acetamide;
116. (S)—N-{3-[4-(4-(5-(4-nitrophenyl)-2H-tetrazol-2-yl)-piperidin-1-yl)-3-fluorophenyl]-2-oxo-oxazolidin-5-ylmethyl}-acetamide;
117. (S)—N-{3-[4-(4-(5-(4-nitrophenyl)-2H-tetrazol-2-yl)-piperidin-1-yl)-3,5-difluorophenyl]-2-oxo-oxazolidin-5-ylmethyl}-acetamide;
118. (S)—N-{3-[4-(4-(5-(4-pyridinyl)-1H-tetrazol-1-yl)-piperidin-1-yl)-3-fluorophenyl]-2-oxo-oxazolidin-5-ylmethyl}-acetamide;
119. (S)—N-{3-[4-(4 (5-(3-pyridinyl)-2H-tetrazol-2-yl)-piperidin-1-yl)-3-fluorophenyl]-2-oxo-oxazolidin-5-ylmethyl}-acetamide;
120. (S)—N-{3-[4-(4-(5-(4-pyridinyl)-1H-tetrazol-1-yl)-piperidin-1-yl)-3,5-difluorophenyl]-2-oxo-oxazolidin-5-ylmethyl}1-acetamide;
121. (S)—N-{3-[4-(4-(5-(3-pyridinyl)-2H-tetrazol-2-yl)-piperidin-1-yl)-3,5-difluorophenyl]-2-oxo-oxazolidin-5-ylmethyl}-acetamide;
122. (S)—N-{3-[4-(4-(5-amino-2H-tetrazol-2-yl)-piperidin-1-yl)-3-fluorophenyl]-2-oxo-oxazolidin-5-ylmethyl}-acetamide;
123. (S)—N-{3-[4-(4-(5-amino-2H-tetrazol-1-yl)-piperidin-1-yl)-3,5-difluorophenyl]-2-oxo-oxazolidin-5-ylmethyl}-acetamide;
124. (S)—N-{3-[4-(4-(5-(diethylamino)-2H-tetrazol-2-yl)-piperidin-1-yl)-3,5-difluorophenyl]-2-oxo-oxazolidin-5-ylmethyl}-acetamide;
125. (S)—N-{3-[4-(4-(5-(piperazin-1-yl)-2H-[1,2,3,4],-tetrazol-2-yl)-piperidin-1-yl)-3-fluorophenyl]-2-oxo-oxazolidin-5-ylmethyl}-acetamide;
126. (S)—N-{3-[4-(4-(5-(piperazin-1-yl)-2H-tetrazol-2-yl)-piperidin-1-yl)-3,5-difluorophenyl]-2-oxo-oxazolidin-5-ylmethyl}-acetamide;
127. (S)—N-{3-[4-(4-(5-(4-methylpiperazin-1-yl)-2H-tetrazol-2-yl)-piperidin-1-yl)-3-fluorophenyl]-2-oxo-oxazolidin-5-ylmethyl}-acetamide;
128. (S)—N-{3-[4-(4-(5-(4-methylpiperazin-1-yl)-2H-tetrazol-2-yl)-piperidin-1-yl)-3,5-difluorophenyl]-2-oxo-oxazolidin-5-ylmethyl}-acetamide;
129. (S)—N-{3-[4-(4-(5-(4-acetyl-piperazin-1-yl)-2H-tetrazol-2-yl)-piperidin-1-yl)-3-fluorophenyl]-2-oxo-oxazolidin-5-ylmethyl}-acetamide;
130. (S)—N-{3-[4-(4-(5-(4-acetyl-piperazin-1-yl)-2H-tetrazol-2-yl)-piperidin-1-yl)-3,5-difluorophenyl]-2-oxo-oxazolidin-5-ylmethyl}-acetamide;
131. (S)—N-{3-[4-(4-(5-(4-cyanopiperazin-1-yl)-2H-tetrazol-2-yl)-piperidin-1-yl)-3-fluorophenyl]-2-oxo-oxazolidin-5-ylmethyl}-acetamide;
132. (S)—N-{3-[4-(4-(5-(4-cyanopiperazin-1-yl)-2H-tetrazol-2-yl)-piperidin-1-yl)-3,5-difluorophenyl]-2-oxo-oxazolidin-5-ylmethyl}-acetamide;
133. (S)—N-{3-[4-(4-(5-(4-benzylpiperazin-1-yl)-2H-tetrazol-2-yl)-piperidin-1-yl)-3-fluorophenyl]-2-oxo-oxazolidin-5-ylmethyl}-acetamide;
134. (S)—N-{3-[4-(4-(5-(4-benzylpiperazin-1-yl)-2H-tetrazol-2-yl)-piperidin-1-yl)-3,5-difluorophenyl]-2-oxo-oxazolidin-5-ylmethyl}-acetamide;
135. (S)—N-{3-[4-(4-1H-tetrazol-5-ylmethyl-piperidin-1-yl)-3-fluoro-phenyl]-2-oxo-oxazolidin-5-ylmethyl}-acetamide;
136. (S)—N-{3-[4-(4-1H-tetrazol-5-ylmethyl-piperidin-1-yl)-3,5-difluorophenyl]-2-oxo-oxazolidin-5-ylmethyl}-acetamide;
137. (S)—N-{3-[4-(4-([1,2,3]-triazol-1-yl-methyl)-piperidin-1-yl)-3-fluoro-phenyl]-2-oxo-oxazolidin-5-ylmethyl}-acetamide;
138. (R)-{3-[4-(4-(1H-tetrazol-1-yl)-piperidin-1-yl)-3-fluorophenyl]-5-(1H-[1,2,3]-triazol-1-ylmethyl)}-oxazolidin-2-one;
139. (R)-{3-[4-(4-(2H-tetrazol-2-yl)-piperidin-1-yl)-3-fluorophenyl]-5-(1H-[1,2,3]-triazol-1-ylmethyl)}-oxazolidin-2-one;
140. (S)-{3-[4-(4-(tetrazol-5-yl)-piperidin-1-yl)-3-fluorophenyl]-5-(1H-[1,2,3]-triazol-1-ylmethyl)}-oxazolidin-2-one;
141. (S)—N-{3-[4-(4-(5-methyl-[1,2,4]-oxadiazol-3-yl)-piperidin-1-yl)-3-fluorophenyl]-2-oxo-oxazolidin-5-ylmethyl}-acetamide;
142. (S)—N-{3-[4-(4-(5-methyl-[1,2,4]-oxadiazol-3-ylmethyl)-piperidin-1-yl)-3-fluoro-phenyl]-2-oxo-oxazolidin-5-ylmethyl}-acetamide;
143. (S)—N-{3-[4-(4-(5-methyl-[1,2,4]-oxadiazol-3-ylmethyl)-piperidin-1-yl)-3,5-difluoro-phenyl]-2-oxo-oxazolidin-5-ylmethyl}-acetamide;
144. (S)—N-{3-[4-(4-(5-phenyl-[1,2,4]-oxadiazol-3-ylmethyl)-piperidin-1-yl)-3-fluoro-phenyl]-2-oxo-oxazolidin-5-ylmethyl}-acetamide;
145. (S)—N-{3-[4-(4-(5-phenyl-[1,2,4]-oxadiazol-3-ylmethyl)-piperidin-1-yl)-3,5-difluoro-phenyl]-2-oxo-oxazolidin-5-ylmethyl}-acetamide;
146. (S)—N-{3-[4-(4-((5-pyridin-3-yl)-[1,2,4]-oxadiazol-3-ylmethyl)-piperidin-1-yl)-3-fluoro-phenyl]-2-oxo-oxazolidin-5-ylmethyl}-acetamide;
147. (S)—N-{3-[4-(4-(5-methyl-[1,3,4]-oxadiazol-2-ylmethyl)-piperidin-1-yl)-3-fluoro-phenyl]-2-oxo-oxazolidin-5-ylmethyl}-acetamide;
148. (S)—N-{3-[4-(4-(5-phenyl-[1,3,4]-oxadiazol-2-ylmethyl)-piperidin-1-yl)-3-fluoro-phenyl]-2-oxo-oxazolidin-5-ylmethyl}-acetamide;
149. (S)—N-{3-[4-(4-(5-Amino-2H-[1,3,4]-thiadiazol-2-yl)-piperidin-1-yl)-3-fluorophenyl]-2-oxo-oxazolidin-5-ylmethyl}-acetamide;
150. (S)—N-{3-[4-(4-(5-Amino-[1,3,4]-thiadiazol-2-yl)-piperidin-1-yl)-3,5-difluoro-phenyl]-2-oxo-oxazolidin-5-ylmethyl}-acetamide;
151. (S)—N-{3-[4-(4-(5-Amino-[1,3,4]-thiadiazol-2-ylmethyl)-piperidin-1-yl)-3,5-difluoro-phenyl]-2-oxo-oxazolidin-5-ylmethyl}-acetamide;
152. (S)—N-{3-[4-(4-(3-ethoxycarbonyl-imidazol-1-yl)-methyl-piperidin-1-yl)-3-fluoro-phenyl]-2-oxo-oxazolidin-5-ylmethyl}-acetamide;
153. (S)—N-{3-[3,5-Difluoro-4-(4-2H-tetrazol-2-yl-piperidin-1-yl)-phenyl]-2-oxo-oxazolidin-5-ylmethyl}-carbamic acid methyl ester;
154. (S)—N-{3-[3,5-Difluoro-4-(4-1H-tetrazol-1-yl-piperidin-1-yl)-phenyl]-2-oxo-oxazolidin-5-ylmethyl}-carbamic acid methyl ester;
155. (S)—N-{3-[3,5-Difluoro-4-(4-[1,2,3]-triazol-1-yl-piperidin-1-yl)-phenyl]-2-oxo-oxazolidin-5-ylmethyl}-carbamic acid methyl ester;
156. (S)—N-{3-[3-fluoro-4-(4-2H-tetrazol-2-yl-piperidin-1-yl)-phenyl]-2-oxo-oxazolidin-5-ylmethyl}-carbamic acid methyl ester;
157. (S)—N-{3-[3-fluoro-4-(4-1H-tetrazol-1-yl-piperidin-1-yl)-phenyl]-2-oxo-oxazolidin-5-ylmethyl}carbamic acid methyl ester;
158. (S)—N-{3-[4-(4-2H-[1,2,3]-triazol-2-yl-piperidin-1-yl)-3,5-difluoro phenyl]-2-oxo-oxazolidin-5-ylmethyl}-carbamic acid methyl ester;
159. (S)—N-{3-[3,5-Difluoro-4-(4-[1,2,3]triazol-2-yl-piperidin-1-yl)-phenyl]-2-oxo-oxazolidin-5-ylmethyl}-2,2-difluoro-acetamide;
160. (S)—N-{3-[3,5-Difluoro-4-(4-[1,2,3]-triazol-1-yl-piperidin-1-yl)-phenyl]-2-oxo-oxazolidin-5-ylmethyl}-2,2-difluoro-acetamide;
161. (S)—N-{3-[3,5-Difluoro-4-(4-tetrazol-2-yl-piperidin-1-yl)-phenyl]-2-oxo-oxazolidin-5-ylmethyl}-2,2-difluoro-acetamide;
162. (S)—N-{3-[3,5-Difluoro-4-(4-[1,2,3,4]-triazol-1-yl-piperidin-1-yl)-phenyl]-2-oxo-oxazolidin-5-ylmethyl}-2,2-difluoro-acetamide;
163. (S)—N-{3-[3,5-Difluoro-4-(4-2H-tetrazole-2-yl-piperidin-1-yl)-phenyl]-2-oxo-oxazolidin-5-ylmethyl}-2,2-dichloro-acetamide;
164. (S)—N-{3-[3-fluoro-4-(4-2H-tetrazol-2-yl-piperidin-1-yl)-phenyl]-2-oxo-oxazolidin-5-ylmethyl}-carbamic acid ethyl ester;
165. (S)—N-{3-[3,5-Difluoro-4-(4-1H-tetrazole-1-yl-piperidin-1-yl)-phenyl]-2-oxo-oxazolidin-5-ylmethyl}-2,2-difluoro-thioacetamide;
166. (S)—N-{3-[3,5-Difluoro-4-(4-1H-tetrazole-1-yl-piperidin-1-yl)-phenyl]-2-oxo-oxazolidin-5-ylmethyl}-cyclopropane carboxamide;
167. (S)—N-{3-[3,5-Difluoro-4-(4-[1,2,3]-triazol-1-yl-piperidin-1-yl)-phenyl]-2-oxo-oxazolidin-5-ylmethyl}-propionamide;
168. (S)—N-{3-[3,5-Difluoro-4-(4-[1,2,3]-triazol-2-yl-piperidin-1-yl)-phenyl]-2-oxo-oxazolidin-5-ylmethyl}-propionamide;
169. (S)—N-{3-[3,5-Difluoro-4-(4-2H-tetrazol-2-yl-piperidin-1-yl)-phenyl]-2-oxo-oxazolidin-5-ylmethyl}-propionamide;
170. (S)—N-{3-[3,5-Difluoro-4-(4-1H-tetrazol-1-yl-piperidin-1-yl)-phenyl]-2-oxo-oxazolidin-5-ylmethyl}-propionamide;
171. (S) N-{3-[3-Fluoro-4-(4-2H-tetrazol-2-yl-piperidin-1-yl)-phenyl]-2-oxo-oxazolidin-5-ylmethyl}-propionamide;
172. (S) N-{3-[3-Fluoro-4-(4-1H-tetrazol-1-yl-piperidin-1-yl)-phenyl]-2-oxo-oxazolidin-5-ylmethyl}-propionamide;
173. (S)—N-{3-[3,5-Difluoro-4-(4-[1,2,3]triazol-2-yl-piperidin-1-yl)-phenyl]-2-oxo-oxazolidin-5-ylmethyl}-propionamide;
174. (S)—N-{3-[3,5-Difluoro-4-(4-[1,2,3]-triazol-1-yl-piperidin-1-yl)-phenyl]-2-oxo-oxazolidin-5-ylmethyl}-propionamide;
175. (S)—N-{3-[3,5-Difluoro-4-(4-(5-methyl-tetrazol-2-yl)-piperidin-1-yl)-phenyl]-2-oxo-oxazolidin-5-ylmethyl}-propionamide;
176. (S)—N-{3-[3,5-Difluoro-4-(4-(5-methyl-tetrazol-1-yl)-piperidin-1-yl)-phenyl]-2-oxo-oxazolidin-5-ylmethyl}-propionamide;
177. (S)—N-{3-[3,5-Difluoro-4-(4-(4-methyl-5-thioxo-4,5-dihydro-tetrazol-1-yl)-piperidin-1-yl)-phenyl]-2-oxo-oxazolidin-5-ylmethyl}-acetamide;
178. (S)—N-{3-[3,5-Difluoro-4-(4-(4-methyl-5-thioxo-4,5-dihydro-[1,2,4]triazol-1-yl)-piperidin-1-yl)-phenyl]-2-oxo-oxazolidin-5-ylmethyl}-acetamide;
179. (S)—N-{3-[3-fluoro-4-(4-(4-methyl-5-thioxo-4,5-dihydro-[1,2,4]triazol-1-yl)-piperidin-1-yl)-phenyl]-2-oxo-oxazolidin-5-ylmethyl}-acetamide;
180. (S)—N-{3-[4-(4-(isoxazol-3-yloxymethyl)-piperidin-1-yl)-3-fluorophenyl]-2-oxo-oxazolidin-5-ylmethyl}-acetamide;
181. (S)—N-{3-[4-(4-([1,2,3]-triazol-1-yl)-methyl-piperidin-1-yl)-3-fluoro-phenyl]-5-(isoxazol-3-yloxymethyl)}-oxazolidin-2-one;
182. (R)-{3-[4-(4-((4-hydroxymethyl)-[1,2,3]-triazol-1-yl)-methyl-piperidin-1-yl)-3-fluoro-phenyl]-5-(isoxazol-3-yloxymethyl)}-oxazolidin-2-one;
183. cis-(R)-{3-[4-(4-(5-ethoxycarbonyl-1H-[1,2,3]-triazol-1-yl)-piperidin-1-yl)-3-fluorophenyl]-5-(5-ethoxycarbonyl-1H-[1,2,3]-triazol-1-ylmethyl)}-oxazolidin-2-one;
184. cis-(R)-{3-[4-(4-(5-ethoxycarbonyl-1H-[1,2,3]-triazol-1-yl)-piperidin-1-yl)-3-fluoro-phenyl]-5-(4-ethoxycarbonyl-1H-[1,2,3]-triazol-1-ylmethyl)}-oxazolidin-2-one;
185. trans-(R)-{3-[4-(4-(5-ethoxycarbonyl-1H-[1,2,3]-triazol-1-yl)-piperidin-1-yl)-3-fluoro-phenyl]-5-(5-ethoxycarbonyl-1H-[1,2,3]-triazol-1-ylmethyl)}-oxazolidin-2-one;
186. trans-(R)-{3-[4-(4-(5-ethoxycarbonyl-1H-[1,2,3]-triazol-1-yl)-piperidin-1-yl)-3-fluoro-phenyl]-5-(4-ethoxycarbonyl-1H-[1,2,3]-triazol-1-ylmethyl)}-oxazolidin-2-one;
187. trans-(R)-{3-[4-(4-(5-hydroxymethyl-1H-[1,2,3]-triazol-1-yl)-piperidin-1-yl)-3-fluoro-phenyl]-5-(4-hydroxymethyl-1H-[1,2,3]-triazol-1-ylmethyl)}-oxazolidin-2-one;
188. trans-(R)-{3-[4-(4-(5-hydroxymethyl-1H-[1,2,3]-triazol-1-yl)-piperidin-1-yl)-3-fluoro-phenyl]-5-(5-hydroxymethyl-1H-[1,2,3]-triazol-1-ylmethyl)}-oxazolidin-2-one;
189. trans-(R)-{3-[4-(4-(5-cyano-1H-[1,2,3]-triazol-1-yl)-piperidin-1-yl)-3-fluoro-phenyl]-5-(5-cyano-1H-[1,2,3]-triazol-1-ylmethyl)}-oxazolidin-2-one;
190. trans-(R)-{3-[4-(4-(5-cyano-1H-[1,2,3]-triazol-1-yl)-piperidin-1-yl)-3-fluoro-phenyl]-5-(4-cyano-1H-[1,2,3]-triazol-1-ylmethyl)}-oxazolidin-2-one;
191. (3R,5S)—N-{3-[4-(Fluoro-4-(1H-[1,2,3]-triazol-1-yl)-piperidin-1-yl)-3-fluoro-phenyl]-2-oxo-oxazolidin-5-ylmethyl}-acetamide;
192. (R)-{3-[4-(4-(5-ethoxycarbonylmethyl-2H-tetrazol-2-yl)-piperidin-1-yl)-3,5-difluorophenyl]-5-(1H-[1,2,3]-triazol-1-ylmethyl)}-oxazolidin-2-one;
193. (S)—N-{3-[4-(4-(5-hydroxyethyl-2H-tetrazol-2-yl)-piperidin-1-yl)-3,5-difluorophenyl]-2-oxo-oxazolidin-5-ylmethyl}-acetamide;
194. (S)—N-{3-[4-(4-(5-acetylaminomethyl-2H-tetrazol-2-yl)-piperidin-1-yl)-3,5-difluorophenyl]-2-oxo-oxazolidin-5-ylmethyl}-acetamide;
195. (R)-{3-(4-phenyl-3-fluorophenyl)-5-1H-[1,2,3]-triazol-1-ylmethyl}-oxazolidin-2-one;
196. (R)-{3-(4-phenyl-3-fluorophenyl)-5-1H-[1,2,4]-triazol-1-ylmethyl}-oxazolidin-2-one;
197. (R)-{3-(4-phenyl-3-fluorophenyl)-5-1H-[1,2,3]-triazol-2-ylmethyl}-oxazolidin-2-one;
198. (R)-{3-[4-(4-(piperidin-1-yl)-3-fluorophenyl)-3-fluorophenyl]-2-oxo-oxazolidin-5-ylmethyl}-methanesulfonate;
199. (S)—N-{3-[4-(4-(piperidin-1-yl)-3-fluorophenyl)-3-fluorophenyl]-2-oxo-oxazolidin-5-ylmethyl}-acetamide;
200. (S)-{3-[4-(4-(piperidin-1-yl)-3-fluorophenyl)-3-fluorophenyl]-5-imidazol-1-ylmethyl}-oxazolidin-2-one;
201. (R)-{3-[4-(4-(piperidin-1-yl)-3-fluorophenyl)-3-fluorophenyl]-5-1H-[1,2,3]-triazol-1-ylmethyl}-oxazolidin-2-one;
202. (R)-{3-[4-(4-(piperidin-1-yl)-3-fluorophenyl)-3-fluorophenyl]-5-1H-[1,2,3]-triazol-2-ylmethyl}-oxazolidin-2-one;
203. (R)-{3-[4-(4-(piperidin-1-yl)-3-fluorophenyl)-3-fluorophenyl]-5-1H-tetrazol-1-ylmethyl}-oxazolidin-2-one;
204. (S)—N-{3-[4-(4-(4,4-dimethoxypiperidin-1-yl)-3-fluorophenyl)-3-fluorophenyl]-2-oxo-oxazolidin-5-ylmethyl}-acetamide;
205. (R)-{3-[4-(4-(1,4-dioxa-8-azaspiro[4.5]dec-8-yl)-3-fluorophenyl)-3-fluorophenyl]-2-oxo-oxazolidin-5-ylmethyl}-methanesulfonate;
206. (S)—N-{3-[4-(4-(1,4-dioxa-8-azaspiro[4.5]dec-8-yl)-3-fluorophenyl)-3-fluorophenyl]-2-oxo-oxazolidin-5-ylmethyl}-acetamide;
207. (S)—N-{3-[4-(6-(1,4-dioxa-8-azaspiro[4.5]dec-8-yl)-pyridin-3-yl)-3-fluorophenyl]-2-oxo-oxazolidin-5-ylmethyl}-acetamide;
208. (R)-{3-[4-(4-(4-oxo-piperidin-1-yl)-3-fluorophenyl)-3-fluorophenyl]-2-oxo-oxazolidin-5-ylmethyl}-methanesulfonate;
209. (S)—N-{3-[4-(4-(4-oxo-piperidin-1-yl)-3-fluorophenyl)-3-fluorophenyl]-2-oxo-oxazolidin-5-ylmethyl}-acetamide;
210. (R)-{3-[4-(4-(4-hydroxy-piperidin-1-yl)-3-fluorophenyl)-3-fluorophenyl]-2-oxo-oxazolidin-5-ylmethyl}-methanesulfonate;
211. (S)—N-{3-[4-(4-(4-hydroxy-piperidin-1-yl)-3-fluorophenyl)-3-fluorophenyl]-2-oxo-oxazolidin-5-ylmethyl}-acetamide;
212. (S)—N-{3-[4-(6-(4-hydroxy-piperidin-1-yl)-pyridin-3-yl)-3-fluorophenyl]-2-oxo-oxazolidin-5-ylmethyl}-acetamide;
213. (R)-{3-[4-(4-(4-methanesulfonyloxy-piperidin-1-yl)-3-fluorophenyl)-3-fluorophenyl]-2-oxo-oxazolidin-5-ylmethyl}-methanesulfonate;
214. (S)—N-{3-[4-(4-(4-methanesulfonyloxy-piperidin-1-yl)-3-fluorophenyl)-3-fluorophenyl]-2-oxo-oxazolidin-5-ylmethyl}-acetamide;
215. (S)—N-{3-[4-(6-(4-methanesulfonyloxy-piperidin-1-yl)-pyridin-3-yl)-3-fluorophenyl]-2-oxo-oxazolidin-5-ylmethyl}-acetamide;
216. (S)—N-{3-[4-(4-(4-cyanomethylidene-piperidin-1-yl)-3-fluorophenyl)-3-fluorophenyl]-2-oxo-oxazolidin-5-ylmethyl}-acetamide;
217. (R)-{3-[4-(4-(4-cyanomethylidene-piperidin-1-yl)-3-fluorophenyl)-3-fluorophenyl]-5-(1H-[1,2,3]-triazol-1-yl)-methyl}-oxazolidin-2-one;
218. (R)-{3-[4-(4-(4-cyanomethylidene-piperidin-1-yl)-3-fluorophenyl)-3-fluorophenyl]-5-(1H-tetrazol-1-yl)methyl}-oxazolidin-2-one;
219. (S)—N-{3-[4-(4-(4-cyanomethyl-3,4-dehydropiperidin-1-yl)-3-fluorophenyl)-3-fluorophenyl]-2-oxo-oxazolidin-5-ylmethyl}-acetamide;
220. (S)—N-{3-[4-(4-(4-cyanomethyl-piperidin-1-yl)-3-fluorophenyl)-3-fluorophenyl]-2-oxo-oxazolidin-5-ylmethyl}-acetamide;
221. (S)—N-{3-[4-(4-(4-hydroxy-4-hydroxymethyl-piperidin-1-yl)-3-fluorophenyl)-3-fluorophenyl]-2-oxo-oxazolidin-5-ylmethyl}-acetamide;
222. (S)—N-{3-[4-(4-(4-azidomethyl-4-hydroxy-piperidin-1-yl)-3-fluorophenyl)-3-fluorophenyl]-2-oxo-oxazolidin-5-ylmethyl}-acetamide;
223. (S)—N-{3-[4-(4-(4-(1H-[1,2,3]-triazol-1-yl)-piperidin-1-yl)-3-fluorophenyl)-3-fluorophenyl]-2-oxo-oxazolidin-5-ylmethyl}-acetamide and isomer thereof;
224. (S)—N-{3-[4-(4-(4-(1H-tetrazol-1-yl)-piperidin-1-yl)-3-fluorophenyl)-3-fluorophenyl]-2-oxo-oxazolidin-5-ylmethyl}-acetamide and isomer thereof;
225. (S)—N-{3-[4-(6-(4-Oxopiperidin-1-yl)-pyridin-3-yl)-3-fluorophenyl]-2-oxo-oxazolidin-5-ylmethyl}-acetamide;
226. (S)—N-{3-[4-(6-(4-Hydroxy-4-methoxymethylpiperidin-1-yl)-pyridin-3-yl)-3-fluorophenyl]-2-oxo-oxazolidin-5-ylmethyl}-acetamide;
227. (S)—N-{3-[4-(6-(4-Hydroxy-4-acetyloxymethylpiperidin-1-yl)-pyridin-3-yl)-3-fluorophenyl]-2-oxo-oxazolidin-5-ylmethyl}-acetamide;
228. (R)-{3-[4-(4-(piperazin-1-yl)-3-fluorophenyl)-3-fluorophenyl]-5-1H-[1,2,3]-triazol-1-ylmethyl}-oxazolidin-2-one;
229. (R)-{3-[4-(4-(piperazin-1-yl)-3-fluorophenyl)-3-fluorophenyl]-5-1H-tetrazol-1-ylmethyl})-oxazolidin-2-one;
230. (R)-{3-[4-(4-(piperazin-1-yl)-3-fluorophenyl)-3-fluorophenyl]-2-oxo-oxazolidin-5-ylmethyl}-methanesulfonate hydrochloride;
231. (R)-{3-[4-(4-(piperazin-1-yl)-3-fluorophenyl)-3-fluorophenyl]-5-(1,2,3-triazol-1-yl)methyl}-oxazolidin-2-one hydrochloride;
232. (S)-{3-[4-(4-(4-hydroxymethylcarbonyl-piperazin-1-yl)-3-fluorophenyl)-3-fluorophenyl]-2-oxo-oxazolidin-5-ylmethyl}-acetamide;
233. (R)-{3-[4-(4-(4-(2-nitrofuran-5-yl-methyl)-piperazin-1-yl)-3-fluorophenyl)-3-fluorophenyl]-2-oxo-oxazolidin-5-ylmethyl}-methanesulfonate;
234. (S)—N-{3-[4-(4-(4-(2-nitrofuran-5-yl-methyl)-piperazin-1-yl)-3-fluorophenyl)-3-fluorophenyl]-2-oxo-oxazolidin-5-ylmethyl}-acetamide;
235. (R)-{3-[4-(4-(4-benzyloxycarbonyl-piperazin-1-yl)-3-fluorophenyl)-3-fluorophenyl]-2-oxo-oxazolidin-5-ylmethyl}-methanesulfonate;
236. (S)-{3-[4-(4-benzyloxycarbonyl-piperazin-1-yl-3-fluorophenyl)-3-fluorophenyl]-5-1H-[1,2,3]-triazol-1-ylmethyl}-oxazolidin-2-one;
237. (S)—N-{3-[4-(4-benzyloxycarbonyl-piperazin-1-yl-3-fluorophenyl)-3-fluorophenyl]-2-oxo-oxazolidin-5-ylmethyl}-acetamide;
238. (S)—N-{3-[4-(4-(1-oxa-6-azaspiro[2.5]oct-6-yl)-3-fluorophenyl)-3-fluorophenyl]-2-oxo-oxazolidin-5-ylmethyl}-acetamide;
239. (S)—N-{3-[4-(4-(morpholin-1-yl)-phenyl)-3-fluorophenyl]-2-oxo-oxazolidin-5-ylmethyl}-acetamide;
240. (S)—N-{3-[4-(4-(thiomorpholin-4-yl)-3-fluorophenyl)-3-fluorophenyl]-2-oxo-oxazolidin-5-ylmethyl}-acetamide;
241. (S)—N-{3-[4-(6-3-(thiomorpholin-4-yl)-pyridin-3-yl)-3-fluorophenyl]-2-oxo-oxazolidin-5-ylmethyl}-methanesulfonate;
242. (S)—N-{3-[4-(6-(thiomorpholin-4-yl)-pyridin-3-yl)-3-fluorophenyl]-2-oxo-oxazolidin-5-ylmethyl}-acetamide;
243. (R)-{3-[4-(6-(Thiomorpholin-4-yl)-pyridin-3-yl)-3-fluorophenyl]-2-oxo-oxazolidin-5-ylmethyl}-methansulphonate;
244. (S)—N-{3-[4-(6-(Thiomorpholin-4-yl)-pyridin-3-yl)-3-fluorophenyl]-2-oxo-oxazolidin-5-ylmethyl}-formamide;
245. (S)—N-{3-[4-(6-(Thiomorpholin-4-yl)-pyridin-3-yl)-3-fluorophenyl]-2-oxo-oxazolidin-5-ylmethyl}-propionamide;
246. (S)-{3-[4-(6-(thiomorpholin-4-yl)-pyridin-3-yl)-3-fluorophenyl]-2-oxo-oxazolidin-5-ylmethyl}-carbamic acid methyl ester;
247. (S)-{3-[4-(6-thiomorpholin-4-yl-pyridin-3-yl)-3-fluorophenyl]-5-[1,2,3]-triazol-1-ylmethyl}-oxazolidin-2-one;
248. (S)—N-{3-[6-(6-(Thiomorpholin-4-yl)-pyridin-3-yl)-pyridin-3-yl]-2-oxo-oxazolidin-5-ylmethyl}-acetamide;
249. (S)—N-{3-[6-(4-(Thiomorpholin-4-yl)-3-fluorophenyl)-pyridin-3-yl]-2-oxo-oxazolidin-5-ylmethyl}-acetamide;
250. (S)—N-{3-[4-(6-(Thiomorpholin-4-yl)-pyridin-3-yl)-phenyl]-2-oxo-oxazolidin-5-yl methyl}-acetamide;
251. (S)—N-{3-[4-(2-(Thiomorpholin-4-yl)-pyrimidin-5-yl)-3-fluorophenyl]-2-oxo-oxazolidin-5-ylmethyl}-carbamic acid methyl ester;
252. (S)—N-{3-[4-(2-(Thiomorpholin-4-yl)-pyrimidin-5-yl)-3-fluorophenyl]-5-hydroxymethyl}-oxazolidin-2-one;
253. (S)—N-{3-[4-(2-(Thiomorpholin-4-yl)-pyrimidin-5-yl)-3-fluorophenyl]-2-oxo-oxazolidin-5-ylmethyl}-acetamide;
254. (S)—N-{3-[4-(2-(Thiomorpholin-4-yl)-pyrimidin-5-yl)-3-fluorophenyl]-5-[1,2,3]-triazol-1-ylmethyl}-oxazolidin-2-one;
255. (S)—N-{3-[4-(6-(Thiomorpholin-4-yl)-3-methylpyridin-3-yl)-3-fluorophenyl]-2-oxo-oxazolidin-5-ylmethyl}-carbamic acid methyl ester;
256. (S)—N-{3-[4-(6-(Thiomorpholin-4-yl)-3-methylpyridin-3-yl)-3-fluorophenyl]-2-oxo-oxazolidin-5-ylmethyl}-acetamide;
257. (S)—N-{3-[4-(6-(Thiomorpholin-4-yl)-3-methylpyridin-3-yl)-3-fluorophenyl]-5-hydroxymethyl}-oxazolidin-2-one;
258. (S)—N-{3-[4-(6-(Thiomorpholin-4-yl)-3-methylpyridin-3-yl)-3-fluorophenyl]-5-[1,2,3]-triazol-1-ylmethyl}-oxazolidin-2-one;
259. (S)—N-{3-[4-(2-(S-Oxo-thiomorpholin-4-yl)-pyrimidin-5-yl)-3-fluorophenyl]-5-hydroxymethyl}-oxazolidin-2-one;
260. (S)—N-{3-[4-(4-(S-oxo-thiomorpholin-4-yl)-3-fluorophenyl)-3-fluorophenyl]-2-oxo-oxazolidin-5-ylmethyl}-acetamide;
261. (S)—N-{3-[6-[6-(S-oxo-thiomorpholin-4-yl)-pyridin-3-yl]-pyridin-3-yl]-2-oxo-oxazolidin-5-ylmethyl}-acetamide;
262. (S)—N-{3-[4-(6-(S-oxo-thiomorpholin-4-yl)-pyridin-3-yl)-3-fluorophenyl]-2-oxo-oxazolidin-5-ylmethyl}-acetamide;
263. (R)-{3-[4-(6-(S-Oxo-thiomorpholin-4-yl)-pyridin-3-yl)-3-fluorophenyl]-2-oxo-oxazolidin-5-ylmethyl}-methansulphonate;
264. (S)—N-{3-[4-(6-(S-Oxo-thiomorpholin-4-yl)-pyridin-3-yl)-phenyl]-2-oxo-oxazolidin-5-ylmethyl}-acetamide;
265. (S)—N-{3-[4-(2-(S-Oxo-thiomorpholin-4-yl)-pyrimidin-5-yl)-3-fluorophenyl]-2-oxo-oxazolidin-5-ylmethyl}-carbamic acid methyl ester;
266. (S)—N-{3-[4-(2-(S-Oxo-thiomorpholin-4-yl)-pyrimidin-5-yl)-3-fluorophenyl]-2-oxo-oxazolidin-5-ylmethyl}-acetamide;
267. (S)—N-{3-[4-(2-(S-Oxo-thiomorpholin-4-yl)-pyrimidin-5-yl)-3-fluorophenyl]-5-[1,2,3]-triazol-1-ylmethyl}-oxazolidin-2-one;
268. (S)—N-{3-[4-(6-(S-Oxo-thiomorpholin-4-yl)-pyrimidin-5-yl)-3-fluorophenyl]-5-hydroxymethyl}-oxazolidin-2-one;
269. (S)—N-{3-[4-(6-(S-Oxo-thiomorpholin-4-yl)-3-methylpyridin-3-yl)-3-fluorophenyl]-2-oxo-oxazolidin-5-ylmethyl}-carbamic acid methyl ester;
270. (S)—N-{3-[4-(6-(S-Oxo-thiomorpholin-4-yl)-3-methylpyridin-3-yl)-3-fluorophenyl]-5-[1,2,3]-triazol-1-ylmethyl}-oxazolidin-2-one;
271. (S)—N-{3-[4-(6-(S-Oxo-thiomorpholin-4-yl)-3-methylpyridin-3-yl)-3-fluorophenyl]-5-hydroxymethyl}-oxazolidin-2-one;
272. (S)—N-{3-[4-(6-(S-Oxo-thiomorpholin-4-yl)-3-methylpyridin-3-yl)-3-fluorophenyl]-2-oxo-oxazolidin-5-ylmethyl}-acetamide;
273. (S)—N-{3-[4-(6-(S-oxa-3-fluoro-thiomorpholin-4-yl)-pyridin-3-yl)-3-fluorophenyl]-2-oxo-oxazolidin-5-ylmethyl}-acetamide;
274. (S)—N-{3-[4-(6-(S,S-dioxo-thiomorpholin-4-yl)-pyridin-3-yl)-3-fluorophenyl]-2-oxo-oxazolidin-5-ylmethyl}-acetamide;
275. (S)—N-{3-[4-(6-(S,S-dioxo-thiomorpholin-4-yl)-pyridin-3-yl)-3,5-difluorophenyl]-2-oxo-oxazolidin-5-ylmethyl}-acetamide;
276. (S)—N-{3-[4-(6-(S,S-dioxo-3-methyl-thiomorpholin-4-yl)-pyridin-3-yl)-3-fluorophenyl]-2-oxo-oxazolidin-5-ylmethyl}-acetamide;
277. (S)—N-{3-[4-(4-(S,S-dioxo-thiomorpholin-4-yl)-3-fluorophenyl)-3-fluorophenyl]-2-oxo-oxazolidin-5-ylmethyl}-acetamide;
278. (S)—N-{3-[6-[6-(S,S-dioxo-thiomorpholin-4-yl)-pyridin-3-yl]-pyridin-3-yl]-2-oxo-oxazolidin-5-ylmethyl}-acetamide;
279. (S)—N-{3-[6-(4-(S,S-dioxo-thiomorpholin-4-yl)-3-fluorophenyl)-pyridin-3-yl]-2-oxo-oxazolidin-5-ylmethyl}-acetamide;
280. (R)-3-{4-[6-(S,S-dioxo-thiomorpholin-4-yl)-pyridin-3-yl]-3-fluorophenyl}-5-hydroxymethyl-oxazolidin-2-one;
281. (2S,5R)-2-Amino-propionic acid 3-{4-[6-(S,S-dioxo-thiomorpholin-4-yl)-pyridin-3-yl]-3-fluoro-phenyl}-2-oxo-oxazolidin-5-ylmethyl ester;
282. (2S,5R)-2-Amino-propionic acid 3-{4-[6-(S,S-dioxo-thiomorpholin-4-yl)-pyridin-3-yl]-3-fluoro-phenyl}-2-oxo-oxazolidin-5-ylmethyl ester methanesulfonic acid salt;
283. (2S,5R)-2-Amino-3-methyl-butyric acid 3-{4-[6-(S,S-dioxo-thiomorpholin-4-yl)-pyridin-3-yl]-3-fluoro-phenyl}-2-oxo-oxazolidin-5-ylmethyl ester;
284. (2S,5R)-2-Amino-3-methyl-butyric acid 3-{4-[6-(S,S-dioxo-thiomorpholin-4-yl)-pyridin-3-yl]-3-fluoro-phenyl}-2-oxo-oxazolidin-5-ylmethyl ester methane sulfonic acid salt;
285. (R)-Acetic acid 3-{4-[6-(S,S-dioxo-thiomorpholin-4-yl)-pyridin-3-yl]-3-fluoro-phenyl}-2-oxo-oxazolidin-5-ylmethyl ester;
286. (S)—N-{3-[4-(6-(S,S-Dioxo-thiomorpholin-4-yl)-pyridin-3-yl)-3-fluorophenyl]-2-oxo-oxazolidin-5-ylmethyl}-formamide;
287. (R)-{3-[4-(6-(S,S-Dioxo-thiomorpholin-4-yl)-pyridin-3-yl)-3-fluorophenyl]-2-oxo-oxazolidin-5-ylmethyl}-methansulphonate;
288. (S)—N-{3-[4-(6-(S,S-Dioxo-thiomorpholin-4-yl)-pyridin-3-yl)-3-fluorophenyl]-2-oxo-oxazolidin-5-ylmethyl}-propionamide;
289. (S)—N-{3-4-(6-(S,S-Dioxo-thiomorpholin-4-yl)-pyridin-3-yl)-3-fluorophenyl)-2-oxo-oxazolidin-5-ylmethyl}-carbamic acid methyl ester;
290. N-Acetyl-(R)—N-{3-4-{2-(S,S-Dioxo-thiomorpholin-4-yl)-pyridyl-5-yl-3-fluorophenyl-1-yl-]}-2-oxo-oxazolidin-5-ylmethyl}-aminocarbonyl-oxymethyl acetate;
291. (S)—N-{3-[4-(6-(S,S-Dioxo-thiomorpholin-4-yl)-pyridin-3-yl)-3-fluorophenyl]-5-[1,2,3]-triazol-1-ylmethyl}-oxazolidin-2-one;
292. (S)—N-{3-[4-(6-(S,S-Dioxo-thiomorpholin-4-yl)-pyridin-3-yl)-3-fluorophenyl]-2-oxo-oxazolidin-5-ylmethyl}-urea;
293. (S)—N-{3-[4-(6-(S,S-Dioxo-thiomorpholin-4-yl)-pyridin-3-yl)-phenyl]-2-oxo-oxazolidin-5-ylmethyl}-acetamide;
294. (S)—N-{3-[6-(6-(S,S-Dioxo-thiomorpholin-4-yl)-pyridin-3-yl)-pyridin-3-yl]-2-oxo-oxazolidin-5-ylmethyl}-acetamide;
295. (S)—N-{3-[4-(2-(S,S-Dioxo-thiomorpholin-4-yl)-pyrimidin-5-yl)-3-fluorophenyl]-2-oxo-oxazolidin-5-ylmethyl}-carbamic acid methyl ester;
296. (S)—N-{3-[4-(2-(S,S-Dioxo-thiomorpholin-4-yl)-pyrimidin-5-yl)-3-fluorophenyl]-5-hydroxymethyl}-oxazolidin-2-one;
297. (S)—N-{3-[4-(2-(S,S-Dioxo-thiomorpholin-4-yl)-pyrimidin-5-yl)-3-fluorophenyl]-2-oxo-oxazolidin-5-ylmethyl}-acetamide;
298. (S)—N-{3-[4-(2-(S,S-Dioxo-thiomorpholin-4-yl)-pyrimidin-5-yl)-3-fluorophenyl]-5-[1,2,3]-triazol-1-ylmethyl}-oxazolidin-2-one;
299. (S)—N-{3-[4-(6-(S,S-Dioxo-thiomorpholin-4-yl)-3-methylpyridin-3-yl)-3-fluorophenyl]-2-oxo-oxazolidin-5-ylmethyl}-carbamic acid methyl ester;
300. (S)—N-{3-[4-(6-(S,S-Dioxo-thiomorpholin-4-yl)-3-methylpyridin-3-yl)-3-fluorophenyl]-5-hydroxymethyl}-oxazolidin-2-one;
301. (S)—N-{3-[4-(6-(S,S-Dioxo-thiomorpholin-4-yl)-3-methylpyridin-3-yl)-3-fluorophenyl]-2-oxo-oxazolidin-5-ylmethyl}-acetamide;
302. (S)—N-{3-[4-(6-(S,S-Dioxo-thiomorpholin-4-yl)-3-methylpyridin-3-yl)-3-fluorophenyl]-5-[1,2,3]-triazol-1-ylmethyl}-oxazolidin-2-one;
303. (S)—N-{3-[4-(6-(S,S-Dioxo-3-methyl-thiomorpholin-4-yl)-pyridin-3-yl)-3-fluorophenyl]-2-oxo-oxazolidin-5-ylmethyl}-difluoroacetamide;
304. (S)—N-{3-[4-(6-(S-Oxo-S—(N-methylimino)-thiomorpholin-4-yl)-pyridyl-3-yl)-3-fluorophenyl-1-yl]-2-oxo-oxazolidin-5-ylmethyl}-acetamide;
305. (S)—N-{3-[4-(6-(S-Oxo-S—(N-methylimino)-thiomorpholin-4-yl)-pyridin-3-yl)-phenyl]-2-oxo-oxazolidin-5-ylmethyl}-acetamide;
306. (S)—N-{3-[4-(6-(S-Oxo-S—(N-acetylimino)-thiomorpholin-4-yl)-pyridyl-3-yl)-3-fluorophenyl-1-yl]-2-oxo-oxazolidin-5-ylmethyl}-acetamide;
307. (S)—N-{3-[4-(6-(S-Oxo-S-(1-chloroethylurido)-thiomorpholin-4-yl)-pyridyl-3-yl)-3-fluorophenyl-1-yl]-2-oxo-oxazolidin-5-ylmethyl}-acetamide;
308. (S)—N-{3-[4-(6-(S-Oxo-S-imino-thiomorpholin-4-yl)-pyridyl-3-yl)-3-fluorophenyl-1-yl]-2-oxo-oxazolidin-5-ylmethyl}-acetamide;
309. (S)—N-{3-[4-(6-(1,4-Dioxa-8-azaspiro[4.5]dec-8-yl)-pyridin-3-yl)-3-fluorophenyl]-2-oxo-oxazolidin-5-ylmethyl}-acetamide;
310. (S)—N-{3-[4-(6-(S-Oxo-3,3-dichloro-thiomorpholin-4-yl)-pyridin-3-yl)-3-fluorophenyl]-2-oxo-oxazolidin-5-ylmethyl}-acetamide;
311. (R)-Phosphoric acid {3-[4-(6-(S,S-dioxo-thiomorpholin-4-yl)-pyridin-3-yl)-3-fluorophenyl]-2-oxo-oxazolidin-5-ylmethyl}ester disodium salt;
312. Phosphoric acid mono (R)—N-{3-{4-{2-(thiomorpholin-4-yl)-pyrimidin-5-yl}-3-fluorophenyl]}-2-oxo-oxazolidin-5-ylmethyl}ester;
313. Phosphoric acid mono (R)—N-{3-{4-{2-(S-oxo-thiomorpholin-4-yl)-pyrimidin-5-yl}-3-fluorophenyl]}-2-oxo-oxazolidin-5-ylmethyl}ester;
314. Phosphoric acid mono (R)—N-{3-{4-{2-(S,S-dioxo-thiomorpholin-4-yl)-pyrimidin-5-yl}-3-fluorophenyl]}-2-oxo-oxazolidin-5-ylmethyl}-ester;
315. (R)-Phosphoric acid mono {3-[4-(6-(S,S-dioxo-thiomorpholin-4-yl)-pyridin-3-yl)-3-fluorophenyl]-2-oxo-oxazolidin-5-ylmethyl}ester;
316. Phosphoric acid mono (R)—N-{3-{4-{2-(S,S-Dioxo thiomorpholin-4-yl)-pyrimidin-5-yl}-3-fluorophenyl]}-2-oxo-oxazolidin-5-ylmethyl}-ester disodium salt;
317. (R)-Phosphoric acid mono {3-[4-(6-(thiomorpholin-4-yl)-3-methylpyridin-3-yl)-3-fluorophenyl]-2-oxo-oxazolidin-5-ylmethyl}ester;
318. (R)-Phosphoric acid mono {3-[4-(6-(S-oxo-thiomorpholin-4-yl)-3-methylpyridin-3-yl)-3-fluorophenyl]-2-oxo-oxazolidin-5-ylmethyl}ester;
319. (R)-Phosphoric acid mono {3-[4-(6-(S,S-dioxo-thiomorpholin-4-yl)-3-methylpyridin-3-yl)-3-fluorophenyl]-2-oxo-oxazolidin-5-ylmethyl}ester;
320. (R)-Phosphoric acid mono {3-[4-(6-(S-oxo-thiomorpholin-4-yl)-3-methylpyridin-3-yl)-3-fluorophenyl]-2-oxo-oxazolidin-5-ylmethyl}ester disodium salt;
321. (5S)—N-[3-(3′-Chloromethyl-2-fluoro-biphenyl-4-yl)-2-oxo-oxazolidin-5-ylmethyl]-acetamide;
322. (5S)—N-[3-(2-Fluoro-3′-fluoromethyl-biphenyl-4-yl)-2-oxo-oxazolidin-5-ylmethyl]-acetamide;
323. (5S)—N-[3-(2-Fluoro-3′-azidomethyl-biphenyl-4-yl)-2-oxo-oxazolidin-5-ylmethyl]-acetamide;
324. (5S)—N-[3-(3′-Aminomethyl-2-fluoro-biphenyl-4-yl)-2-oxo-oxazolidin-5-ylmethyl]-acetamide;
325. (5S)-2-({4′-[5-(Acetylamino-methyl)-2-oxo-oxazolidin-3-yl]-2′-fluoro-biphenyl-3-ylmethyl}-amino)-acetamide;
326. (5S)-2-({4′-[5-(Acetylamino-methyl)-2-oxo-oxazolidin-3-yl]-2′-fluoro-biphenyl-3-ylmethyl}-carbamoylmethyl-amino)-acetamide;
327. (5S)-{3-[2-Fluoro-1,1′-biphenyl-3′-methoxy-4-yl]-2-oxo-oxazolidin-5yl-methyl}-acetamide;
328. (5S)-{3-[(2-Fluoro-1,1′-biphenyl-3′-yl)methanol]-2-oxo-oxazolidin-5yl-methyl}-acetamide;
329. (5S)-{3-[(3,5-Difluoro-4-[6-(4-thiomorpholin-1,1-dioxide-4-yl)-3-pyridinyl]phenyl]-2-oxo-oxazolidin-5yl-methyl}-acetamide;

Particularly more preferred compounds of the invention of formula I are
(S)—N-{3-[4-(4-(1H-[1,2,3]-triazol-1-yl)-piperidin-1-yl)-3-fluorophenyl]-2-oxo-oxazolidin-5-ylmethyl}-acetamide;
(S)—N-{3-[4-(4-(1H-[1,2,3]-triazol-1-yl)-piperidin-1-yl)-3,5-difluorophenyl]-2-oxo-oxazolidin-5-ylmethyl}-acetamide;
(S)—N-{3-[4-(4-(1H-[1,2,3]-triazol-1-yl)-3-fluoropiperidin-1-yl)-3,5-difluorophenyl]-2-oxo-oxazolidin-5-ylmethyl}-acetamide;
(S)—N-{3-[4-(4-(1H-[1,2,3]-triazol-1-yl)-3-fluoropiperidin-1-yl)-3-fluorophenyl]-2-oxo-oxazolidin-5-ylmethyl}-acetamide;
(S)—N-{3-[4-(4-(1H-tetrazol-1-yl)-piperidin-1-yl)-3-fluorophenyl]-2-oxo-oxazolidin-5-ylmethyl}-acetamide;
(S)—N-{3-[4-(4-(2H-tetrazol-2-yl)-piperidin-1-yl)-3-fluorophenyl]-2-oxo-oxazolidin-5-ylmethyl}-acetamide;
(S)—N-{3-[4-(4-(1H-tetrazol-1-yl)-3-fluoropiperidin-1-yl)-3,5-difluorophenyl]-2-oxo-oxazolidin-5-ylmethyl}-acetamide;
(S)—N-{3-[4-(4-(4-formyl-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-formyl-1H-[1,2,3]-triazol-1-yl)-piperidin-1-yl)-3,5-difluorophenyl]-2-oxo-oxazolidin-5-ylmethyl}-acetamide;
(S)—N-{3-[4-(4-(4-formyl-1H-[1,2,3]-triazol-1-yl)-3-fluoropiperidin-1-yl)-3-fluorophenyl]-2-oxo-oxazolidin-5-ylmethyl}-acetamide;
(R)-{3-[4-(4-(4-formyl-1H-[1,2,3]-triazol-1-yl)-piperidin-1-yl)-3-fluorophenyl]-5-(1H-[1,2,3]-triazol-1-ylmethyl)}-oxazolidin-2-one;
(S)—N-{3-[4-(4-(4-difluoromethyl-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-[1,3]-Dioxolan-2-yl-1H-[1,2,3]-triazol-1-yl)-piperidin-1-yl)-3-fluorophenyl]-5-(1H-[1,2,3]-triazol-1-ylmethyl)}-oxazolidin-2-one;
(S)—N-{3-[4-(4-(5-ethoxycarbonyl-1H-[1,2,3]-triazol-1-yl)-methyl-piperidin-1-yl)-3-fluoro-phenyl]-2-oxo-oxazolidin-5-ylmethyl}-acetamide;
(S)—N-{3-[4-(4-(4-ethoxycarbonyl-1H-[1,2,3]-triazol-1-yl)-3-fluoropiperidin-1-yl)-3-fluorophenyl]-2-oxo-oxazolidin-5-ylmethyl}-acetamide;
(S)—N-{3-[4-(4-(4-ethoxycarbonyl-1H-[1,2,3]-triazol-1-yl)-piperidin-1-yl)-3,5-difluorophenyl]-2-oxo-oxazolidin-5-ylmethyl}-acetamide;
(R)-{3-[4-(4-(4-ethoxycarbonyl-1H-[1,2,3]-triazol-1-yl)-piperidin-1-yl)-3-fluorophenyl]-5-(1-[1,2,3]-triazol-1-ylmethyl)}-oxazolidin-2-one;
(R)-{3-[4-(4-(4-ethoxycarbonyl-1H-[1,2,3]-triazol-1-yl)-piperidin-1-yl)-3,5-difluorophenyl]-5-(1H-[1,2,3]-triazol-1-ylmethyl)}-oxazolidin-2-one;
(S)—N-{3-[4-(4-(4-carboxamido-1H-[1,2,3]-triazol-1-yl)-piperidin-1-yl)-3-fluorophenyl]-2-oxo-oxazolidin-5-ylmethyl}-acetamide;
(R)-{3-[4-(4-(4-carboxamido-1H-[1,2,3]-triazol-1-yl)-piperidin-1-yl)-3-fluorophenyl]-5-(1H-[1,2,3]-triazol-1-ylmethyl)}-oxazolidin-2-one;
(S)—N-{3-[4-(4-(4-carboxamido-1H-[1,2,3]-triazol-1-yl)-methyl-piperidin-1-yl)-3-fluoro-phenyl]-2-oxo-oxazolidin-5-ylmethyl}-acetamide;
(S)—N-{3-[4-(4-(5-carboxamido-1H-[1,2,3],-triazol-1-yl)-methyl-piperidin-1-yl)-3-fluoro-phenyl]-2-oxo-oxazolidin-5-ylmethyl}-acetamide;
(S)—N-{3-[4-(4-(4-acetyl-1H-[1,2,3]-triazol-1-yl)-piperidin-1-yl)-3-fluorophenyl]-2-oxo-oxazolidin-5′-ylmethyl}-acetamide;
(S)—N-{3-[4-(4-(4-acetyl-1H-[1,2,3]-triazol-1-yl)-piperidin-1-yl)-3,5-difluorophenyl]-2-oxo-oxazolidin-5-ylmethyl}-acetamide;
(S)—N-{3-[4-(4-((4-((E/Z)-2-cyano-vinyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-hydroxyimino-methyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(1-methoxyimino-methyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(1-methoxyimino-methyl)-1H-[1,2,3]-triazol-1-yl)-piperidin-1-yl)-3,5-difluorophenyl]-2-oxo-oxazolidin-5-ylmethyl}-acetamide;
(S)—N-{3-[4-(4-(2H-tetrazol-2-ylmethyl)-[1,2,3]-triazol-1-yl)-piperidin-1-yl)-3-fluorophenyl]-2-oxo-oxazolidin-5-ylmethyl}-acetamide;
(R)-{3-[4-(4-(1H-[1,2,3]-triazol-1-yl)-piperidin-1-yl)-3-fluorophenyl]-5-(1H-[1,2,3]-triazol-1-ylmethyl)}-oxazolidin-2-one;
(R)-{3-[4-(4-(1H-[1,2,3]-triazol-1-yl)-piperidin-1-yl)-3,5-difluorophenyl]-5-(1H-[1,2,3]-triazol-1-ylmethyl)}-oxazolidin-2-one;
(R)-{3-[4-(4-(1H-[1,2,3]-triazol-1-yl)-3-fluoropiperidin-1-yl)-3,5-difluorophenyl]-5-(1H-[1,2,3]-triazol-1-ylmethyl)}-oxazolidin-2-one;
(S)—N-{3-[4-(4-([1,2,3]-triazol-1-yl)-piperidin-1-yl)-3-fluorophenyl]-2-oxo-oxazolidin-5-ylmethyl}-thioacetamide;
(S)—N-{3-[4-(4-(2-methyl-2H-tetrazol-5-yl)-piperidin-1-yl)-3,5-difluorophenyl]-2-oxo-oxazolidin-5-ylmethyl}-acetamide;
(S)—N-{3-[4-(4-(1-methyl-1H-tetrazol-5-yl)-piperidin-1-yl)-3,5-difluorophenyl]-2-oxo-oxazolidin-5-ylmethyl}-acetamide;
(S)—N-{3-[4-(4-(2-methyl)-2H-tetrazol-5-ylmethyl-piperidin-1-yl)-3,5-difluoro-phenyl]-2-oxo-oxazolidin-5-ylmethyl}-acetamide;
(S)—N-{3-[4-(4-(5-methylsulfanylmethyl-1H-tetrazol-1-yl)-piperidin-1-yl)-3,5-fluorophenyl]-2-oxo-oxazolidin-5-ylmethyl}-acetamide;
(S)—N-{3-[4-(4-(5-methylsulfonylmethyl-1H-tetrazol-1-yl)-piperidin-1-yl)-3,5-difluorophenyl]-2-oxo-oxazolidin-5-ylmethyl}-acetamide;
(S)—N-{3-[4-(4-(5-(thiophen-2-yl-methyl)-1H-tetrazol-1-yl)-piperidin-1-yl)-3,5-difluorophenyl]-2-oxo-oxazolidin-5-ylmethyl}-acetamide;
(S)—N-{3-[4-(4-(5-(4-pyridinyl)-1H-tetrazol-1-yl)-piperidin-1-yl)-3-fluorophenyl]-2-oxo-oxazolidin-5-ylmethyl}-acetamide;
(S)—N-{3-[4-(4-(5-(4-pyridinyl)-1H-tetrazol-1-yl)-piperidin-1-yl)-3,5-difluorophenyl]-2-oxo-oxazolidin-5-ylmethyl}-acetamide;
(S)—N-{3-[4-(4-(5-amino-2H-tetrazol-1-yl)-piperidin-1-yl)-3,5-difluorophenyl]-2-oxo-oxazolidin-5-ylmethyl}-acetamide;
(R)-{3-[4-(4-(1H-tetrazol-1-yl)-piperidin-1-yl)-3-fluorophenyl]-5-(1H-[1,2,3]-triazol-1-ylmethyl)}-oxazolidin-2-one;
(R)-{3-[4-(4-(2H-tetrazol-2-yl)-piperidin-1-yl)-3-fluorophenyl]-5-(1H-[1,2,3]-triazol-1-ylmethyl)}-oxazolidin-2-one;
(S)—N-{3-[4-(4-(5-methyl-[1,2,4]-oxadiazol-3-ylmethyl)-piperidin-1-yl)-3,5-difluoro-phenyl]-2-oxo-oxazolidin-5-ylmethyl}-acetamide;
(S)—N-{3-[4-(4-((5-pyridin-3-yl)-[1,2,4]-oxadiazol-3-ylmethyl)-piperidin-1-yl)-3-fluoro-phenyl]-2-oxo-oxazolidin-5-ylmethyl}-acetamide;
(S)—N-{3-[4-(4-(5-phenyl-[1,3,4]-oxadiazol-2-ylmethyl)-piperidin-1-yl)-3-fluoro-phenyl]-2-oxo-oxazolidin-5-ylmethyl}-acetamide;
(S)—N-{3-[4-(4-(5-Amino-[1,3,4]-thiadiazol-2-yl)-piperidin-1-yl)-3,5-difluoro-phenyl]-2-oxo-oxazolidin-5-ylmethyl}-acetamide;
(S)—N-{3-[4-(4-([1,2,4]-oxadiazol-3-yl)-piperidin-1-yl)-3,5-difluoro-phenyl]-2-oxo-oxazolidin-5-ylmethyl}-acetamide;
(S)—N-{3-[4-(4-(5-hydroxyethyl-2H-tetrazol-2-yl)-piperidin-1-yl)-3,5-difluorophenyl]-2-oxo-oxazolidin-5-ylmethyl}-acetamide;
(S)—N-{3-[4-(4-(5-acetylaminomethyl-2H-tetrazol-2-yl)-piperidin-1-yl)-3,5-difluorophenyl]-2-oxo-oxazolidin-5-ylmethyl}-acetamide;
(S)—N-{3-[4-(4-(1H-tetrazol-1-yl)-piperidin-1-yl)-3,5-difluorophenyl]-2-oxo-oxazolidin-5-ylmethyl}-acetamide;
(S)—N-{3-[4-(4-(2H-tetrazol-2-yl)-piperidin-1-yl)-3,5-difluorophenyl]-2-oxo-oxazolidin-5-ylmethyl}-acetamide;
(S)—N-{3-[4-(4-(2H-benzotriazol-2-yl)-piperidin-1-yl)-3,5-difluorophenyl]-2-oxo-oxazolidin-5-ylmethyl}-acetamide;
(S)—N-{3-[3,5-Difluoro-4-(4-2H-tetrazol-2-yl-piperidin-1-yl)-phenyl]-2-oxo-oxazolidin-5-ylmethyl}-propionamide;
(S)—N-{3-[3,5-Difluoro-4-(4-1H-tetrazol-1-yl-piperidin-1-yl)-phenyl]-2-oxo-oxazolidin-5-ylmethyl}-propionamide;
(S)—N-{3-[3,5-Difluoro-4-(4-[1,2,3]triazol-1-yl-piperidin-1-yl)-phenyl]-2-oxo-oxazolidin-5-ylmethyl}-propionamide;
(S)—N-3-{3,5-Difluoro-4-[4-(5-methyl-tetrazol-2-yl)-piperidin-1-yl]-phenyl}-2-oxo-oxazolidin-5-ylmethyl)-propionamide;
(S)—N-3-{3,5-Difluoro-4-[4-(5-methyl-tetrazol-1-yl)-piperidin-1-yl]-phenyl}-2-oxo-oxazolidin-5-ylmethyl)-propionamide;
(S)—N-{3-[3,5-Difluoro-4-(4-[1,2,3]triazol-1-yl-piperidin-1-yl)-phenyl]-2-oxo-oxazolidin-5-ylmethyl}-propionamide;
(S)—N-{3-[3,5-Difluoro-4-(4-1H-tetrazol-1-yl-piperidin-1-yl)-phenyl]-2-oxo-oxazolidin-5-ylmethyl}-carbamic acid methyl ester;
(S)—N-{3-[3,5-Difluoro-4-(4-[1,2,3]triazol-1-yl-piperidin-1-yl)-phenyl]-2-oxo-oxazolidin-5-ylmethyl}-carbamic acid methyl ester;
(S)—N-{3-[3,5-Difluoro-4-(4-[1,2,3]triazol-1-yl-piperidin-1-yl)-phenyl]-2-oxo-oxazolidin-5-ylmethyl}-2,2-difluoro-acetamide;
(S)—N-(3-{3,5-Difluoro-4-[4-(4-methyl-5-thioxo-4,5-dihydro-tetrazol-1-yl)-piperidin-1-yl]-phenyl}-2-oxo-oxazolidin-5-ylmethyl)-acetamide;
(S)—N-{3-[3-fluoro-4-(4-2H-tetrazol-2-yl-piperidin-1-yl)-phenyl]-2-oxo-oxazolidin-5-ylmethyl}-carbamic acid methyl ester;
(S)—N-{3-[3-fluoro-4-(4-1H-tetrazol-1-yl-piperidin-1-yl)-phenyl]-2-oxo-oxazolidin-5-ylmethyl}-carbamic acid methyl ester;
(R)-{3-(4-phenyl-3-fluorophenyl)-5-1H-[1,2,3]-triazol-1-ylmethyl}-oxazolidin-2-one;
(S)—N-{3-[4-(4-(piperidin-1-yl)-3-fluorophenyl)-3-fluorophenyl]-2-oxo-oxazolidin-5-ylmethyl}-acetamide;
(S)—N-{3-[4-(4-(4,4-dimethoxypiperidin-1-yl)-3-fluorophenyl)-3-fluorophenyl]-2-oxo-oxazolidin-5-ylmethyl}-acetamide;
(S)—N-{3-[4-(4-(1,4-dioxa-8-azaspiro[4,5]dec-8-yl)-3-fluorophenyl)-3-fluorophenyl]-2-oxo-oxazolidin-5-ylmethyl}-acetamide;
(S)—N-{3-[4-(4-(4-oxo-piperidin-1-yl)-3-fluorophenyl)-3-fluorophenyl]-2-oxo-oxazolidin-5-ylmethyl}-acetamide;
(S)—N-{3-[4-(4-(4-hydroxy-piperidin-1-yl)-3-fluorophenyl)-3-fluorophenyl]-2-oxo-oxazolidin-5-ylmethyl}-acetamide;
(S)—N-{3-[4-(4-(4-methansulfonyloxy-piperidin-1-yl)-3-fluorophenyl)-3-fluorophenyl]-2-oxo-oxazolidin-5-ylmethyl}-acetamide;
(S)—N-{3-[4-(4-(4-cyanomethylidene-piperidin-1-yl)-3-fluorophenyl)-3-fluorophenyl]-2-oxo-oxazolidin-5-ylmethyl}-acetamide;
(S)—N-{3-[4-(4-(4-cyanomethyl-3,4-dehydropiperidin-1-yl)-3-fluorophenyl)-3-fluorophenyl]-2-oxo-oxazolidin-5-ylmethyl}-acetamide;
(S)—N-{3-[4-(4-(4-cyanomethyl-piperidin-1-yl)-3-fluorophenyl)-3-fluorophenyl]-2-oxo-oxazolidin-5-ylmethyl}-acetamide;
(S)—N-{3-[4-(4-(4-azidomethyl-4-hydroxy-piperidin-1-yl)-3-fluorophenyl)-3-fluorophenyl]-2-oxo-oxazolidin-5-ylmethyl}-acetamide;
(S)—N-{3-[4-(4-(4-(1H-[1, 2,3]-triazol-1-yl)-piperidin-1-yl)-3-fluorophenyl)-3-fluorophenyl]-2-oxo-oxazolidin-5-ylmethyl}-acetamide and isomer thereof;
(R)-{3-[4-(4-(piperazin-1-yl)-3-fluorophenyl)-3-fluorophenyl]-5-(1,2,3-triazol-1-yl)methyl}-oxazolidin-2-one hydrochloride;
(S)—N-{3-[4-(4-(4-(2-nitrofuran-5-yl-methyl)-piperazin-1-yl)-3-fluorophenyl)-3-fluorophenyl]-2-oxo-oxazolidin-5-ylmethyl}-acetamide;
(S)—N-{3-[4-(4-(thiomorpholin-4-yl)-3-fluorophenyl)-3-fluorophenyl]-2-oxo-oxazolidin-5-ylmethyl}-acetamide;
(S)—N-{3-[4-(4-(S-oxo-thiomorpholin-4-yl)-3-fluorophenyl)-3-fluorophenyl]-2-oxo-oxazolidin-5-ylmethyl}-acetamide;
(S)—N-{3-[4-(4-(S,S-dioxo-thiomorpholin-4-yl)-3-fluorophenyl)-3-fluorophenyl]-2-oxo-oxazolidin-5-ylmethyl}-acetamide;
(S)—N-{3-[4-(6-(thiomorpholin-4-yl)-pyridin-3-yl)-3-fluorophenyl]-2-oxo-oxazolidin-5-ylmethyl}-acetamide;
(S)—N-{3-[4-(6-(S-oxo-thiomorpholin-4-yl)-pyridin-3-yl)-3-fluorophenyl]-2-oxo-oxazolidin-5-ylmethyl}-acetamide;
(S)—N-{3-[4-(6-(S,S-dioxo-thiomorpholin-4-yl)-pyridin-3-yl)-3-fluorophenyl]-2-oxo-oxazolidin-5-ylmethyl}-acetamide;
(S)—N-{3-[4-(6-(S,S-dioxo-3-methyl-thiomorpholin-4-yl)-pyridin-3-yl)-3-fluorophenyl]-2-oxo-oxazolidin-5-ylmethyl}-acetamide;
(S)—N-{3-[4-(6-(1,4-Dioxa-8-azaspiro[4.5]dec-8-yl)-pyridin-3-yl)-3-fluorophenyl]-2-oxo-oxazolidin-5-ylmethyl}-acetamide;
(S)—N-{3-[4-(6-(4-Oxopiperidin-1-yl)-pyridin-3-yl)-3-fluorophenyl]-2-oxo-oxazolidin-5-ylmethyl}-acetamide;
(S)—N-{3-[4-(6-(4-Hydroxy-4-methoxymethylpiperidin-1-yl)-pyridin-3-yl)-3-fluorophenyl]-2-oxo-oxazolidin-5-ylmethyl}-acetamide;
(R)-3-{4-[6-(S,S-Dioxo-thiomorpholin-4-yl)-pyridin-3-yl]-3-fluorophenyl}-5-hydroxymethyl-oxazolidin-2-one;
(R)-Phosphoric acid mono {3-[4-(6-(S,S-dioxo-thiomorpholin-4-yl)-pyridin-3-yl)-3-fluorophenyl]-2-oxo-oxazolidin-5-ylmethyl}ester;
(R)-Phosphoric acid {3-[4-(6-(S,S-dioxo-thiomorpholin-4-yl)-pyridin-3-yl)-3-fluorophenyl]-2-oxo-oxazolidin-5-ylmethyl}ester disodium salt;
(2S,5R)-2-Amino-propionic acid 3-{4-[6-(S,S-dioxo-thiomorpholin-4-yl)-pyridin-3-yl]-3-fluoro-phenyl}-2-oxo-oxazolidin-5-ylmethyl ester;
(2S,5R)-2-Amino-propionic acid 3-{4-[6-(S,S-dioxo-thiomorpholin-4-yl)-pyridin-3-yl]-3-fluoro-phenyl}-2-oxo-oxazolidin-5-ylmethyl ester methanesulphonic acid salt;
(2S,5R)-2-Amino-3-methyl-butyric acid 3-{4-[6-(S,S-dioxo-thiomorpholin-4-yl)-pyridin-3-yl]-3-fluoro-phenyl}-2-oxo-oxazolidin-5-ylmethyl ester;
(2S,5R)-2-Amino-3-methyl-butyric acid 3-{4-[6-(S,S-dioxo-thiomorpholin-4-yl)-pyridin-3-yl]-3-fluoro-phenyl}-2-oxo-oxazolidin-5-ylmethyl ester methanesulphonic acid salt;
(5R)-Acetic acid 3-{4-[6-(S,S-dioxo-thiomorpholin-4-yl)-pyridin-3-yl]-3-fluoro-phenyl}-2-oxo-oxazolidin-5-ylmethyl ester;
(R)-{3-[4-(6-(Thiomorpholin-4-yl)-pyridin-3-yl)-3-fluorophenyl]-2-oxo-oxazolidin-5-ylmethyl}-methansulphonate;
(R)-{3-[4-(6-(S-Oxothiomorpholin-4-yl)-pyridin-3-yl)-3-fluorophenyl]-2-oxo-oxazolidin-5-ylmethyl}-methansulphonate;
(R)-{3-[4-(6-(S,S-Dioxo-thiomorpholin-4-yl)-pyridin-3-yl)-3-fluorophenyl]-2-oxo oxazolidin-5-ylmethyl}-methansulphonate;
(S)—N-{3-[4-(6-(Thiomorpholin-4-yl)-pyridin-3-yl)-3-fluorophenyl]-2-oxo-oxazolidin-5-ylmethyl}-formamide;
(S)—N-{3-[4-(6-(S,S-Dioxo-thiomorpholin-4-yl)-pyridin-3-yl)-3-fluorophenyl]-2-oxo-oxazolidin-5-ylmethyl}-formamide;
(S)—N-{3-[4-(6-(S-Oxo-S—(N-methylimino)-thiomorpholin-4-yl)-pyridyl-3-yl)-3-fluorophenyl-1-yl]-2-oxo-oxazolidin-5-ylmethyl}-acetamide;
(S)—N-{3-[4-(6-(S-Oxo-S—(N-acetylimino)-thiomorpholin-4-yl)-pyridyl-3-yl)-3-fluorophenyl-1-yl]-2-oxo-oxazolidin-5-ylmethyl}-acetamide;
(S)—N-{3-[4-(6-(S-Oxo-S-(1-chloroethylurido)-thiomorpholin-4-yl)-pyridyl-3-yl)-3-fluorophenyl-1-yl]-2-oxo-oxazolidin-5-ylmethyl}-acetamide;
(S)—N-{3-[4-(6-(S-Oxo-S—(N-acetylimino)-thiomorpholin-4-yl)-pyridyl-3-yl)-3-fluorophenyl-1-yl]-2-oxo-oxazolidin-5-ylmethyl}-acetamide;
(S)—N-{3-[4-(6-(S,S-Dioxo-thiomorpholin-4-yl)-pyridin-3-yl)-3-fluorophenyl]-2-oxo-oxazolidin-5-ylmethyl}-propionamide;
(S)-{3-[4-(6-(thiomorpholin-4-yl)-pyridin-3-yl)-3-fluorophenyl]-2-oxo-oxazolidin-5-ylmethyl}-carbamic acid methyl ester;
(S)—N-{3-[4-(6-(S,S-Dioxo-thiomorpholin-4-yl)-pyridin-3-yl)-3-fluorophenyl]-2-oxo-oxazolidin-5-ylmethyl}-carbamic acid methyl ester;
N-Acetyl-(R)—N-{3-4-{2-(S,S-Dioxo thiomorpholin-4-yl)-pyridyl-5-yl-3-fluorophenyl-1-yl-]}-2-oxo-oxazolidin-5-ylmethyl}-aminocarbonyl-oxy methyl acetate;
(S)-{3-[4-(6-thiomorpholin-4-yl-pyridin-3-yl)-3-fluorophenyl]-5-[1,2,3]-triazol-1-ylmethyl}-oxazolidin-2-one;
(S)—N-{3-[4-(6-(S,S-Dioxo-thiomorpholin-4-yl)-pyridin-3-yl)-3-fluorophenyl]-5-[1,2,3]-triazol-1-ylmethyl}-oxazolidin-2-one;
(S)—N-{3-[4-(6-(S-Oxo-3,3-dichloro-thiomorpholin-4-yl)-pyridin-3-yl)-3-fluorophenyl]-2-oxo-oxazolidin-5-ylmethyl}-acetamide;
(S)—N-{3-[4-(6-(S,S-Dioxo-thiomorpholin-4-yl)-pyridin-3-yl)-3-fluorophenyl]-2-oxo-oxazolidin-5-ylmethyl}-urea;
(S)—N-{3-[4-(6-(Thiomorpholin-4-yl)-pyridin-3-yl)-phenyl]-2-oxo-oxazolidin-5-yl methyl}-acetamide;
(S)—N-{3-[4-(6-(S-Oxo-thiomorpholin-4-yl)-pyridin-3-yl)-phenyl]-2-oxo-oxazolidin-5-ylmethyl}-acetamide;
(S)—N-{3-[4-(6-(S,S-Dioxo-thiomorpholin-4-yl)-pyridin-3-yl)-phenyl]-2-oxo-oxazolidin-5-ylmethyl}-acetamide;
(S)—N-{3-[4-(6-(S-Oxo-S—(N-methylimino)-thiomorpholin-4-yl)-pyridin-3-yl)-phenyl]-2-oxo-oxazolidin-5-ylmethyl}-acetamide;
(S)—N-{3-[4-(2-(S,S-Dioxo-thiomorpholin-4-yl)-pyrimidin-5-yl)-3-fluorophenyl]-2-oxo-oxazolidin-5-ylmethyl}-carbamic acid methyl ester;
(R)-Phosphoric acid N-{3-{4-{2-(Thiomorpholin-4-yl)-pyrimidin-5-yl}-3-fluorophenyl]}-2-oxo-oxazolidin-5-ylmethyl}-ester;
(R)-Phosphoric acid N-{3-{4-{2-(S-Oxo thiomorpholin-4-yl)-pyrimidin-5-yl}-3-fluorophenyl]}-2-oxo-oxazolidin-5-ylmethyl}-ester;
(R)-Phosphoric acid N-{3-{4-{2-(S,S-Dioxo thiomorpholin-4-yl)-pyrimidin-5-yl}-3-fluorophenyl]}-2-oxo-oxazolidin-5-ylmethyl}-ester;
(R)-Phosphoric acid N-{3-{4-{2-(S,S-Dioxo thiomorpholin-4-yl)-pyrimidin-5-yl}-3-fluorophenyl]}-2-oxo-oxazolidin-5-ylmethyl}-ester disodium salt;
(S)—N-{3-[4-(2-(S,S-Dioxo-thiomorpholin-4-yl)-pyrimidin-5-yl)-3-fluorophenyl]-2-oxo-oxazolidin-5-ylmethyl}-acetamide;
(S)—N-{3-[4-(2-(S,S-Dioxo-thiomorpholin-4-yl)-pyrimidin-5-yl)-3-fluorophenyl]-5-[1,2,3]-triazol-1-ylmethyl}-oxazolidin-2-one;
(S)—N-{3-[4-(6-(S,S-Dioxo-thiomorpholin-4-yl)-3-methylpyridin-3-yl)-3-fluorophenyl]-2-oxo-oxazolidin-5-ylmethyl}-carbamic acid methyl ester;
(S)—N-{3-[4-(6-(S,S-Dioxo-thiomorpholin-4-yl)-3-methylpyridin-3-yl)-3-fluorophenyl]-5-hydroxymethyl}-oxazolidin-2-one;
(R)-Phosphoric acid mono {3-[4-(6-(S,S-dioxo-thiomorpholin-4-yl)-3-methylpyridin-3-yl)-3-fluorophenyl]-2-oxo-oxazolidin-5-ylmethyl}ester;
(R)-Phosphoric acid mono {3-[4-(6-(S-oxo-thiomorpholin-4-yl)-3-methylpyridin-3-yl)-3-fluorophenyl]-2-oxo-oxazolidin-5-ylmethyl}ester disodium salt;
(S)—N-{3-[4-(6-(S,S-Dioxo-thiomorpholin-4-yl)-3-methylpyridin-3-yl)-3-fluorophenyl]-2-oxo-oxazolidin-5-ylmethyl}-acetamide;
(S)—N-{3-[4-(6-(S,S-Dioxo-thiomorpholin-4-yl)-3-methylpyridin-3-yl)-3-fluorophenyl]-5-[1,2,3]-triazol-1-ylmethyl}-oxazolidin-2-one;
(5S)-{3-[(3,5-Difluoro-4-(6-(4-S,S-dioxo-thiomorpholin-4-yl)-3-pyridinyl)phenyl]-2-oxo-oxazolidin-5-yl-methyl}-acetamide.

A further embodiment of the invention is to provide methods of preparation of the compound of the invention.

Following schemes describe the preparation of compounds of Formula I of the present invention. All of the starting materials are prepared by procedures described in U.S. Pat. No. 5,668,286 and in PCT patent application PCT/IN03/00237, PC/IN03/00238 or by procedures that would be well known to one of ordinary skill in organic chemistry. The variables used in the following Schemes are as defined above. Optically pure material could be obtained either by one of a number of asymmetric synthesis or alternatively by resolution from a racemic mixture.

Oxazolidinone compounds bearing substituted heteroaryl and substituted heteroarylmethyl piperidine moiety may be prepared as per schemes described below:

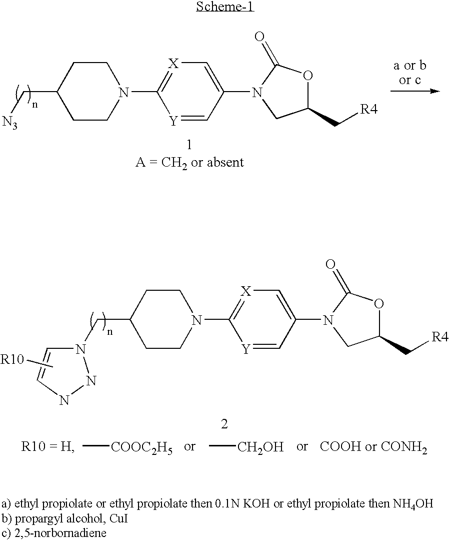

As per scheme-1, oxazolidinone compound of formula I is heated with ethyl propiolate in a solvent such as toluene or xylene for 3 to 14 hours at a temperature between 100-120° C. to provide oxazolidinone compound 2, wherein R₁₀ is CO₂C₂H₅. On further hydrolysis in presence of a base such as potassium hydroxide or sodium hydroxide in tetrahydrofuran, water mixture to provide oxazolidinone compound 2 wherein R₁₀ is CO₂H. Alternately the compound 2 with R₁₀ as CO₂C₂H₅ on further hydrolysis in presence of a base such as ammonium hydroxide in tetrahydrofuran, water mixture to provide oxazolidinone compound 2 wherein R₁₀ is CONH₂. Optionally heteroarylmethyl oxazolidinone compound was reacted with 2,5-norbornadiene in dioxane at a temperature 0-80° C. to provide oxazolidinone compound 2 of the invention.

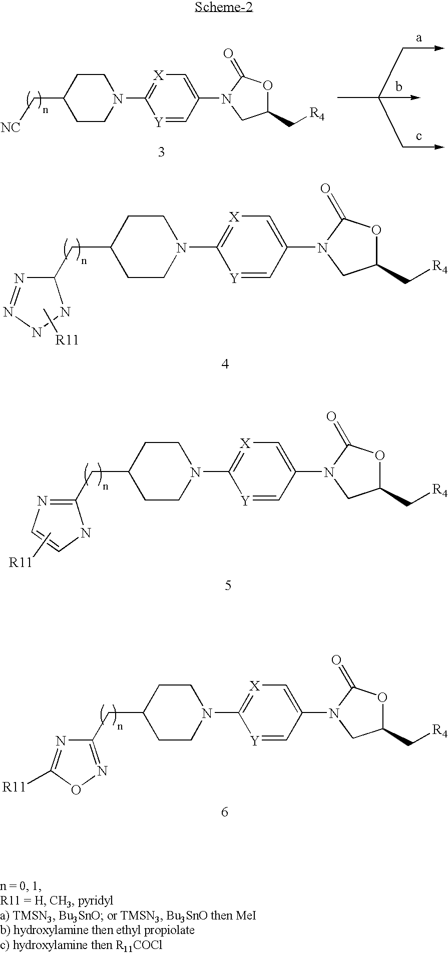

As per scheme-2, oxazolidinone compound of formula 3 is heated with trimethylsilylazide and tributyltinoxide in a solvent such as toluene or xylene for 3 to 14 hours at a temperature between 100-120° C. to provide oxazolidinone compound 4 of the invention. Optionally oxazolidinone compound was alkylated with alkylhalide such as methyliodide in presence of base such as sodium hydride in tetrahydrofuran to provide oxazolidinone compound 4 of the invention.

Oxazolidinone compound 3 was reacted with hydroxylamine hydrochloride in presence of a base such as sodium bicarbonate in methanol at a temperature 30-50° C. to provide hydroxyliminomethylpieridino oxazolidinone compound which subsequently upon reaction with ethylpropiolate in diphenyl ether solvent at reflux temperature provided oxazolidinone compound 5 of the invention.

Optionally oxazolidinone compound was subsequently reacted with organic acid chloride in presence of base such as sodium bicarbonate provided oxazolidinone compound 6 of the invention.

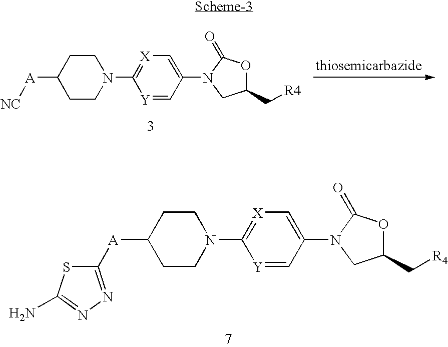

As per scheme-3, oxazolidinone compound of formula 3 is reacted with thiosemicarbazide in methanesulfonic acid for 3 to 14 hours at a temperature between 40-80° C. to provide oxazolidinone compound 7 of the invention.

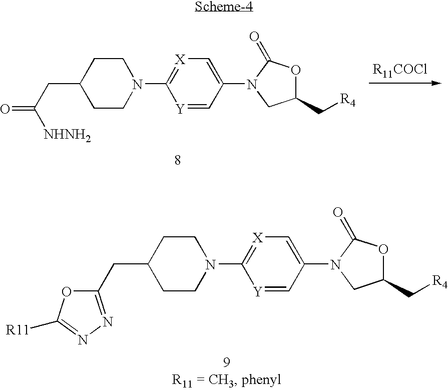

As per scheme-4, oxazolidinone compound of formula 8 is reacted with organic acid chloride in presence of base for example potassium carbonate, sodium carbonate, triethylamine for 3 to 14 hours at a temperature between 0-50° C. to provide oxazolidinone compound 9 of the invention.

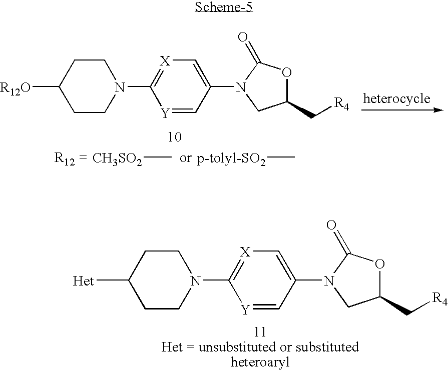

For the synthesis of substituted heteroarylpiperidine wherein the point of attachment is via the nitrogen atom of the heteroaryl, alkylsulfonyloxy or arylsulfonyloxy substituted piperidino oxazolidinone compound of formula 10 is reacted with unsubstituted or substituted heteroaryl for example imidazole, triazole, tetrazole in the presence of a base such as potassium carbonate for 3 to 24 hours at a temperature between 50-100° C. to provide oxazolidinone compound 11 of the invention (Scheme 5).

Oxazolidinone compounds bearing biaryl moieties may be prepared as per schemes described below:

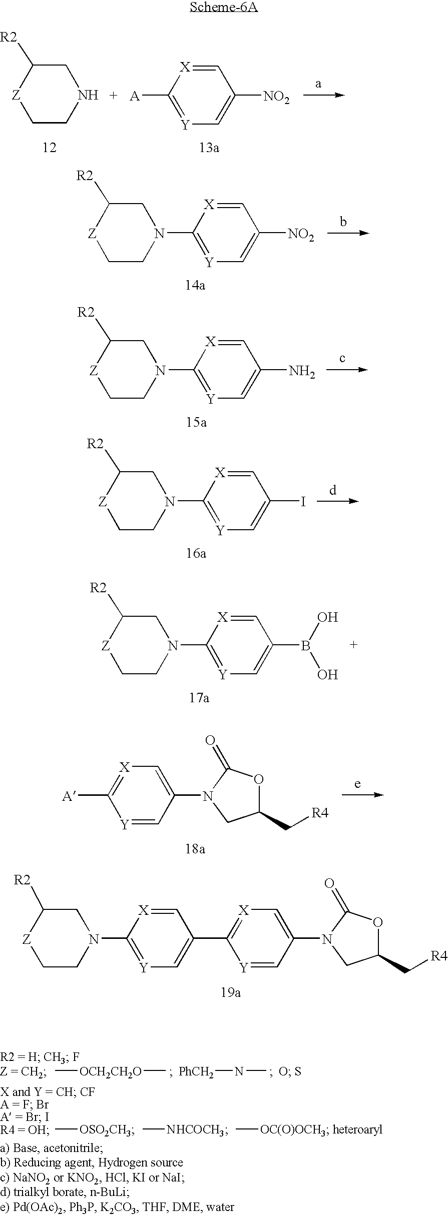

As per scheme-6A, nitrogen bearing heterocycle 12 (Z selected from CH₂, —OCH₂CH₂O—, PhCH₂N—, O, S) is reacted with compound 13a (A is either fluorine or bromine and X and Y selected from CH, CF) in presence of base such as triethylamine or diisopropylethylamine in solvent such as acetonitrile or dimethyl formamide or tetrahydrofuran, or mixture thereof at a temperature between 70-85° C. for 1 to 12 hours to provide compound 14a.

The compound 14a is reduced in presence of catalyst such as 5% or 10% palladium on carbon or 20% palladium hydroxide on carbon in presence of hydrogen source such as hydrogen gas optionally under pressure, or cyclohexene or ammonium formate in the presence of solvents such as methanol or ethanol or ethyl acetate at a temperature between 30-65° C. for 1 to 12 hours to provide heterocyclic aromatic amino compound 15a. Optionally, the reduction is carried out in presence of metal such as iron or zinc in presence of hydrochloric acid in a solvent such as methanol or ethanol at a temperature between 30-65° C. for 1 to 12 hours to provide compound 15a.

The compound 15a is diazotized with sodium nitrite or potassium nitrite in the presence of hydrochloric acid in a solvent such as water or ethanol or mixture thereof at a temperature between 0-10° C. for 1 to 2 hours, which upon further treatment with potassium iodide or sodium iodide in water at a temperature between 0-10° C. for 1 to 12 hours provided compound 16a.

The compound 16a is treated with trialkyl borate such as trimethyl borate or triethyl borate or tributyl borate in the presence of n-butyllithium in a solvent such as tetrahydrofuran or toluene or mixture thereof at a temperature between 0-40° C. for 1 to 24 hours there upon optional aqueous hydrochloric acid treatment furnished aryl boronic acid 17a.

The heterocyclic aromatic boronic acid 17a is reacted with compound 18a in presence of catalyst such as a mixture of palladium acetate and triphenyl phosphine or tetrakistriphenylphosphine or bisdiphenylphosphine palladium acetate in presence of base such as potassium carbonate or triethylamine, in a solvent such as dimethoxyethane or tetrahydrofuran or toluene, or aqueous ethanol, or mixture thereof at a temperature between 30-90° C. for 1 to 24 hours to furnish oxazolidinone compound 19a of the invention of formula I.

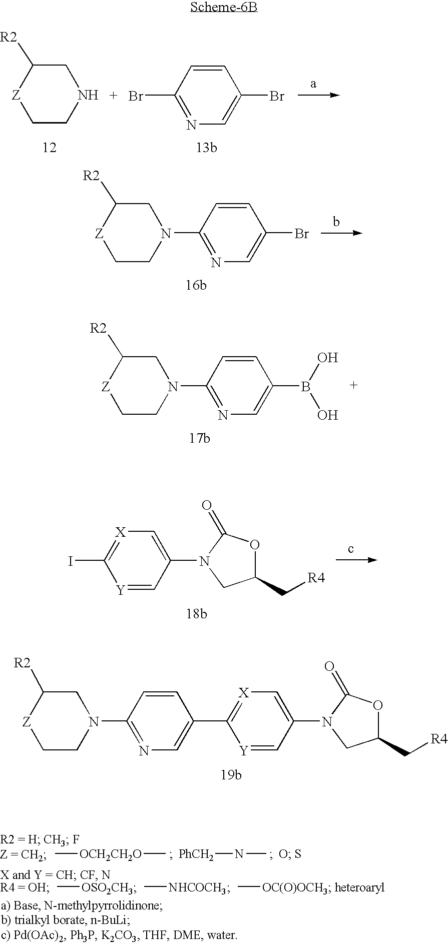

As per scheme-6B, nitrogen bearing heterocycle 12 (Z selected from CH₂, —OCH₂CH₂O—, PhCH₂N—, O, S) is reacted with 2,5-dibromo-pyridine (13b) in presence of base such as triethylamine or diisopropylethylamine in solvent such as N-methylpyrrolidinone, acetonitrile, dimethyl formamide, or mixture thereof at a temperature between 70-85° C. for 1 to 12 hours to provide compound 16b.

The compound 16b is treated with trialkyl borate such as trimethyl borate or triethyl borate, tributyl borate in the presence of n-butyllithium in a solvent such as tetrahydrofuran, toluene or mixture thereof at a temperature between 0-40° C. for 1 to 24 hours there upon optional aqueous hydrochloric acid treatment furnished boronic acid 17b.

The boronic acid 17b is reacted with compound 18b in presence of catalyst such as a mixture of palladium acetate and triphenyl phosphine or tetrakistriphenylphosphine or bisdiphenylphosphine palladium acetate in presence of base such as potassium carbonate or triethylamine, in a solvent such as dimethoxyethane or tetrahydrofuran, or mixture thereof at a temperature between 30-90° C. for 1 to 24 hours to furnish oxazolidinone compound 19b of the invention of formula I.

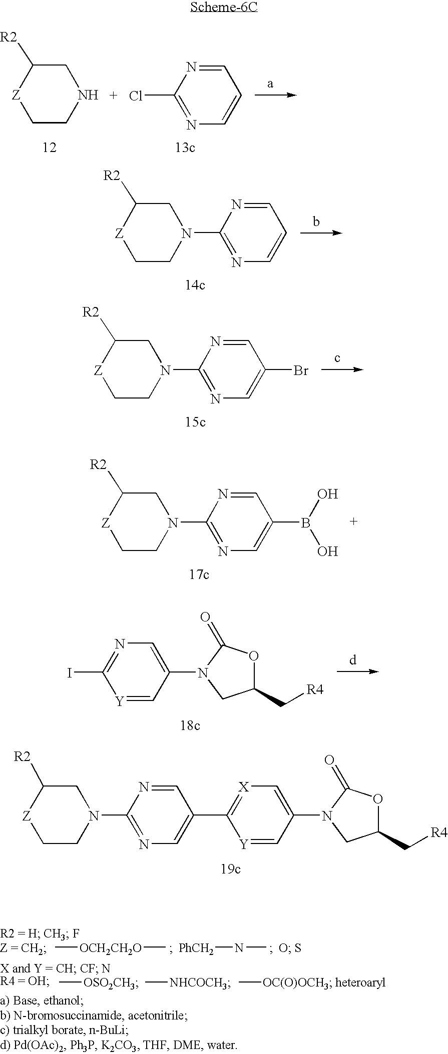

As per scheme-6C, nitrogen bearing heterocycle 12 (Z selected from CH₂, —OCH₂CH₂O—, PhCH₂N—, O, S) is reacted with 2-chloro-pyrimidine (13c) in presence of base such as triethylamine or diisopropylethylamine in solvent such as ethanol, or n-butanol or mixture thereof at a temperature between 70-100° C. for 1 to 12 hours to provide compound 14c.

The compound 14c is reacted with brominating agent such as N-bromosuccinamide in a solvent such as acetonitrile or chloroform or mixture thereof at a temperature 25-45° C. for 5-14 hours to furnish compound 15c.

The compound 15c is treated with trialkyl borate such as trimethyl borate or triethyl borate, tributyl borate in the presence of n-butyllithium in a solvent such as tetrahydrofuran or toluene or mixture thereof at a temperature between 0-40° C. for 1 to 24 hours there upon optional aqueous hydrochloric acid treatment furnished boronic acid 17c.

The boronic acid 17c is reacted with compound 18c in presence of catalyst such as a mixture of palladium acetate and triphenyl, phosphine or tetrakistriphenylphosphine or bisdiphenylphosphine palladium acetate in presence of base such as potassium carbonate or triethylamine, in a solvent such as dimethoxyethane or tetrahydrofuran, or mixture thereof at a temperature between 30-90° C. for 1 to 24 hours to furnish oxazolidinone compound 19c of the invention of formula I.

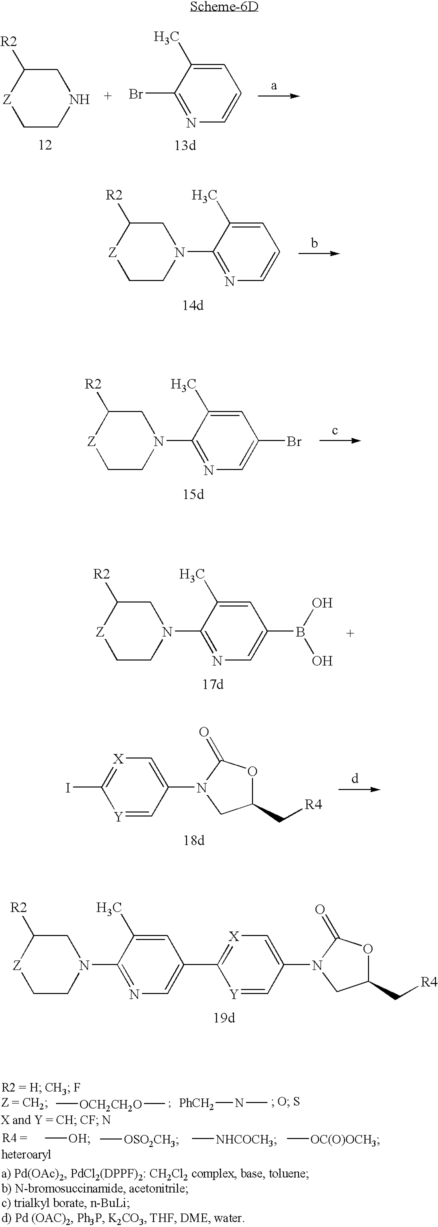

As per scheme-6D, nitrogen bearing heterocycle 12 (Z selected from CH₂, —OCH₂CH₂O)—, PhCH₂N—, O, S) is reacted with 2-bromo-3-methyl-pyridine (13d) in presence of palladium acetate and catalyst such as [1,1-bis(diphenylphosphino)-ferrocene]-dichloropalladium (II) complex with dichloromethane 1:1 also known as PdCl₂(DPPF)₂:CH₂Cl₂ complex and base such as sodium tert-butoxide in a solvent such as toluene or xylene or mixture thereof at a temperature between 70-140° C. for 5 to 12 hours to provide compound 14d.

The compound 14d is reacted with brominating agent such as N-bromosuccinamide in a solvent such as acetonitrile or chloroform or mixture thereof at a temperature 25-45° C. for 5-14 hours to furnish compound 15d.

The compound 15d is treated with trialkyl borate such as trimethyl borate or triethyl borate, tributyl borate in the presence of n-butyllithium in a solvent such as tetrahydrofuran, toluene or mixture thereof at a temperature between 0-40° C. for 1 to 24 hours there upon optional aqueous hydrochloric acid treatment furnished heterocyclic aryl boronic acid 17d.

The boronic acid 17d is reacted with compound 18d in presence of catalyst such as a mixture of palladium acetate and triphenyl phosphine or tetrakistriphenylphosphine or bisdiphenylphosphine palladium acetate in presence of base such as potassium carbonate or triethylamine, in a solvent such as dimethoxyethane or tetrahydrofuran, or mixture thereof at a temperature between 30-90° C. for 1 to 24 hours to furnish oxazolidinone compound 19d of the invention of formula I.

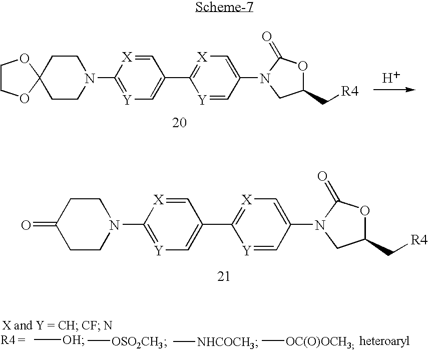

As per scheme-7, the oxazolidinone 20 (prepared as described in scheme-6A to 6D) is stirred with organic acid such as p-toluene sulfonic acid or inorganic acid such as dilute hydrochloric acid or dilute sulfuric acid in solvent such as acetone or water or tetrahydrofuran, or mixture thereof at a temperature between 30-80° C. for 1 to 12 hours to provide oxazolidnone compound 21 of the invention.

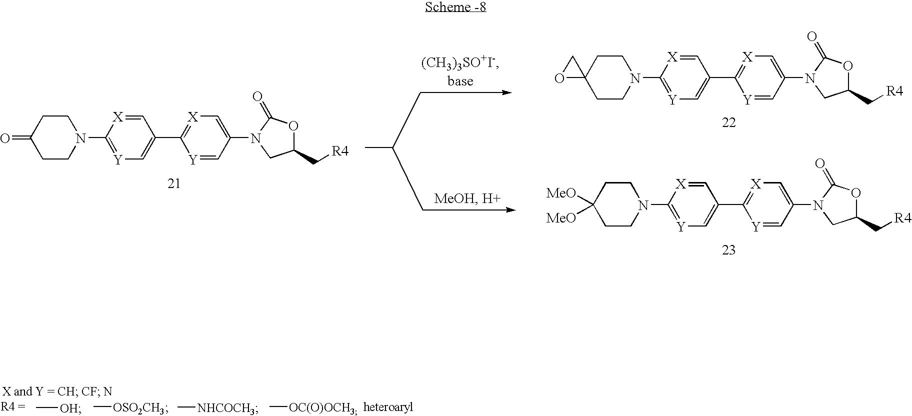

As per scheme 8, the oxazolidinone compound 21 (prepared as described above) is reacted with trimethylsulphoxonium iodide ((CH₃)₃SO⁺I⁻) or trimethylsulphonium iodide ((CH₃)₃S⁺I⁻) in the presence of a base such as sodium hydride or potassium tert-butoxide or lithium diisopropylamine or n-butyl lithium in a solvent such as dimethyl formamide or tetrahydrofuran or mixture thereof at a temperature between 0-85° C. for 1 to 12 hours to provide oxazolidinone compound 22 of the invention.

As per scheme-8, the oxazolidinone compound 21 is stirred with p-toluenesulfonic acid in methanol at a temperature between 0-85° C. for 1 to 12 hours to provide oxazolidinone compounds 23 of the invention.

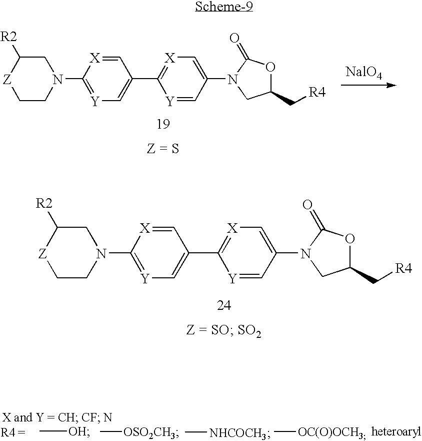

As per Scheme-9, the oxazolidinone 19 is optionally oxidized with sodium peroidate in solvent such as aqueous methanol or rectified spirit at a temperature between 0-80° C. for 1 to 48 hours to provide oxazolidinone compound 24 of the invention.

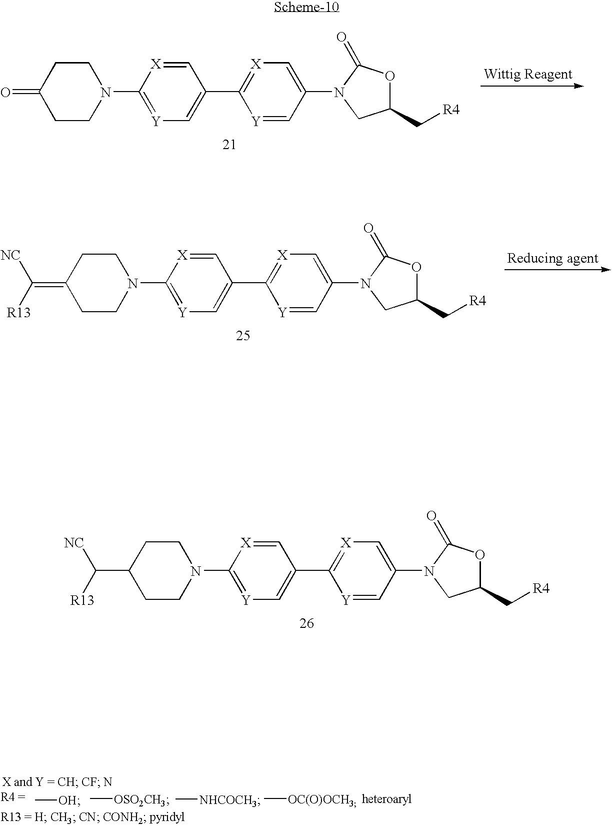

As per scheme-10, the oxazolidinone compound 21 (prepared as described above) is reacted with appropriate Wittig reagent such as diethylcyanomethylphosphonate or diethyl-(1-cyanoethyl)-phosphonate or diethyl(1-cyano-2-carboxamidomethyl)-phosphonate or diethyl(1-cyano-2-(3-pyridylmethyl)-phosphonate in the presence of base such as triethylamine or diisopropylethylamine and lithium bromide in a solvent such as dimethyl formamide or tetrahydrofuran or mixture thereof at a temperature between 0-85° C. for 1 to 12 hours to provide oxazolidinone compound 25 of the invention.

Compound 25 is optionally reduced with reducing agent such as 5% or 10% palladium on carbon or 20% palladium hydroxide on carbon in presence of hydrogen source such as hydrogen gas optionally under pressure, or cyclohexene or ammonium formate in the presence of solvents such as methanol or ethanol or ethyl acetate at a temperature between 30-65° C. for 1 to 12 hours to provide oxazoldinone compound 26 of the invention.

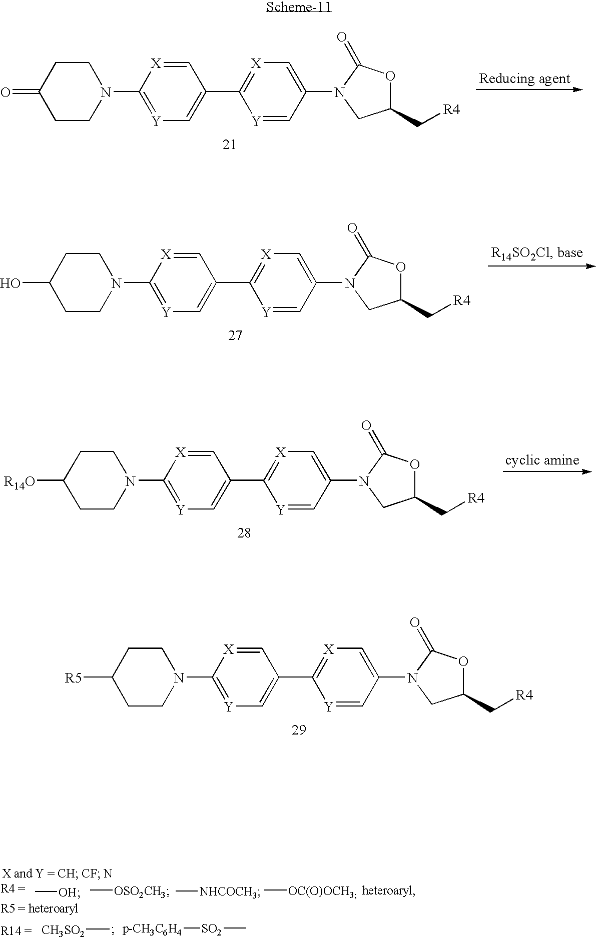

As per scheme-11, the oxazolidinone compound 21 (prepared as described above) is reacted with appropriate reducing reagent such as sodium borohydride in a solvent such as methanol or ethanol at a temperature between 0-35° C. for 1 to 12 hours to provide oxazolidinone compound 27 of the invention.

Compound 27 is optionally reacted with alkylsulfonyl chloride such as methanesulfonylchloride or ethanesulfonylchloride or aryl sulfonylchloride such as p-toleune sulfonylchloride or p-bromosulfonylchloride in presence of base such as triethylamine or pyridine or diisopropylethylamine in a solvent such as dichloromethane or chloroform or tetrahydrofuran at a temperature between 0-35° C. for 1 to 12 hours to provide oxazolidinone compound 28 of the invention.

Compound 28 is optionally reacted with heteroaryl amine such as pyrrole, or pyrazole, or imidazole or triazole or tetrazole or aliphatic cyclic amine such as pyrrolidine or piperidine or piperazine or morpholine in presence of base such as potassium carbonate or sodium carbonate in a solvent such as dimethylformamide or dioxane at a temperature between 35-80° C. for 1 to 12 hours to provide oxazoldinone compound 28 of the invention.

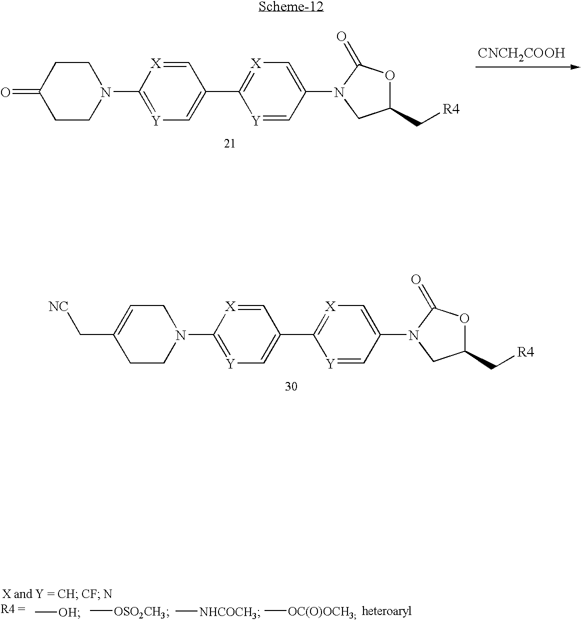

As per scheme-12, the oxazolidinone compound 21 (prepared as described above) is reacted with cyanoacetic acid and base such as pyridine or triethylamine or piperidine in presence of ammonium acetate in a solvent such as toleune or xylene at a temperature between 80-120° C. for 1 to 12 hours to provide oxazolidinone compounds 30 of the invention.

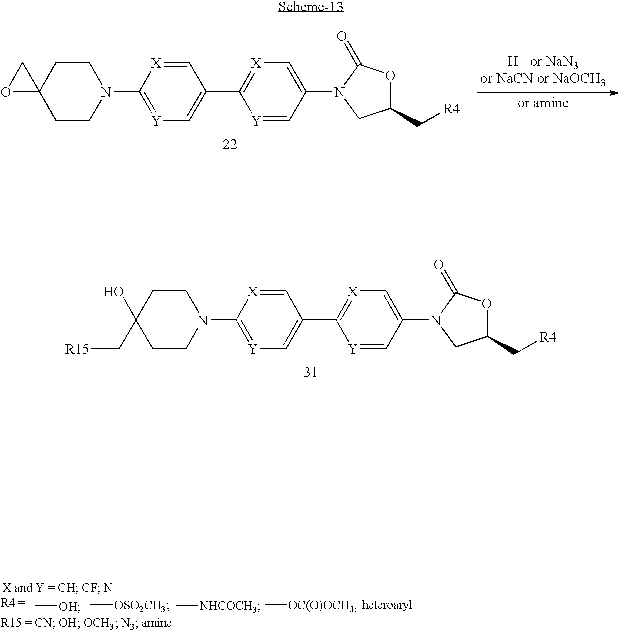

In accordance with scheme-13, oxazolidinone 22 (prepared as per procedure described above) is reacted with appropriate acid catalyst such as p-toluene sulfonic acid or hydrochloric acid or sulfuric acid or with a nucleophilic reagent such as sodium azide, sodium cyanide, sodium methoxide or amine such as methyl amine dimethyl amine or with a cyclic amines, such as pyrrolidine or piperidine, or with an aromatic amine such as imidazole or triazole or tetrazole in a solvent such as dimethylformamide or dimethylacetamide or methanol or ethanol or mixture thereof and stirred for 3 to 48 hours at a temperature between 10-100° C. to provide oxazolidinone 31 of the invention.

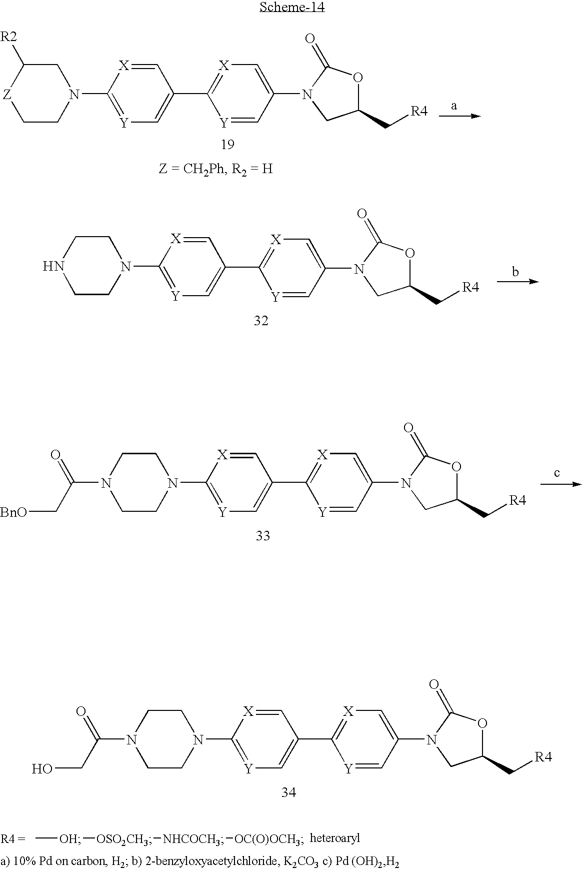

In accordance with scheme-14, oxazolidinone compound 19 (prepared as per procedure described above) is stirred in hydrogen atmosphere in presence of catalyst such as 5% or 10% palladium on carbon or 20% palladium hydroxide on carbon in a solvent such as methanol or ethanol or ethylacetate for 3 to 48 hours at a temperature between 35-60° C. to provide oxazolidinone 32 of the invention.

Optionally compound 32 is optionally reacted with ethanolic hydrochloric acid to provide hydrochloride salt of compound 32 of the invention.

Optionally compound 32 is reacted with 2-benzyloxyacetylchloride in presence of potassium carbonate or sodium carbonate in a acetone, water mixture 1 to 14 hours at a temperature between 0-35° C. to provide oxazolidinone 33 of the invention.

Optionally compound 33 is stirred in hydrogen atmosphere in presence of catalyst such as 5% or 10% palladium on carbon or palladium hydroxide in a solvent such as methanol or ethanol or ethyl acetate for 3 to 48 hours at a temperature between 35-60° C. to provide oxazolidinone 34 of the invention.

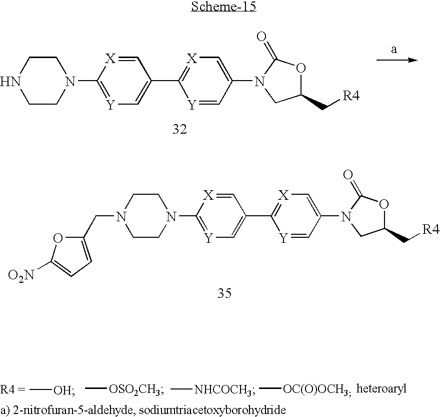

In accordance with scheme-15, oxazolidinone 32 (prepared as per procedure described above) is stirred with 2-nitro-furan-aldehyde in a solvent such as dichloromethane or chloroform or methanol or ethanol followed by addition of sodiumtriacetoxyborohydride for 3 to 10 hours at a temperature between 35-60° C. to provide oxazolidinone 35 of the invention.

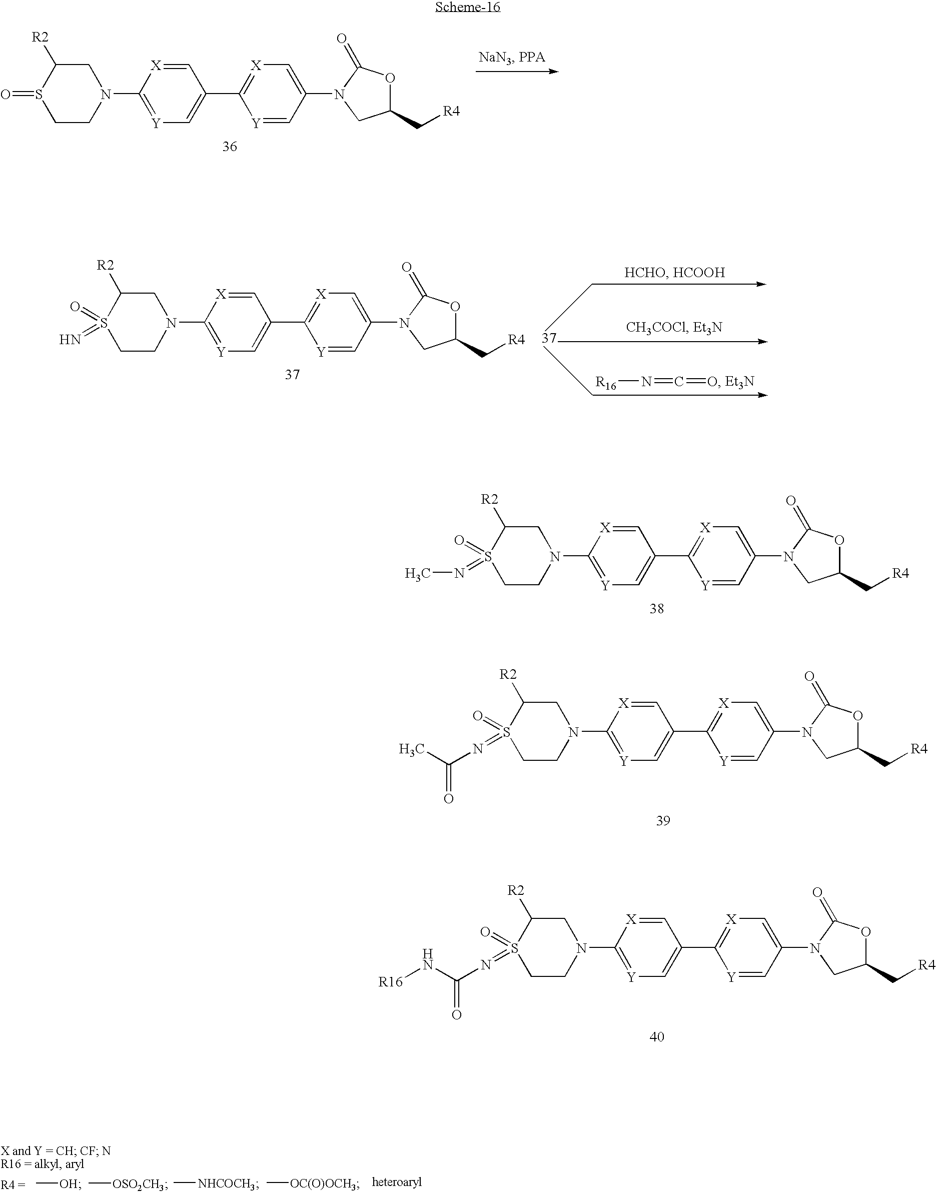

In accordance with scheme-16, oxazolidinone compound 36 (prepared as per procedure described above) is stirred with sodium azide and polyphosphoric acid (PPA) for 10 to 14 hours at a temperature between 50-60° C. to provide oxazolidinone compound 37 of the invention.

The oxazolidinone compound 37, upon reacting with formic acid and aqueous formaldehyde mixture for 10 to 14 hours at a temperature between 60-80° C. provided oxazolidinone compound 38 of the invention.

The oxazolidinone compound 37, upon reacting with alkyl chloride such as acetyl chloride and base such as triethylamine or diisopropyl ethylamine mixture for 10 to 14 hours at a temperature between 0-35° C. provided oxazolidinone compound 39 of the invention.

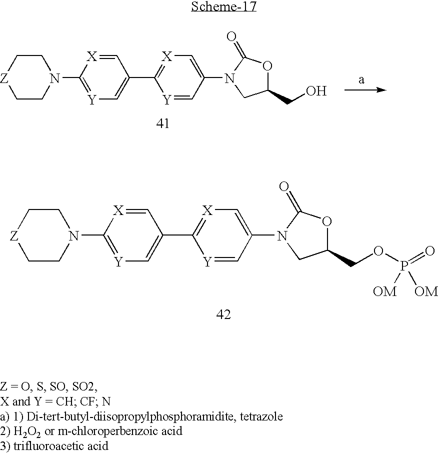

In accordance with scheme-17, oxazolidinone compound 41 (prepared as per procedure described above) is stirred with a mixture of tetrazole and di-tert-butyl-diisopropylphosphoramidite in a mixture of tetrahydrofuran and dichloromethane, for 10 to 14 hours at a temperature between 30-45° C. The reaction mixture is stirred with oxidizing agent such as hydrogen peroxide or m-chloroperbenzoic acid at 0-20° C. temperature for additional 5-6 hours. The crude product is isolated by work up and the isolated product is stirred with trifluoroacetic acid to provide oxazolidinone compound 42 of the invention. The di-sodium salt of the compound 42 is prepared by dissolving compound 42 in aqueous sodium hydroxide and evaporating to the dryness.

Alternatively, compound 42, by using mixture of tetrazole and di-benzyl-diisopropylphosphoramidite, m-chloroperbenzoic acid followed by removal of benzyl group by using catalytic amount of 5-10% Pd on carbon in presence of hydrogen atmosphere. Alternatively, compound 42, by using mixture of phosphorous trichloride, benzyl alcohol and iodine.

Optionally, the compound 41 can be treated with Cl—P(O)(OM)₂, wherein M is methyl, ethyl, t-butyl, phenyl, in presence of triethylamine, N,N-dimethylaminopyridine in a solvent such as dichloromethane at rt for 1-24 h to afford the compound 42, wherein M is methyl, ethyl, t-butyl or phenyl.

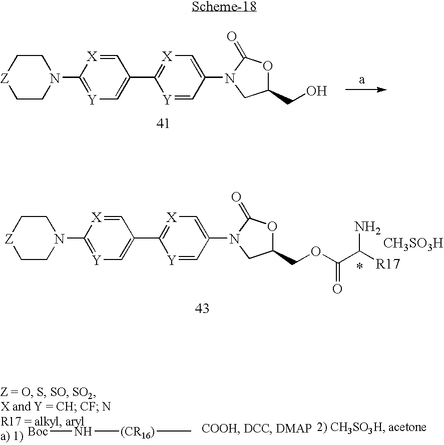

In accordance with scheme-18, oxazolidinone compound 41 (prepared as per procedure described above) is stirred with a mixture of suitably protected amino acid such as N-Boc protected amino acid, dicyclohexylcarbodimide and N,N-dimethylaminopyridine in a solvent such as chloroform and dichloromethane, for 2 to 14 hours at a temperature between −10° C. to 30° C. The crude product is isolated by work up and the isolated product is stirred with methane sulfonic acid in solvent such as acetone, ethylmethyl ketone to provide methane sulfonic acid salt of amino acid ester oxazolidinone compound 42 of the invention.

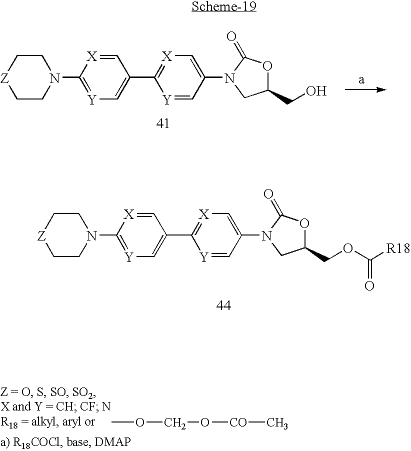

In accordance with scheme-19, oxazolidinone compound 41 (prepared as per procedure described above) is stirred with a mixture of suitable alkyl or aryl or substituted formyl acid chloride such as acetyl chloride or benzoyl chloride or acetoxymethyloxycarbonyl chloride and N,N-dimethylaminopyridine in presence of base such as triethylamine or pyridine or diisopropylethylamine in a solvent such as chloroform and dichloromethane, for 2 to 14 hours at a temperature between 0° C. to 30° C. to provide methane sulfonic acid salt of amino acid ester oxazolidinone compound 44 of the invention.

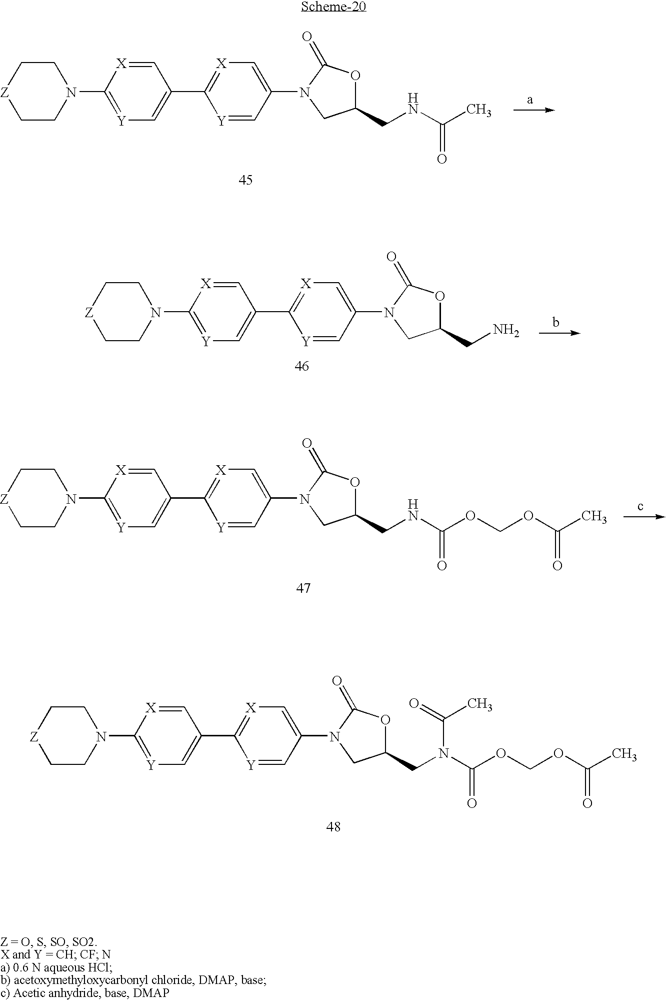

In accordance with scheme-20, oxazolidinone compound 45 (prepared as per procedure described above) is stirred with a 0.6N aqueous hydrochloric acid in a solvent such as methanol or ethanol for 2 to 8 hours at a temperature between 60° C. to 80° C. to provide oxazolidinone compound 46.

The oxazolidinone compound 46 is treated with acetoxymethyloxycarbonyl chloride and N,N-dimethylaminopyridine in presence of base such as triethylamine or pyridine or diisopropylethylamine in a solvent such as chloroform and dichloromethane, for 2 to 14 hours at a temperature between 0° C. to 30° C. to provide oxazolidinone compound 47.

The oxazolidinone compound 47 is treated with acetic anhydride and N,N-dimethylaminopyridine in presence of base such as triethylamine or pyridine or diisopropylethylamine in a solvent such as chloroform and dichloromethane, for 10 to 14 hours at a temperature between 30° C. to 50° C. to provide oxazolidinone compound 48 of the invention.

The oxazolidinone antibacterial agents of this invention have potential for treatment of microbial infections. The microbial infection can be caused by Gram-positive including multi-resistant bacteria, Gram-negative bacteria, anaerobic organism, acid-fast organism. In contrast to compounds of the prior art, they demonstrate therapeutically useful activity against different resistant microorganisms and in particular different strains of Enterococcus faecalis. In addition they display activity against linezolid-resistant S. aureus strains, linezolid-resistant E. faecalis strains and in particular linezolid-resistant S. pneumoniae strains. These compounds are useful for the treatment of Gram-positive or Gram-negative microbial infections in animals and human. The animals which can be treated by bacterial infections include but not limited to birds, mammals, fishes. These compounds are useful for the treatment of microbial infections by either parenteral, oral or topical administration. The infection in human and animals can be systemic or topical.

The compounds of this invention may be used to prevent infections caused by Gram-positive and Gram-negative bacteria by administering the compound to a subject that is at risk for developing an infection caused by Gram-positive or Gram-negative bacteria. A subject at risk for developing an infection may be a health care worker, surgical patient and the like.

The compositions of the present invention include compositions such as suspensions, solutions, elixirs, aerosols, and solid dosage forms. Carriers as described in general above are commonly used in the case of oral solid preparations (such as powders, capsules and tablets), with the oral solid preparations being preferred over the oral liquid preparations. The most preferred oral solid preparation is tablets.

Because of their ease of administration, tablets and capsules represent the most advantageous oral dosage unit form, in which case solid pharmaceutical carriers are employed. Examples of suitable carriers include excipients such as lactose, white sugar, sodium chloride, glucose solution, urea, starch, calcium carbonate, kaolin, crystalline cellulose and silicic acid, binders such as water, ethanol, propanol, simple syrup, glucose, starch solution, gelatin solution, carboxymethyl cellulose, shellac, methyl cellulose, potassium phosphate and polyvinyl pyrrolidone, disintegrants such as dried starch, sodium alginate, agar powder, laminaria powder, sodium hydrogen carbonate, calcium carbonate, Tween (fatty acid ester of polyoxyethylenesorbitan), sodium lauryl sulfate, stearic acid monoglyceride, starch, and lactose, disintegration inhibitors such as white sugar, stearic acid glyceryl ester, cacao butter and hydrogenated oils, absorption promoters such as quaternary ammonium bases and sodium lauryl sulfate, humectants such as glycerol and starch, absorbents such as starch, lactose, kaolin, bentonite and colloidal silicic acid, and lubricants such as purified talc, stearic acid salts, boric acid powder, polyethylene glycol and solid polyethylene glycol.

The tablet, if desired, can be coated, and made into sugar-coated tablets, gelatin-coated tablets, enteric-coated tablets, film-coated tablets, or tablets comprising two or more layers.

If desired, tablets may be coated by standard aqueous or non-aqueous techniques. In molding the pharmaceutical composition into pills, a wide variety of conventional carriers known in the art can be used. Examples of suitable carriers are excipients such as glucose, lactose, starch, cacao butter, hardened vegetable oils, kaolin and talc, binders such as gum arabic powder, tragacanth powder, gelatin, and ethanol, and disintegrants such as laminaria and agar.

In molding the pharmaceutical composition into a suppository form, a wide variety of carriers known in the art can be used. Examples of suitable carriers include polyethylene glycol, cacao butter, higher alcohols, gelatin, and semi-synthetic glycerides.

A second preferred method is parenterally for intramuscular, intravenous or subcutaneous administration.

A third preferred route of administration is topically, for which creams, ointments, shampoos, lotions, dusting powders and the like are well suited. Generally, an therapeutically effective amount of the compound according to this invention in a topical form is from about 0.1% w/w to about 10% w/w of the total composition. Preferably, the therapeutically effective amount of the compound of the invention is 1% w/w of the total composition.

In addition to the common dosage forms set out above, the compounds of the present invention may also be administered by controlled release means and/or delivery devices such as those described in U.S. Pat. Nos. 3,845,770; 3,916,899; 3,536,809; 3,598,123 and 4,008,719; the disclosures of which are hereby incorporated by reference.

Desirably, each tablet contains from about 200 mg to about 1500 mg of the active ingredient. Most preferably, the tablet, cachet or capsule contains either one of four dosages, about 200 mg, about 400 mg, 600 mg or about 800 mg of the active ingredient.

When the pharmaceutical composition is formulated into an injectable preparation, in formulating the pharmaceutical composition into the form of a solution or suspension, all diluents customarily used in the art can be used. Examples of suitable diluents are water, ethyl alcohol, polypropylene glycol, ethoxylated isostearyl alcohol, polyoxyethylene sorbitol, and sorbitan esters. Sodium chloride, glucose or glycerol may be incorporated into a therapeutic agent.

The antimicrobial pharmaceutical composition may further contain ordinary dissolving aids, buffers, pain-alleviating agents, and preservatives, and optionally coloring agents, perfumes, flavors, sweeteners, and other drugs.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g. preservatives, antioxidants, stabilizers, wetting agents, buffers or salts for influencing osmotic pressure, etc. For topical application, also suitable are sprayable aerosol preparations wherein the active ingredient preferably in combination with a solid or liquid inert carrier material.

A specific embodiment of the invention is the preparation of storage stable compositions of the compounds of the invention of formula I. Such stable compositions can be advantageously made through the use of selective stabilizers. Different stabilizers are known to those skilled in the art of making pharmaceutical compositions. Of special utility for making storage stable compositions of the compound of the invention of formula I, stabilizers such as disodium ethylenediaminetetraacetic acid (EDTA), tromethamine, cyclodextrins such as gamma-cyclodextrin, hydroxy-propyl-gamma-cyclodextrin have been found to be useful.

In a specific embodiment of the invention, the pharmaceutical compositions contain an therapeutically effective amount of the active compounds of the invention, its derivatives, salts or hydrates thereof described in this specification as hereinbefore described in admixture with a pharmaceutically acceptable carrier, diluent or excipients, and optionally other therapeutic ingredients.

The invention is further defined by reference to the following examples describing in detail the preparation of the composition of the present invention as well as their utility. It will be apparent to those skilled in the art that many modifications, both to materials and methods may be practiced without departing from the purpose and scope of this invention.

The compounds of this invention are useful antimicrobial agents effective against various humans and veterinary pathogens specially including Linezolid-resistant strains.

Examples of infections that may be treated with the compounds of the present invention include central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, mastitis, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. Specifically, infectious diseases that may be treated with the compounds of the present invention are gram-positive infections such as osteomyelitis, endocarditis and diabetic foot.

The compounds described herein are useful for the treatment or prophylaxis of Gram-positive or Gram-negative microbial infections in humans and other warm-blooded animals. The oxazolidinone antibacterial compounds of this invention are useful for treatment of Gram-positive infections including those, which result from multi-resistant strains. The compounds of this invention are useful antimicrobial agents effective against various humans and veterinary pathogens specially included Linezolid-resistant strains.

In contrast to Linezolid, the compounds described herein demonstrate bactericidal activity against different resistant microorganisms and in particular different strains of Enterococcus faecalis. In addition they display activity against linezolid-resistant S. aureus strains.

For the purpose of this invention the pharmaceutical compositions may contain the active compounds of the invention, their derivatives, salts and hydrates thereof, in a form to be administered alone, but generally in a form to be administered in admixture with a pharmaceutical carrier selected with regard to the intended route of administration and standard pharmaceutical practice. Suitable carriers which can be used are, for example, diluents or excipients such as fillers, extenders, binders, emollients, wetting agents, disintegrants, surface active agents and lubricants which are usually employed to prepare such drugs depending on the type of dosage form.

Any suitable route of administration may be employed for providing the patient with an effective dosage of the compound of the invention their derivatives, salts and hydrates thereof. For example, oral, rectal, vaginal, parenteral (subcutaneous, intramuscular, intravenous), nasal, transdermal, topical and like forms of administration may be employed. Dosage forms include (solutions, suspensions, etc) tablets, pills, powders, troches, dispersions, suspensions, emulsions, solutions, capsules, injectable preparations, patches, ointments, creams, lotions, shampoos and the like.

The prophylactic or therapeutic dose of the compounds of the invention, their derivatives, salts or hydrates thereof, in the acute or chronic management of disease will vary with the severity of condition to be treated, and the route of administration. In addition, the dose, and perhaps the dose frequency, will also vary according to the age, body weight and response of the individual patient. In general, the total daily dose range, for the compounds of the invention, the derivatives, salts or hydrates thereof, for the conditions described herein, is from about 200 mg to about 1500 mg, in single or divided doses. Preferably, a daily dose range should be between about 400 mg to 1200 mg, in single or divided dosage, while most preferably a daily dose range should be between about 500 mg to about 1000 mg in divided dosage. While intramuscular administration may be a single dose or up to 3 divided doses, intravenous administration can include a continuous drip. It may be necessary to use dosages outside these ranges in some cases as will be apparent to those skilled in the art.

Further, it is noted that the clinician or treating physician will know how and when to interrupt, adjust, or terminate therapy in conjunction with individual patient's response. The term “an amount sufficient to eradicate such infections but insufficient to cause undue side effects” is encompassed by the above-described dosage amount and dose frequency schedule. “Antibacterially effective amount” is the amount required to provide a desirable biological effect of restricting the growth of bacteria or killing bacteria.

Pharmaceutical compositions of the present invention suitable for oral administration may be presented as discrete units such as capsules, cachets, or tablets, or aerosol sprays, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the active ingredient with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation.

### Biological Activity

The compounds of this invention are useful antimicrobial agents, effective against various human and veterinary pathogens, including multiple-resistant staphylococci and streptococci, enteroccoci, as well as anaerobic organisms such bacteroides and clostridia species, and acid resistant organisms such as Mycobacterium tuberculosis and Mycobacterium avium.

### TEST EXAMPLE 1

### MIC Test Method

Overnight grown cultures of S. aureus organisms in Tryptic Soya broth were diluted in Mueller Hinton Broth to give optical density matching with MacFarland tube 0.5 standard. Cultures were further diluted 1:10 in Mueller Hinton broth. Using Denley's multipoint inoculator, 10⁴ cells were deposited on Mueller Hinton agar (Difco) containing range of 2 fold dilutions of test compounds. These plates were incubated for 24 hrs at 35° C. and MIC results recorded. MIC is defined as minimum drug concentration that inhibits test organisms. For determining MIC of test compounds against Streptococcus pneumoniae, Mueller Hinton agar containing 5% sheep blood was employed. The following strains were used to screen the compounds of invention

Strain

Staphylococcus aureus ATCC 25923
Staphylococcus aureus 014
Staphylococcus epidermidis 110
Staphylococcus haemolyticus ATCC 25923
Enterococcus faecalis 401
Enterococcus faecium 303
Streptococcus pneumoniae 49619
Streptococcus pneumoniae 706
Streptococcus pyogenes 801
Streptococcus pyogenes 805
Haemophilus influenzae 49247
Mycobacterium tuberculosis
Mycobacterium avium

The MIC values of the selected oxazolidinone compounds of the invention have displayed antibacterial activity for Staphylococcus aureus ATCC 25923 is 0.5 to ≧8 mcg/ml; for Staphylococcus aureus 014 is 0.5 to ≧8 mcg/ml; Staphylococcus epidermidis 110 is 0.5 to ≧8 mcg/ml; Staphylococcus haemolyticus ATCC 25923 is 0.5 to ≧8 mcg/ml; Enterococcus faecalis 401 is 0.5 to ≧8 mcg/ml; Enterococcus faecium. 303 is 0.5 to ≧8 mcg/ml; Streptococcus pneumoniae 49619 is 0.25 to ≧8 mcg/ml; Streptococcus pneumoniae 706 is 0.5 to ≧8 mcg/ml; Streptococcus pyogenes 801; is 0.25 to ≧8 mcg/ml; Streptococcus pyogenes 805 is 0.25 to ≧8 mcg/ml; Haemophilus influenzae 49247 is 2.0 to ≧8 mcg/ml; Mycobacterium avium is 2.0 to ≧8 mcg/ml.

The following examples are provided to further illustrate this invention but they should not be taken as limiting.

### EXPERIMENTAL

### Example-1

### (5S)—N-{3-[4-(4-(2RS-Cyanoaziridin-1-yl)-piperidin-1-yl)-3-fluorophenyl]-2-oxo-oxazolidin-5-ylmethyl}-acetamide;

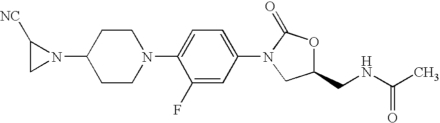

To a solution of (S)—N-{3-[4-(4-amino-piperidin-1-yl)-3-fluoro-phenyl]-2-oxo-oxazolidin-5-ylmethyl}-acetamide (1.37 g, 3.91 mmol) in tetrahydrofuran was added triethylamine (1.18 g, 11.74 mmol) followed by the 2,3-dibromopropionitrile (0.99 g, 4.69 mmol) and the resulting solution stirred at 80° C. for 16 hours. The solvent was removed completely in vacuum and 50 ml water was added to residue and extracted with 3×50 ml ethyl acetate. The combined organic layers were washed with brine, dried (Na₂SO₄) and concentrated to give crude compound. Purification by column chromatography eluting with CHCl₃:CH₃OH (98:2) afforded the pure compound in 75% yield. M.P. 118-120° C. and MS (M+1)=402 (MH⁺, 100%) M.F.=C₂₀H₂₄FN₅O₃.

### Example-2

### (S)—N-{3-[4-(4-pyrrol-1-yl-piperidin-1-yl)-3-fluorophenyl]-2-oxo-oxazolidin-5-ylmethyl}-acetamide;

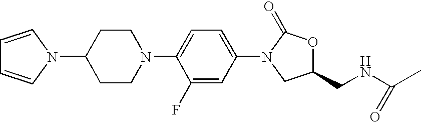

To a solution of (S)—N-{3-[4-(4-Amino-piperidin-1-yl)-3-fluoro-phenyl]-2-oxo-oxazolidin-5-ylmethyl}-acetamide (1.25 mmol) in acetic acid (2.5 ml), water (5 ml) and dichloroethane (10 ml) was added 2,5 dimethoxy tetrahydrofuran (1.25 mmol) at 25° C. and the resulting mixture heated at 80° C. for 3 hour. The organic layer was separated, washed with distilled water (10 ml) and evaporated under reduced pressure to obtain the product as off-white solid, in 70% yield.

M.P. 208-210° C. and MS (M+1)=401 (MH⁺, 100%) M.F.=C₂₀H₂₅FN₄O₃

### Example-3

### (S)—N-{3-[3,5-Difluoro-4-(4-pyrrol-1-yl-piperidin-1-yl)-phenyl]-2-oxo-oxazolidin-5-ylmethyl}-acetamide;

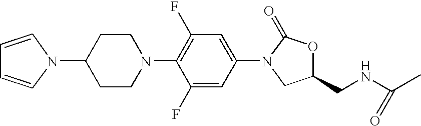

To a solution of (S)—N-{3-[4-(4-amino-piperidin-1-yl)-3,5-difluoro-phenyl]-2-oxo-oxazolidin-5-ylmethyl}-acetamide (1.25 mmol) in acetic acid (2.5 ml), water (5 ml) and dichloroethane (10 ml) was added 2,5 dimethoxy tetrahydrofuran (1.25 mmol) at 25° C. and the resulting mixture heated at 80° C. for 3 hour. The organic layer was separated, washed with distilled water (10 ml) and evaporated under reduced pressure to obtain the product as off-white solid, in 80% yield. M.P. 186-188° C. and MS (M+1)=419 (MH⁺, 100%) M.F.=C₂₀H₂₄F₂N₄O₃

### Example-4

### (S)—N-{3-[4-(4-(1H-imidazol-1-yl)-piperidin-1-yl)-3-fluorophenyl]-2-oxo-oxazolidin-5-ylmethyl}-acetamide;

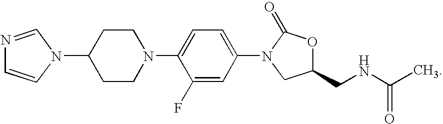

To the solution of (S)—N-{3-[4-(4-(p-toluenesulfonyloxy)-piperidin-1-yl)-3-fluorophenyl]-2-oxo-oxazolidin-5-ylmethyl}-acetamide (0.5 mmol) in dimethyl formamide (5 ml) was added K2CO3 (85 mg, 0.6 mmol) and 1H-[1,2,3]-triazole (45 μl, 0.6 mmol) at room temperature and it was further heated at 70° C. for 15 h. The reaction mixture was slowly poured on the crushed ice and stirred for 15 min. A solid precipitated out was filtered and dried. The crude solid upon purification over silica gel has afforded a white solid in 40% yield. M.P. 160-162° C. and MS (M+1)=402 (MH⁺, 100%) M.F.=C₂₀H₂₄FN₅O₃.

### Example-5 and 6

### (S)—N-{3-[4-(4-(1H-[1,2,3]-triazol-1-yl)-piperidin-1-yl)-3-fluorophenyl]-2-oxo-oxazolidin-5-ylmethyl}-acetamide; and

### (S)—N-{3-[4-(4-(2H-[1,2,3]-triazol-2-yl)-piperidin-1-yl)-3-fluorophenyl]-2-oxo-oxazolidin-5-ylmethyl}-acetamide;

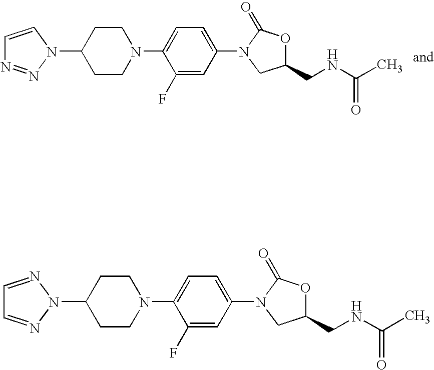

To the solution of (S)—N-{3-[4-(4-(p-toluenesulfonyloxy)-piperidin-1-yl)-3-fluorophenyl]-2-oxo-oxazolidin-5-ylmethyl}-acetamide (0.5 mmol) in DMF (5 ml) was added K₂CO₃ (85 mg, 0.6 mmol) and 1H-[1,2,3]-triazole (45 μl, 0.6 mmol) at room temperature and it was further heated at 70° C. for 15 h. The reaction mixture was slowly poured on the crushed ice and stirred for 15 min. A solid precipitated out was filtered and dried. The crude solid upon purification over silica gel has afforded a non polar isomer as white solid in 52% yield. M.P. 200-1° C. and MS (M+1)=403 (MH⁺, 100%) M.F.=C₁₉H₂₃FN₆O₃ and on further elution afforded a polar isomer as a white solid in 43% yield, polar Isomer M.P. 197° C. and MS (M+1)=403 (MH⁺, 100%) M.F.=C₁₉H₂₃FN₆O₃.

### Example-7

### (S)—N-{3-[4-(4-(1H-[1,2,3]-triazol-1-yl)-piperidin-1-yl)-3,5-difluorophenyl]-2-oxo-oxazolidin-5-ylmethyl}-acetamide;

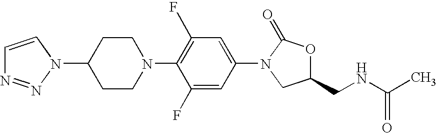

To the solution of (S)—N-{3-[4-(4-(p-toluenesulfonyloxy)-3-fluoro-piperidin-1-yl)-3,5-difluorophenyl]-2-oxo-oxazolidin-5-ylmethyl}-1,2,3-triazol-1-yl (0.5 mmol) in DMF (5 ml) was added K₂CO₃ (0.6 mmol) and 1H-[1,2,3]-triazole (0.6 mmol) at room temperature and it was further heated at 70° C. for 15 h. The reaction mixture was slowly poured on the crushed ice and stirred for 15 min. A solid precipitated out was filtered and dried. The crude solid upon purification over silica gel has afforded a white solid in 55% yield. MP. 188-90° C. and MS (M+1)=421 (MH⁺, 100%) M.F.=C₁₉H₂₂F₂N₆O₃.

### Example-8

### (S)—N-{3-[4-(4-(1H-[1,2,3]-triazol-1-yl)-3-fluoropiperidin-1-yl)-3,5-difluorophenyl]-2-oxo-oxazolidin-5-ylmethyl}-acetamide;

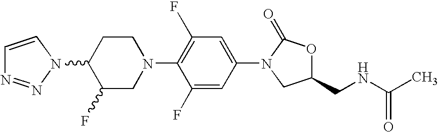

To the solution of (S)—N-{3-[4-(4-(p-toluenesulfonyloxy)-3-fluoro-piperidin-1-yl)-3,5-difluorophenyl]-2-oxo-oxazolidin-5-ylmethyl}-acetamide (0.5 mmol) in DMF (5 ml) was added K₂CO₃ (0.6 mmol) and 1H-[1,2,3]-triazole (0.6 mmol) at room temperature and it was further heated at 70° C. for 15 h. The reaction mixture was slowly poured on the crushed ice and stirred for 15 min. A solid precipitated out was filtered and dried. The crude solid upon purification over silica gel has afforded a pale yellow solid was obtained in 42% yield M.P. 176-78° C. and MS (M+1)=439 (MH⁺, 100%) M.F.=C₁₉H₂₁F₃N₆O₃.

### Example-9

### (S)—N-{3-[4-(4-(1H-[1,2,3]-triazol-1-yl)-3-fluoropiperidin-1-yl)-3-fluorophenyl]-2-oxo-oxazolidin-5-ylmethyl}-acetamide;

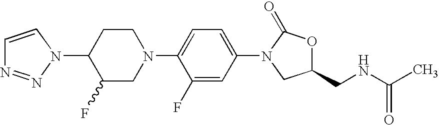

The title compound was prepared by using the method as described above. A white solid was obtained in 48% yield. M.P. 158-60° C. and MS (M+1)=421 (MH⁺, 100%) M.F.=C₁₉H₂₂F₂N₆O₃.

### Example-10

### (S)—N-{3-[4-(4-(tetrazol-5-yl)-piperidin-1-yl)-3-fluorophenyl]-2-oxo-oxazolidin-5-ylmethyl}-acetamide;

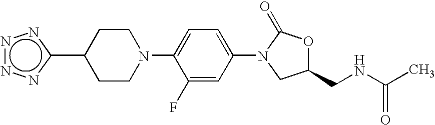

To the solution of N-{3-[4-(4-cyanomethyl-piperidin-1-yl)-3-fluoro-phenyl]-2-oxo-oxazolidin-5-ylmethy}-acetamide (0.8 mmol) in toluene (7 ml) was added trimethylsilyl azide (210 μl, 1.6 mmol) and dibutyltin oxide (40 mg, 0.16 mmol) and the reaction mixture was refluxed at 120° C. for 24 h. The solvent was evaporated under reduced pressure and the residue obtained was purified by the column chromatography to provide a white solid in 66% yield. M.P. 148-150° C. and MS (M+1)=404 (MH⁺, 100%) M.F.=C₁₈H₂₂FN₇O₃.

### Example-11

### (S)—N-{3-[4-(4-(tetrazol-5-yl)-piperidin-1-yl)-3,5-difluorophenyl]-2-oxo-oxazolidin-5-ylmethyl}-acetamide;

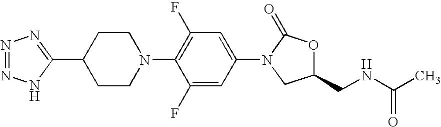

To the solution of N-{3-[4-(4-cyanomethyl-piperidin-1-yl)-3,5-difluoro-phenyl]-2-oxo-oxazolidin-5-ylmethyl}-acetamide (0.8 mmol) in toluene (7 ml) was added trimethylsilyl azide (210 ml, 1.6 mmol) and dibutyltin oxide (40 mg, 0.16 mmol) and the reaction mixture was refluxed at 120° C. for 24 h. The solvent was evaporated under reduced pressure and the residue was purified by the column chromatography, to provide a white solid was obtained in 65% yield.

M.P. 180-82° C. and MS (M+1)=422 (MH⁺, 100%) M.F.=C₁₈H₂₁F₂N₇O₃.

### Example-12 and 13

### (S)—N-{3-[4-(4-(1H-tetrazol-1-yl)-piperidin-1-yl)-3-fluorophenyl]-2-oxo-oxazolidin-5-ylmethyl}-acetamide and

### (S)—N-{3-[4-(4-(2H-tetrazol-2-yl)-piperidin-1-yl)-3-fluorophenyl]-2-oxo-oxazolidin-5-ylmethyl}-acetamide

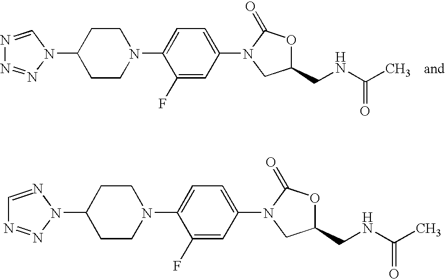

To a stirred solution of (S)—N-{3-[4-(4-(p-toluenesulfonyloxy)-piperidin-1-yl)-3-fluorophenyl]-2-oxo-oxazolidin-5-ylmethyl}-acetamide (1.0 gm, 1.9 mmol) and 1H-tetrazole (0.133 gm, 1.9 mmol) in DMF was added powdered K₂CO₃ (0.262 mg, 1.9 mmol) and heated at 70° C. for 18 hrs. Heating was stopped and cold water was added to the reaction mixture under stirring and then extracted with ethyl acetate (2×75 ml). Combined organic layer was then washed with brine, dried over Na₂SO₄ and evaporated under vacuum. Crude product upon purification by column chromatography over a silica gel using (9.8:0.2) as an eluent afforded a white solid (0.2 g, 25% yield). M.P. 186-188° C. and MS (M+1)=404 (MH⁺, 100%) M.F.=C₁₈H₂₂FN₇O₃. Further elution of the column afforded the polar isomer as a white solid in 17% yield as a cream colored solid. M.P. 189-90° C. and MS (M+1)=404 (MH⁺, 100%) M.F.=C₁₈H₂₂FN₇O₃.

### Example-14 and 15

### (S)—N-{3-[4-(4-(2H-tetrazol-2-yl)-piperidin-1-yl)-3,5-difluorophenyl]-2-oxo-oxazolidin-5-ylmethyl}-acetamide and

### (S)—N-{3-[4-(4-(1H-tetrazol-1-yl)-piperidin-1-yl)-3,5-difluorophenyl]-2-oxo-oxazolidin-5-ylmethyl}-acetamide

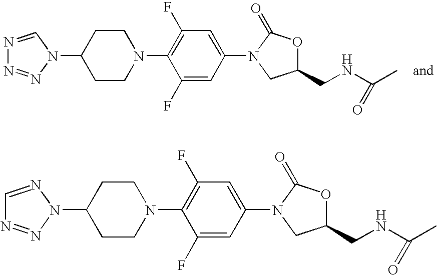

A mixture of (S)—N-{3-[4-methanesulphonyloxy piperidin-1-yl)-3,5-difluorophenyl]-2-oxo-oxazolidin-5-ylmethyl}-acetamide (1.12 mM), tetrazole (1.68 mM), and K₂CO₃ (1.68 mM) in DMF (6 ml) was heated for 22 hrs at 85° C. The resulting mixture was poured into ice-water mixture, stirred for 30 min. And the separated solid was purified by column chromatography to obtain two isomeric products in 18% and 12% yields respectively.

Isomer A: M.P. 234-237° C.; MS (M+1)-422; M.F. C₁₈H₂₁F₂N₇O₃

Isomer B: M.P. 214-217° C.; MS (M+1)-422; M.F. C₁₈H₂₁F₂N₇O₃

### Example-16

### (S)—N-{3-[4-(4-(1H-tetrazol-1-yl)-3-hydroxypiperidin-1-yl)-3-fluorophenyl]-2-oxo-oxazolidin-5-ylmethyl}-acetamide and isomer thereof

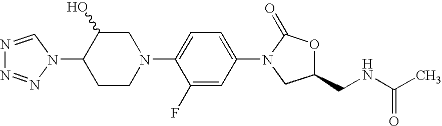

To a stirred solution of (S)—N-{3-[4-(4-toluenesulfonyloxy-3-hydroxypiperidin-1-yl)-3-fluorophenyl]-2-oxo-oxazolidin-5-ylmethyl}-acetamide (2.0 mmol) and 1H-tetrazole (2.5 mmol) in DMF was added powdered K₂CO₃ (2.5 mmol) and heated at 70° C. for 18 hrs. Heating was stopped and cold water was added to the reaction mixture under stirring and then extracted with ethyl acetate (2×75 ml). Combined organic layer was then washed with brine and dried over Na₂SO₄ and evaporated under vacuum. Crude product upon purification by column chromatography over a silica gel using (9.8:0.2) as an eluent afforded white solid in 32% yield. MS (M+1)=420 (MH⁺, 100%) M.F.=C₁₈H₂₂FN₇O₄.

### Example-17

### (5S)—N-{3-[4-(4-(1H-tetrazol-1-yl)-(3RS)-fluoropiperidin-1-yl)-3,5-difluorophenyl]-2-oxo-oxazolidin-5-ylmethyl}-acetamide;

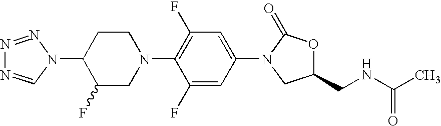

To a stirred solution of (S)—N-{3-[4-(4-toluenesulfonyloxy-3-fluoro-piperidin-1-yl)-3,5-difluorophenyl]-2-oxo-oxazolidin-5-ylmethyl}-acetamide (1.9 mmol) and 1H-tetrazole (1.9 mmol) in DMF was added powdered K₂CO₃ (1.9 mmol) and heated at 70° C. for 18 hrs. Heating was stopped and cold water was added to the reaction mixture under stirring and then extracted with ethyl acetate (2×75 ml). Combined organic layer was then washed with brine and dried over Na₂SO₄ and evaporated under vacuum. Crude product upon purification by column chromatography over a silica gel using (9.8:0.2) as an eluent afforded white solid in 50% yield. M.P. 194-96° C. and MS (M+1)=440 (MH⁺, 100%) M.F.=C₁₈H₂₀F₃N₇O₃.

### Example-18 and 19

### (S)—N-{3-[4-(4-(1-methyl-1H-tetrazol-5-ylmethyl)-piperidin-1-yl)-3-fluoro-phenyl]-2-oxo-oxazolidin-5-ylmethyl}-acetamide; and

### (S)—N-{3-[4-(4-(2-methyl-2H-tetrazol-5-ylmethyl)-piperidin-1-yl)-3-fluoro-phenyl]-2-oxo-oxazolidin-5-ylmethyl}-acetamide;

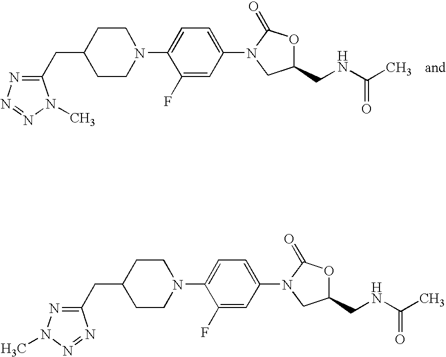

To the solution of N-(3-{3-Fluoro-4-[4-(1H-tetrazol-5-ylmethyl)-piperidin-1-yl]-phenyl}-2-oxo-oxazolidin-5-ylmethyl)-acetamide (150 mg, 0.4 mmol) in THF (3 ml) was added sodium hydride (20 mg, 0.5 mmol) at an ambient temperature and stirred for 15 min. Methyl iodide (27 μl, 0.5 mmol) in THF (1 ml) was added slowly. The reaction mixture was stirred for 15 h. The reaction mixture was quenched by adding ice cold water and it was extracted into ethyl acetate (3×15 ml). The combined organic layer was washed with brine and dried over Na₂SO₄. The residue was obtained on the removal of the solvent was purified by the column chromatography over silica gel using CHCl₃:MeOH (98:2) as an eluent. A white solid was obtained (100 mg, 40%). M.P. 136-138° C. and MS (M+1)=432 (MH⁺, 100%) M.F.=C₂₀H₂₆FN₇O₃.

Upon further elution of the column by CHCl₃:MeOH (95:5) the polar isomer was obtained as a white solid (40 mg, 26%). M.P. 154-56° C. and MS (M+1)=432 (MH⁺, 100%) M.F.=C₂₀H₂₆FN₇O₃.

Using the above procedure and appropriate starting material the following compounds there prepared.

| Example | | | Molecular |
|---|---|---|---|
| No. | Compound | M + 1 | formula |
| 20 | 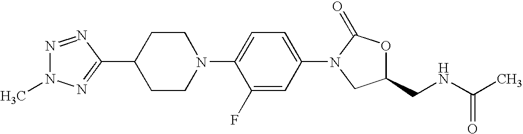 | 418 | C₁₉H₂₄FN₇O₃ |
| 21 | 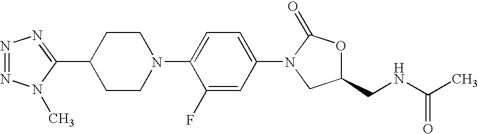 | 418 | C₁₉H₂₄FN₇O₃ |
| 22 | 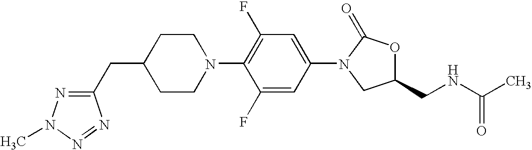 | 450 | C₂₀H₂₅F₂N₇O₃ |
| 23 | 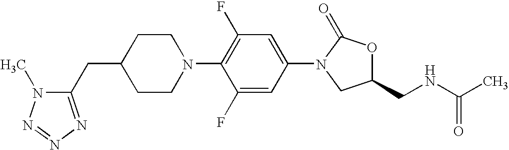 | 450 | C₂₀H₂₅F₂N₇O₃ |
| 24 | 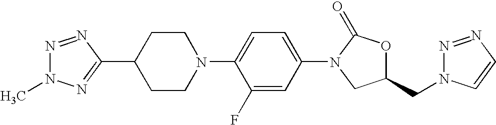 | 428 | C₁₉H₂₂FN₉O₂ |
| 25 | 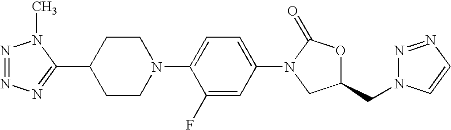 | 428 | C₁₉H₂₂FN₉O₂ |
| 26 | 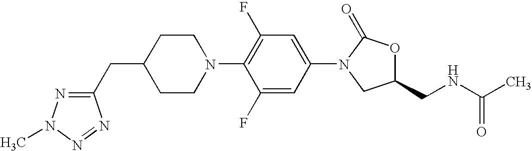 | 450 | C₂₀H₂₅F₂N₇O₃ |
| 27 | 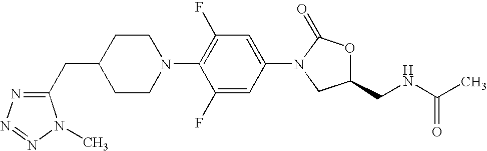 | 450 | C₂₀H₂₅F₂N₇O₃ |

### Example-28

### (S)—N-{3-[4-(4-(4-hydroxymethyl-1H-[1,2,3]-triazol-1-yl)-piperidin-1-yl)-3-fluorophenyl]-2-oxo-oxazolidin-5-ylmethyl}-acetamide

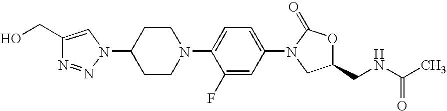

To a stirred solution of (S)—N-{3-[4-(4-(4-ethoxycarbonyl-1,2,3-tetrazol-1-yl)-piperidin-1-yl)-3-fluoro-phenyl]-2-oxo-oxazobidin-5-ylmethyl}-acetamide (0.2 gm, 0.4 mmol) in EtOH (5 ml) was added NaBH₄ (31 mg, 0.8 mmol) and continue to stir for 4 hr. Solvent was evaporated under vacuum. ice-cold water was added to the resulting sticky mass, solid separated was filtered under vacuum and dried. The title compounds was obtained a white solid (0.140 gm). M.P. 160-62° C. and MS (M+1)=433 (MH⁺, 100%) M.F.=C₂₀H₂₅FN₆O₄.

Using the above procedure and appropriate starting material the following compounds were prepared.

| Example | | | Molecular |
|---|---|---|---|
| No. | Compound | M + 1 | formula |
| 29 | 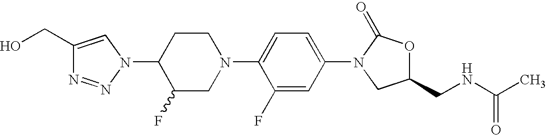 | 451 | C₂₀H₂₄F₂N₆O₄ |
| 30 | 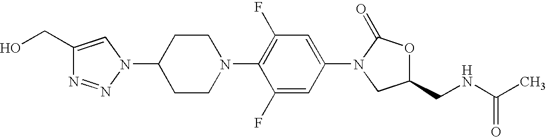 | 451 | C₂₀H₂₄F₂N₆O₄ |

### Example-31

### (R)-{3-[4-(4-(4-hydroxymethyl-1H-[1,2,3]-triazol-1-yl)-piperidin-1-yl)-3-fluorophenyl]-5-(1H-[1,2,3]-triazol-1-ylmethyl)}-oxazolidin-2-one;

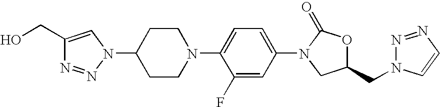

To the stirred solution of (R)-3-[4-(4-azido-piperidin-1-yl)-3-fluoro-phenyl]-5-1,2,3]triazol-1-ylmethyl-oxazolidin-2-one (0.3 g, 0.77 mmol) in toluene (3 ml) was added ethyl propiolate (0.110 □l, 1.2 mmol) and the resulting solution was heated at 120° C. for 15 h. The solvent was removed under reduced pressure and the residual sticky mass on trituration with the ether provided a pale brown solid (180 mg). The solid obtained was taken into the methanol (5 ml) and sodium borohydride (30 mg, 0.7 mmol) was added to it and stirred for 3 h at room temperature. Water was added to the reaction mixture and pH was adjusted to 6. It was then extracted with ethyl acetate (3×15 ml) and the combine organic layer was dried over Na₂SO₄. The residual mass obtained on evaporation of the solvent was subjected to the column chromatographic purification over silica gel using CHCl3:MeOH as an eluent. A white solid was obtained (140 mg, 77%).

M.P. 210-212° C. and MS (M+1)=443 (MH⁺, 100%) M.F.=C₂₀H₂₃FN₈O₃.

### Example-32

### (R)-{3-[4-(4-(4-hydroxymethyl-1H-[1,2,3]-triazol-1-yl)-piperidin-1-yl)-3,5-difluorophenyl]-5-(1H-[1,2,3]-triazol-1-ylmethyl)}-oxazolidin-2-one;

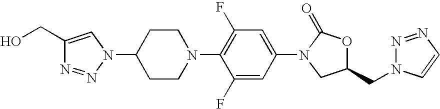

The title compound was prepared by using the procedure mentioned for the above compound, (R)-3-[4-(4-Azido-piperidin-1-yl)-3,5-difluoro-phenyl]-5-1,2,3]triazol-1-ylmethyl-oxazolidin-2-one (0.3 g, 0.70 mmol) has afforded cream colored solid (160 mg, 67%) M.P. 238-40° C. and MS (M+1)=461 (MH⁺, 100%) M.F.=C₂₀H₂₂F₂N₈O₃.

### Example-33

### (S)—N-{3-[4-(4-(4-formyl-1H-[1,2,3]-triazol-1-yl)-piperidin-1-yl)-3-fluorophenyl]-2-oxo-oxazolidin-5-ylmethyl}-acetamide;

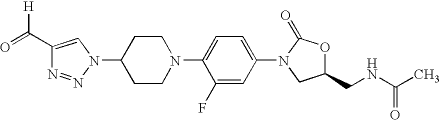

To the solution of (S)—N-{3-[4-(4-(4-(hydroxmethyl-1H-[1,2,3]-triazol-1-yl)-piperidin-1-yl)-3-fluorophenyl]-2-oxo-oxazolidin-5-ylmethyl}-acetamide (0.2 g, 0.46 mmol) in dichloromethane (10 ml) was added Dess-Martin periodinane (0.214 g, 0.5 mmol) and stirred for 2 h at an ambient temperature. The organic layer was washed with water (2×20 ml), dried over Na₂SO₄ and evaporated. The crude product obtained was purified by the column chromatography using CHCl₃:MeOH (9.5:0.5) as an eluent. The white solid was obtained (0.150 g, 75%). M.P. 168-70° C. and MS (M+1)=431 (MH⁺, 100%) M.F.=C₂₀H₂₃FN₆O₄.

Using the above procedure and appropriate starting material the following compounds were prepared.

| Example | | | Molecular |
|---|---|---|---|
| No. | Compound | M + 1 | formula |
| 34 | 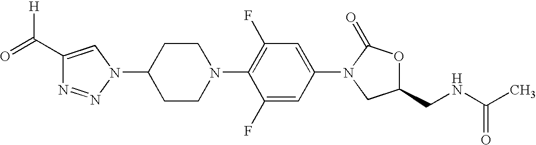 | 449 | C₂₀H₂₂F₂N₆O₄ |
| 35 | 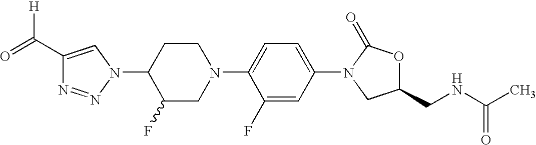 | 449 | C₂₀H₂₂F₂N₆O₄ |
| 36 | 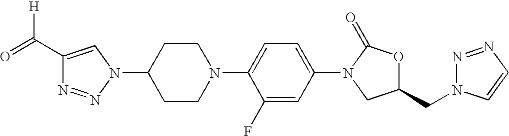 | 441 | C₂₀H₂₁FN₈O₃ |

### Example-37

### (S)—N-{3-[4-(4-(4-difluoromethyl-1H-[1,2,3]-triazol-1-yl)-piperidin-1-yl)-3-fluorophenyl]-2-oxo-oxazolidin-5-ylmethyl}-acetamide;

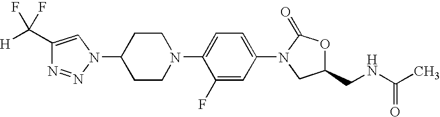

To a stirred solution of (S)—N-{3-[4-(4-(4-formyl-[1,2,3]-triazol-1-yl)-piperidin-1-yl)-3-fluorophenyl]-2-oxo-oxazolidin-5-ylmethyl}-acetamide (0.2 g, 0.46 mmol) in dichloromethane was added DAST (285 ul, 1.76 mmol) at 10° C. slowly and stirred at ambient temperature for 12 h. Reaction mixture was quenched with satd. NaHCO₃ solution and extracted with dichloromethane (15 ml×2). Combined organic layer was dried over sodium sulphate and evaporated under vacuum the residue obtained was purified by column chromatography over silica gel using (95:5) CHCl₃-MeOH as an eluent to provide the title compound as an off-white solid (120 mg) in 60% yield. M.P. 186-88° C. and MS (M+1)=453 (MH⁺, 100%) M.F.=C₂₀H₂₃F₃N₆O₃.

### Example-38

### (S)—N-{3-[4-(4-(4-difluoromethyl-1H-[1,2,3]-triazol-1-yl)-piperidin-1-yl)-3,5-difluorophenyl]-2-oxo-oxazolidin-5-ylmethyl}-acetamide;

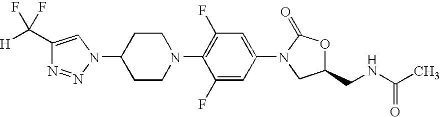

The title compound was prepared by using procedure as described above and by using (S)—N-{3-[4-(4-(4-formyl-[1,2,3]-triazol-1-yl)-piperidin-1-yl)-3-fluorophenyl]-2-oxo-oxazolidin-5-ylmethyl}-acetamide in 55% yield after silica gel column chromatographic purification.

### Example-39

### (S)—N-{3-[4-(4-(4-[1,3]-Dioxolan-2-yl-1H-[1,2,3]-triazol-1-yl)-piperidin-1-yl)-3-fluorophenyl]-5-(1H-[1,2,3]-triazol-1-ylmethyl)}-oxazolidin-2-one;

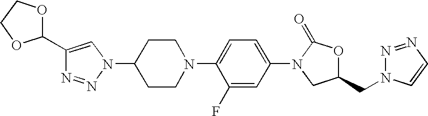

To the solution of (S)—N-{3-[4-(4-(4-formyl-1,2,3-trizoly-1-yl)-piperidin-1-yl)-3,5-difluorophenyl]-2-oxo-oxazolidin-5-ylmethyl}-[1,2,3]-triazol-1-yl (0.25 g, 0.55 mmol) in toluene (5 ml) was added p-toluene sulfonic acid (0.11 mmol) and ethylene glycol (1.1 mmol) The reaction mixture was heated at 120° C. for 3 h. The solvent was removed under reduced pressure and the residue obtained was taken into the ethyl acetate and washed with the aqueous solution of sodium bicarbonate and followed by the brine. The combined organic layer was dried (Na₂SO₄) and the solvent was removed under reduced pressure. The crude solid obtained was purified by the column chromatography over the silica gel by using CHCl₃:MeOH as an eluent. A white solid was obtained in 80% yield. M.P. 198-200° C. and MS (M+1)=485 (MH⁺, 100%) M.F.=C₂₂H₂₅FN₈O₄.

### Example-40a and 40b

### (S)—N-{3-[4-(4-(4-ethoxycarbonyl-1H-[1,2,3]-triazol-1-yl)-methyl-piperidin-1-yl)-3-fluoro-phenyl]-2-oxo-oxazolidin-5-ylmethyl}-acetamide; and

### (S)—N-{3-[4-(4-(5-ethoxycarbonyl-1H-[1,2,3]-triazol-1-yl)-methyl-piperidin-1-yl)-3-fluoro-phenyl]-2-oxo-oxazolidin-5-ylethyl}-acetamide;

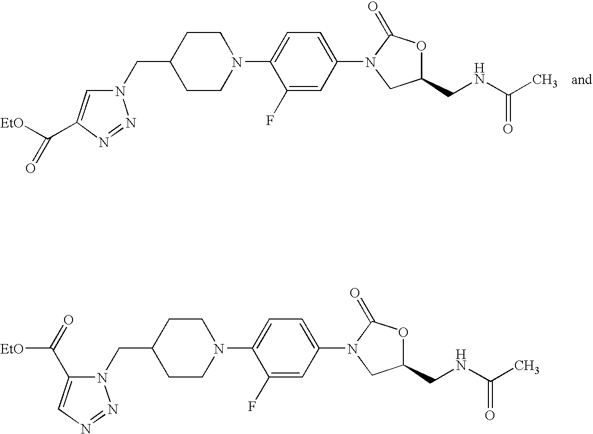

To the stirred solution of (S)—N-{3-[4-(4-azidomethyl-piperidin-1-yl)-3-fluoro-phenyl]-2-oxo-oxazolidin-5-ylmethyl}-acetamide (3 g, 7.69 mmol) in toluene (30 ml) was added ethyl propiolate (2.26 g, 23.07 mmol) and the resulting solution was heated at 115° C. for 5 h. The solvent was removed in-vacuum and 50 ml water was added to the residue. It was extracted with ethyl acetate (3×50 ml) and the combined organic layer was washed with brine, dried (Na₂SO₄) and concentrated to give crude compound as a mixture of two regioisomers. Purification by column chromatography eluting with CHCl₃:CH₃OH (98:2) afforded the non-polar isomer in 1 g quantity along with the other polar isomer in 2.1 g quantity. Non polar isomer: M.P. 75-77° C. and MS (M+1)=489 (MH⁺, 100%) M.F.=C₂₃H₂₉FN₆O₅. Polar isomer: M.P. 150-52° C. and MS (M+1)=489 (MH⁺, 100%) M.F.=C₂₃H₂₉FN₆O₅.

### Example-41

### (S)—N-{3-[4-(4-(4-cyano-1H-[1,2,3]-triazol-1-yl)-piperidin-1-yl)-3-fluorophenyl]-2-oxo-oxazolidin-5-ylmethyl}-acetamide;

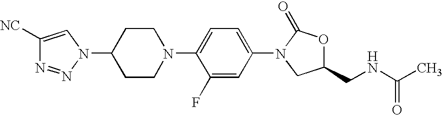

To a stirred solution of (S)—N-{3-[4-(4-(4-(p-toluenesulfonyloxy)-piperidin-1-yl)-3-fluorophenyl]-2-oxo-oxazolidin-5-ylmethyl}-acetamide (1.9 mmol) and 4-cyano-1H-[1,2,3]-triazole (1,9 mmol) in DMF was added powdered K₂CO₃ (0.262 mg, 1.9 mmol) and heated at 70° C. for 18 his. Heating was stopped and cold water was added to the reaction mixture under stirring and then extracted with ethyl acetate (2×75 ml). Combined organic layer was then washed with brine and dried over Na₂SO₄ and evaporated under vacuum. Crude product upon purification by column chromatography over a silica gel using (9.8:0.2) as an eluent afforded white solid in 60% yield. M.P. 148-150° C. and MS (M+1)=428 (MH⁺, 100%) M.F.=C₂₀H₂₂FN₇O₃.

### Example-42

### (S)—N-{3-[4-(4-(4-carboxamido-1H-[1,2,3]-triazol-1-yl)-piperidin-1-yl)-3-fluorophenyl]-2-oxo-oxazolidin-5-ylmethyl}-acetamide;

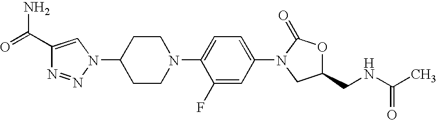

To the solution of N-(3-{4-[4-(4-cyano-1H-[1,2,3]-triazol-1-yl)-piperidin-1-yl]-3-fluoro-phenyl}-2-oxo-oxazolidin-5-ylmethyl)-acetamide (150 g, 0.35 mmol) in t-butyl alcohol (3 ml) was added potassium hydroxide (20 mg, 0.35 mmol) and heated at 80° C. for 1.5 h. To the reaction mixture water (10 ml) was added and the pH was adjusted to 6. It was extracted into ethyl acetate (3×10 ml). The combine organic layer was dried (Na₂SO₄) and evaporation of the solvent provided a crude solid, which was recrystallized from methylenechloride and n-hexane to furnish the pale yellow solid (80 mg, 53%). M.P. 260° C. and MS (M+1)=446 (MH⁺, 100%) M.F.=C₂₀H₂₄FN₇O₄.

### Example-43

### (R)-{3-[4-(4-(4-carboxamido-1H-[1,2,3]-triazol-1-yl)-piperidin-1-yl)-3-fluorophenyl]-5-(1H-[1,2,3]-triazol-1-ylmethyl)}-oxazolidin-2-one;

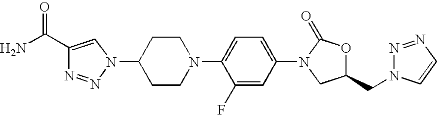

The title compound was prepared by using the same as described for the above compound. A pale yellow solid was obtained in 61% yield. M.P. 284-86° C. and MS (M+1)=456 (MH⁺, 100%) M.F.=C₂₀H₂₂FN₉O₃.

### Example-44

### (S)—N-{3-[4-(4-(4-carboxamido-1H-[1,2,3]-triazol-1-yl)-methyl-piperidin-1-yl)-3-fluoro-phenyl]-2-oxo-oxazolidin-5-ylmethyl}-acetamide;

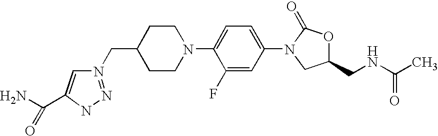

To the stirred solution of (S)—N-{3-[4-(4-(4-ethoxycarbonyl-[1,2,3]-triazol-1-yl)-methyl-piperidin-1-yl)-3-fluoro-phenyl]-2-oxo-oxazolidin-5-ylmethyl}-acetamide (0.9 gm) in acetonitrile (30 ml) was added aqueous ammonia (30 ml) and the resulting solution was stirred at 60° C. for 5 h. The solvent was removed in vacuum & the aqueous layer was extracted with ethyl acetate (3×50 ml). The combined organic layers was washed with brine, dried (Na₂SO₄) and concentrated to give crude compound. The crude compound was triturated and stirred with ether (3×25 ml), filtered, washed with excess of ether & dried completely to get pure compound as white solid (0.2 g). M.P. 165-167° C. and MS (M+1)=460 (MH⁺, 100%) M.F.=C₂₁H₂₆FN₇O₄.

### Example-45

### (S)—N-{3-[4-(4-(5-carboxamido-1H-[1,2,3],-triazol-1-yl)-methyl-piperidin-1-yl)-3-fluoro-phenyl]-2-oxo-oxazolidin-5-ylmethyl}-acetamide;

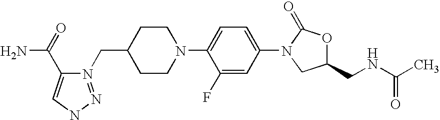

The title compound was prepared by using the same as described for the above compound. A white solid was obtained in 56% yield. MS (M+1)=460 (MH⁺, 100%) M.F.=C₂₁H₂₆FN₇O₄.

### Example-46

### (S)—N-{3-[4-(4-(4-hydroxycarbonyl-1H-[1,2,3]-triazol-1-yl)-methyl-piperidin-1-yl) 3-fluoro-phenyl]-2-oxo-oxazolidin-5-ylmethyl}-acetamide;

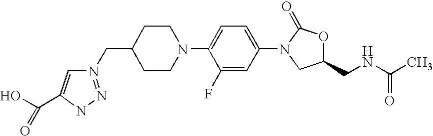

The title compound was obtained by stirring (S)—N-{3-[4-(4-(4-ethoxycarbonyl-1H-[1,2,3]-tetrazol-1-yl)-methyl-piperidin-1-yl)-3-fluoro-phenyl]-2-oxo-oxazolidin-5-ylmeth yl}-acetamide with 0.1 N KOH in tetrahydrofuran at 35° C. for 24 h followed by purification over silica gel by column chromatography in 46% yield. M.P. 172-74° C. and MS (M+1)=461 (MH⁺, 100%) M.F.=C₂₁H₂₅FN₆O₅.

### Example-47

### (S)—N-{3-[4-(4-(5-hydroxycarbonyl-1H-[1,2,3]-triazol-1-yl)-methyl-piperidin-1-yl)-3-fluoro-phenyl]-2-oxo-oxazolidin-5-ylmethyl}-acetamide;

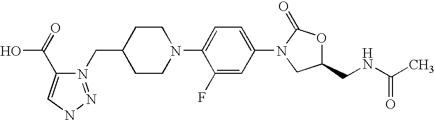

The title compound was prepared by using the same as described for the above compound. A white solid was obtained in 41% yield. M.P. 245-247° C. and MS (M+1)=461 (MH⁺, 100%) M.F.=C₂₁H₂₅FN₆O₅.

### Example-48

### (S)—N-{3-[4-(4-(4-acetyl-1H-[1,2,3]-triazol-1-yl)-piperidin-1-yl)-3-fluorophenyl]-2-oxo-oxazolidin-5-ylmethyl}-acetamide;

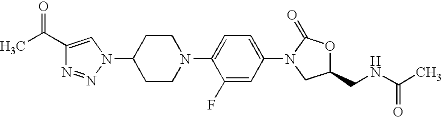

To the solution of (S)—N-{3-[4-(4-azido-piperidin-1-yl)-3-fluorophenyl]-2-oxo-oxazolidin-5-ylmethyl}-acetamide (0.5 mmol) in toluene (5 ml) was added butyne-2-one (50 μl, 0.75 mmol) and heated at 120° C. for 6 h. The solvent was removed under reduced pressure and the residue was purified by the column chromatography over silica gel. A white solid (140 mg, 70%) was obtained. M.P. 220-22° C. and MS (M+1)=445 (MH⁺. 100%) M.F.=C₂₁H₂₅FN₆O₄.

Using the above procedure the following compounds were synthesized

| Example | | | Molecular |
|---|---|---|---|
| No. | Compound | M + 1 | formula |
| 49 | 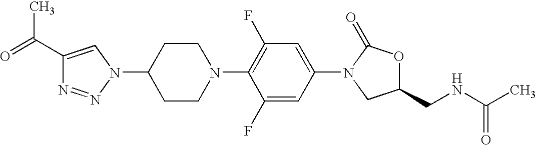 | 463 | C₂₁H₂₄F₂N₆O₄ |
| 50 | 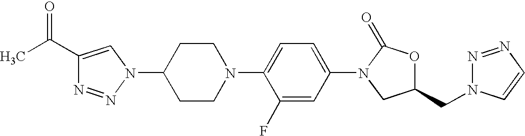 | 455 | C₂₁H₂₃FN₈O₃ |

### Example-51

### (S)—N-{3-[4-(4-(4-acetyl-1H-[1,2,3]-trizol-1-ylmethyl)-1H-[1,2,3]-triazol-1-yl)-piperidin-1-yl)-3-fluorophenyl]-2-oxo-oxazolidin-5-ylmethyl}-acetamide;

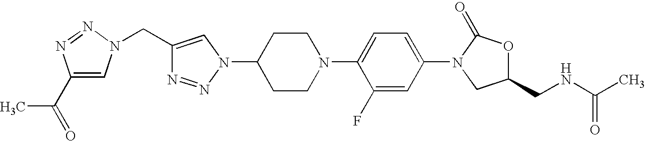

To the solution of (S)—N-(3-{4-[4-(4-azidomethyl-[1,2,3]-triazol-1-yl)-piperidin-1-yl]-3-fluoro-phenyl}-2-oxo-oxazolidin-5-ylmethyl)-acetamide (0.5 mmol) in toluene (5 ml) was added butyne-2-one (0.75 mmol) and heated at 120° C. for 6 h. The solvent was removed under reduced pressure and the residue was purified by the column chromatography over silica gel to afford off-white solid. (160 mg, 60%). M.P. 206-208° C. and MS (M+1)=526 (MH⁺, 100%) M.F.=C₂₄H₂₈FN₉O₄.

### Example-52

### (S)—N-{3-[4-(4-(4-(1RS-hydroxyethyl)-1H-[1,2,3]-triazol-1-yl)-piperidin-1-yl)-3-fluorophenyl]-2-oxo-oxazolidin-5-ylmethyl}-acetamide;

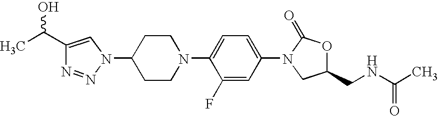

To the solution of (S)—N-(3-{4-[4-(4-acetyl-[1,2,3]triazol-1-yl)-piperidin-1-yl]-3-fluoro-phenyl}-2-oxo-oxazolidin-5-ylmethyl)-acetamide (0.2 g, 0.4 mmol) in methanol (3 ml) was added sodium borohydride (35 mg, 0.8 mmol) at an ambient temperature and stirred for 2 h. The solvent was removed and the residue was taken into the water and the pH was adjusted to 6 and extracted with ethyl acetate (3×15 ml). The combined organic layer was dried (Na₂SO₄) and evaporated. The crude compound obtained was purified by column chromatography over silica gel using CHCl₃:MeOH (98:2) as an eluent. A white solid (115 mg, 58%) was obtained. M.P. 194-96° C. and MS (M+1)=447 (MH⁺, 100%) M.F.=C₂₁H₂₇FN₆O₄.

Using the above procedure the following compounds were synthesized

| Example | | | Molecular |
|---|---|---|---|
| No. | Compound | M + 1 | formula |
| 53 | 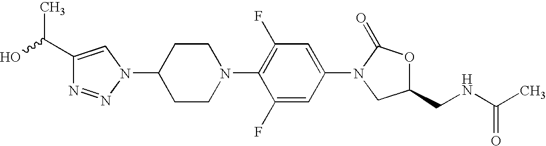 | 465 | C₂₁H₂₆F₂N₆O₄ |
| 54 | 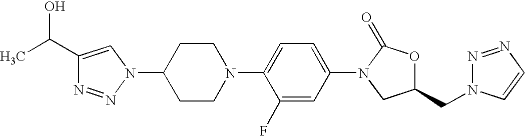 | 457 | C₂₁H₂₅FN₈O₃ |

### Example-55

### (S)—N-{3-[4-(4-(4-(2-methyl-oxiranyl)-1H-[1,2,3]-triazol-1-yl)-piperidin-1-yl)-3-fluorophenyl]-2-oxo-oxazolidin-5-ylmethyl}-acetamide;

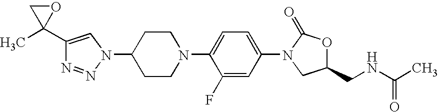

To the solution sodium hydride (60%, 27 mg, 0.66 mmol) in DMSO (1.0 ml) was added trimethylsulfoxonium iodide (132 mg, 0.6 mmol) at an ambient temperature and stirred for 20 min. To this (S)—N-(3-{4-[4-(4-acetyl-[1,2,3]-triazol-1-yl)-piperidin-1-yl]-3-fluoro-phenyl}-2-oxo-oxazolidin-5-yl-methyl)-acetamide (0.25 g, 0.5 mmol) at an ambient temperature and stirred for 2 h. The reaction mixture was quenched by adding crushed ice and extracted with ethyl acetate (3×10 ml). The combined organic layer was dried (Na2SO4) and evaporated. The crude compound obtained was purified by column chromatography over silica gel using CHCl₃:MeOH (98:2) as an eluent. A white solid (0.17 g, 70%) was obtained. M.P. 158-160° C. and MS (M+1)=459 (MH⁺, 100%) M.F.=C₂₂H₂₇FN₆O₄.

### Example-56

### (R)-{3-[4-(4-(4-(2-methyl-oxiranyl)-1H-[1,2,3]-triazol-1-yl)-piperidin-1-yl)-3-fluorophenyl]-5-(1H-[1,2,3]-triazol-1-ylmethyl)}-oxazolidin-2-one;

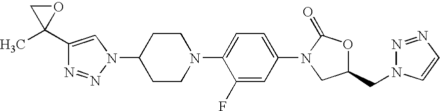

To the solution sodium hydride (60%, 27 mg, 0.66 mmol) in DMSO (1.0 ml) was added trimethylsulfoxonium iodide (132 mg, 0.6 mmol) at an ambient temperature and stirred for 20 min. To this (R)—N-{3-[4-(4-(4-acetyl)-1H-[1,2,3]-triazol-1-yl)-piperidin-1-yl)-3,5-difluorophenyl]-2-oxo-oxazolidin-5-ylmethyl}-1H-[1,2,3]-triazol-1-yl (0.5 mmol) at an ambient temperature and stirred for 2 h. The reaction mixture was quenched by adding crushed ice and extracted with ethyl acetate (3×10 ml). The combined organic layer was dried (Na₂SO₄) and evaporated. The crude compound obtained was purified by column chromatography over silica gel using CHCl₃:MeOH (98:2) as an eluent to provide title compound in 62% yield. M.P. 138-140° C. and MS (M+1)=469 (MH⁺, 100%) M.F.=C₂₂H₂₅FN₈O₃.

### Example-57

### (S)—N-{3-[4-(4-((4-((E/Z)-2-cyano-vinyl)-1H-[1,2,3]-triazol-1-yl)-piperidin-1-yl)-3-fluorophenyl]-2-oxo-oxazolidin-5-ylmethyl}-acetamide;

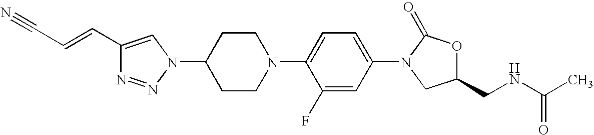

To the solution of (S)—N-{3-[4-(4-(4-formyl)-[1,2,3]-triazol-1-yl)-piperidin-1-yl)-3-fluorophenyl]-2-oxo-oxazolidin-5-ylmethyl}-acetamide (0.2 g, 0.46 mmol) in THF (3.0 ml) was added lithium bromide (60 mg, 0.70 mmol), triethylamine (100 μl, 0.70 mmol) and followed by the diethylcyanomethyl phosphonate (85 ul, 0.50 mmol) and stirred at room temperature for 2 h. The solvent was removed and the residue was taken into chloroform and washed with water and followed by the brine, dried (Na₂SO₄) and on removal of the solvent provided the white solid (0.170 g, 85%). M.P. 214-216° C. and MS (M+1)=454 (MH⁺, 100%) M.F.=C₂₂H₂₄FN₇O₃.

### Example-58

### (S)—N-{3-[4-(4-(4-hydroxyimino-methyl)-1H-[1,2,3]-triazol-1-yl)-piperidin-1-yl)-3-fluorophenyl]-2-oxo-oxazolidin-5-ylmethyl}-acetamide;

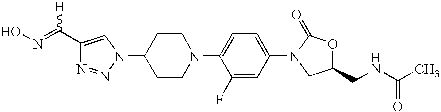

To the solution of the (S)—N-{3-[4-formyl-[1,2,3]-triazol-1-yl)-piperidin-1-yl)-3-fluoro phenyl]-2-oxo-oxazolidin-5-ylmethyl}-acetamide (0.2 g, 0.46 mmol) in methanol (3 ml) was added hydroxylamine hydrochloride (64 mg, 0.92 mmol) and stirred at an ambient temperature for 4 h. Upon evaporation of the solvent under reduced pressure provided a gummy mass, which was taken up into ice cold water and resulting white solid was filtered and dried to provide the oxime (0.160 g, 80%). M.P. 194-96° C. and MS (M+1)=446 (MH⁺, 100%) M.F.=C₂₀H₂₄FN₇O₄.

### Example-59

### (S)—N-{3-[4-(4-(1-methoxyimino-ethyl)-1H-[1,2,3]-triazol-1-yl)-piperidin-1-yl)-3,5-difluorophenyl]-2-oxo-oxazolidin-5-ylmethyl}-acetamide;

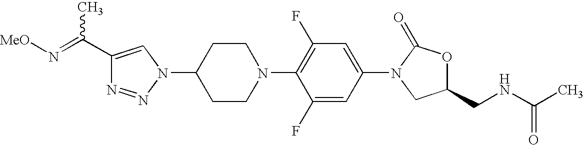

To the solution of (S)—N-{3-[4-(4-(4-(acetyl)-[1,2,3]-triazol-1-yl)-piperidin-1-yl)-3,5-difluorophenyl]-2-oxo-oxazolidin-5-ylmethyl}-acetamide (0.46 mmol) in methanol (3 ml) was added hydroxylamine hydrochloride (0.92 mmol) and stirred at an ambient temperature for 4 h. Upon evaporation of the solvent under reduced pressure provided a gummy mass, which was taken up into ice cold water and resulting white solid was filtered and dried to provide title compound was obtained in 69% yield as a white solid. M.P. 164-166° C. and MS (M+1)=492 (MH⁺, 100%) M.F.=C₂₂H₂₇F₂N₇O₄.

Using the above procedure the following compounds were prepared

| Example | | | Molecular |
|---|---|---|---|
| No. | Compound | M + 1 | formula |
| 60 | 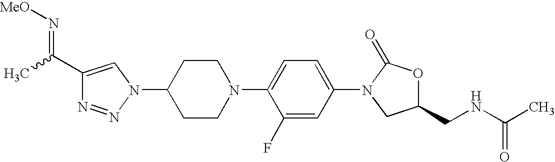 | 474 | C₂₂H₂₈FN₇O₄ |
| 61 | 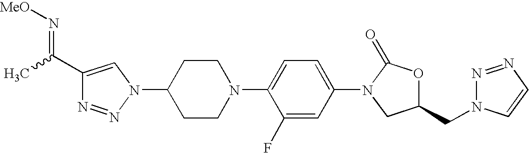 | 484 | C₂₂H₂₆FN₉O₃ |
| 62 | 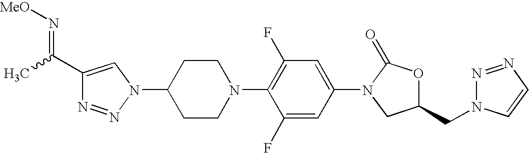 | 502 | C₂₂H₂₅F₂N₉O₃ |

### Example-63

### (S)—N-{3-[4-(4-(1-methoxyimino-methyl)-1H-[1,2,3]-triazol-1-yl)-piperidin-1-yl)-3-fluorophenyl]-2-oxo-oxazolidin-5-ylmethyl}-acetamide;

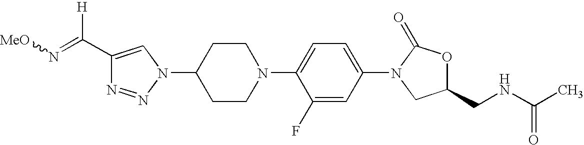

To the solution of (S)—N-{3-[4-(4-(4-(formyl)-[1,2,3]-triazol-1-yl)-piperidin-1-yl)-3-fluorophenyl]-2-oxo-oxazolidin-5-ylmethyl}-acetamide (0.46 mmol) in methanol (3 ml) was added methoxylamine hydrochloride (0.92 mmol) and stirred at an ambient temperature for 4 h. Upon evaporation of the solvent under reduced pressure provided a gummy mass, which was taken up into ice cold water and resulting white solid was filtered and dried to provide title compound in 86% yield. M.P. 198-200° C. and MS (M+1)=460 (MH⁺, 100%) M.F.=C₂₁H₂₆FN₇O₄.

### Example-64

### (S)—N-{3-[4-(4-(1-methoxyimino-methyl)-1H-[1,2,3]-triazol-1-yl)-piperidin-1-yl)-3,5-difluorophenyl]-2-oxo-oxazolidin-5-ylmethyl}-acetamide;

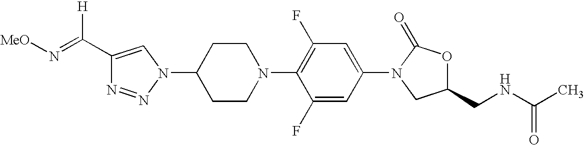

To the solution of (S)—N-{3-[4-(4-(4-(formyl)-[1,2,3]-triazol-1-yl)-piperidin-1-yl)-3,5-difluorophenyl]-2-oxo-oxazolidin-5-ylmethyl}-acetamide (0.46 mmol) in methanol (3 ml) was added methoxylamine hydrochloride (0.92 mmol) and stirred at an ambient temperature for 4 h. Upon evaporation of the solvent under reduced pressure provided a gummy mass, which was taken up into ice cold water and resulting white solid was filtered and dried to provide title compound in 72% yield as buff colored solid. M.P. 154-56° C. and MS (M+1)=478 (MH⁺, 100%) M.F.=C₂₁H₂₅F₂N₇O₄.

### Example-65

### (S)—N-{3-[4-(4-(1-benzyloxyimino-methyl)-1H-[1,2,3]-triazol-1-yl)-piperidin-1-yl)-3-fluorophenyl]-2-oxo-oxazolidin-5-ylmethyl}-acetamide;

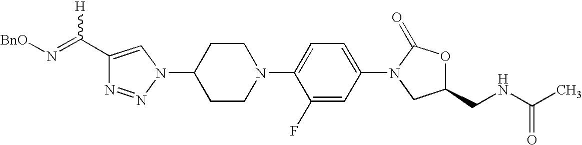

To the solution of (S)—N-{3-[4-(4-(4-(formyl)-[1,2,3]-triazol-1-yl)-piperidin-1-yl)-3-fluorophenyl]-2-oxo-oxazolidin-5-ylmethyl}-acetamide (0.46 mmol) in methanol (3 ml) was added benzyloxyamine hydrochloride (0.92 mmol) and stirred at an ambient temperature for 4 h. Upon evaporation of the solvent under reduced pressure provided a gummy mass, which was taken up into ice cold water and resulting white solid was filtered and dried to provide title compound in 65% yield. M.P. 224-26° C. and MS (M+1)=536 (MH⁺, 100%) M.F.=C₂₇H₃₀FN₇O₄.

### Example-66

### (S)—N-{3-[4-(4-(4-hydroxyiminoamino-methyl)-1H-[1,2,3]-triazol-1-yl)-piperidin-1-yl)-3-fluorophenyl]-2-oxo-oxazolidin-5-ylmethyl}-acetamide;

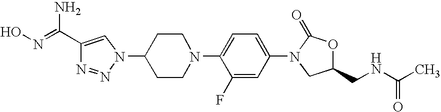

To the solution of (S)—N-{3-[4-(4-cyano-[1,2,3]-triazol-1-yl)-piperidin-1-yl)-3-fluorophenyl]-2-oxo-oxazolidin-5-ylmethyl}-acetamide (0.45 mmol) in methanol was added hydroxylamine hydrochloride (0.9 mmol) and the reaction mixture was heated for 5 h. The solvent was removed under reduced pressure and resulting sticky mass was taken into water (15 ml) and stirred for 10 min. The white solid separated out was filtered and dried to give the title compound (0.16 g, 80%). M.P. 242-44° C. and MS (M+1)=461 (MH⁺, 100%) M.F.=C₂₀H₂₅FN₈O₄.

### Example-67

### (S)—N-{3-[4-(4-(4-N,N-dimethylaminoethylaminomethyl)-1H-[1,2,3]-triazol-1-yl)-piperidin-1-yl)-3-fluorophenyl]-2-oxo-oxazolidin-5-ylmethyl}-acetamide;

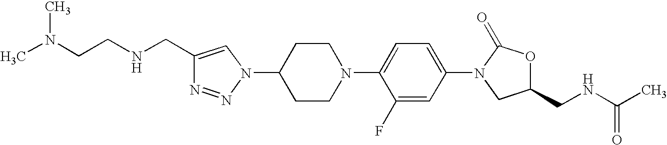

To the solution of (S)—N-{3-[4-(4-(formyl-[1,2,3]-triazol-1-yl)-piperidin-1-yl)-3-fluorophenyl]-2-oxo-oxazolidin-5-ylmethyl}-acetamide (0.5 mmol) in methanol (5.0 ml) was added N,N-dimethylethylene diamine (0.6 mmol) and stirred for 2 h at room temperature. Sodium cyanoborohydride (0.6 mmol) was added to the reaction mixture and further stirred for 5 h at room temperature. The solvent was removed and the residue obtained was taken into the brine and extracted with ethyl acetate (3×10 ml). The combined organic layer was dried (Na₂SO₄) and evaporated to the crude solid which was on successive trituration with the ether has afforded a white solid (160 mg, 80%), M.P. 270(d)° C. and MS (M+1)=503 (MH⁺, 100%) M.F.=C₂₄H₃₅FN₈O₃.

### Example-68 and 69

### (S)—N-{3-[4-(4-(2H-benzotriazol-2-yl)-piperidin-1-yl)-3,5-difluorophenyl]-2-oxo-oxazolidin-5-ylmethyl}-acetamide and

### (S)—N-{3-[4-(4-(1H-benzotriazol-1-yl)-piperidin-1-yl)-3,5-difluorophenyl]-2-oxo-oxazolidin-5-ylmethyl}-acetamide

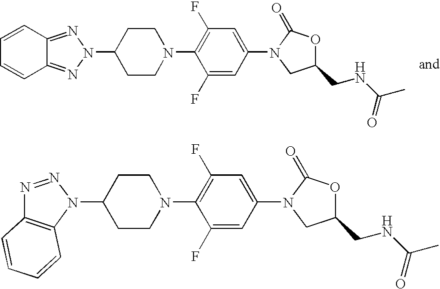

To the solution of (S)—N-{3-[4-(4-Methanesulfonyloxy-piperidin-1-yl)-3,5-difluoro phenyl]-2-oxo-oxazolidin-5-ylmethyl}-acetamide (1.37 mmol) in DMF (10 ml) benzotriazole (2.20 mmol) & K₂CO₃(2.20 mmol) were added and the mixture heated at 80° C. for 14 hour. The reaction mixture was poured in to crushed ice and the separated solid was purified by silica gel column chromatography to obtain two isomeric products in 35 and 30% yield.

Isomer A: M.P. 167-169° C. and MS (M+1)=471.1 (MH⁺, 100%) for M.F.=C₂₃H₂₄F₂N₆O₃.

Isomer B: M.P. 187-189° C. and MS (M+1)=471.1 (MH⁺, 100%) for M.F.=C₂₃H₂₄F₂N₆O₃.

### Example-70 and 71

### (S)—N-{3-[4-(Benzotriazol-2-yl-piperidin-1-yl)-3-fluorophenyl]-2-oxo-oxazolidin-5-ylmethyl}-acetamide and

### (S)—N-{3-[4-(4-Benzotriazol-1-yl-piperidin-1-yl)-3-fluorophenyl]-2-oxo-oxazolidin-5-ylmethyl}-acetamide

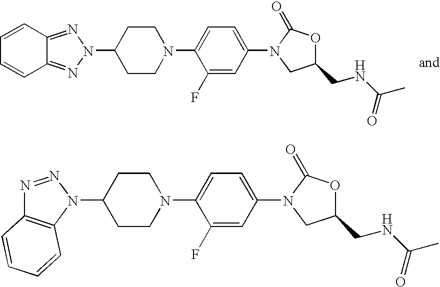

Following the procedure described above and using (S)—N-{3-[4-(4-methanesulfonyloxy-2-yl-piperidin-1-yl)-3-fluorophenyl]-2-oxo-oxazolidin-5-ylmethyl}-acetamide the two products were obtained as isomeric products in 32% and 27% yields respectively Isomer A: M.P. 144-146° C.; MS (M+1)=453.1 (MH⁺, 100%) for M.F. C₂₃H₂₅FN₆O₃.

Isomer B: M.P. −172-174° C. MS (M+1)=453.1 (MH⁺, 100%) for M.F.C₂₃H₂₅FN₆O₃.

### Example-72 and 73

### (S)—N-{3-[4-(4-(4-(1H-tetrazol-1-ylmethyl)-1H-[1,2,3]-triazol-1-yl)-piperidin-1-yl)-3-fluorophenyl]-2-oxo-oxazolidin-5-ylmethyl}-acetamide; and

### (S)—N-{3-[4-(4-(2H-tetrazol-2-ylmethyl)-[1,2,3]-triazol-1-yl)-piperidin-1-yl)-3-fluorophenyl]-2-oxo-oxazolidin-5-ylmethyl}-acetamide;

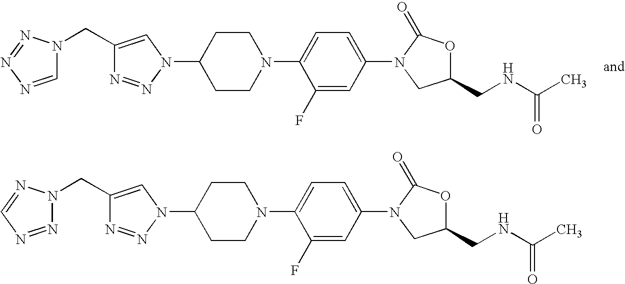

To a stirred solution of (S)—N-{3-[4-(4-(4-p-toluensulfonyloxy methyl)-[1,2,3]-triazol-1-yl)-piperidin-1-yl)-3-fluorophenyl]-2-oxo-oxazolidin-5-ylmethyl}-acetamide (1.9 mmol) and 1H-tetrazole (01.9 mmol) in DMF (5 ml) was added powdered K₂CO₃ (1.9 mmol) and heated at 70° C. for 18 hrs. Heating was stopped and cold water was added to the reaction mixture under stirring and then extracted with ethyl acetate (2×75 ml). Combined organic layer was then washed with brine and dried over Na₂SO₄ and evaporated under vacuum. Crude product upon purification by column chromatography over a silica gel a non polar product A as white solid in 15% yield, M.P. 166-68° C., MS (M+1)=485 (MH⁺, 100%) M.F.=C₂₁H₂₅FN₁₀O₃ and a polar product B in 10% yield, M.P. 154-56° C., MS (M+1)=485 (MH⁺, 100%) M.F.=C₂₁H₂₅FN₁₀O₃.

### Example-74

### (S)—N-{3-[4-(4-(1H-imidazol-1-ylmethyl)-[1,2,3]-triazol-1-yl)-piperidin-1-yl)-3-fluorophenyl]-2-oxo-oxazolidin-5-ylmethyl}-acetamide;

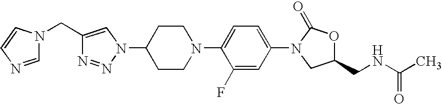

To a stirred solution of (S)—N-{3-[4-(4-(4-p-toluensulfonyloxy methyl)-[1,2,3]-triazol-1-yl)-piperidin-1-yl)-3-fluorophenyl]-2-oxo-oxazolidin-5-ylmethyl}-acetamide (1.9 mmol) and 1H-imidazole (01.9 mmol) in DMF (5 ml) was added powdered K₂CO₃ (1.9 mmol) and heated at 70° C. for 18 hrs. Heating was stopped and cold water was added to the reaction mixture under stirring and then extracted with ethyl acetate (2×75 ml). Combined organic layer was then washed with brine and dried over Na₂SO₄ and evaporated under vacuum. Crude product upon purification by column chromatography over a silica gel provided off white solid in 62% yield M.P. 132-34° C. and MS (M+1)=483 (MH⁺, 100%) M.F.=C₂₃H₂₇FN₈O₃.

### Example-75

### (R)-{3-[4-(4-(1H-[1,2,3]-triazol-1-yl)-3-fluoropiperidin-1-yl)-3-fluorophenyl]-5-(1H-[1,2,3]-triazol-1-ylmethyl)}-oxazolidin-2-one;

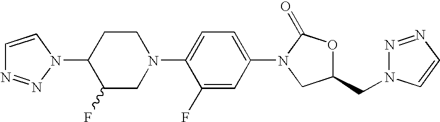

To the solution of (S)—N-{3-[4-(4-(p-toluenesulfonyloxy)-3-fluoro-piperidin-1-yl)-3,5-difluorophenyl]-2-oxo-oxazolidin-5-ylmethyl}-[1,2,3]-triazol-1-yl (0.5 mmol) in DMF (5 ml) was added K2CO3 (0.6 mmol) and 1H-[1,2,3]-triazole (0.6 mmol) at room temperature and it was further heated at 70° C. for 15 h. The reaction mixture was slowly poured on the crushed ice and stirred for 15 min. A solid precipitated out was filtered and dried. The crude solid upon purification over silica gel has afforded a white solid in 35% yield.

M.P. 226-228(d)° C. and MS (M+1)=431 (MH⁺, 100%) M.F.=C₁₉H₂₀F₂N₈O₂.

Using the above procedure the following compounds were prepared

| Example | | | Molecular |
|---|---|---|---|
| No. | Compound | M + 1 | formula |
| 76 | 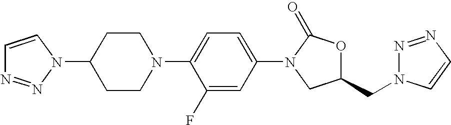 | 413 | C₁₉H₂₁FN₈O₂ |
| 77 | 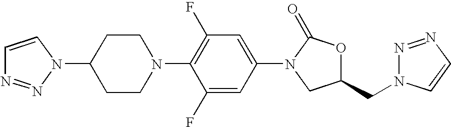 | 431 | C₁₉H₂₀F₂N₈O₂ |
| 78 | 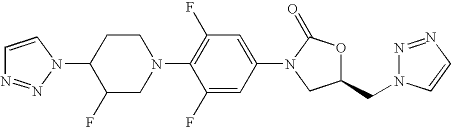 | 449 | C₁₉H₁₉F₃N₈O₂ |

### Example-79

### (S)—N-{3-[4-(4-([1,2,3]-triazol-1-yl)-piperidin-1-yl)-3-fluorophenyl]-2-oxo-oxazolidin-5-ylmethyl}-thioacetamide;

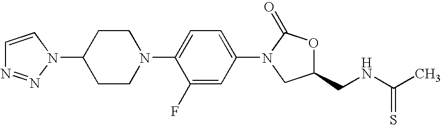

To a stirred solution of (S)—N-{3-[4-(4-([1,2,3]-triazol-1-yl)-piperidin-1-yl)-3-fluorophenyl]-2-oxo-oxazolidin-5-ylmethyl}-acetamide (0.2 gm, 0.55 mmol) in THF (10 ml) was added Lawesson's reagent (0.23 gm, 0.55 mmol). Reaction mixture was stirred at 50-60° C. temperature for 4 hrs. Solvent was evaporated under vacuum and desired compound was purified by column chromatography using CHCl₃-MeOH (98:2) as an eluent affording pale yellow solid in (140 mg) 70% yield. M.P. 171-172° C. and MS (M+1)=419(MH⁺, 100%) M.F.=C₁₉H₂₃FN₆O₂S.

### Example-80 and 81

### (S)—N-{3-[4-(4-(5-methyl-2H-tetrazol-2-yl)-piperidin-1-yl)-3-fluorophenyl]-2-oxo-oxazolidin-5-ylmethyl}-acetamide; and

### (S)—N-{3-[4-(4-(5-methyl-1H-tetrazol-1-yl)-piperidin-1-yl)-3,5-difluorophenyl]-2-oxo-oxazolidin-5-ylmethyl}-acetamide;

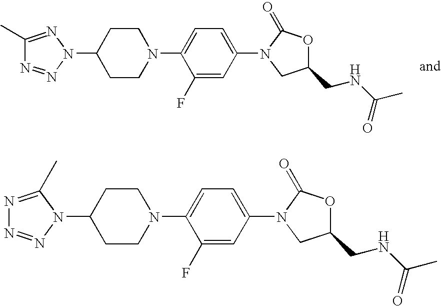

To a solution of (S)—N-{3-[4-(4-methanesulfonyloxy-2-yl-piperidin-1-yl)-3-fluorophenyl]-2-oxo-oxazolidin-5-ylmethyl}-acetamide (1.37 mmol) in DMF (10 ml) were added 5-methyl-1H-tetrazole (2.20 mmol) & K₂CO₃ (2.20 mmol) and the mixture heated at 80° C. for 14 hour. The reaction mixture was poured in to crushed ice and the separated solid was purified by silica gel column chromatography to obtain two isomeric products in 36% and 31% yields.

Isomer A: M.P. 184-186° C.; MS (M+1)=418 (MH⁺, 100%) for M.F. C₁₉H₂₄FN₇O₃

Isomer B: M.P. −205-207° C.; MS (M+1)=418 (MH⁺, 100%) for M.F. C₁₉H₂₄FN₇O₃

### Example-82 and 83

### (S)—N-{3-[4-(4-(5-methyl-1H-tetrazol-1-yl)-piperidin-1-yl)-3,5-difluorophenyl]-2-oxo-oxazolidin-5-ylmethyl}-acetamide; and

### (S)—N-{3-[4-(4-(5-methyl-2H-tetrazol-2-yl)-piperidin-1-yl)-3,5-difluorophenyl]-2-oxo-oxazolidin-5-ylmethyl}-acetamide;

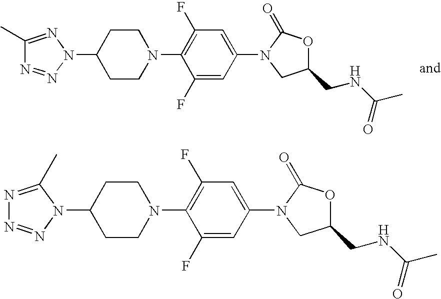

To a solution of (S)—N-{3-[4-(4-methanesulfonyloxy-piperidin-1-yl)-3,5-difluoro phenyl]-2-oxo-oxazolidin-5-ylmethyl}-acetamide (1.37 mmol) in DMF (10 ml were added 5-methyl-1H tetrazole (2.20 mmol) & K₂CO₃ (2.20 mmol) and the mixture heated at 80° C. for 14 hour. The mixture was poured in to crushed ice and the separated solid was purified by silica gel column chromatography to obtain two isomeric products in 29% and 23% yields respectively.

Isomer A: M.P. 214-216° C.; (M+1)=435 (MH⁺, 100%) for M.F. C₁₉H₂₃F₂N₇O₄

Isomer B: M.P. −232-234° C.; (M+1)=435 (MH⁺, 100%) for M.F. CO₉H₂₃F₂N₇O₄.

### Example-84 and 85

### (S)—N-{3-[4-(4-(5-methylsulfanylmethyl-2H-tetrazol-2-yl)-piperidin-1-yl)-3-fluorophenyl]-2-oxo-oxazolidin-5-ylmethyl}-acetamide; and

### (S)—N-{3-[4-(4-(5-methylsulfanylmethyl-1H-tetrazol-1-yl)-piperidin-1-yl)-3-fluorophenyl]-2-oxo-oxazolidin-5-ylmethyl}-acetamide;

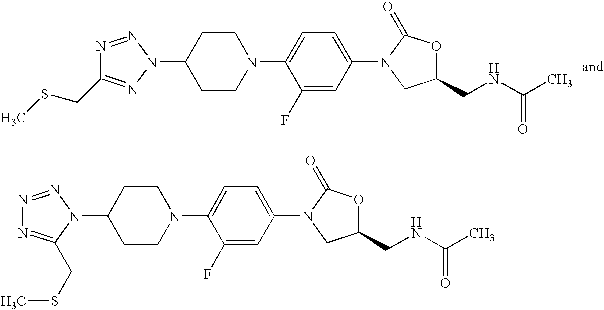

To a stirred solution of (S)—N-{3-[4-(4-p-toluensulfonyloxy-piperidin-1-yl)-3-fluoro phenyl]-2-oxo-oxazolidin-5-ylmethyl}-acetamide (1.0 gm, 1.9 mmol) and 5-methylsulfanylmethyl-1H-tetrazole (0.26 g, 0.2 mol) in DMF (5 ml) was added powdered K₂CO₃ (0.262 mg, 1.9 mmol). The reaction mixture was heated at 70° C. for 18 h. Heating was stopped and cold water was added to the reaction mixture under stirring and then extracted with ethyl acetate (2×75 ml). Combined organic layer was then washed with brine and dried over Na₂SO₄ and evaporated under vacuum. Crude product upon purification by column chromatography over a silica gel using (9.8:0.2) as an eluent afforded white solid (non-polar isomer, 150 mg, 15%) as a cream colored solid. M.P. 118-119° C. and MS (M+1)=464 (MH⁺, 100%) M.F.=C₂₀H₂₆FN₇O₃S, and second isomer was obtained in 12% yield as a pale solid. M.P. 199-200° C. and MS (M+1)=464 (MH⁺, 100%) M.F.=C₂₀H₂₆FN₇O₃S.

### Example-86 and 87

### (S)—N-{3-[4-(4-(5-benzylsulfanyl-1H-tetrazol-1-yl)-piperidin-1-yl)-3,5-difluorophenyl]-2-oxo-oxazolidin-5-ylmethyl}-acetamide; and

### (S)—N-{3-[4-(4-(5-(benzylsulfanyl)-2H-tetrazol-2-yl)-piperidin-1-yl)-3,5-difluorophenyl]-2-oxo-oxazolidin-5-ylmethyl}-acetamide;

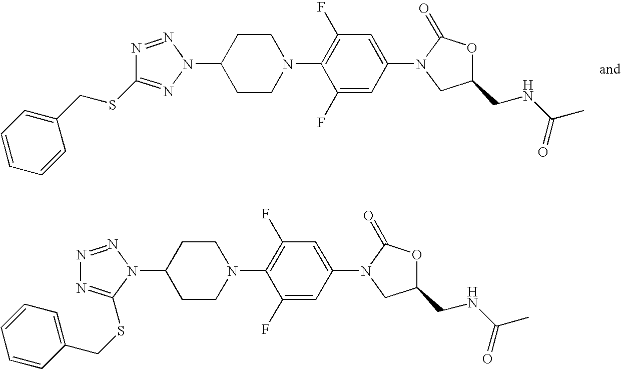

To a solution of (S)—N-{3-[4-(4-Methanesulfonyloxy-2-yl-piperidin-1-yl)-3,5-di fluorophenyl]-2-oxo-oxazolidin-5-ylmethyl}-acetamide (1.37 mmol) in DMF (10 ml) were added 5(thiobenzyl)-1H-tetrazole (3.01 mmol)&K₂CO₃ (2.05 mmol) and the mixture heated at 80° C. for 14 hour. The reaction mixture was poured in to crushed ice and the separated solid was purified by silica gel column chromatography to obtain two isomeric products in 30% and 25% yields respectively.

Isomer A: M.P. 152-154° C. and MS (M+1)=544 (MH⁺, 100%) for M.F. C₂₅H₂₇F₂N₇O₃S

Isomer B: M.P. 146-148° C. and MS (M+1)=544(MH⁺, 100%) for M.F. C₂₅H₂₇F₂N₇O₃S

### Example-88 and 89

### (S)—N-{3-[4-(4-(5-(benzylsulfanyl)-2H-tetrazol-2-yl)-piperidin-1-yl)-3-fluorophenyl]-2-oxo-oxazolidin-5-ylmethyl}-acetamide; and

### (S)—N-{3-[4-(4-(5-(benzylsulfanyl)-2H-tetrazol-2-yl)-piperidin-1-yl)-3-fluorophenyl]-2-oxo-oxazolidin-5-ylmethyl}-acetamide;

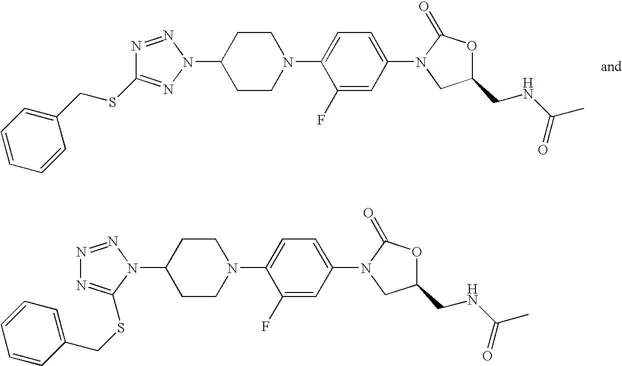

To a solution of (S)—N-{3-[4-(4-methanesulfonyloxy-2-yl-piperidin-1-yl)-3-fluorophenyl]-2-oxo-oxazolidin-5-ylmethyl}-acetamide (1.37 mmol) in DMF (10 ml) were added 5-(thiobenzyl)-1H-tetrazole (3.01 mmol) & K₂CO₃ (2.05 mmol) and the mixture heated at 80° C. for 14 hour. The reaction mixture was poured in to crushed ice and the separated solid was purified by silica gel column chromatography to obtain two isomeric products in 28% and 26% yields respectively

Isomer A: M.P. 178-180° C. and MS (M+1)=525 (MH⁺, 100%) for M.F. C₂₅H₂₈FN₇O₃S

Isomer B: M.P. 144-146° C. and MS (M+1)=525(MH⁺, 100%) for M.F. C₂₅H₂₈FN₇O₃S

### Example-90

### (S)—N-{3-[4-(4-(5-methylsulfonylmethyl-1H-tetrazol-1-yl)-piperidin-1-yl)-3,5-difluorophenyl]-2-oxo-oxazolidin-5-ylmethyl}-acetamide;

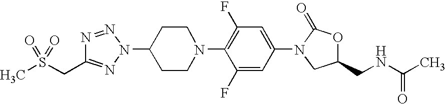

By following the procedure as described in above example and by using (S)—N-{3-[4-(4-p-toluensulfonyloxy-piperidin-1-yl)-3,5-difluorophenyl]-2-oxo-oxazolidin-5-ylmethyl}-acetamide, 5-methylsulfonylmethyl-1H-tetrazole and K₂CO₃, followed by purification by column chromatography over a silica gel using (9.8:0.2) as an eluent afforded white solid non-polar isomer, in 15% yield as a cream colored solid. M.P. 192° C. and MS (M+1)=514 (MH⁺, 100%) M.F. C₂₀H₂₅F₂N₇O₅S.

### Example-91

### (S)—N-{3-[4-(4-(5-methylsulfonylmethyl-1H-tetrazol-1-yl)-piperidin-1-yl)-3-fluorophenyl]-2-oxo-oxazolidin-5-ylmethyl}-acetamide

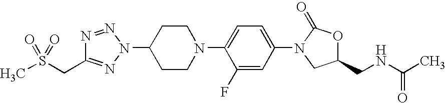

By following the procedure as described in above example and by using (S)—N-{3-[4-(4-p-toluensulfonyloxy-piperidin-1-yl)-3-fluorophenyl]-2-oxo-oxazolidin-5-ylmethyl}-acetamide, 5-methylsulfonylmethyl-1H-tetrazole and K₂CO₃, followed by purification by column chromatography over a silica gel using (9.8:0.2) as an eluent afforded white solid non-polar isomer, in 15% yield as a cream colored solid. M.P. 192° C. and MS (M+1)=496 (MH⁺, 100%) M.F.=C₂₀H₂₆FN₇O₅S.

### Example-92 and 93

### (S)—N-{3-[4-(4-(5-(thiophen-2-yl-methyl)-2H-tetrazol-2-yl)-piperidin-1-yl)-3-fluorophenyl]-2-oxo-oxazolidin-5-ylmethyl}-acetamide; and

### (S)—N-{3-[4-(4-(5-(thiophen-2-yl-methyl)-1H-tetrazol-1-yl)-piperidin-1-yl)-3-fluorophenyl]-2-oxo-oxazolidin-5-ylmethyl}-acetamide;

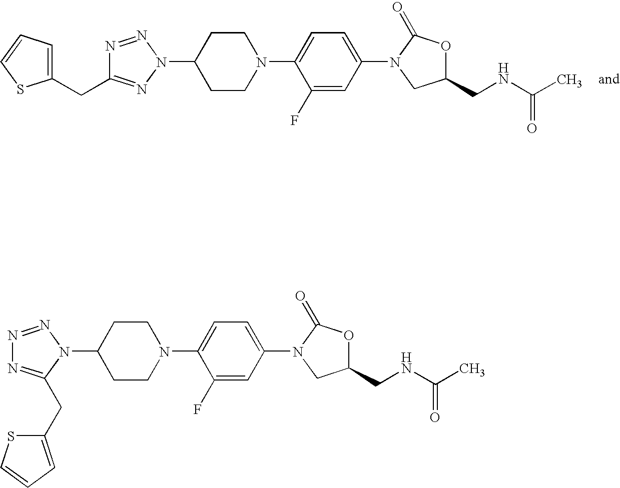

By following the procedure as described in above example and by using (S)—N-{3-[4-(4-p-toluensulfonyloxy-piperidin-1-yl)-3-fluorophenyl]-2-oxo-oxazolidin-5-ylmethyl}-acetamide, 5-thiophen-2-ylmethyl-1H-tetrazole and K₂CO₃, followed by purification by column chromatography over a silica gel using (9.8:0.2) as an eluent afforded white solid non-polar isomer, in 20% yield as a cream colored solid. M.P. 149-150° C. and MS (M+1)=500 (MH⁺, 100%) M.F.=C₂₃H₂₆FN₇O₃S.

and further elution gave second compound in 15% yield. M.P. 171-172° C. and MS (M+1)=500 (MH⁺, 100%) M.F.=C₂₃H₂₆FN₇O═S.

### Example-94 and 95

### (S)—N-{3-[4-(4-(5-(thiophen-2-yl-methyl)-1H-tetrazol-1-yl)-piperidin-1-yl)-3,5-difluorophenyl]-2-oxo-oxazolidin-5-ylmethyl}-acetamide; and

### (S)—N-{[4-(4-(5-(thiophen-2-yl-methyl)-2H-tetrazol-2-yl)-3,5-difluorophenyl]-2-oxo-oxazolidin-5-ylmethyl}-acetamide;

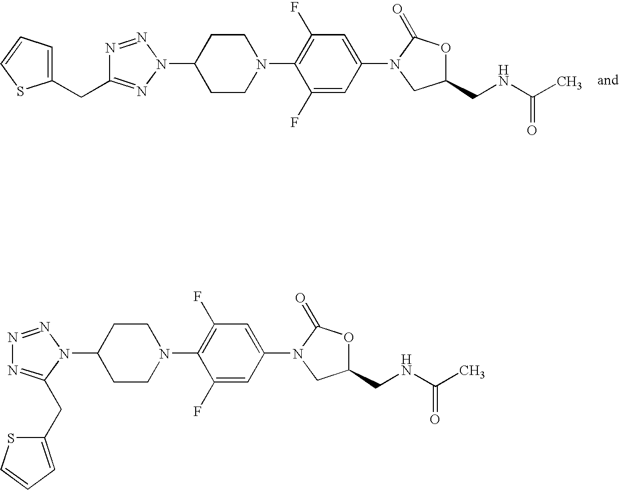

By following the procedure as described in above example and by using (S)—N-{3-[4-(4-p-toluensulfonyloxy-piperidin-1-yl)-3,5-difluorophenyl]-2-oxo-oxazolidin-5-ylmethyl}-acetamide, 5-thiophene2-methyl-1H-tetrazole and K₂CO₃, followed by purification by column chromatography over a silica gel using (9.8:0.2) as an eluent afforded white solid non-polar isomer, in 28% yield as a cream colored solid. MP 142-144° C. and MS (M+1)=518 (MH⁺, 100%) M.F.=C₂₃H₂₅F₂N₇O₃S.

Further elution afforded polar isomer as white solid in 12% yield. M.P. 204-206° C. and MS (M+1)=518 (MH⁺, 100%) M.F.=C₂₃H₂₅F₂N₇O₃S.

### Example-96

### (S)—N-{3-[4-(4-(5-(morpholinocarbonylmethyl)-1H-tetrazol-1-yl)-piperidin-1-yl)-3-fluorophenyl]-2-oxo-oxazolidin-5-ylmethyl}-acetamide;

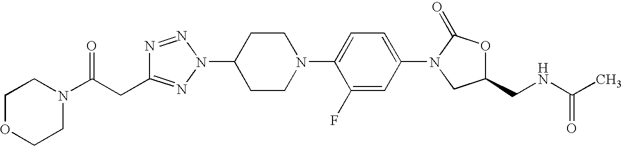

By following the procedure as described in above example and by using (S)—N-{3-[4-(4-p-toluensulfonyloxy-piperidin-1-yl)-3-fluorophenyl]-2-oxo-oxazolidin-5-ylmethyl}-acetamide, 1-Morpholin-4-yl-2-(2H-tetrazol-5-yl)-ethanone and K₂CO₃, followed by purification by column chromatography over a silica gel using (9.8:0.2) as an eluent afforded white solid non-polar isomer, in 20% yield as a cream colored solid. M.P. 146-48° C. and MS (M+1)=532 (MH⁺, 100%) M.F.=C₂₄H₃₁FN₈O₅.

### Example-97

### (S)—N-{3-[4-(4-(5-(morpholinocarbonylmethyl)-1H-tetrazol-1-yl)-piperidin-1-yl)-3,5-difluorophenyl]-2-oxo-oxazolidin-5-ylmethyl}-acetamide;

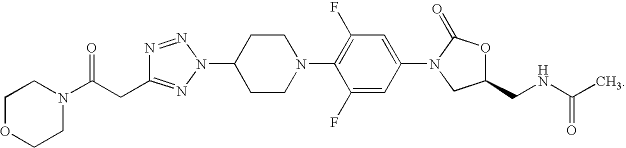

The title compound is prepared as per the procedure mentioned for the above compound. Use of toluene-4-sulfonic acid 1-{4-[5-(acetylamino-methyl)-2-oxo-oxazolidin-3-yl]-3, 5-difluoro-phenyl}-piperidin-4-yl ester (1.0 gm, 2.0 mm) and 1-Morpholin-4-yl-2-(2H-tetrazol-5-yl)-ethanone (0.430 g, 2.2 mol) in DMF was added powdered K₂CO₃ (0.276 mg, 2.0 mm) has afforded the title compound as non-polar isomer (165 mg, 17%) as white solid. M.P. 128-130° C. and MS (M+1)=549 (MH⁺, 100%) M.F.=C₂₄H₃₀F₂N₈O₅.

### Example-98

### (S)—N-{3-[4-(4-(5-(morpholin-4-yl)-2H-tetrazol-2-yl)-piperidin-1-yl)-3-fluorophenyl]-2-oxo-oxazolidin-5-ylmethyl}-acetamide;

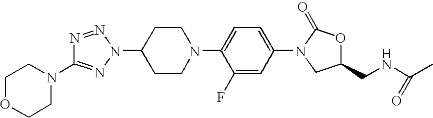

To a solution of (S)—N-{3-[4-(4-methanesulfonyloxy-2-yl-piperidin-1-yl)-3-fluoro phenyl]-2-oxo-oxazolidin-5-ylmethyl}-acetamide (1.37 mmol) in DMF (10 ml) were added 5-Morpholin-4-yl-1H-1-tetrazole (3.01 mmol) & K₂CO₃ (2.05 mmol) and the mixture heated at 80° C. for 14 hour. The reaction mixture was poured in to crushed ice and the separated solid was purified by silica gel column chromatography to obtain the product in 35% yield M.P. 210-214° C. and MS (M+1)=489 (MH⁺, 100%) for M.F.=C₂₂H₂₅FN₈O₄.

### Example-99

### (S)—N-{3-[4-(4-(5-(morpholin-4-yl)-2H-tetrazol-2-yl)-piperidin-1-yl)-3,5-difluorophenyl]-2-oxo-oxazolidin-5-ylmethyl}-acetamide;

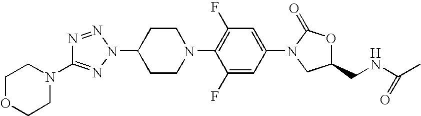

To a solution of (S)—N-{3-[4-(4-methanesulfonyloxy-2-yl-piperidin-1-yl)-3,5-difluorophenyl]-2-oxo-oxazolidin-5-ylmethyl}-acetamide (1.37 mmol) in DMF (10 ml) were added 5-morpholin-4-yl-1H-tetrazole (3.01 mmol) & K₂CO₃ (2.05 mmol) and the mixture heated at 80° C. for 14 hour. The reaction mixture was poured in to crushed ice and the separated solid was purified by silica gel column chromatography to obtain the product in 60% yield. M.P. 232-234° C. and MS (M+1)=507 (MH⁺, 100%) for M.F.=C₂₂H₂₇F₂N₈O₄.

### Example-100

### ((S)—N-{3-[4-(4-(5-phenyl-2H-tetrazol-2-yl)-piperidin-1-yl)-3-fluorophenyl]-2-oxo-oxazolidin-5-ylmethyl}-acetamide;

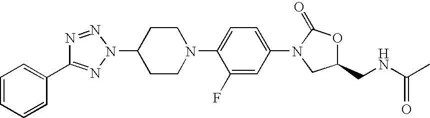

To a solution of (S)—N-{3-[4-(4-methanesulfonyloxy-2-yl-piperidin-1-yl)-3-fluorophenyl]-2-oxo-oxazolidin-5-ylmethyl}-acetamide (1.37 mmol) in DMF (10 ml) were added phenyl 1H-tetrazole (3.01 mmol) & K₂CO₃ (2.05 mmol) and the mixture heated at 80° C. for 14 hour. The reaction mixture was poured in to crushed ice and the separated solid was purified by silica gel column chromatography to obtain the product in 45% yield M.P. 212-214° C. and MS (M+1)=480(MH⁺, 100%) for M.F.=C₂₄H₂₅FN₇O₃.

### Example-101

### (S)—N-{3-[4-(4-(5-(4-Nitrophenyl tetrazol)-2-yl-piperidin-1-yl)-3-fluorophenyl]-2-oxo-oxazolidin-5-ylmethyl}-acetamide;

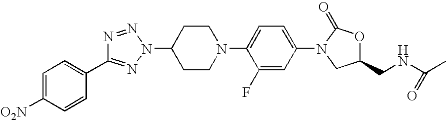

To a solution of (S)—N-{3-[4-(4-methanesulfonyloxy-2-yl-piperidin-1-yl)-3-fluorophenyl]-2-oxo-oxazolidin-5-ylmethyl}-acetamide (1.37 mmol) in DMF (10 ml) were added 4-nitrophenyl-1H-tetrazole (3.01 mmol) & K₂CO₃ (2.05 mmol) and the mixture heated at 80° C. for 14 hour. The reaction mixture was poured in to crushed ice and the separated solid was purified by silica gel column chromatography to obtain the product in 56% yield. M.P. 184-186° C. and MS (M+1)=525 (MH⁺, 100%) for M.F.=C₂₄H₂₅FN₈O₅.

### Example-102

### (S)—N-{3-[4-(5-(4-Nitrophenyl-tetrazol)-2-yl-piperidin-1-yl)-3,5-difluorophenyl]-2-oxo-oxazolidin-5-ylmethyl}-acetamide;

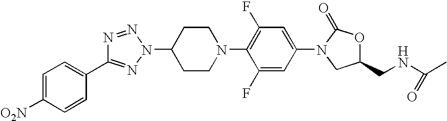

To a solution of (S)—N-{3-[4-(4-methanesulfonyloxy-2-yl-piperidin-1-yl)-3,5-difluoro phenyl]-2-oxo-oxazolidin-5-ylmethyl}-acetamide (1.37 mmol) in DMF (10 ml) were added 5(4-nitrophenyl)-1H-tetrazole (3.01 mmol) & K₂CO₃ (2.05 mmol) and the mixture heated at 80° C. for 14 hour. The reaction mixture was poured in to crushed ice and the separated solid was purified by silica gel column chromatography to obtain the product in 68% yield. M.P. 203-205° C. and MS (M+1)=543 (MH⁺, 100%) for M.F.=C₂₄H₂₄F₂N₈O₅.

### Example-103

### (S)—N-{3-[4-(4-(5-Pyridin-3-yl-tetrazol)-2-yl-piperidin-1-yl)-3-fluorophenyl]-2-oxo-oxazolidin-5-ylmethyl}-acetamide;

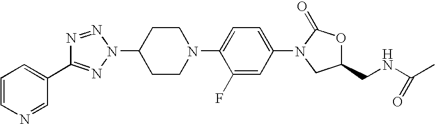

To a solution of (S)—N-{3-[4-(4-methanesulfonyloxy-2-yl-piperidin-1-yl)-3-fluorophenyl]-2-oxo-oxazolidin-5-ylmethyl}-acetamide (1.37 mmol) in DMF (10 ml) were added 5-pyridin-3-yl-1H-tetrazole (3.01 mmol) & K₂CO₃ (2.05 mmol) and the mixture heated at 80° C. for 14 hour. The reaction mixture was poured in to crushed ice and the separated solid was purified by silica gel column chromatography to obtain the product in 49% yield M.P. 184-186° C. and MS (M+1)=481 (MH⁺, 100%) for M.F.=C₂₃H₂₅FN₈O₃

### Example, 104

### (S)—N-{3-[4-(4-(5-Pyridin-3-yl-tetrazol)-2-yl-piperidin-1-yl)-3,5-difluorophenyl]-2-oxo-oxazolidin-5-ylmethyl}-acetamide;

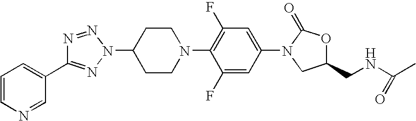

To a solution of (S)—N-{3-[4-(4-methanesulfonyloxy-2-yl-piperidin-1-yl)-3-fluoro phenyl]-2-oxo-oxazolidin-5-ylmethyl}-acetamide (1.37 mmol) in DMF (10 ml) were added 5-pyridin-3-yl-1H-tetrazole (3.01 mmol) & K₂CO₃ (2.05 mmol) and the mixture heated at 80° C. for 14 hour. The reaction mixture was poured in to crushed ice and the separated solid was purified by silica gel column chromatography to obtain the product in 49% yield. M.P. 214-217° C. and MS (M+1)=499 (MH⁺, 100%) for M.F.=C₂₃H₂₄F₂N₈O₃.

### Example-105

### (S)—N-{3-[4-(4-(5-(4-pyridinyl)-tetrazol-1-yl)-piperidin-1-yl)-3-fluorophenyl]-2-oxo-oxazolidin-5-ylmethyl}-acetamide

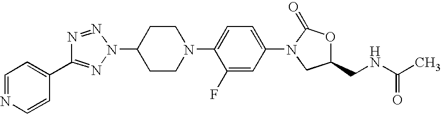

By following the procedure as described in above example and by using (S)—N-{3-[4-(4-p-toluensulfonyloxy-piperidin-1-yl)-3-fluorophenyl]-2-oxo-oxazolidin-5-ylmethyl}-acetamide, 4-(1H-tetrazol-5-yl)-pyridine and K₂CO₃, followed by purification by column chromatography over a silica gel using (9.8:0.2) as an eluent afforded white solid non-polar isomer, in 17% yield as a cream colored solid.

M.P. 182-84° C. and MS (M+1)=481 (MH⁺, 100%) M.F.=C₂₃H₂₅FN₈O₃.

### Example-106

### (S)—N-{3-[4-(4-(5-(4-pyridinyl)-tetrazol-1-yl)-piperidin-1-yl)-3,5-difluorophenyl]-2-oxo-oxazolidin-5-ylmethyl}-acetamide

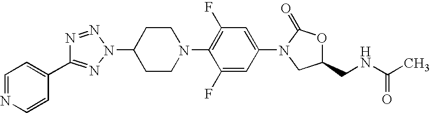

By following the procedure as described in above example and by using (S)—N-{3-[4-(4-p-toluensulfonyloxy-piperidin-1-yl)-3,5-difluorophenyl]-2-oxo-oxazolidin-5-ylmethyl}-acetamide, 4-(1H-tetrazol-5-yl)-pyridine and K₂CO₃, followed by purification by column chromatography over a silica gel using (9.8:0.2) as an eluent afforded white solid non-polar isomer, in 22% yield as a cream colored solid. M.P. 202-204° C. and MS (M+1)=499 (MH⁺, 100%) M.F.=C₂₃H₂₄F₂N₈O₃.

### Example-107

### (S)—N-{3-[4-(4-(5-amino-1H-tetrazol-1-yl)-piperidin-1-yl)-3-fluorophenyl]-2-oxo-oxazolidin-5-ylmethyl}-acetamide

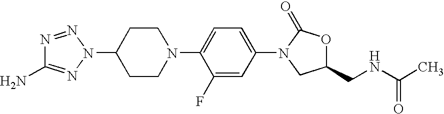

By following the procedure as described in above example and by using (S)—N-{3-[4-(4-p-toluensulfonyloxy-piperidin-1-yl)-3-fluorophenyl]-2-oxo-oxazolidin-5-ylmethyl}-acetamide, 5-amino-1H tetrazole and K₂CO₃, followed by purification by column chromatography over a silica gel using (9.8:0.2) as an eluent afforded white solid (polar isomer), in 12% yield as a cream colored solid. M.P. 174-176° C. and MS (M+1)=419 (MH⁺, 100%) M.F.=C₁₈H₂₃FN₈O₃.

### Example-108

### (S)—N-{3-[4-(4-5-amino-1H-tetrazol-1-yl)-piperidin-1-yl)-3,5-difluorophenyl]-2-oxo-oxazolidin-5-ylmethyl}-acetamide

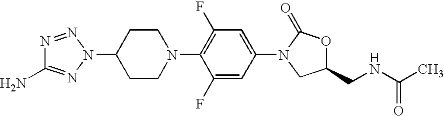

By following the procedure as described in above example and by using (S)—N-{3-[4-(4-p-toluensulfonyloxy-piperidin-1-yl)-3,5-difluorophenyl]-2-oxo-oxazolidin-5-ylmethyl}-acetamide, 5-amino-1H-tetrazole and K₂CO₃, followed by purification by column chromatography over a silica gel using (9.8:0.2) as an eluent afforded white solid (polar isomer), in 12% yield as a cream colored solid. M.P. 218-220° C. and MS (M+1)=437 (MH⁺, 100%) M.F.=C₁₈H₂₂F₂N₈O₃.

### Example-109

### (S)—N-{3-[4-(4-(5-(diethylamino)-2H-tetrazol-2-yl)-piperidin-1-yl)-3,5-difluorophenyl]-2-oxo-oxazolidin-5-ylrmethyl}-acetamide

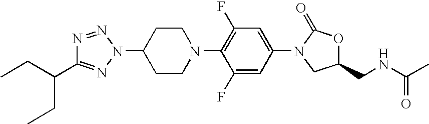

To a solution of (S)—N-{3-[4-(4-Methanesulfonyloxy-2-yl-piperidin-1-yl)-3,5-difluoro phenyl]-2-oxo-oxazolidin-5-ylmethyl}-acetamide (1.37 mmol) in DMF (10 ml) were added 5diethylamino-1H-tetrazole (3.01 mmol) & K₂CO₃ (2.05 mmol) and the mixture heated at 80° C. for 14 hour. The reaction mixture was poured in to crushed ice and the separated solid was purified by silica gel column chromatography to obtain the product in 55% yield M.P. 197-199° C. and MS (M+1)=507 (MH⁺, 100%) for M.F.=C₂₂H₃₀F₂N₈O₃.

### Example-110

### (S)—N-{3-[4-(4-(5-(piperazin-1-yl)-2H-[1,2,3,4],-tetrazol-2)-yl)-piperidin-1-yl)-3-fluorophenyl]-2-oxo-oxazolidin-5-ylmethyl}-acetamide

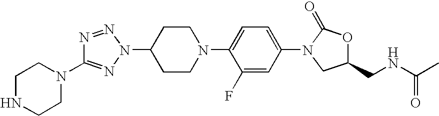

(S)—N-[3-(4-{4-[5-(4-Benzyl-piperazin-1-yl)-tetrazol-2-yl]-piperidin-1-yl}-3-fluoro-phenyl)-2-oxo-oxazolidin-5-ylmethyl]-acetamide (0.86 mmoles) was reacted with Ammonium formate (6.9 mmoles) and 10% palladium charcoal (0.5% w/w) in methanol (25 ml) under reflux for 2 h. The mixture was filtered through celite and the filtrate was concentrated to obtain the product in 90% yield.

M.P. 184-186° C. and MS (M+1)=488 (MH⁺, 100%) for M.F.=C₂₂H₃₀FN₉O₃.

### Example-111

### (S)—N-{3-[4-(4-(5-(piperazin-1-yl)-2H-tetrazol-2-yl)-piperidin-1-yl)-3,5-difluorophenyl]-2-oxo-oxazolidin-5-ylmethyl}-acetamide

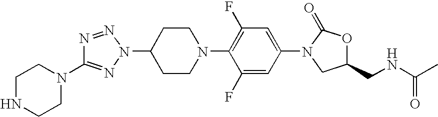

(S)—N-[3-(4-{4-[5-(4-Benzyl-piperazin-1-yl)-tetrazol-2-yl]-piperidin-1-yl}-3,4-difluoro-phenyl)-2-oxo-oxazolidin-5-ylmethyl]-acetamide (0.83 mmoles) was reacted with Ammonium formate (6.7 mmoles) and 10% palladium charcoal (0.5% w/w) in methanol 25 ml under reflux for 2 hour. The mixture was filtered through celite and the filtrate was concentrated to obtain the product (90% yield). M.P. 180-185° C. and MS (M+1)=506 (MH⁺, 100%) for M.F.=C₂₂H₂₉F₂N₉O₃.

### Example-112

### (S)—N-[3-(3-Fluoro-4-{4-[5-(4-methyl-piperazin-1-yl)-tetrazol-2-yl]-piperidin-1-yl}-phenyl)-2-oxo-oxazolidin-5-ylmethyl]-acetamide;

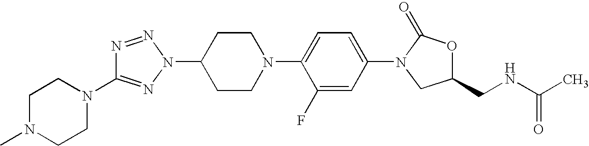

To a solution of (S)—N-(3-{3-Fluoro-4-[4-(5-piperazin-1-yl-tetrazol-2-yl)-piperidin-1-yl]-phenyl}-2-oxo-oxazolidin-5-ylmethyl)-acetamide (0.2 mmoles) in 10 ml dry tetrahydro furan, at 0-5° C., was added sodium hydride (0.24 mmoles) in small portions. After 30 min, was added methyl iodide and the resulting mixture for 3 h at the same temp. The solvent vas evaporated under reduced pressure and the residue chromatographed on a column of silica gel to obtain the product in 56% yield. M.P. 174-176° C. and MS (M+1)=502.2 (MH⁺, 100%) for M.F.=C₂₃H₃₂FN₉O₃.

### Example-113

### (S)—N-{3-[4-(4-(5-(4-methylpiperazin-1-yl)-2H-tetrazol-2-yl)-piperidin-1-yl)-3,5-difluorophenyl]-2-oxo-oxazolidin-5-ylmethyl}-acetamide;

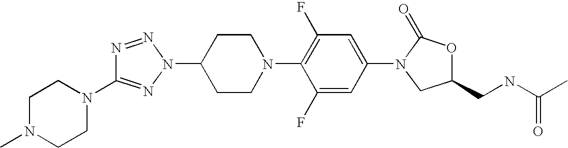

(S)—N-(3-{3,5-difluoro-4-[4-(5-piperazin-1-yl-tetrazol-2-yl)-piperidin-1-yl]-phenyl}-2-oxo-oxazolidin-5-ylmethyl)-acetamide (0.44 mmoles) was added portionwise to a stirred solution of aq. 88% formic acid (3.1 mmoles) and 37% aq. formaldehyde (2.68 mmoles) in 10 ml water at 5° C. The solution is heated under reflux for 12 hours, cooled basified with 10% aq. Na₂CO₃ and extracted with ethyl acetate. Evaporation of ethyl acetate layer under reduced pressure afforded titled compound in 60% yield. M.P. 184-188° C. and MS (M+1)=520.2 (MH⁺, 100%) for M.F.=C₂₃H₃₁F₂N₉O₃.

### Example-114

### (S)—N-{3-[4-(4-(5-(4-acetyl-piperazin-1-yl)-2H-tetrazol-2-yl)-piperidin-1-yl)-3-fluorophenyl]-2-oxo-oxazolidin-5-ylmethyl}-acetamide;

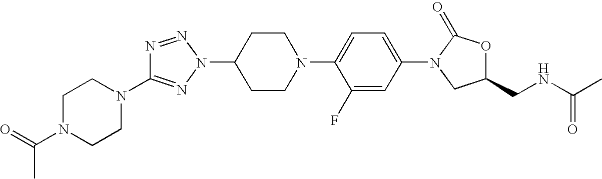

(S)—N-{3-(3-Fluoro-4-[4-(5-piperazin-1-yl-tetrazol-2-yl)-piperidin-1-yl]-phenyl}-2-oxo-oxazolidin-5-ylmethyl)-acetamide (2.0 mmoles) was reacted with acetic anhydride (2.4 mmoles) at 25-30° C. in presence of triethyl amine (3.2 mmol) in 25 ml of THF for 2 hours. The solvent was evaporated under reduced pressure and residue was treated with 20 ml water. The separated solid was filtered and dried to obtain the product in 55% yield. M.P. 216-218° C. and MS (M+1)=530 (MH⁺, 100%) for M.F.=C₂₄H₃₃FN₉O₄

### Example-115

### (S)—N-{3-[4-(4-(5-(4-Acetyl-piperazin-1-yl)-tetrazol-2-yl)-piperidin-1-yl)-3,5-difluoro-phenyl]-2-oxo-oxazolidin-5-ylmethyl}-acetamide;

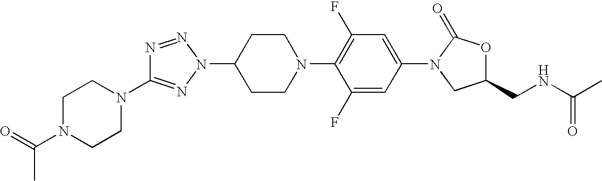

(S)—N-(3-{3,5-Difluoro-4-[4-(5-piperazin-1-yl-tetrazol-2-yl)-piperidin-1-yl]-phenyl}-2-oxo-oxazolidin-5-ylmethyl)-acetamide (1.97 mmol) was reacted with Acetic anhydride (2.37 mmoles) at 25-30° C. in presence of base triethyl amine (3.16 mmoles) in 25 ml tetrahydrofuran for 2 hours. The solvent was evaporated under reduced pressure and residue was treated with 20 ml of water. The separated solid was filtered and dried to obtain the product in 41% yield. M.P. 200-205° C. and MS (M+1)=548 (MH⁺, 100%) for M.F.=C₂₄H₃₁FN₉O₄.

### Example-116

### (S)—N-{3-[4-(4-(5-(4-Cyano-piperazin-1-yl)-tetrazol-2-yl)-piperidin-1-yl)-3-fluoro-phenyl]-2-oxo-oxazolidin-5-ylmethyl}-acetamide;

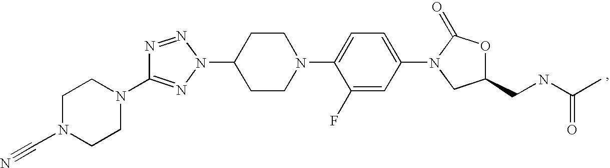

(S)—N-(3-{3-Fluoro-4-[4-(5-piperazin-1-yl-tetrazol-2-yl)-piperidin-1-yl]-phenyl}-2-oxo-oxazolidin-5-ylmethyl)-acetamide (2.0 mmoles) was reacted with Cyanogen bromide (2.2 mmoles) at 25-30° C. in presence of sodium hydrogen carbonate (3.2 mmoles) in 25 ml methanol for 5 hours. The solvent was evaporated under reduced pressure and residue was treated with 20 ml of water. The separated solid was filtered and dried to obtain the product in 57% yield. M.P. 208-210° C. and MS (M+1)=513.2 (MH⁺, 100%) for M.F.=C₂₃H₂₉FN₁₀O₃

### Example-117

### (S)—N-[3-(4-{4-[5-(4-Cyano-piperazin-1-yl)-tetrazol-2-yl]-piperidin-1-yl}-3,5-difluoro-phenyl)-2-oxo-oxazolidin-5-ylmethyl]-acetamide;

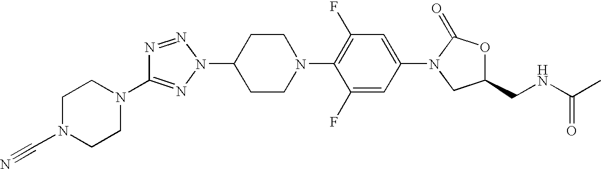

(S)—N-(3-{3,5-Difluoro-4-[4-(5-piperazin-1-yl-tetrazol-2-yl)-piperidin-1-yl]-phenyl}-2-oxo-oxazolidin-5-ylmethyl)-acetamide (1.98 mmoles) was reacted with Cyanogen bromide (2.17 mmoles) at 25-30° C. in presence of sodium hydrogen carbonate (3.16 mmoles) in 25 ml methanol for 5 hours. The solvent was evaporated under reduced pressure and residue was treated with 20 ml of water. The separated solid was filtered and dried to obtain the product in 57% yield. M.P. 198-200° C. and MS (M+1)=531.1 (MH⁺, 100%) for M.F.=C₂₃H₂₈F₂N₁₀O₃.

### Example-118

### (S)—N-{3-[4-(4-(5-(4-Benzyl-piperazin-1-yl)-tetrazol-2-yl)-piperidin-1-yl)-3-fluoro-phenyl]-2-oxo-oxazolidin-5-ylmethyl}-acetamide;

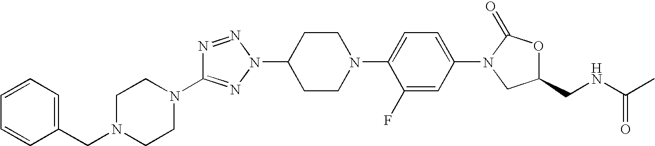

To a solution of (S)—N-{3-[3-fluoro-4-(4-hydroxy-4-methanesulphonyloxymethyl-piperidin-1-yl)-phenyl]-2-oxo-oxazolidin-5-ylmethyl}-acetamide (2.32 mmol) in N,N-dimethylformamide (10 ml), 1-benzyl-4-(2H-tetrazol-5-yl)piperazine (3.72 mmol) and K₂CO₃ (3.72 mmol) were added. The resulting mixture was stirred at 90° for 18 h. The reaction mixture was poured on to crushed ice. The separated solid was filtered and purified by column chromatography over silica gel to obtain the product as a white solid in 46% yield. M.P. 202-204° C. and MS (M+1)=578.1 (MH⁺, 100%) M.F.=C₂₉H₃₆FN₉O₃.

### Example-119

### (S)—N-{3-[4-(4-(5-(4-Benzyl-piperazin-1-yl)-tetrazol-2-yl)-piperidin-1-yl)-3,5-difluoro-phenyl]-2-oxo-oxazolidin-5-ylmethyl}-acetamide;

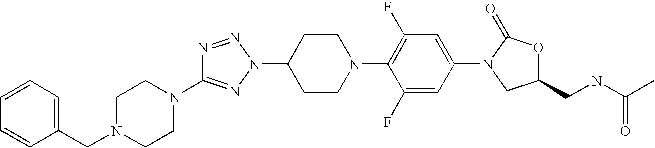

(S)—N-{3-[3,5-difluoro-4-(4-methanesulphonyloxymethyl-piperidin-1-yl)-phenyl]-2-oxo-oxazolidin-5-ylmethyl}-acetamide (2.2 mmol) was reacted with 1-Benzyl-4-(2H-tetrazol-5-yl)-piperazine. (3.56 mmol) and K₂CO₃ (3.56 mmol) in N,N-dimethylformamide (10 ml) at 25° C. for 18 hour and by purifying the compound by silica gel column chromatography the product was obtained as off white solid in 47% yield. M.P. 188-190° C. and MS (M+1)=597 (MH⁺, 100%) for M.F.=C₂₉H₃₅F₂N₉O₃.

### Example-120

### (S)—N-{3-[4-(4-(1H-tetrazol-5-ylmethyl)-piperidin-1-yl)-3-fluoro-phenyl]-2-oxo-oxazolidin-5-ylmethyl}-acetamide

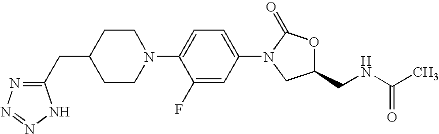

To the solution of N-{3-[4-(4-cyanomethyl-piperidin-1-yl)-3-fluoro-phenyl]-2-oxo-oxazolidin-5-ylmethyl}-acetamide (0.3 g, 0.8 mmol) in toluene (7 ml) was added trimethylsilyl azide (210 μl, 1.6 mmol) and dibutyltin oxide (40 mg, 0.16 mmol) and the reaction mixture was refluxed at 110° C. for 24 h. The solvent was evaporated under reduced pressure and the residue was purified by the column chromatography. A white solid was obtained (0.2 g, 66%) as a title compound.

M.P. 164-166° C. and MS (M+1)=418 (MH⁺, 100%) M.F.=C₁₉H₂₄FN₇O₃.

### Example-121

### (S)—N-{3-[4-(4-(1H-tetrazol-5-ylmethyl)-piperidin-1-yl)-3,5-difluorophenyl]-2-oxo-oxazolidin-5-ylmethyl}-acetamide

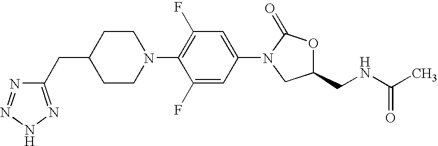

The title compound was obtained as per procedure described above and by using N-{3-[4-(4-cyanopiperidin-1-yl)-3,4-difluoro-phenyl]-2-oxo-oxazolidin-5-ylmethyl}-acetamide and purification over silica gel column to provide a white solid in 66% yield. M.P. 176-78° C. and MS (M+1)=436 (MH⁺, 100%) M.F.=C₁₉H₂₃F₂N₇O₃.

### Example-122

### (R)-{3-[4-(4-(1H-tetrazol-1-yl)-piperidin-1-yl)-3-fluorophenyl]-5-(1H-[1,2,3]-triazol-1-ylmethyl)}-oxazolidin-2-one;

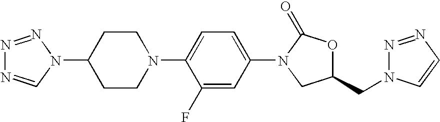

To a stirred solution of (S)—N-{3-[4-(4-toluenesulfonyloxy-piperidin-1-yl)-3-fluorophenyl]-2-oxo-oxazolidin-5-ylmethyl}-[1,2,3]-thiazol-1-yl (2.0 mmol) and 1H-tetrazole (2.1 mmol) in DMF was added powdered K₂CO₃ (2.5 mmol) and heated at 70° C. for 18 hrs. Heating was stopped and cold water was added to the reaction mixture under stirring and then extracted with ethyl acetate (2×75 ml). Combined organic layer was then washed with brine and dried over Na₂SO₄ and evaporated under vacuum. Crude product upon purification by column chromatography over a silica gel using (9.8:0.2) as an eluent afforded white solid. Non-polar isomer: M.P. 192-194° C. and MS (M+1)=414 (MH⁺, 100%) M.F.=C₁₉H₂₀FN₉O₂.

### Example-122 and 123

### (R)-{3-[4-(4-(1H-tetrazol-1-yl)-piperidin-1-yl)-3-fluorophenyl]-5-(1H-[1,2,3]-triazol-1-ylmethyl)}-oxazolidin-2-one; and

### (S)—N-{3-[4-(4-(tetrazol-2-yl)-piperidin-1-yl)-3-fluorophenyl]-2-oxo-oxazolidin-5-ylmethyl}-[1,2,3]-triazol-1-yl;

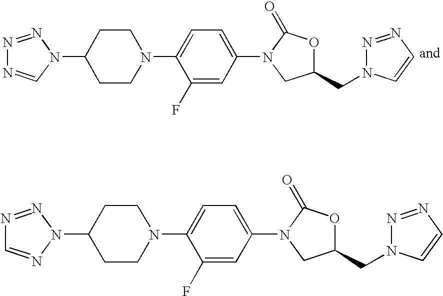

To a stirred solution of (R)—N-{3-[4-(4-toluenesulfonyloxy-piperidin-1-yl)-3-fluorophenyl]-2-oxo-oxazolidin-5-ylmethyl}-[1,2,3]-triazol-1-yl (2.0 mmol) and 1H-tetrazole (2.1 mmol) in DMF was added powdered K₂CO₃ (2.5 mmol) and heated at 70° C. for 18 hrs. Heating was stopped and cold water was added to the reaction mixture under stirring and then extracted with ethyl acetate (2×75 ml). Combined organic layer was then washed with brine and dried over Na₂SO₄ and evaporated under vacuum. Crude product upon purification by column chromatography over a silica gel using (9.8:0.2) as an eluent afforded white solid. Non-polar isomer: M.P. 192-194° C. and MS (M+1)=414 (MH⁺, 100%) M.F.=C₁₈H₂₀FN₉O₂. Polar isomer: M.P. 184-186° C. and MS (M+1)=414 (MH⁺, 100%) M.F.=C₁₈H₂₀FN₉O₂.

### Example-124

### (R)—N-{3-[4-(4-(tetrazol-5-yl)-piperidin-1-yl)-3-fluorophenyl]-5-(1H-[1,2,3]-triazol-1-ylmethyl)}-oxazolidin-2-one;

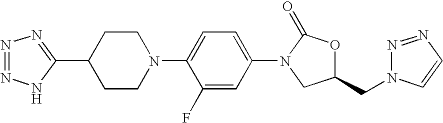

To the solution of N-{3-[4-(4-cyanomethyl-piperidin-1-yl)-3-fluoro-phenyl]-2-oxo-oxazolidin-5-ylmethyl}-[1,2,3]-triazol-1-yl (0.8 mmol) in toluene (7 ml) was added trimethylsilyl azide (210 ml, 1.6 mmol) and dibutyltin oxide (40 mg, 0.16 mmol) and the reaction mixture was refluxed at 120° C. for 24 h. The solvent was evaporated under reduced pressure and the residue was purified by the column chromatography. Purification over silica gel provided a white solid in 62% yield. M.P. 208(d)° C. and MS (M+1)=414 (MH⁺, 100%) M.F.=C₁₈H₂₀FN₉O₂.

### Example-125

### (S)—N-{3-[4-(4-([1,2,3]-triazol-1-yl)-methyl-piperidin-1-yl)-3-fluoro-phenyl]-5-(isoxazol-3-yloxymethyl)}-oxazolidin-2-one;

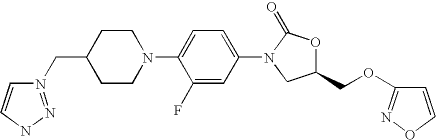

To the stirred solution of (R)-3-[4-(4-azidomethyl-piperidin-1-yl)-3-fluoro-phenyl]-5-(isoxazol-3-yloxymethyl)-oxazolidin-2-one (1 gm, 2.4 mmoles) in dioxane (20 ml) was added 2,5-norbornadiene (0.33 gm, 3.6 mmoles) and the resulting solution was stirred at 80° C. for 16 hours. Solvent was removed in-vacuum & the crude compound was purified by column eluting with CHCl₃:CH₃OH (99:1) to afford the pure compound as off-white solid. M.P. 123-25° C. and MS (M+1)=445 (MH⁺, 100%) M.F.=C₂₁H₂₅FN₆O₄.

### Example-126

### (R)-{3-[4-(4-((4-hydroxymethyl)-[1,2,3]-triazol-1-yl)-methyl-piperidin-1-yl)-3-fluoro-phenyl]-5-(isoxazol-3-yloxymethyl)}-oxazolidin-2-one;

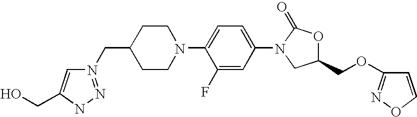

To the stirred solution of (R)-3-[4-(4-azidomethyl-piperidin-1-yl)-3-fluoro-phenyl]-5-(isoxazol-3-yloxymethyl)-oxazolidin-2-one (1 gm, 2.4 mmoles) in acetonitrile (10 ml) was added propargyl alcohol (0.161 gm, 2.88 mmoles) and CuI (0.09 gm, 0.48 mmoles) and the resulting solution was stirred under nitrogen at 25° C. for 16 hours. Solvent was removed in-vacuum & the crude compound was purified by column eluting with CHCl₃:CH₃OH (98:2) to afford the pure compound as white solid, 65% yield. M.P. 154-157° C. and MS (M+1)=473(MH⁺, 100%) M.F.=C₂₂H₂₅FN₆O₅.

### Example-127

### (S)—N-{3-[4-(4-(5-methyl-[1,2,4]-oxadiazol-3-ylmethyl)-piperidin-1-yl)-3-fluoro-phenyl]-2-oxo-oxazolidin-5-ylmethyl}-acetamide;

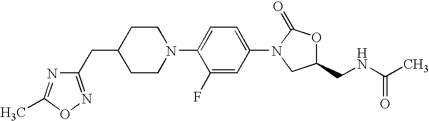

To the solution of (S)—N-{3-[4-(4-cyanomethyl-piperidin-1-yl)-3-fluoro-phenyl]-2-oxo-oxazolidin-5-ylmethyl}-acetamide (0.3 g, 0.8 mmol) in methanol (10 ml) was added hydroxylamine hydrochloride (330 mg, 4.8 mmol) and sodium bicarbonate (435 mg, 4.2 mmol) at room temperature. The reaction mixture was then heated under reflux for 4 h.

The solvent was removed and the crude mass obtained was taken into the water stirred for 15 min., filtered and dried. The solid (250 mg, 0.6 mmol) obtained was then reacted with the acetyl chloride (50 ul, 0.66 mmol) in the presence of K₂CO₃ (90 mg, 0.66 mmol) in dioxane (3 ml) at room temperature for 15 h. To then reaction mixture water (10 ml) was added and extracted with the ethyl acetate (3×10 ml). The combined organic layer was dried (Na₂SO₄) and evaporated under reduced pressure to furnish the pale yellow solid (200 mg). This solid was then taken into toluene and heated under reflux for 16 h. The solvent was removed under reduce pressure. The residue obtained was purified by the column chromatography over silica gel using CHCl₃:MeOH (98:2) as an eluent to furnish the white solid (150 mg, 65%). M.P. 136-138° C. and MS (M+1)=432 (MH⁺, 100%) M.F.=C₂₁H₂₆FN₅O₄.

### Example-128

### (S)—N-{3-[4-(4-(5-methyl-1,2,4-oxadiazol-3-ylmethyl)-piperidin-1-yl)-3,5-difluoro-phenyl]-2-oxo-oxazolidin-5-ylmethyl}-acetamide;

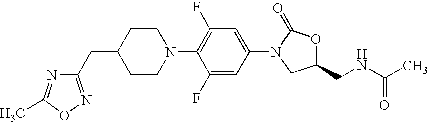

The title compound was obtained by using procedure as described in above example and by using (S)—N-{3-[4 (4-cyanomethyl-piperidin-1-yl)-3,5-difluoro-phenyl]-2-oxo-oxazolidin-5-ylmethyl}-acetamide in the place of N-{3-[4-(4-cyanomethyl-piperidin-1-yl)-3-fluoro-phenyl]-2-oxo-oxazolidin-5-ylmethyl}-acetamide in 67% yield as a pale yellow solid. M.P. 134-136° C. and MS (M+1)=450 (MH⁺, 100%) M.F.=C₂₁H₂₅F₂N₅O₄.

### Example-129

### (S)—N-{3-[4-(4-(5-methyl-[1,2,4]-oxadiazol-3-yl)-piperidin-1-yl)-3-fluoro-phenyl]-2-oxo-oxazolidin-5-ylmethyl}-acetamide;

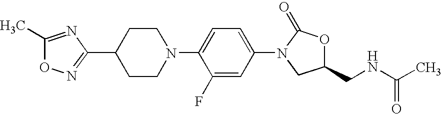

To the solution of (S)—N-{3-[4-(4-cyano-piperidin-1-yl)-3-fluoro-phenyl]-2-oxo-oxazolidin-5-ylmethyl}-acetamide (0.3 g, 0.8 mmol) in methanol (10 ml) was added hydroxyl amine hydrochloride (330 mg, 4.8 mmol) and sodium bicarbonate (435 mg, 4.2 mmol) at room temperature. The reaction mixture was then heated under reflux for 4 h. The solvent was removed and the crude mass obtained was taken into the water stirred for 15 min., filtered and dried. The solid (250 mg, 0.6 mmol) obtained was then reacted with the acetyl chloride (50 μl, 0.66 mmol) in the presence of K₂CO₃ (90 mg, 0.66 mmol) in dioxane (3 ml) at room temperature for 15 h. To then reaction mixture water (10 ml) was added and extracted with the ethyl acetate (3×10 ml). The combined organic layer was dried (Na₂SO₄) and evaporated under reduced pressure to furnish the pale yellow solid (200 mg). This solid was then taken into toluene and heated under reflux for 16 h. The solvent was removed under reduce pressure. The residue obtained was purified by the column chromatography over silica-gel using CHCl₃:MeOH (98:2) as an eluent to furnish the white solid (150 mg, 65%).

M.P. 136-138° C. and MS (M+1)=418 (MH⁺, 100%) M.F.=C₂₀H₂₄FN₅O₄.

### Example-130

### (S)—N-{3-[4-(4-(5-phenyl-[1,2,4]-oxadiazol-3-ylmethyl)-piperidin-1-yl)-3-fluorophenyl]-2-oxo-oxazolidin-5-ylmethyl}-acetamide;

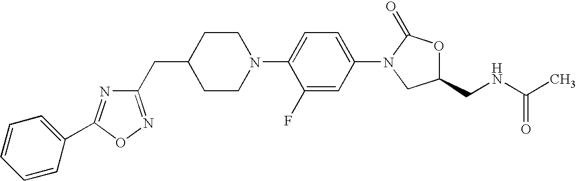

The title compound was obtained by using procedure as described in above example and by using N-{3-[4-(4-cyanomethyl-piperidin-1-yl)-3-fluoro-phenyl]-2-oxo-oxazolidin-5-ylmethyl}-acetamide and benzoyl chloride in 65% yield as a white solid. M.P. 144-46° C. and MS (M+1)=494 (MH⁺, 100%) M.F.=C₂₆H₂₉FN₅O₄.

### Example-131

### (S)—N-{3-[4-(4-(5-phenyl-1,2,4-oxadiazol-3-ylmethyl)-piperidin-1-yl)-3,5-difluoro-phenyl]-2-oxo-oxazolidin-5-ylmethyl}-acetamide;

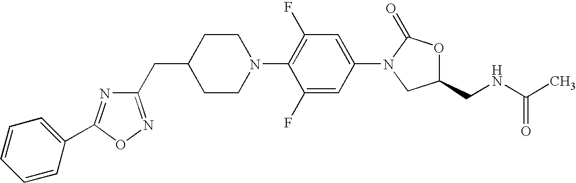

The title compound was obtained by using procedure as described in above example and by using (S)—N-{3-[4-(4-cyanomethyl-piperidin-1-yl)-3,5-difluoro-phenyl]-2-oxo-oxazolidin-5-ylmethyl}-acetamide and benzoyl chloride in 65% yield as a white solid. M.P. 152-54° C. and MS (M+1)=512 (MH⁺, 100%) M.F.=C₂₆H₂₇F₂N₅O₄.

### Example-132

### (S)—N-{3-[4-(4-(4-(5-pyridin-3-yl)-[1,2,4]-oxadiazol-3-ylmethyl)-piperidin-1-yl)-3-fluoro-phenyl]-2-oxo-oxazolidin-5-ylmethyl}-acetamide;

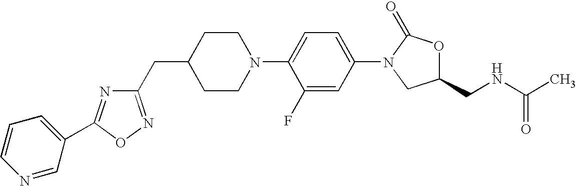

The title compound was obtained by using procedure as described in above example and by using N-{3-[4-(4-cyanomethyl-piperidin-1-yl)-3,5-difluoro-phenyl]-2-oxo-oxazolidin-5-ylmethyl}-acetamide and nicotinyl chloride in 70% yield as a white solid. M.P. 150-152° C. and MS (M+1)=495 (MH⁺, 100%) M.F.=C₂₅H₂₇FN₆O₄.

### Example-133

### (S)—N-{3-[4-(4-(5-methyl-1,3,4-oxadiazol-2-ylmethyl)-piperidin-1-yl)-3-fluoro-phenyl]-2-oxo-oxazolidin-5-ylmethyl}-acetamide;

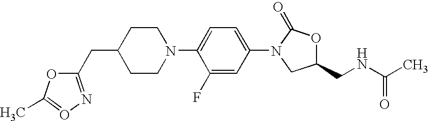

To the solution of N-{3-[4-(4-hydrazinocarbonylmethyl-piperidin-1-yl)-3-fluorophenyl]-2-oxo-oxazolidin-5-ylmethyl}-acetamide (0.2 g, 0.5 mmol) in THF (5 ml) was added acetyl chloride (55 ul, 0.73 mmol) and refluxed for 2 h. The residue obtained after the evaporation of the solvent was taken into the water and extracted with the ethyl acetate (3×10 ml). The combined organic layer was dried (Na₂SO₄) and it was evaporated to give the crude solid, which was purified by the column chromatography over silica gel. A white solid (110 mg, 55%) was obtained as a title compound. M.P. ° C. and MS (M+1)=432 (MH⁺, 100%) M.F.=C₂₁H₂₆FN₅O₄.

### Example-134

### (S)—N-{3-[4-(4-(5-phenyl-[1,3,4]-oxadiazol-2-ylmethyl)-piperidin-1-yl)-3-fluoro-phenyl]-2-oxo-oxazolidin-5-ylmethyl}-acetamide;

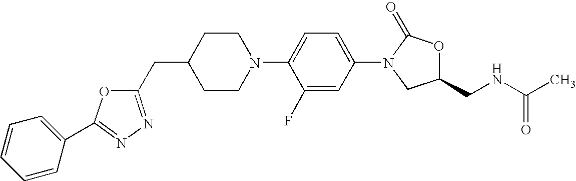

By using the same method as described above was used to obtain the title compound in 45% yield. M.P. 199-200° C. and MS (M+1)=494 (MH⁺, 100%) M.F.=C₂₆H₂₈FN₅O₄.

### Example-135

### (S)—N-{3-[4-(4-(5-Amino-1,3,4-thiadiazol-2-ylmethyl)-piperidin-1-yl)-3-fluoro-phenyl]-2-oxo-oxazolidin-5-ylmethyl}-acetamide

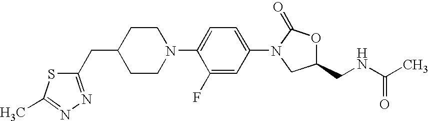

To a stirred solution of (S)—N-{3-[4-(4-cyanomethylpiperidin-1-yl)-3-fluorophenyl]-2-oxo-1,3-oxazolidin-5-ylmethyl}-acetamide (250 mg, 0.7 mmol) and thiosemicarbazide (67 mg, 0.7 mmol) in methane sulphonic acid (3.0 ml) was stirred at an ambient temperature for 6 hr. The mixture was poured to the crushed ice and extracted with ethyl acetate (20 ml×3) organic layer was evaporated under vacuum yielding crude solid was recrystallized from MeOH-Ether resulting off white solid in (65 mg) 45% yield. M.P. 206-207° C. and MS (M+1)=449 (MH⁺, 100%) M.F.=C₂₀H₂₅FN₆O₃S.

### Example-136

### (S)—N-{3-[4-(4-(5-Amino-2H-1,3,4-thiadiazol-2-yl)-piperidin-1-yl)-3-fluorophenyl]-2-oxo-oxazolidin-5-ylmethyl}-acetamide

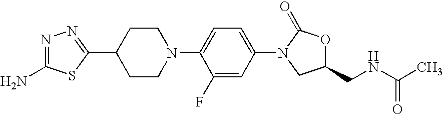

Title compound was prepared as by using (S)—N-{3-[4-(4-cyanopiperidin-1-yl)-3-fluorophenyl]-2-oxo-1,3-oxazolidin-5-yl}methyl)acetamide and thiouarea following the procedure as mentioned in synthesis of above example in 42% yield. M.P. 168-69° C. and MS (M+1)=435 (MH⁺, 100%) M.F.=C₁₉H₂₃FN₆O₃S.

### Example-137

### (S)—N-{3-[4-(4-(3-ethoxycarbonyl-imidazol-1-yl)-methyl-piperidin-1-yl)-3-fluorophenyl]-2-oxo-oxazolidin-5-ylmethyl}-acetamide

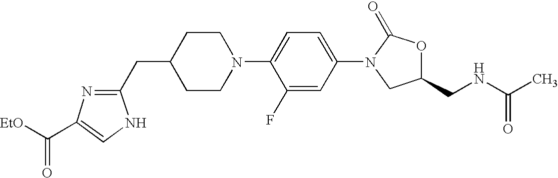

To a solution of (S)—N-{3-{4-[4-(N-hydroxyiminoamino-methyl)-piperidin-1-yl)-3-fluorophenyl]-2-oxo-oxazolidin-5-ylmethyl}-acetamide (0.2 gm, 0.5 mmol) and ethyl propiolate (0.1 ml, 1.0 mmol) in diphenyl ether (8 ml) was heated at 170° C. for 3 hrs. After completion of reaction solvent was evaporated and compound was purified by column chromatography over silica gel using CHCl₃:MeOH (98:2) as an eluent yielding off white solid 80 mg (25%), M.P. 198-200° C. and MS (M+1)=488 (MH⁺, 100%) M.F.=C₂₄H₃₀FN₅O₅.

### Example-138

### (S)—N-{3-[3,5-Difluoro-4-(4-2H-tetrazol-2-yl-piperidin-1-yl)-phenyl]-2-oxo-oxazolidin-5-ylmethyl}-carbamic acid methyl ester

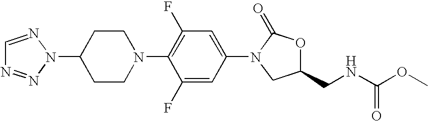

A solution of (S)-5-Aminomethyl-3-[3,5-difluoro-4-(4-2H-tetrazol-2-yl-piperidin-1-yl)-phenyl]-oxazolidin-2-one (1.25 mmol) in dichloromethane (10 ml), containing triethyl amine (3.75 mmol) was cooled to 0° C. Methyl chloroformate (2.0 mmol) was added dropwise. The resulting mixture was stirred for 5 h at room temp. The solvent was evaporated under reduced pressure and the residue chromatographed on a column on silica gel to obtain the product as white solid in 90% yield. M.P. 168-170° C. and MS (M+1)=438 (MH⁺, 100%) M.F.=C₁₈H₂₁F₂N₇O₄.

Using the above procedure the following compounds were prepared

| Example | | | Molecular |
|---|---|---|---|
| No. | Compound | M + 1 | formula |
| 139 | 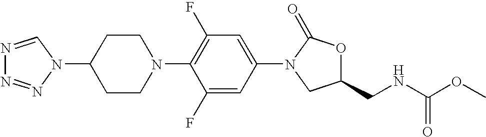 | 438 | C₁₈H₂₁F₂N₇O₄ |
| 140 | 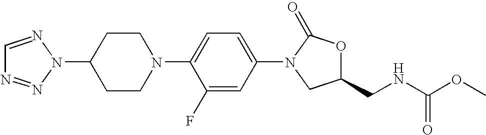 | 420 | C₁₈H₂₂FN₇O₄ |
| 141 | 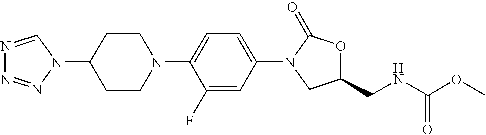 | 420 | C₁₈H₂₂FN₇O₄ |
| 142 | 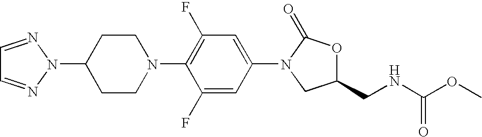 | 437 | C₁₉H₂₂F₂N₆O₄ |
| 143 | 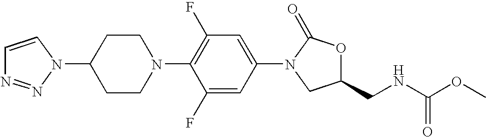 | 437 | C₁₉H₂₂F₂N₆O₄ |

### Example-144

### (S)—N-{3-[3,5-Difluoro-4-(4-[1,2,3]triazol-2-yl-piperidin-1-yl)-phenyl]-2-oxo-oxazolidin-5-ylmethyl}-2,2-difluoro-acetamide

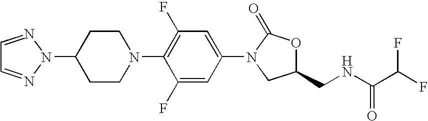

To a solution of (S)-5-Aminomethyl-3-[3,5-difluoro-4-(4-[1,2,3]triazol-2-yl-piperidin-1-yl)-phenyl]-oxazolidin-2-one (1.25 mmol) in dimethylformamide (10 ml), were added successively difluoroacetic acid (1.25 mmol), HOBt (1.25 mmol), EDC.HCl (1.50 mmol) and NMM (1.75 mmol). The reaction mixture was stirred for 14 h at room temp. The resulting mixture was poured in ice-water, stirred well and extracted with ethyl acetate. The extract was washed with water, dried and evaporated under reduced pressure. The residue obtained was chromatographed on a column on silica gel to obtain the product as white solid in 55% yield.

M.P. 254-255° C. and MS (M+1)=437 (MH⁺, 100%) M.F.=C₁₉H₂₀F₄N₆O₃

Using the above procedure the following compounds were prepared

| Example | | | Molecular |
|---|---|---|---|
| No. | Compound | M + 1 | formula |
| 145 | 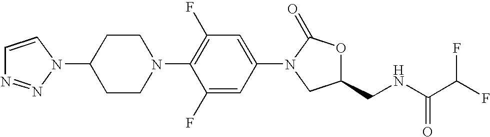 | 437 | C₁₉H₂₀F₄N₆O₃ |
| 146 | 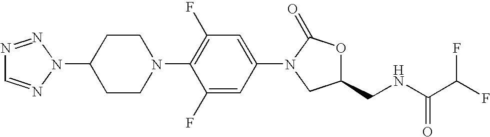 | 457 | C₁₈H₁₉F₄N₇O₃ |
| 147 | 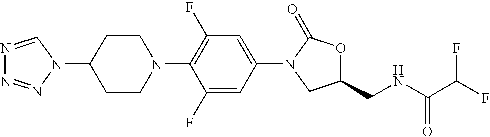 | 457 | C₁₈H₁₉F₄N₇O₃ |

### Example-148

### (S)—N-{3-[3,5-Difluoro-4-(4-2H-tetrazole-2-yl-piperidin-1-yl)-phenyl]-2-oxo-oxazolidin-5-ylmethyl}-2,2-dichloro-acetamide

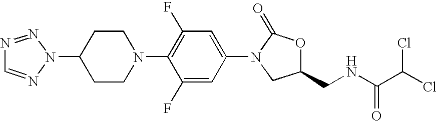

To a solution of (S)-5-Aminomethyl-3-[3,5-difluoro-4-(4-2H-tetrazole-2-yl-piperidin-1-yl)-phenyl]-oxazolidin-2-one (1.25 mmol) in dimethyl formamide (10 ml), were added successively dichloroacetic acid (1.25 mmol), HOBt (1.25 mmol), EDC.HCl (1.50 mmol) and NMM (1.75 mmol). The reaction mixture was stirred for 14 h. at room temp. The resulting mixture was poured in ice-water, stirred well and extracted with ethyl acetate. The extract was washed with water, dried and evaporated under reduced pressure. The residue obtained was chromatographed on a column on silica gel to obtain the product as white solid in 80% yield.

M.P. 160-162° C. and MS (M+1)=490 (MH⁺, 100%) M.F.=C₁₈H₁₉Cl₂F₂N₇O₃

### Example-149

### (S)—N-{3-[3-fluoro-4-(4-2H-tetrazol-2-yl-piperidin-1-yl)-phenyl]-2-oxo-oxazolidin-5-ylmethyl}-carbamic acid ethyl ester

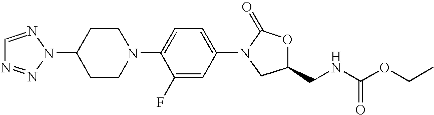

A solution of (S)-5-Aminomethyl-3-[3-fluoro-4-(4-2H-tetrazol-2-yl-piperidin-1-yl)-phenyl]-oxazolidin-2-one (1.25 mmol) in dichloromethane (10 ml), containing triethyl amine (3.75 mmol) was cooled to 0° C. Ethyl chloroformate (2.0 mmol) was added dropwise. The resulting mixture was stirred for 5 h at room temp. The solvent was evaporated under reduced pressure and the residue chromatographed on a column on silica gel to obtain the product as white solid in 70% yield.

M.P. 176-178° C. and MS (M+1)=443 (MH⁺, 100%) M.F.=C₁₈H₂₂FN₇O₄

### Example-150

### (S)—N-{3-[3,5-Difluoro-4-(4-1H-tetrazole-1-yl-piperidin-1-yl)-phenyl]-2-oxo-oxazolidin-5-ylmethyl}-2,2-difluoro-thioacetamide

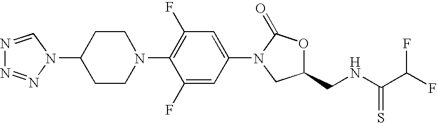

To a solution of (S)—N-{3-[3,5-Difluoro-4-(4-1H-tetrazole-1-yl-piperidin-1-yl)-phenyl]-2-oxo-oxazolidin-5-ylmethyl}-2,2-difluoro-acetamide (1.25 mmol) in tetrahydrofuran (10 ml), was added Lawesson's reagent (4.0 mmol), and the solution was heated under reflux for 10 h. The solvent was evaporated under reduced pressure and the residue chromatographed on a column on silica gel to obtain the product in 40% yield.

M.P. 156-158° C. and MS (M+1)=456 (MH⁺, 100%) M.F.=C₁₈H₂₀F₂N₇O₂S.

### Example-151

### (S)—N-{3-[3,5-Difluoro-4-(4-1H-tetrazole-1-yl-piperidin-1-yl)-phenyl]-2-oxo-oxazolidin-5-ylmethyl}-cyclopropane carboxamide

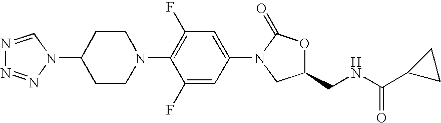

To a solution of (S)-5-Aminomethyl-3-[3,5-difluoro-4-(4-1H-tetrazole-1-yl-piperidin-1-yl)-phenyl]-oxazolidin-2-one (1.25 mmol) in dimethyl formamide (10 ml), were added successively cyclopropane carboxylic acid (1.25 mmol), HOBt (1.25 mmol), EDC.HCl (1.50 mmol) and NMM (1.75 mmol). The reaction mixture was stirred for 14 h at room temp. The resulting mixture was poured in ice-water, stirred well and extracted with ethyl acetate. The extract was washed with water, dried and evaporated under reduced pressure. The residue obtained was chromatographed on a column on silica gel to obtain the product as white solid in 72% yield.

M.P. 162-164° C. and MS (M+1)=448 (MH⁺, 100%) M.F.=C₂₀H₂₃F₂N₇O₃.

### Example-152 and 153

### (S)—N-{3-[3,5-Difluoro-4-(4-[1,2,4]triazol-1-yl-piperidin-1-yl)-phenyl]-2-oxo-oxazolidin-5-ylmethyl}-propionamide; and

### (S)—N-{3-[3,5-Difluoro-4-(4-[1,3,4]triazol-1-yl-piperidin-1-yl)-phenyl]-2-oxo-oxazolidin-5-ylmethyl}-propionamide;

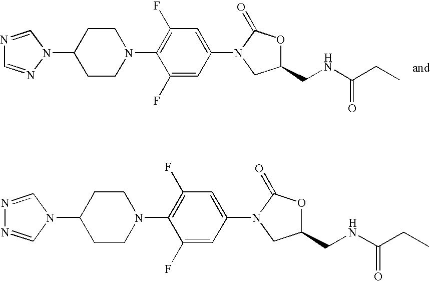

A mixture of (S)—N-{3-[4-methanesulphonyloxy-piperidin-1-yl)-3,5-difluorophenyl]-2-oxo-oxazolidin-5-ylmethyl}-propionamide (1.12 mM), 1,2,4-triazole (1.68 mM), and K₂CO₃ (1.68 mM) in DMF (10 ml) was heated for 22 hrs at 85° C. The resulting mixture was poured into ice-water and stirred for 30 min. The separated solid was purified by column chromatography to obtain two isomeric products in 18% and 22% yields respectively.

Isomer A: M.P. 156-158° C.; MS (M+1)-435; M.F. C₂₀H₂₄F₂N₆O₃

Isomer B: M.P. 136-138° C.; MS (M+1)-435; M.F. C₁₉H₂₂F₂N₆O₃

### Example-154 and 155

### (S)—N-{3-[3,5-Difluoro-4-(4-2H-tetrazol-2-yl-piperidin-1-yl)-phenyl]-2-oxo-oxazolidin-5-ylmethyl}-propionamide; and

### (S)—N-{3-[3,5-Difluoro-4-(4-1H-tetrazol-1-yl-piperidin-1-yl)-phenyl]-2-oxo-oxazolidin-5-ylmethyl}-propionamide;

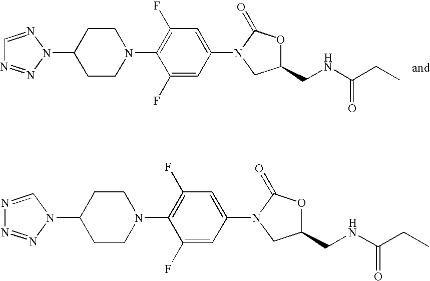

A mixture of (S)—N-{3-[4-methanesulphonyloxy piperidin-1-yl)-3,5-difluorophenyl]-2-oxo-oxazolidin-5-ylmethyl}-propionamide (1.12 mm), tetrazole (1.68 mm), and K₂CO₃ (1.68 mm) in DMF (10 ml) was heated for 22 hrs at 85° C. The resulting mixture was poured into ice-water and stirred for 30 min. The separated solid was purified by column chromatography to obtain two isomeric products in 28% and 22% yields respectively.

Isomer A: M.P. 190-192° C.; MS (M+1)-436 M.F. C₁₉H₂₁F₂N₇O₄

Isomer B: M.P. 158-160° C.; MS (M+1)-436; M.F. C₁₉H₂₁F₂N₇O₄.

### Example-156 and 157

### (S)N-{3-[3-Fluoro-4-(4-2H-tetrazol-2-yl-piperidin-1-yl)-phenyl]-2-oxo-oxazolidin-5-ylmethyl}-propionamide; and

### (S)N-{3-[3-Fluoro-4-(4-1H-tetrazol-1-yl-piperidin-1-yl)-phenyl]-2-oxo-oxazolidin-5-ylmethyl}-propionamide;

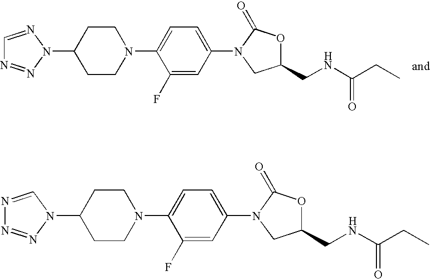

A mixture of (S)—N-{3-[4-methanesulphonyloxy piperidin-1-yl)-3-fluorophenyl]-2-oxo-oxazolidin-5-ylmethyl}-propionamide (1.12 mM), tetrazole (1.68 mM), and K₂CO₃ (1.68 mM) in DMF (10 ml) was heated for 22 hrs at 85° C. The resulting mixture was poured into ice-water and stirred for 30 min. The separated solid was purified by column chromatography to obtain two isomeric products in 28% and 22% yields respectively.

Isomer A: M.P. 170-172° C.; MS (M+1)-418; M.F. C₁₉H₂₂FN₇O₄

Isomer B: M.P. 156-160° C.; MS (M+1)-418; M.F. C₁₉H₂₂FN₇O₄

### Example-158 and 159

### (S)—N-{3-[3,5-Difluoro-4-(4-[1,2,3]-triazol-2-yl-piperidin-1-yl)-phenyl]-2-oxo-oxazolidin-5-ylmethyl}-propionamide; and

### (S)—N-{3-[3,5-Difluoro-4-(4-[1,2,3]-triazol-1-yl-piperidin-1-yl)-phenyl]-2-oxo-oxazolidin-5-ylmethyl}-propionamide;

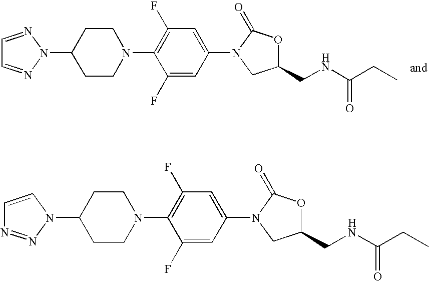

A mixture of (S)—N-{3-[4-methanesulphonyloxy piperidin-1-yl)-3,5-difluorophenyl]-2-oxo-oxazolidin-5-ylmethyl}-propionamide (1.12 mM), [1,2,3]-triazole (1.68 mM), and K₂CO₃ (1.68 mM) in DMF (10 ml) was heated for 22 hrs at 85° C. The resulting mixture was poured into ice-water and stirred for 30 min. The separated solid was purified by column chromatography to obtain two isomeric products in 18% and 22% yields respectively.

Isomer A: M.P. 208-210° C.; MS (M+1)-435 M.F. C₂₀H₂₄F₂N₆O₃

Isomer B: M.P. 182-184° C.; MS (M+1)-435 M.F. C₁₉H₂₂F₂N₆O₃

### Example-160 and 161

### (S)—N-{3-[3,5-Difluoro-4-(4-(5-methyl-tetrazol-2-yl)-piperidin-1-yl)-phenyl]-2-oxo-oxazolidin-5-ylmethyl}-propionamide; and

### (S)—N-{3-[3,5-Difluoro-4-(4-(5-methyl-tetrazol-1-yl)-piperidin-1-yl)-phenyl]-2-oxo-oxazolidin-5-ylmethyl}-propionamide;

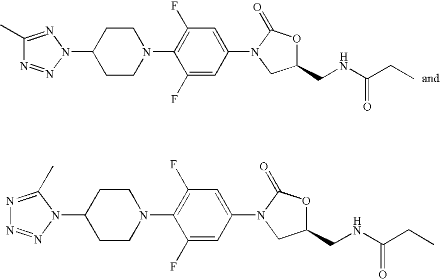

A mixture of (S)—N-{3-[4-methanesulphonyloxy piperidin-1-yl)-3,5-difluorophenyl]-2-oxo-oxazolidin-5-ylmethyl}-propionamide (1.12 mM),tetrazole (1.68 mM), and K₂CO₃ (1.68 mM) in DMF (10 ml) was heated for 22 hrs at 85° C. The resulting mixture was poured into ice-water and stirred for 30 min. The separated solid was purified by column chromatography to obtain two isomeric products in 16% and 15% yields respectively.

Isomer A: M.P. 220-222° C.; MS (M+1)-450; M.F. C₂₀H₂₅F₂N₇O₃

Isomer B: M.P. 206-208° C.; MS (M+1)-450; M.F. C₂₀H₂₅F₂N₇O₄.

### Example-162-165

### cis-(R)-{3-[4-(4-(5-ethoxycarbonyl-1H-[1,2,3]-triazol-1-yl)-piperidin-1-yl)-3-fluorophenyl]-5-(5-ethoxycarbonyl-1H-[1,2,3]-triazol-1-ylmethyl)}-oxazolidin-2-one; and

### cis-(R)-{3-[4-(4-(5-ethoxycarbonyl-1H-[1,2,3]-triazol-1-yl)-piperidin-1-yl)-3-fluoro-phenyl]-5-(4-ethoxycarbonyl-1H-[1,2,3]-triazol-1-ylmethyl)}-oxazolidin-2-one; and

### trans-(R)-{3-[4-(4-(5-ethoxycarbonyl-1H-[1,2,3]-triazol-1-yl)-piperidin-1-yl)-3-fluoro-phenyl]-5-(5-ethoxycarbonyl-1H-[1,2,3]-triazol-1-ylmethyl)}-oxazolidin-2-one; and

### trans-(R)-{3-[4-(4-(5-ethoxycarbonyl-1H-[1,2,3]-triazol-1-yl)-piperidin-1-yl)-3-fluoro-phenyl]-5-(4-ethoxycarbonyl-1H-[1,2,3]-triazol-1-ylmethyl)}-oxazolidin-2-one;

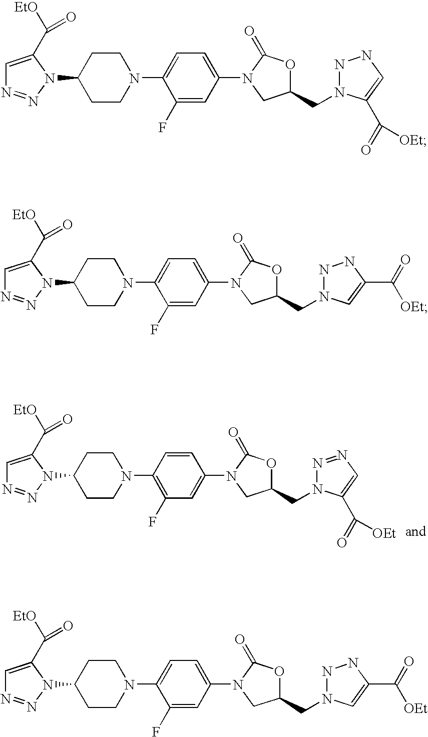

To the solution of 5-azidomethyl-3-[4-(4-azido-piperidin-1-yl)-3-fluoro-phenyl]-oxazolidin-2-one (0.5 g, 1.4 mmol) in the toluene (8 ml) was added ethyl propiolate (0.3 ml, 3 mmol) was added and the reaction mixture was heated at 120° C. for 6 h. The solvent was removed under reduced pressure and the resulting residue was chromatographed over silica gel using CHCl₃:MeOH (98:2) as an eluent by gradient method, which has afforded the four isomers.

Isomer-1: 25 mg, 7%, M.P. 128-30° C. and MS (M+1)=559 (MH⁺, 100%) M.F.=C₂₅H₃₁FN₈O₆.

Isomer-2: 125 mg, 30%, M.P. 154-56° C. and MS (M+1)=559 (MH⁺, 100%) M.F.=C₂₅H₃₁FN₈O₆.

Isomer-3: 60 mg, 15%, M.P. 134-36° C. and MS (M+1)=559 (MH⁺, 100%) M.F.=C₂₅H₃₁FN₈O₆.

Isomer-4: 200 mg, 48%, M.P. 216-218° C. and MS (M+1)=559 (MH⁺, 100%) M.F.=C₂₅H₃₁FN₈O₆.

### Example-166

### trans-(R)-{3-[4-(4-(5-hydroxymethyl-1H-[1,2,3]-triazol-1-yl)-piperidin-1-yl)-3-fluoro-phenyl]-5-(4-hydroxymethyl-1H-[1,2,3]-triazol-1-ylmethyl)}-oxazolidin-2-one;

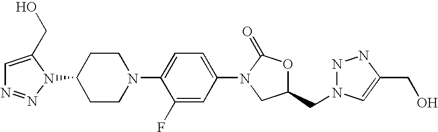

The isomer-4 was (100 mg, 0.2 mmol) was taken into ethanol (3 ml) was treated with the sodium borohydride (15 mg, 0.4 mmol) at room temperature. The reaction mixture was stirred for 2 h. The reaction was quenched by adding the ice-cold solution of the ammonium chloride and extracted with the ethyl acetate (2×10 ml). The combined organic layer was washed with the brine and dried over Na2SO4. Upon removal of the solvent a white solid was obtained.

M.P. 190-92° C. and MS (M+1)=475 (MH⁺, 100%) M.F.=C₂₁H₂₇FN₈O₄.

### Example-167

### trans-(R)-{3-[4-(4-(5-hydroxymethyl-1H-[1,2,3]-triazol-1-yl)-piperidin-1-yl)-3-fluoro-phenyl]-5-(5-hydroxymethyl-1H-[1,2,3]-triazol-1-ylmethyl)}-oxazolidin-2-one;

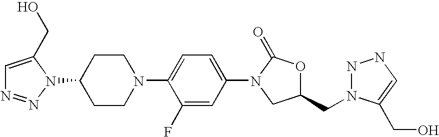

The isomer-3 was also converted to the alcohol using the same procedure as described above. M.P. 208-210° C. and MS (M+1)=475 (MH⁺, 100%) M.F.=C₂₁H₂₇FN₈O₄.

### Example-168

### trans-(R)-{3-[4-(4-(5-cyano-1H-[1,2,3]-triazol-1-yl)-piperidin-1-yl)-3-fluoro-phenyl]-5-(5-cyano-1H-[1,2,3]-triazol-1-ylmethyl)}-oxazolidin-2-one;

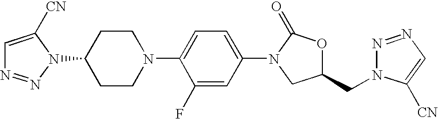

The isomer-3 was reacted with the aqueous ammonia to the ester into the amide and which was further reacted with the trifluoroacetic anhydride at room temperature for overnight. The solvent was removed and the crude mass obtained was purified by the column chromatography over silica gel using CHCl₃:MeOH (9.5:0.5) as an eluent to afford the desired compound as a white solid (0.120 mg, 65%).

M.P. 192-94° C. and MS (M+1)=463 (MH⁺, 100%) M.F.=C₂₁H₁₉FN₁₀O₂.

### Example-169

### trans-(R)-{3-[4-(4-(5-cyano-1H-[1,2,3]-triazol-1-yl)-piperidin-1-yl)-3-fluoro-phenyl]-5-(4-cyano-1H-[1,2,3]-triazol-1-ylmethyl)}-oxazolidin-2-one;

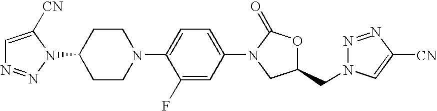

Using the isomer-4 and by the same procedure described as above the desired compound was obtained (90 mg, 60%)

M.P. 220-222° C. and MS (M+1)=463 (MH⁺, 100%) M.F.=C₂₁H₁₉FN₁₀O₂.

### Example-170

### (S)—N-{3-[3,5-Difluoro-4-(4-(4-methyl-5-thioxo-4,5-dihydro-tetrazol-1-yl)-piperidin-1-yl)-phenyl]-2-oxo-oxazolidin-5-ylmethyl}-acetamide;

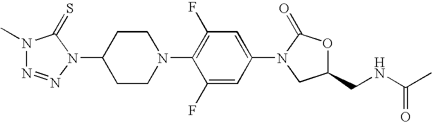

A mixture of (S)—N-{3-[4-methanesulphonyloxy piperidin-1-yl)-3,5-difluorophenyl]-2-oxo-oxazolidin-5-ylmethyl}-acetamide (1.12 mm), 5-mercapto-1-methyltetrazole (1.68 mm), and K₂CO₃ (1.68 mm) in DMF (6 ml) was heated for 22 hrs at 85° C. The resulting mixture was poured into ice-water mixture, stirred for 30 min. and the separated solid was purified by column chromatography to obtain product in 32% yield.

M.P. 182-184° C.; MS (M+1)-467; M.F. C₁₉H₂₃F₂N₇O₃S

### Example-171

### (S)—N-{3-[3,5-Difluoro-4-(4-(4-methyl-5-thioxo-4,5-dihydro-[1,2,4]triazol-1-yl)-piperidin-1-yl)-phenyl]-2-oxo-oxazolidin-5-ylmethyl}-acetamide;

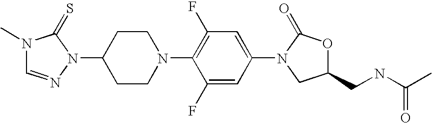

A mixture of (S)—N-{3-[4-methanesulphonyloxy piperidin-1-yl)-3,5-difluorophenyl]-2-oxo-oxazolidin-5-ylmethyl}-acetamide (1.12 mm), 4-methyl-4H-[1,3,4]-triazole-3-thiol (1.68 mm), and K₂CO₃ (1.68 mm) in DMF (6 ml) was heated for 22 hrs at 85° C. The resulting mixture was poured into ice-water mixture, stirred for 30 min. and the separated solid was purified by column chromatography to obtain product in 38% yield respectively. M.P. 168-170° C.; MS (M+1)-466; M.F. C₂₀H₂₄F₂N₆O₃S

### Example-172

(S)—N-{3-[3-fluoro-4-(4-(4-methyl-5-thioxo-4,5-dihydro-[1,2,4]triazol-1-yl)-piperidin-1-yl)-phenyl]-2-oxo-oxazolidin-5-ylmethyl}-acetamide;

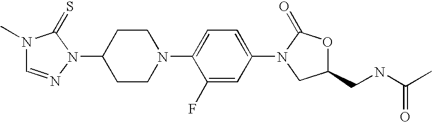

A mixture of (S)—N-{3-[4-methanesulphonyloxy piperidin-1-yl)-3-fluorophenyl]-2-oxo-oxazolidin-5-ylmethyl}-acetamide (1.12 mmole), 4-methyl 4H-[1,3,4]-triazole-3-thiol (1.68 mmole), and K₂CO₃ (1.68 mmole) in DMF (6 ml) was heated for 22 hrs at 85° C. The resulting mixture was poured into ice-water mixture, stirred for 30 min. and the separated solid was purified by column chromatography to obtain product in 22% yield respectively. M.P. 182-185° C.; MS (M+1)-448; M.F. C₂₀H₂₅FN₆O₃S

### Example-173

### (S)—N-{3-[4-(4-(isoxazol-3-yloxymethyl)-piperidin-1-yl)-3-fluorophenyl]-2-oxo-oxazolidin-5-ylmethyl}-acetamide;

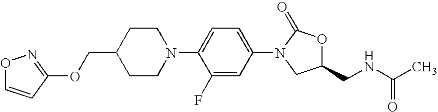

To the stirred solution of (S)—N-{-3-[3-Fluoro-4-(4-hydroxymethyl-piperidin-1-yl)-phenyl]-2-oxo-oxazolidin-5-ylmethyl}-acetamide (1.5 g, 4.1 mmol) in THF was added 3-hydroxy isoxazole (0.38 g, 4.52 mmol) and triphenylphospine (1.396 g, 5.33 mmol) and the resulting solution was stirred under nitrogen at rt for 10 minutes. DIAD (0.99 g, 4.92 nmoles) was added and stirred at an ambient temperature for 3 h. After completion, the reaction was quenched with 100 ml water & extracted with ethyl acetate (3×50 ml). The combined organic layers were washed with brine, dried (Na₂SO₄) and concentrated to give crude compound. Purification by column chromatography eluting with CHCl₃:CH₃OH (98:2) afforded the pure compound in 80% yield. M.P. 163-165° C. and MS (M+1)=433 (MH⁺, 100%) M.F.=C₂₁H₂₅FN₄O₅.

### Example-174

### (S)—N-{3-[4-(4-(1,4-dioxa-8-azaspiro[4.5]dec-8-yl-)-3-fluorophenyl)-3-fluorophenyl]-2-oxo-oxazolidin-5-ylmethyl}-acetamide;

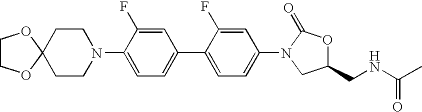

A solution of triphenylphosphine (440 mg, 1.6 mmol) in dimethoxyethane and tetrahydrofuran mixture (8:2, 40 ml) was stirred at room temperature under inert atmosphere, to it was added palladium acetate (90 mg, 0.4 mmol) and stirred for 30 min. (1,4-dioxa-8-azaspiro[4.5]dec-8-yl)-3-fluorophenyl)-boronic acid (3.54 gm, 12.6 mmol) was added and stirred for 30 min. Then water (190 μl, 10.4 mmol) was added and stirring continued, after 30 min powdered potassium carbonate (3.6 gm, 26 mmol) and (S)—N-3-{4-iodo-3-fluorophenyl]-2-oxo-oxazolidin-5-ylmethyl}-acetamide (3.92 gm, 10.4 mmol) was added under stirring. Reaction mixture was then heated to reflux for 14 hr. and title compound was obtained by purifying the crude compound on silica gel column chromatography in 72% yield.

MS (M+1)=488 (MH⁺, 100%) for M.F.=C₂₅H₂₇F₂N₃O₅.

Similarly, following compounds were synthesized.

| Example | | | Mol. |
|---|---|---|---|
| No. | Structure | M + 1 | formula |
| 175 | 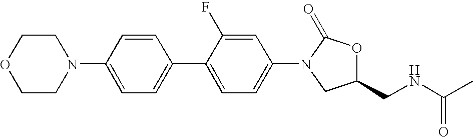 | 414 | C₂₂H₂₄FN₃O₄ |
| 176 | 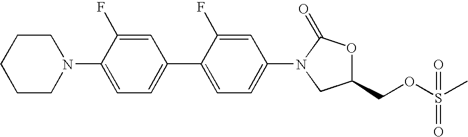 | 467 | C₂₂H₂₄F₂N₂O₅S₂ |
| 177 | 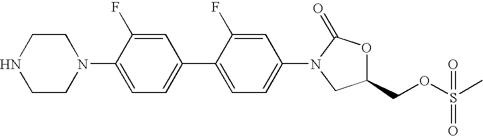 | 468 | C₂₁H₂₃F₂N₃O₅S |
| 178 | 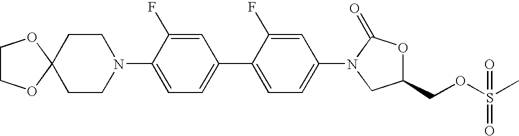 | 525 | C₂₄H₂₆F₂N₂O₇S |
| 179 | 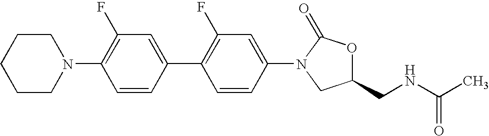 | 430 | C₂₃H₂₅F₂N₃O₃ |
| 180 | 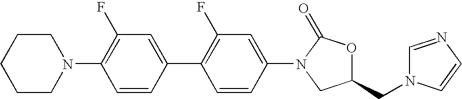 | 439 | C₂₄H₂₄F₂N₄O₂ |
| 181 | 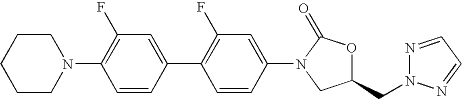 | 440 | C₂₃H₂₃F₂N₅O₂ |
| 182 | 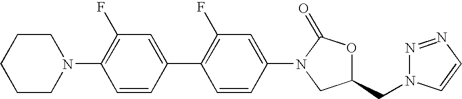 | 440 | C₂₃H₂₃F₂N₅O₂ |
| 183 | 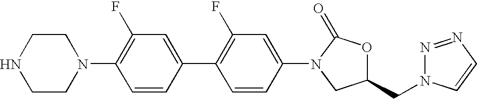 | 441 | C₂₂H₂₂F₂N₆O₂ |
| 184 | 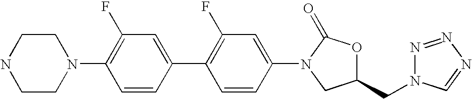 | 442 | C₂₁H₂₁F₂N₇O₂ |
| 185 | 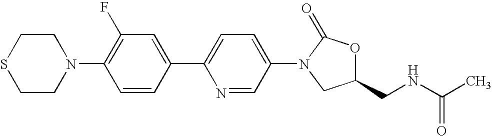 | 431 | C₂₁H₂₃FN₄O₃S |

### Example-186

### (S)—N-{3-[4-(6-(Thiomorpholin-1-yl)-pyridin-3-yl)-3-fluorophenyl]-2-oxo-oxazolidin-5-ylmethyl}-acetamide;

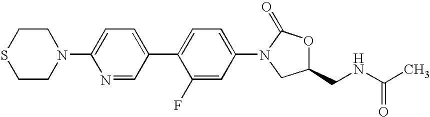

A solution of triphenylphosphine (220 mg, 0.8 mmol) in dimethoxyethane and tetrahydrofuran mixture (8:2, 20 ml) was stirred at room temperature under inert atmosphere, to it was added palladium acetate (45 mg, 0.2 mmol) and stirred for 30 min. (2-Thiomorpholin-1-yl-pyridin-5-yl)-boronic acid (1.41 gm, 6.3 mmol) was added and stirred for 30 min. Then water (94 μl, 5.2 mmol) was added and stirring continued, after 30 min powdered potassium carbonate (1.8 gm, 13 mmol) and (S)—N-3-{4-iodo-3-fluorophenyl]-2-oxo-oxazolidin-5-ylmethyl}-acetamide (1.96 gm, 5.2 mmol) was added under stirring. Reaction mixture was then heated to reflux for 14 hr. and title compound was obtained by purifying the crude compound on silica gel column chromatography in 72% yield.

MS (M+1)=431 (MH⁺, 100%) for M.F.=C₂₁H₂₃FN₄O₃S.

Similarly, following compounds were synthesized.

| | | | Molecular |
|---|---|---|---|
| Example | Structure | M + 1 | formula |
| 187 | 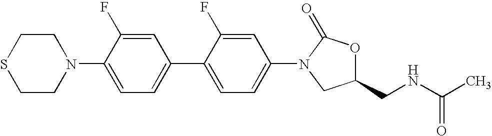 | 448 | C₂₂H₂₃F₂N₃O₃S |
| 188 | 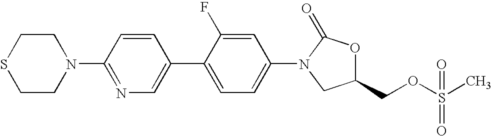 | 468 | C₂₀H₂₂FN₃O₅S₂ |
| 189 | 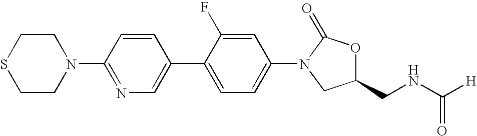 | 417 | C₂₀H₂₁FN₄O₃S |
| 190 | 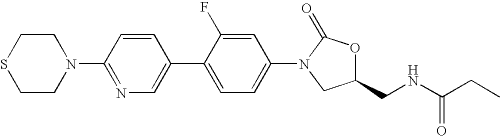 | 445 | C₂₂H₂₅FN₄O₃S |
| 191 | 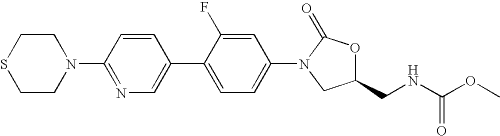 | 447 | C₂₁H₂₃FN₄O₄S |
| 192 | 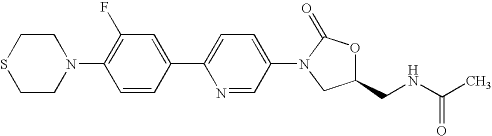 | 432 | C₂₁H₂₃FN₄O₃S |
| 193 | 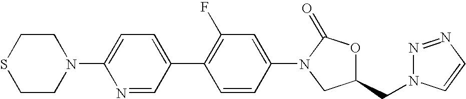 | 442 | C₂₁H₂₁FN₆O₂S |
| 194 | 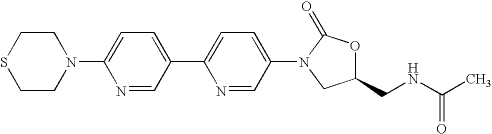 | 445 | C₂₀H₂₃N₅O₃S |
| 195 | 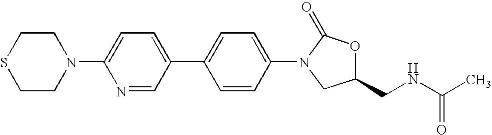 | 413 | C₂₁H₂₄N₄O₃S |
| 196 | 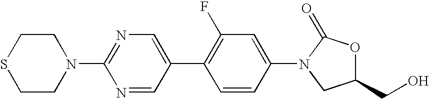 | 391 | C₁₈H₁₉FN₄O₃S |
| 197 | 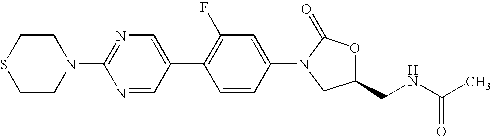 | 432 | C₂₀H₂₂FN₅O₃S |
| 198 | 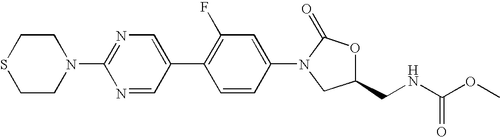 | 448 | C₂₀H₂₂FN₅O₄S |
| 199 | 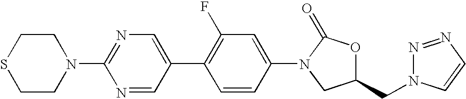 | 442 | C₂₀H₂₀FN₇O₂S |
| 200 | 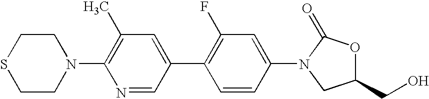 | 404 | C₂₀H₂₂FN₃O₃S |
| 201 | 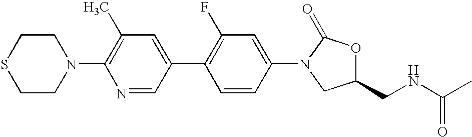 | 445 | C₂₂H₂₅FN₄O₃S |
| 202 | 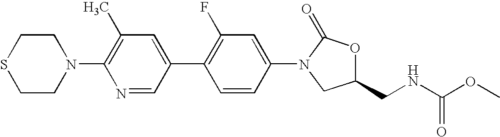 | 461 | C₂₂H₂₅FN₄O₄S |
| 203 | 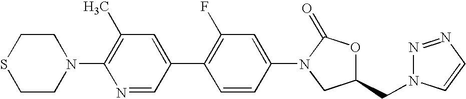 | 455 | C₂₂H₂₃FN₆O₂S |
| 204 | 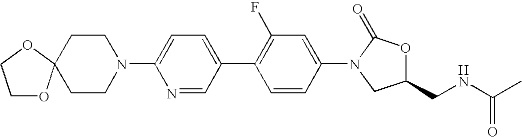 | 471 | C₂₄H₂₇FN₄O₅ |

### Example-205

### (S)—N-{3-[4-(6-(S-Oxo thiomorpholin-1-yl)-pyridyl-3-yl)-3-fluorophenyl]-2-oxo-oxazolidin-5-ylmethyl}-acetamide;

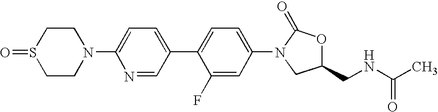

The title compound was prepared by reacting (S)—N-{3-[4-(6-(thiomorpholin-1-yl)-pyridin-3-yl)-3-fluorophenyl]-2-oxo-oxazolidin-5-ylmethyl}-acetamide (1.25 gm, 2.9 mmol) with sodium metaperiodate (0.68 gm, 3.2 mmol) in methanol water mixture (8:2, 15 ml) at 35° C. for 4 hours and by purifying the crude compound on silica gel column chromatography in 76% yield. MS (M+1)=447 (MH⁺, 100%) for M.F.=C₂₁H₂₃FN₄O₄S.

Similarly, following compounds were synthesize.

| | | | Molecular |
|---|---|---|---|
| Example | Structure | M + 1 | Formula |
| 206 | 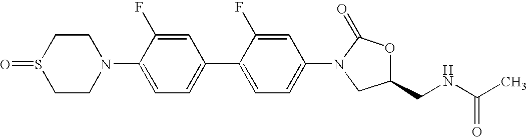 | 464 | C₂₂H₂₃F₂N₃O₄S |
| 207 | 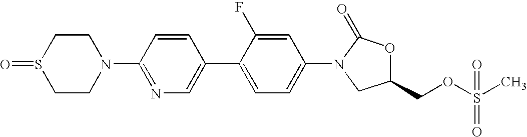 | 484 | C₂₀H₂₂FN₃O₆S₂ |
| 208 | 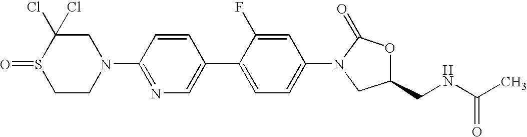 | 515 | C₂₁H₂₁Cl₂FN₄O₄S |
| 209 | 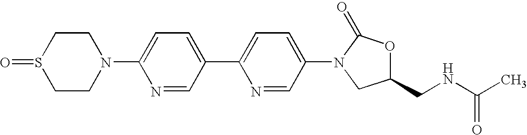 | 430 | C₂₀H₂₃N₅O₄S |
| 210 | 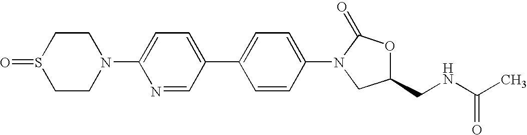 | 429 | C₂₁H₂₄N₄O₄S |
| 211 | 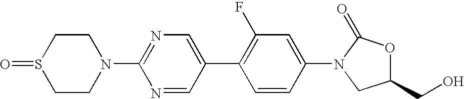 | 407 | C₁₆H₁₉FN₄O₄S |
| 212 | 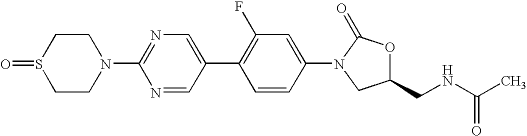 | 448 | C₂₀H₂₂FN₅O₄S |
| 213 | 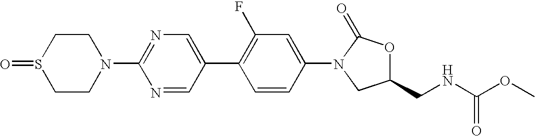 | 464 | C₂₀H₂₂FN₅O₅S |
| 214 | 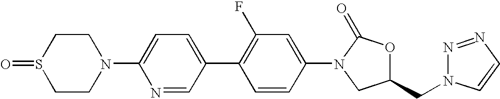 | 457 | C₂₁H₂₁FN₆O₃S |
| 215 | 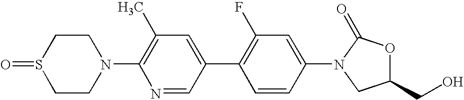 | 420 | C₂₀H₂₂FN₃O₄S |
| 216 | 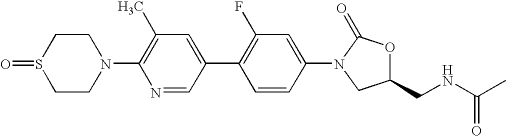 | 461 | C₂₂H₂₅FN₄O₄S |
| 217 | 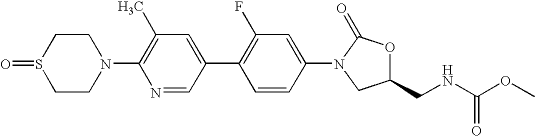 | 477 | C₂₂H₂₅FN₄O₅S |
| 218 | 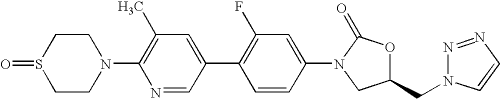 | 471 | C₂₂H₂₃FN₆O₃S |

### Example-219

### (S)—N-{3-[4-(2-(S,S-Dioxo-thiomorpholin-1-yl)-pyridyl-3-yl)-3-fluorophenyl]-2-oxo-oxazolidin-5-ylmethyl}-acetamide;

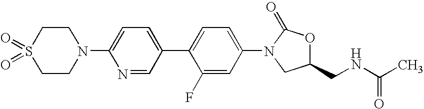

The title compound was prepared by reacting (S)—N-{3-[4-(2-(thiomorpholin-1-yl)-pyridin-5-yl)-3-fluorophenyl]-2-oxo-oxazolidin-5-ylmethyl}-acetamide (1.25 gm, 2.9 mmol) with sodium metaperiodate (1.37 gm, 6.4=mol) in methanol water mixture (8:2, 15 ml) at a temperature 70° C. for 6 hours and by purifying the crude compound on silica gel column chromatography in 87% yield. MS (M+1)=463 (MH⁺, 100%) for M.F.=C₂₁H₂₃FN₄O₅S.

Similarly, following compounds were synthesized

| | | | Molecular |
|---|---|---|---|
| Example | Structure | M + 1 | Formula |
| 220 | 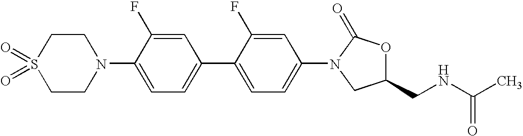 | 480 | C₂₂H₂₃F₂N₃O₅S |
| 221 | 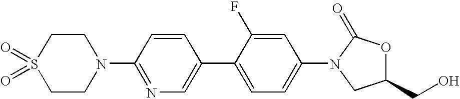 | 422 | C₁₉H₂₀FN₃O₅S |
| 222 | 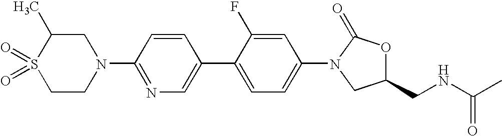 | 477 | C₂₂H₂₅FN₄O₅S |
| 223 | 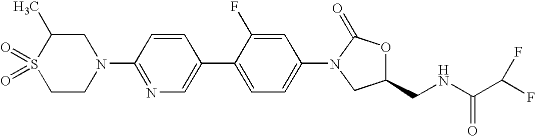 | 513 | C₂₂H₂₃F3N₄O₅S |
| 224 | 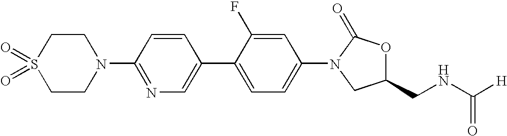 | 449 | C₂₀H₂₁FN₄O₅S |
| 225 | 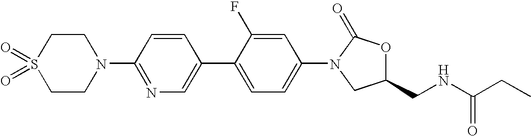 | 477 | C₂₂H₂₅FN₄O₅S |
| 226 | 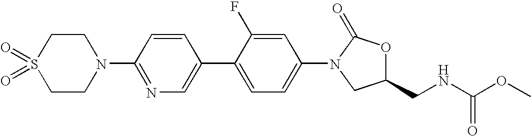 | 479 | C₂₁H₂₃FN₄O₆S |
| 227 | 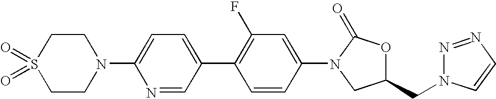 | 473 | C₂₁H₂₁FN₆O₄S |
| 228 | 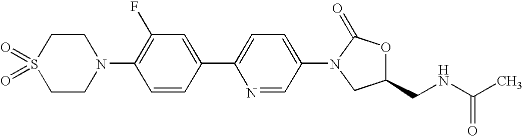 | 464 | C₂₁H₂₃FN₄O₅S |
| 229 | 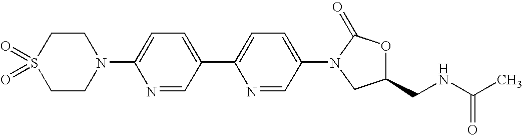 | 446 | C₂₀H₂₃N₅O₅S |
| 230 | 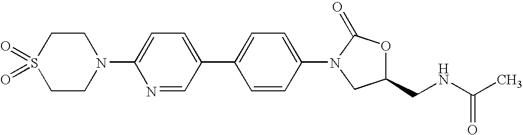 | 446 | C₂₁H₂₄N₄O₅S |
| 231 | 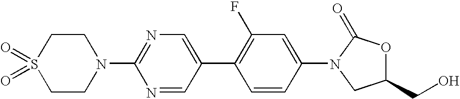 | 423 | C₁₈H₁₉FN₄O₅S |
| 232 | 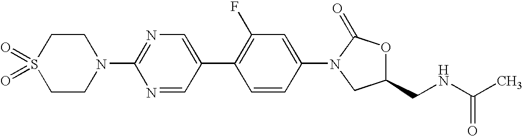 | 464 | C₂₀H₂₂FN₅O₅S |
| 233 | 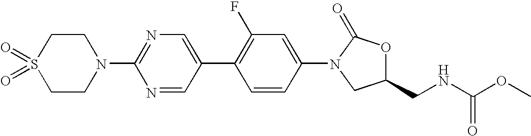 | 480 | C₂₀H₂₂FN₅O₆S |
| 234 | 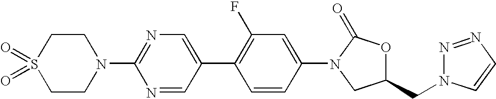 | 474 | C₂₀H₂₀FN₇O₄S |
| 235 | 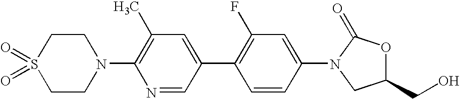 | 436 | C₂₀H₂₂FN₃O₅S |
| 236 | 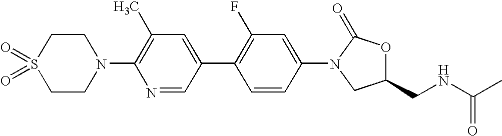 | 477 | C₂₂H₂₅FN₄O₅S |
| 237 | 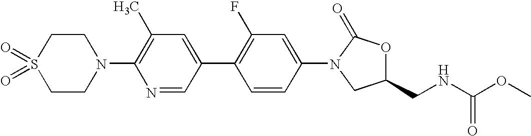 | 493 | C₂₂H₂₅FN₄O₆S |
| 238 | 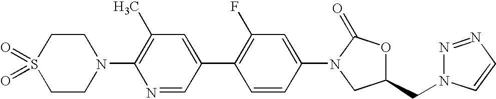 | 487 | C₂₂H₂₃FN₆O₄S |

### Example-239

### (S)—N-{3-{4-{6-(4-Oxopiperidin-1-yl-pyridin-3-yl)-3-fluorophenyl}-2-oxo-oxazolidin-5-ylmethyl}-acetamide;

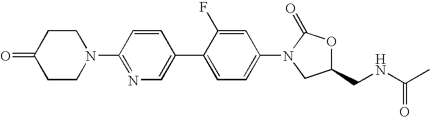

(S)—N-{3-{4-(2-(1,4-Dioxa-8-azaspiro[4.5]dec-8-yl)-pyridin-5-yl)-3-fluorophenyl}-2-oxo-oxazolidin-5-ylmethyl}-acetamide (469 mg, 100 mmol), p-toluene sulphonic acid (380 mg, 200 mmol) in acetone:water mixture (6:4) 10 ml, was refluxed for 5 hrs. The reaction mixture was concentrated under vacuum and neutralized with sodium bicarbonate. Obtained precipitate was filtered and purified on silica column to afford the title compound in 81% yield.

Ms (M+1)=427 (MH⁺, 100%), M. F.=C₂₂H₂₃FN₄O₄.

Similarly, following compounds were synthesized.

| | | | Molecular |
|---|---|---|---|
| Example | Structure | M + 1 | Formula |
| 240 | 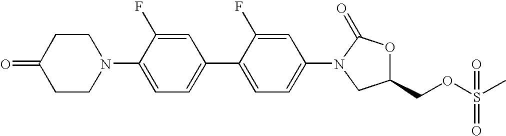 | 481 | C₂₂H₂₂F₂N₂O₆S |
| 241 | 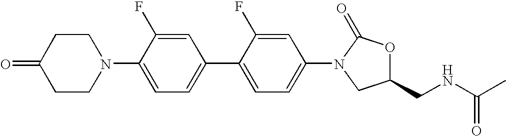 | 444 | C₂₃H₂₃F₂N₃O₄ |

### Example-242

### (S)—N-{3-[4-(6-(4-Hydroxy-piperidin-1-yl)-pyridin-3-yl)-3-fluorophenyl]-2-oxo-oxazolidin—5-ylmethyl}-acetamide

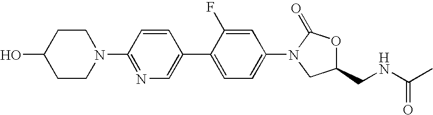

(S)—N-{3-{4-{2-(4-Oxopiperidin-1-yl-pyridin-5-yl)-3-fluorophenyl}-2-oxo-oxazolidin-5-ylmethyl}-acetamide (636 mg, 150 mmol), was treated with sodium borohydride (66 mg, 177 mmol) in 15 ml methanol at 5° C. and stirred for 1 hr at room temperature. The reaction mixture was concentrated under vacuum and treated with water, extracted with ethyl acetate. Organic layer separated, dried over sodium bicarbonate and evaporated to give title compound in 87% yield. Ms (M+1)=429 (MH⁺, 100%), M. F.=C₂₂H₂₅FN₄O₄.

Similarly, following compounds were synthesized.

| | | | Molecular |
|---|---|---|---|
| Example | Structure | M + 1 | Formula |
| 243 | 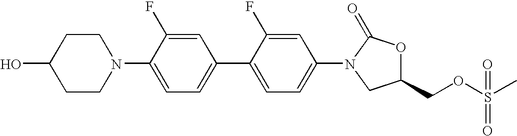 | 483 | C₂₂H₂₄F₂N₂O₆S |
| 244 | 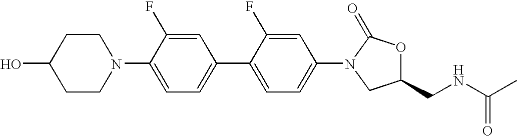 | 446 | C₂₃H₂₅F₂N₃O₄ |

### (S)—N-{3-[4-(6-(4-methanesulfonyloxy piperidin-1-yl)-pyridin-3-yl)-3-fluorophenyl]-2-oxo-oxazolidin-5-ylmethyl}-acetamide

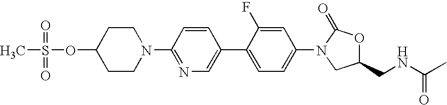

(S)—N-[3-{4-hydroxypiperidin-1-yl)-pyridin-3-yl]-3-fluoro-phenyl}-2-oxa-oxazolidin-5-yl]-acetamide (213 mg, 50 mmol) in 10 ml dichloromethane was treated with triethyl amine (175 μl, 125 mmol) and methanesulphonyl chloride (48 μl, 60 mmol) for 1 hr. The reaction mixture was washed with 10 ml water. Organic layer was separated dried over sodium sulfate and evaporated under vacuum to afford title compound in 91% yield.

Ms (M+1)=509 (MH⁺, 100%), M. F.=C₂₂H₂₅FN₄O₇S.

Similarly, following compounds were synthesized.

| | | | Molecular |
|---|---|---|---|
| Example | Structure | M + 1 | Formula |
| 246 | 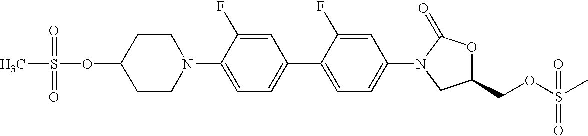 | 563 | C₂₂H₂₄F₂N₂O₉S₂ |
| 247 | 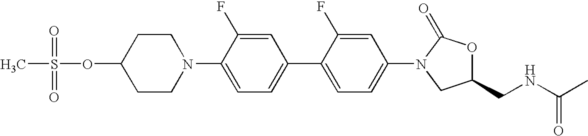 | 526 | C₂₃H₂₅F₂N₃O₇S |

### Example-248

### (S)—N-{3-{4-[4-(1-oxa-6-aza-spiro[2.5]oct-6-yl)-3-fluorophenyl]-3-fluorophenyl}-2-oxo-oxazolidin-5-ylmethyl}-acetamide;

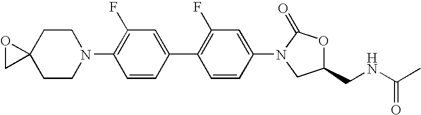

Sodium hydride (0.03 gm, 1.25 mmol) was added to stirred dimethyl sulphoxide 10 ml at room temperature, trimethylsulphoxonium iodide (0.33 gm, 1.5 mmol) was added to it after 10 min, Suspension became clear after 30 min of stirring. Then added (S)—N-{3-{4-{2-(4-Oxopiperidin-1-yl-3-fluorophenyl)-3-fluorophenyl}-2-oxo-oxazolidin-5-ylmethyl}-acetamide (0.44 gm, 1 mmol) and stirred for 1 hr. at room temperature. Quenched by using water, extracted with ethyl acetate and organic layer separated, dried and evaporated to give title compound in 88% yield.

Ms (M+1)=490 (MH⁺, 100%), M. F.=C₂₅H₂₉F₂N₃O₅

### Example-249

### (S)—N-{3-[4-(4-(4,4-dimethoxypiperidin-1-yl)-3-fluorophenyl)-3-fluorophenyl]-2-oxo-oxazolidin-5-ylmethyl}-acetamide;

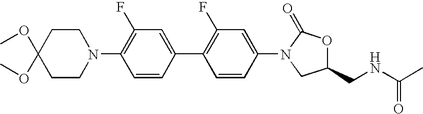

(S)—N-{3-{4-{2-(4-Oxopiperidin-1-yl-3-fluorophenyl}-3-fluorophenyl)-2-oxo-oxazolidin-5-ylmethyl}-acetamide (0.44 gm, 1 mmol) was dissolved in methanol 10 ml and p-toluene sulphonic acid (0.34 gm, 2 mmol). Trimethyl orthoformate (0.21 gm, 2 mmol) was added to it and stirred for 12 hr. at room temperature. Methanol evaporated up to dryness and reaction mixture neutralized by aqueous sodium bicarbonate, extracted with ethyl acetate and organic layer separated, dried and evaporated to give title compound in 68% yield. Ms (M+1)=458 (MH⁺, 100%), M. F.=C₂₄H₂₅F₂N₃O₄

### Example-250

### (S)—N-{3-[4-(4-(4-Cyanomethylidene piperidin-1-yl)-3-fluorophenyl)-3-fluorophenyl]-2-oxo-oxazolidin-5-ylmethyl}-acetamide;

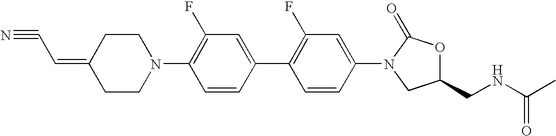

(S)—N-{3-{4-{2-(4-Oxopiperidin-1-yl-3-fluorophenyl)-3-fluorophenyl}-2-oxo-oxazolidin-5-ylmethyl}-acetamide (0.44 gm, 1 mmol) was dissolved in tetrahydrofuran (15 ml), to it was added triethyl amine (0.27 ml, 2 mmol), lithium bromide (0.11 gm, 1.2 mmol) and then diethylcyanomethyl phosphonate (0.22 ml, 1.2 mmol). The reaction mixture was heated at 80° C. for 12 hr. Solvent removed on rota evaporator and reaction mixture was treated with water. Extracted with ethyl acetate, organic layer separated, dried and evaporated to give title compound in 72% yield

Ms (M+1)=467 (MH⁺, 100%), M. F.=C₂₅H₂₄F₂N₄O₃.

Similarly, following compounds were synthesized.

| | | | Molecular |
|---|---|---|---|
| Example | Structure | M + 1 | Formula |
| 251 | 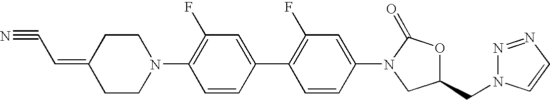 | 477 | C₂₅H₂₂F₂N₆O₂ |
| 252 | 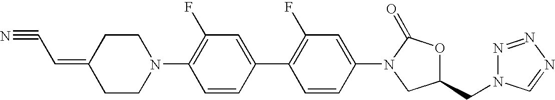 | 478 | C₂₄H₂₁F₂N₇O₂ |

### Example-253

### (S)—N-{3-[4-(4-cyanomethyl-piperidin-1-yl)-3-fluorophenyl}-3-fluorophenyl]-2-oxo-oxazolidin-5-ylmethyl}-acetamide

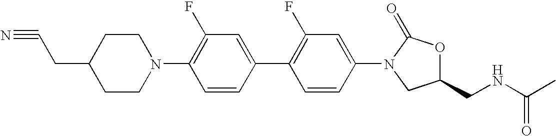

(S)—N-{3-[4-(4-(4-Cyanomethylidene piperidin-1-yl)-3-fluorophenyl)-3-fluoro phenyl]-2-oxo-oxazolidin-5-ylmethyl}-acetamide (0.47 gm, 1 mmol) was dissolved in methanol 25 ml to it was added 40 mg 10% palladium on carbon and under 60 PSI hydrogen pressure reaction was put on Parr shaker for 12 hr. After completion catalyst was filter through celite, solvent evaporated to give title compound in 94% yield. Ms (M+1)=469 (MH⁺, 100%), M. F.=C₂₅H₂₆F₂N₄O₃.

### Example-254

### (S)—N-{3-[4-(4-(4-cyanomethyl-3,4-dehydropiperidin-1-yl)-3-fluorophenyl)-3-fluorophenyl]-2-oxo-oxazolidin-5-ylmethyl}-acetamide

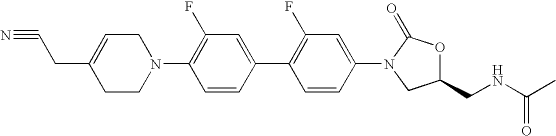

(S)—N-{3-{4-{2-(4-Oxopiperidin-1-yl-3-fluorophenyl)-3-fluorophenyl}-2-oxo-oxazolidin-5-ylmethyl}-acetamide (0.44 gm, 1 mmol) was suspended in toluene 15 ml, to it was added ammonium acetate (38 mg, 0.5 mmol), and cyanoacetic acid (94 mg, 1.1 mmol). The reaction mixture was heated at 110° C. for 12 hr. Solvent removed on rota evaporator and reaction mixture as such was put on silica column to give pure title compound in 66% yield

Ms (M+1)=467 (MH⁺, 100%), M.F.=C₂₅H₂₄F₂N₄O₃.

### Example-255

### (S)—N-{3-[4-(4-(4-(1H-[1,2,3]-triazol-1-yl)-piperidin-1-yl)-3-fluorophenyl)-3-fluorophenyl]-2-oxo-oxazolidin-5-ylmethyl}-acetamide;

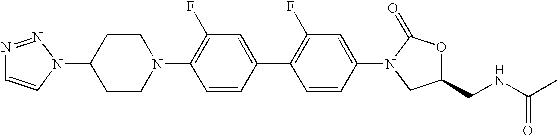

(S)—N-{3-[4-(4-(4-methansulfonyloxypiperidin-1-yl)-3-fluorophenyl)-3-fluoro phenyl]-2-oxo-oxazolidin-5-ylmethyl}-acetamide (0.53 gm, 1 mmol) was suspended in dimethylformamide 15 ml, and potassium carbonate (0.28 gm, 2 mmol), at room temperature. The reaction mixture was heated at 75° C. for 12 hr. Reaction mixture was filtered solvent removed on rota evaporator and treated with water, extracted with ethyl acetate organic layer separated and purified on silica column to give pure title compound in 54% yield. Ms (M+1)=497 (MH⁺, 100%), M.F.=C₂₅H₂₆F₂N₆O₃.

Similarly, following compounds were synthesized.

| | | | Molecular |
|---|---|---|---|
| Example | Structure | M + 1 | Formula |
| 256 | 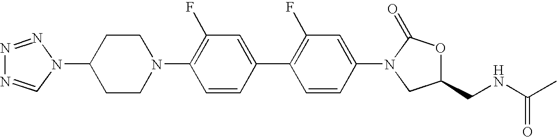 | 498 | C₂₄H₂₅F₂N₇O₃ |

### Example-257

### (S)—N-{3-[4-(4-benzyloxycarbonylpiperazin-1-yl-3-fluorophenyl)-3-fluorophenyl]-2-oxo-oxazolidin-5-ylmethyl}-acetamide;

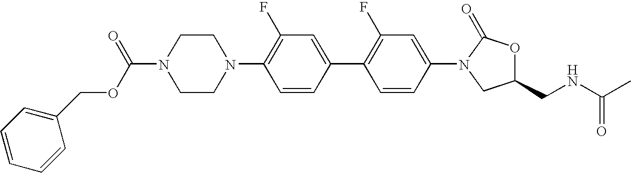

(S)—N-{3-[4-(piperazin-1-yl-3-fluorophenyl)-3-fluorophenyl]-2-oxo-oxazolidin-5-ylmethyl}-acetamide (0.43 gm, 1 mmol) was suspended in acetone:water mixture (8:2) 15 ml, potassium carbonate (0.28 gm, 2 mmol), at room temperature and then 2-benzyloxyacetyl chloride (0.24 ml, 1.4 mmol) was added to it. The reaction mixture was stirred at 35° C. for 12 hr. Reaction mixture was filtered, solvent removed on rota evaporator and treated with water, extracted with ethyl acetate organic layer separated and purified on silica column to give pure title compound in 78% yield Ms (M+1) 565 (MH⁺, 100%), M.F.=C₃₀H₃₀F₂N₄O₅.

Similarly, following compounds were synthesized.

| | | | Molecular |
|---|---|---|---|
| Example | Compound | M + 1 | Formula |
| 258 | 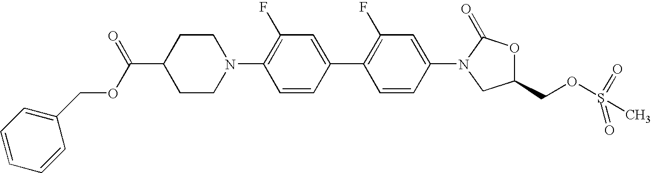 | 602 | C₂₉H₂₉F₂N₃O₇S |
| 259 | 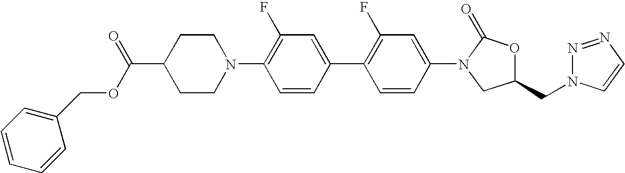 | 575 | C₃₀H₂₈F₂N₆O₄ |

### Example-260

### (S)—N-{3-[4-(4-(4-(2-nitrofuran-5-yl-methyl)-piperazin-1-yl)-3-fluorophenyl)-3-fluorophenyl]-2-oxo-oxazolidin-5-ylmethyl}-acetamide;

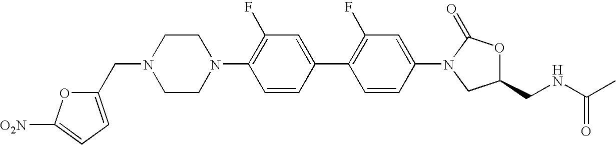

(S)—N-{3-[4-(piperazin-1-yl-3-fluorophenyl)-3-fluorophenyl]-2-oxo-oxazolidin-5-ylmethyl}-acetamide (0.43 gm, 1 mmol) was dissolved in dichloromethane 15 ml, 2-nitro furan aldehyde (0.28 gm, 2 mmol) was then added and stirred at room temperature for 12 hr and then sodium triacetoxyborohydride (0.24 ml, 1.4 mmol) was added to it at 0-5° C. The reaction mixture was stirred at 30° C. for 3 hr. solvent removed on rota evaporator and treated with water, extracted with ethyl acetate organic layer separated and purified on silica column to give pure title compound in 66% yield

Ms (M+1)=556 (MH⁺, 100%), M.F.=C₂₇H₂₇F₂N₅O₆.

Similarly, following compounds were synthesized.

| | | | Molecular |
|---|---|---|---|
| Example | Compound | M + 1 | Formula |
| 261 | 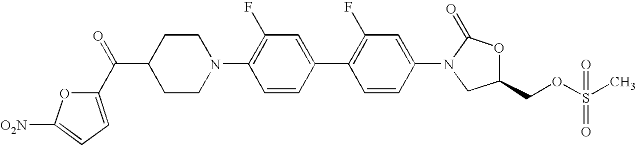 | 593 | C₂₆H₂₆F₂N₄O₈S |

### Example-262

### (S)—N-{3-[4-(4-(4-hydroxy-4-hydroxymethylpiperidin-1-yl)-3-fluorophenyl)-3-fluorophenyl]-2-oxo-oxazolidin-5-ylmethyl}-acetamide;

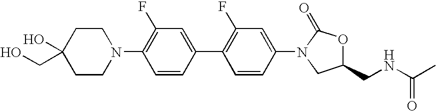

The title compound was prepared by treating (S)—N-{3-[4-(4-(1-Oxa-6-aza-spiro[2.5]oct-6-yl)-3-fluorophenyl)-3-fluorophenyl]-2-oxo-oxazolidin-5-ylmethyl}-acetamide (1.03 gm, 2.1 mmol) in 0.5 N hydrochloric acid (5 ml) at a temperature 80° C. for 2 hours and by purifying the crude compound by silica gel column chromatography in 46% yield.

MS (M+1)=476 (MH⁺, 100%) for M.F.=C₂₄H₂₇F₂N₃O₅

Similarly, following compounds were synthesized.

| | | | Molecular |
|---|---|---|---|
| Example | Structure | M + 1 | Formula |
| 263 | 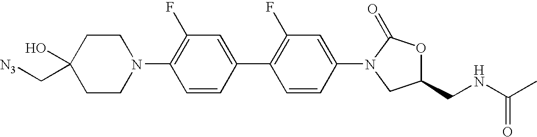 | 501 | C₂₄H₂₆F₂N₆O₄ |

### Example-264

### (S)—N-{3-[4-(6-(S-Oxo-S-imino-thiomorpholin-4-yl)-pyridyl-3-yl)-3-fluorophenyl-1-yl]-2-oxo-oxazolidin-5-ylmethyl}-acetamide;

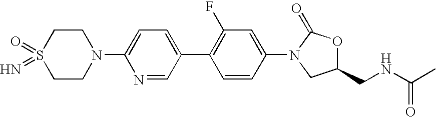

(S)—N-{3-4-{2-(S-Oxo thiomorpholin-4-yl)-pyridyl-5-yl-3-fluorophenyl}-]}-2-oxo-oxazolidin-5-ylmethyl}-acetamide (0.44 gm, 1 mmol) was dissolved in polyphosphoric acid 5 ml at room temperature, sodium azide (0.1 gm, 1.5 mmol) was then added and stirred at 60° C. for 12 hr. Cooled to room temperature, treated with water and neutralized with sodium bicarbonate, extracted with chloroform. Organic layer separated, solvent removed on rota evaporator and purified on silica column to give pure title compound in 56% yield. Ms (M+1)=462 (MH⁺, 100%), M.F.=C₂₁H₂₄FN₅O₄S.

Similarly, following compounds were synthesized.

| | | | Molecular |
|---|---|---|---|
| Example | Structure | M + 1 | Formula |
| 265 | 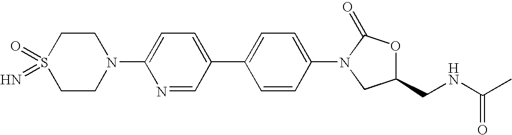 | 444 | C₂₁H₂₅N₅O₄S |

### Example-266

### (S)—N-{3-[4-(6-(S-Oxo-S—(N-methylimino)-thiomorpholin-4-yl)-pyridyl-3-yl)-3-fluorophenyl-1-yl]-2-oxo-oxazolidin-5-ylmethyl}-acetamide;

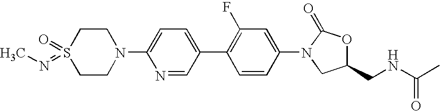

(S)—N-{3-[4-{2-(S-Oxo-1-iminothiomorpholin-4-yl)-pyridyl-5-yl-3-fluorophenyl-1-yl-}-]}-2-oxo-oxazolidin-5-ylmethyl}-acetamide (0.46 gm, 1 mmol) was dissolved in formic acid 5 ml at room temperature, formaldehyde (0.15 ml, 5 mmol) was then added and stirred at 80° C. for 12 hr. Cooled to room temperature, treated with water extracted with ethyl acetate. Organic layer separated, solvent removed on rota evaporator and purified on silica column to give pure title compound in 74% yield

Ms (M+1)=476 (MH⁺, 100%), M.F.=C₂₂H₂₆FN₅O₄S

### Example-267

### (S)—N-{3-[4-(6-(S-Oxo-S—(N-acetylimino)-thiomorpholin-4-yl)-pyridyl-3-yl)-3-fluorophenyl-1-yl]-2-oxo-oxazolidin-5-ylmethyl}-acetamide;

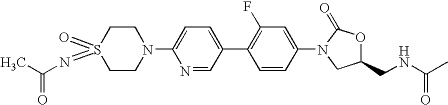

(S)—N-{3-4-{2-(S-Oxo-1-iminothiomorpholin-4-yl)-pyridyl-5-yl-3-fluorophenyl-1-yl-}-]}-2-oxo-oxazolidin-5-ylmethyl}-acetamide (0.46 gm, 1 mmol) was dissolved in dichloromethane 15 ml, triethyl amine (0.34 ml, 2.5 mmol) was then added and cooled to 0° C., then added acetyl chloride (88 μl, 1.1 mmol) and stirred at room temperature for 1 hr. Treated with water extracted with ethyl acetate. Organic layer separated, solvent removed on rota evaporator and purified on silica column to give pure title compound in 74% yield

Ms (M+1)=504 (MH⁺, 100%), M.F.=C₂₃H₂₆FN₅O₅S.

### Example-268

### (S)—N-{3-[4-(6-(S-Oxo-S-(1-chloroethylurido)-thiomorpholin-4-yl)-pyridyl-3-yl)-3-fluorophenyl-1-yl]-2-oxo-oxazolidin-5-ylmethyl}-acetamide;

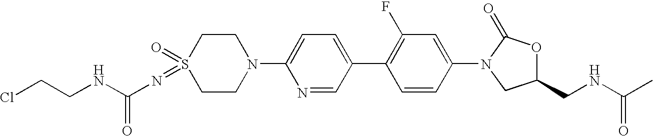

(S)—N-{3-4-{2-(S-Oxo-1-iminothiomorpholin-4-yl)-pyridyl-5-yl-3-fluorophenyl-1-yl-}-]}-2-oxo-oxazolidin-5-ylmethyl}-acetamide (0.46 gm, 1 mmol) was dissolved dichloromethane 15 ml, triethyl amine (0.34 ml, 2.5 mmol) was then added and cooled to 0° C., then added 1-chloroethylisocynate (126 mg, 1.2 mmol) and stirred at room temperature for 1 hr. at room temperature, treated with diethyl ether solid separated, filtered and purified on silica column to give pure title compound in 66% yield

Ms (M+1)=567 (MH⁺, 100%), M.F.=C₂₄H₂₈ClFN₆O₅S.

### Example-269

### (R)-Phosphoric acid mono {3-[4-(6-(S,S-dioxo-thiomorpholin-4-yl)-pyridin-3-yl)-3-fluorophenyl]-2-oxo-oxazolidin-5-ylmethyl}ester;

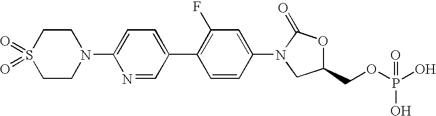

To a stirred solution of (S)—N-{3-{4-{2-(S,S-Dioxo thiomorpholin-4-yl)-pyridin-5-yl}-3-fluorophenyl]}-2-oxo-oxazolidin-5-yl methyl}-alcohol (0.84 gm, 2 mmol) and di-tert-butyl N,N-diisopropylphosphoramidite (1.2 gm, 3 mmol) in anhydrous dichloromethane 40 ml was added 1H-Tetrazole (0.48 gm, 7 mmol). After 1 hr of stirring mixture was cooled to 0° C. and 0.3 ml of 30% hydrogen peroxide was added. The mixture was stirred at room temperature for 1 hr, diluted with dichloromethane 80 ml and washed with 10% sodium metabisulfide and saturated sodium bicarbonate and water. Organic layer was separated concentrated to ⅓ volume and to it was added trifluoroacetic acid 3 ml. Stirred for 3.5 hr and evaporated up to dryness. Purification on silica column gave pure title product in 52% yield.

Ms (M+1)=502 (MH⁺, 100%), M.F.=C₁₉H₂₁FN₃O₈PS.

Similarly, following compounds were synthesized.

| | | | Molecular |
|---|---|---|---|
| Example | Structure | M + 1 | Formula |
| 270 | 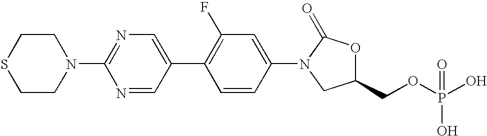 | 471 | C₁₈H₂₀FN₄O₆PS |
| 271 | 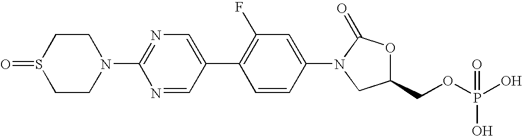 | 487 | C₁₈H₂₀FN₄O₇PS |
| 272 | 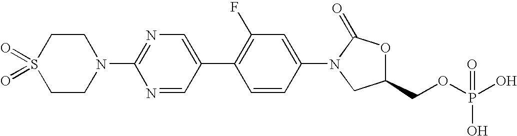 | 503 | |
| 273 | 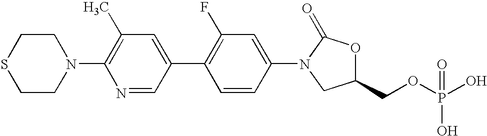 | 484 | |
| 274 | 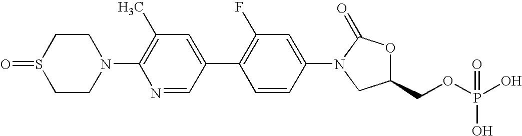 | 500 | |
| 275 | 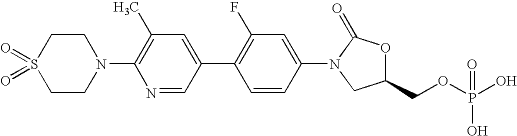 | 516 | |

### Example-276

### (2S,5R)-2-Amino-propionic acid 3-{4-[6-(S,S-dioxo-thiomorpholin-4-yl)-pyridin-3-yl]-3-fluoro-phenyl}-2-oxo-oxazolidin-5-ylmethyl ester methanesulfonic acid salt;

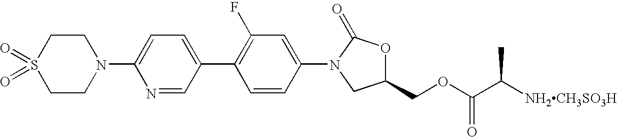

To a stirred solution of Boc alanine (0.35 gm, 1.85 mmol) in anhydrous dichloromethane 40 ml was added N,N-dicyclohexylcarbodiimide (0.48 gm, 7 mmol) and (S)—N-{3-{4-{2-(S,S-Dioxo thiomorpholin-4-yl)-pyridin-5-yl}-3-fluorophenyl]}-2-oxo-oxazolidin-5-ylmethyl}-alcohol (0.4 gm, 0.95 mmol) at −5° C. After 1 hr of stirring mixture was cooled to 0° C. and (65 mg, 0.5 mmol) N,N-dimethylpyridine was added. The mixture was stirred at 0° C. for 2 hr, diluted with dichloromethane 40 ml and washed with water. Organic layer was separated, dried and evaporated up to dryness. 25 ml acetone and (150 μl, 1.6 mmol) methanesulphonic acid was added and heated to 65° C. for 12 hr, Solid separated was filtered and washed with diethyl ether to yield title compound in 80% yield. Ms (M+1)=516 (MH⁺, 100%), M.F.=C₂₃H₂₆FN₃O₆S.

Similarly, following compounds were synthesized.

| | | | Molecular |
|---|---|---|---|
| Example | Structure | M + 1 | Formula |
| 277 | 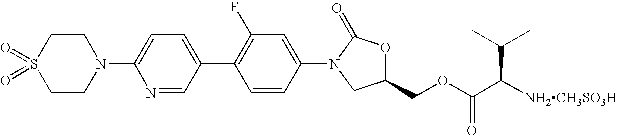 | 617 | C₂₆H₃₄FN₃O₉S₂ |

### Example-278

### (2S,5R)-Acetic acid 3-{4-[6-(S,S-dioxo-thiomorpholin-4-yl)-pyridin-3-yl]-3-fluoro-phenyl}-2-oxo-oxazolidin-5-ylmethyl ester;

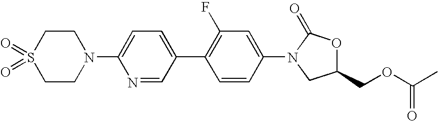

To a stirred solution of (S)—N-{3-{4-{2-(S,S-Dioxo thiomorpholin-4-yl)-pyridin-5-yl}-3-fluorophenyl]}-2-oxo-oxazolidin-5-ylmethyl}-alcohol (0.2 gm, 0.48 mmol) and pyridine (80 μl, 1 mmol) in anhydrous dichloromethane 20 ml was added acetic anhydride (58 μl, 0.56 mmol) and (29 mg, 0.5 mmol) N,N-dimethylpyridine was added at 0° C. The mixture was stirred at room temperature for 12 hr, diluted with dichloromethane 40 ml and washed with water. Organic layer was separated and evaporated up to dryness. Purification on silica column gave pure title product in 92% yield. Ms (M+1)=464 (MH⁺, 100%), M.F.=C₂₁H₂₂FN₃O₆S.

Similarly, following compounds were synthesized.

| | | | Molecular |
|---|---|---|---|
| Example | Compound | M + 1 | Formula |
| 279 | 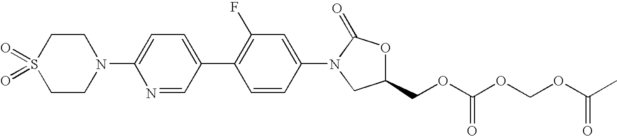 | 538 | C₂₃H₂₄FN₃O₉S |

### Example-280

### (5S)-{3-[2-Fluoro-1,1′-biphenyl-3′-methoxy-4-yl]-2-oxo-oxazolidin-5yl-methyl}-acetamide

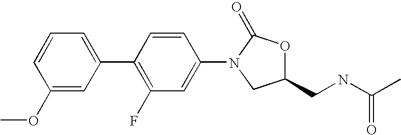

N-[3-(3-Fluoro-4-iodo-phenyl)-2-oxo-oxazolidin-5-ylmethyl]-acetamide (1.322 mmol) and tetrakis(triphenylphosphine)palladium (0.05 g) were added to a r. b. flask containing tetrabutylammonium bromide (1.5 g) and the contents were stirred and heated to 120° C. 3-Methoxyphenylboronic acid (1.45 mmol) and aq. potassium carbonate (0.35 g potassium carbonate dissolved in 1.5 ml water) were added to the r.b. flask and the reaction mixture was stirred for about 3 hours, till the TLC showed completion of the reaction. Water (50 ml) was added to the reaction. mixture and the contents were, extracted with ethyl acetate (25 ml×2). The organic layer was dried over anhy. sodium sulfate and evaporated under vacuum to obtain a crude compound, which was purified by flash silica gel column chromatography to get the title compound in 34% yield. M.P. 98-100° C., M.F.=C₁₉H₁₉FN₂O₄, M+1=359.

Using the procedure described for the above compound the title compound was prepared.

| | | | Molecular |
|---|---|---|---|
| Example | Compound | M + 1 | Formula |
| 281 | 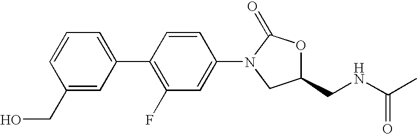 | 359 | C₁₉H₁₉FN₂O₄ |

### Example-283

### (5S)-{3-[(3,5-Difluoro-4-[6-(4-thiomorpholin-1,-dioxide-4-yl)-3-pyridinyl]phenyl]-2-oxo-oxazolidin-5-yl-methyl}-acetamide;

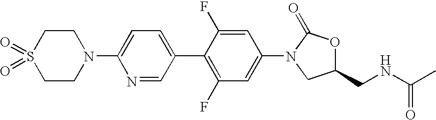

N-{3-[3,55-Difluoro-4-(6-thiomorpholin-4-yl-pyridin-3-yl)-phenyl]-2-oxo-oxazolidin-5-ylmethyl}-acetamide (0.446 mmol) was dissolved in aq. methanol (1:1, 5 ml) and sodium periodate (1.33 mmol) was added at R. T. The reaction mixture was stirred for about 1 hour, till the TLC showed completion of the reaction. Methanol was evaporated under vacuum and the solid was filtered to obtain the crude compound. It was purified by flash silica gel column chromatography to get the title compound in 40% yield.

M.P.=146-148° C.; M.F.=C₂₁H₂₂F₂N₄O₅S; M+1=481.

### Example-284

### (5S)—N-[3-(3′-Chloromethyl-2-fluoro-biphenyl-4-yl)-2-oxo-oxazolidin-5-ylmethyl]-acetamide;

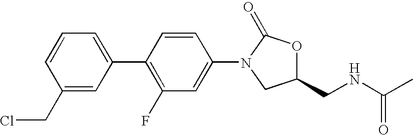

To the solution of (5S)-{3-[(2-fluoro-1,1′-biphenyl-3′-yl)methanol]-2-oxo-oxazolidin-5yl-methyl}-acetamide (1.8 mmol) in dichloromethane (25 ml) was added triethyl amine (5 mmol) and the contents were stirred at R. T. Methanesulfonic acid (3 mmol) was added drop-wise to the solution at R. T. and the reaction mixture was stirred for about 3 hours, till the TLC showed disappearance of the starting material. The reaction mixture was evaporated under vacuum and water (25 ml) was added to the residue. The crude product thus obtained was filtered, washed with water and dried under vacuum. The crude product was purified by flash silica gel column chromatography to get the title compound in 60% yield. M.P.=128-130° C.; M.F.=C₁₉H₁₈ClFN₂O₃; M+1=377.

### Example-285

### (5S)—N-[3-(2-Fluoro-3′-fluoromethyl-biphenyl-4-yl)-2-oxo-oxazolidin-5-ylmethyl]-acetamide;

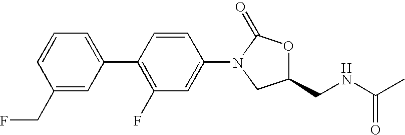

The solution of (5S)-{3-[(2-fluoro-1,1′-biphenyl-3′-yl)methanol]-2-oxo-oxazolidin-5yl-methyl}-acetamide (2.23 mmol) in dichloromethane (250 ml) was cooled to 5° C. and (diethylamino)sulfur trifluoride (DAST, 2.23 mmol) was added to the above solution. The contents were stirred at 5° C. for about 4 hours, till the TLC showed disappearance of the starting material. The reaction mixture was evaporated under vacuum and water (50 ml) was added to the residue. The crude product thus obtained was filtered, washed with water and dried under vacuum. The crude product was purified by flash silica gel column chromatography to get the title compound in 25% yield.

M.P.=120-122° C.; M.F.=C₁₉H₁₈F₂N₂O₃; M+1=361.

### Example-286

(5S)—N-[3-(2-Fluoro-3′-azidomethyl-biphenyl-4-yl)-2-oxo-oxazolidin-5-ylmethyl]-acetamide;

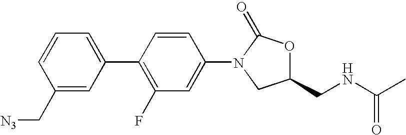

(5S)—N-[3-(2-Fluoro-3′-chloromethyl-biphenyl-4-yl)-2-oxo-oxazolidin-5-ylmethyl]-acetamide (2.7 mmol) was dissolved in N,N-DMF (30 ml) and sodium azide (8.1 mmol) was added to the above solution. The contents were stirred at 70° C. for about 6 hours, till the TLC showed disappearance of the starting material. The reaction mixture was poured in water (100 ml), the precipitated product was filtered and suck dried to obtain the title compound. M.F.=C₁₉H₁₈FN₅O₃; M+1=384.

### Example-287

### (5S)—N-[3-(3′-Aminomethyl-2-fluoro-biphenyl-4-yl)-2-oxo-oxazolidin-5-ylmethyl]-acetamide;

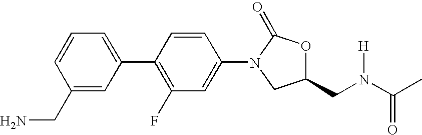

(5S)—N-[3-(2-Fluoro-3′-azidomethyl-biphenyl-4-yl)-2-oxo-oxazolidin-5-ylmethyl]-acetamide (2.61 mmol), Palladium on carbon (Pd/C, 100 mg) and methanol (30 ml) were charged in a r. b. flask and the contents were stirred under hydrogen atmosphere (rubber balloon) at RT. After about 5 hours, the TLC showed completion of the reaction. The Pd/C was filtered on celite bed and the bed was washed with methanol (50 ml). The combined filtrate was evaporated under vacuum to obtain a crude product, which was purified by flash silica gel column chromatography to get the title compound in 68% yield. M.P.=170° C.; M.F.=C₁₉H₂₀FN₃O₃, M+1=358.

### Example-288

### (5S)-2-({4′-[5-(Acetylamino-methyl)-2-oxo-oxazolidin-3-yl]-2′-fluoro-biphenyl-3-ylmethyl}-amino)-acetamide;

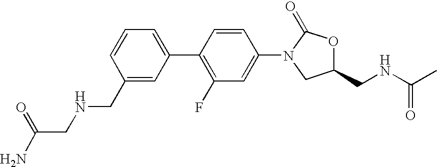

To the solution of (5S)—N-[3-(3′-aminomethyl-2-fluoro-biphenyl-4-yl)-2-oxo-oxazolidin-5-ylmethyl]-acetamide (1.12 mmol) in methanol (10 ml) and dichloromethane (10 ml), was added Hunig's base (1.12 mmol) and the contents were heated to 80° C. Bromoacetamide (1.12 mmol) was charged to the above solution and the reaction mixture was stirred at 80° C. for about 7 hours, till the TLC showed completion of the reaction. The solvents were removed under vacuum and water (20 ml) was added to the residue to obtain a crude product. The crude product was purified by flash silica gel column chromatography to get the title compound in 32.3% yield. M.F.=C₂₁H₂₃FN₄O₄, M+1=415.

### Example-289

### (5S)-2-({4′-[5-(Acetylamino-methyl)-2-oxo-oxazolidin-3-yl]-2′-fluoro-biphenyl-3-ylmethyl}-carbamoylmethyl-amino)-acetamide

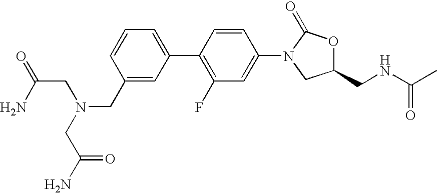

To the solution of (5S)—N-[3-(3′-aminomethyl-2-fluoro-biphenyl-4-yl)-2-oxo-oxazolidin-5-ylmethyl]-acetamide (1.12 mmol) in methanol (10 ml) and dichloromethane (10 ml), was added Hunig's base (4.12 mmol) and die contents were heated to 80° C. Bromoacetamide (4.12 mmol) was charged to the above solution and the reaction mixture was stirred at 80° C. for about 7 hours, till the TLC showed completion of the reaction. The solvents were removed under vacuum and water (20 ml) was added to the residue to obtain a crude product. The crude product was purified by flash silica gel column chromatography to get the title compound in 11% yield. M.P.=213° C., M.F.=C₂₂H₂₆F N₅O₅, M+1=472.

We claim:

1. An oxazolidinone compound of Formula I. 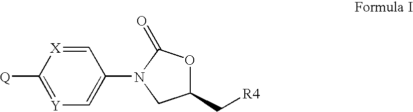
wherein Q is 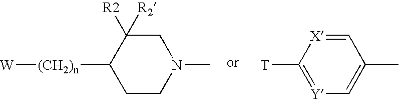
X and Y may be same or different, represent, CH, CF, N;
X′ and Y′ may be same or different, represent,
CH, CF, N, C—OCH₃, C—CH₂—Rₐ; wherein Rₐ is hydrogen, amino, halogen, hydroxyl, azido, carboxamido, NHCH₂CONH₂, N(CH₂CONH₂)₂;
R₂ and R_{2′} may be same or different, represent, hydrogen, methyl, hydroxyl, C₁-C₆ alkoxy or halogen;
R₄ is selected from the group comprising
a) O—R₁,
b) NR_{b}R_{c};
wherein R_{b} is selected from hydrogen, C₁-C₆ alkylcarbonyl;
R_{c} is selected from hydrogen, C(O)OCH₂OC(O)R_{d} or CH(CH₃)OC(O)Rₑ;
wherein R_{d} is hydrogen, C₁-C₆ alkyl, substituted C₁-C₆ alkyl with substitutents selected from amino or C₁-C₆ alkylamido
Rₑ is selected from hydrogen, C₁-C₆ alkyl, substituted C₁-C₆ alkyl;
c) formamide;
d) carbamate;
e) thiocarbamate;
f) urea;
g) C₁-C₆ alkylamido;
h) C₁-C₆ haloalkylamido;
i) C₁-C₆ thioalkylamido;
j) substituted C₁-C₆ thioalkylamido, wherein the alkyl group is substituted with halo;
k) unsubstituted or substituted heteroaryl, wherein substituents of the heteroaryl to are selected from the group comprising C₁-C₆ alkyl, nitro, cyano, hydroxyl, halo, amino, C₁-C₆ alkylcarbonyl, C₁-C₆ alkoxycarbonyl, carboxy;
wherein R₁ is selected from the group comprising hydrogen, C₁-C₆ alkylsulfonyl, C₁-C₆-alkylcarbonyl, —P(O)(OM)₂, wherein M is hydrogen, methyl, ethyl, t-butyl, phenyl or an alkali metal ion such as Li, Na, K;
or R₁ is an amino acid residue derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, attached to the oxygen via carbonyl of the amino acid to form a ester linkage;
When Q is 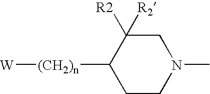
wherein n is 0, 1 or 2;
W represents 3-7 membered heterocyclyl bearing one or more heteroatom selected from N; O, S, optionally substituted with one or more substitutents selected from the group comprising C₁-C₆ alkyl, C₁-C₆ alkylcarbonyl, C₁-C₆ alkoxycarbonyl, carboxamide, cyano, hydroxyl, amino, heterocyclyl, substituted heterocyclyl, aryl, substituted aryl, heteroaryl, substituted heteroaryl
or
heteroaryl bearing one or more heteroatom selected from N, O S, optionally substituted with one or more substitutents selected from the group comprising
i. substituted or unsubstituted C₁-C₆ alkyl with substituents selected from the group hydroxyl, halo, nitro, cyano, aryl or heteroaryl;
ii. substituted or unsubstituted C₂-C₆ alkenyl with substituents selected from the group hydroxyl, halo, nitro, cyano, aryl or heteroaryl;
iii. C₁-C₆ alkylcarbonyl;
iv. C₁-C₆ alkoxycarbonyl;
v. C₁-C₆ alkoxycarbonylalkyl;
vi. C₁-C₆ alkylcarbonylalkyl;
vii. C₁-C₆ haloalkyl;
viii formyl;
ix. carboxy;
x. carboxamide;
xi. cyano;
xii. amino;
xiii nitro;
xiv. hydroxyl;
xv halo;
xvi. morpholinocarbonylalkyl;
xvii. hydroxyiminoalkyl;
xviii. alkylcarbonylaminoalkyl;
xix. alkoxyiminoalkyl;
xx. aralkyloxyiminoalkyl;
xxi. hydroiminoaminoalkyl;
xxii. aryl, substituted aryl, with substituents selected from C₁-C₆ alkyl, nitro, cyano, hydroxyl, halo, amino, heterocyclyl;
xxiii. substituted or unsubstituted heteroaryl, with substituents selected from C₁-C₆ alkyl, nitro, cyano, hydroxyl, halo, amino, heterocyclyl;
xxiv. substituted or unsubstituted heterocyclyl, with substituents selected from C₁-C₆ alkyl, C₁-C₆ alkylcarbonyl, aralkyl, nitro, cyano, hydroxyl, halo, amino;
xxv. C₁-C₆ alkylthio;
xxvi. C₁-C₆ alkylsulfanylalkyl;
xxvii. C₁-C₆ aralkylsulfanyl;
xxviii. C₁-C₆ alkylsulfonylmethyl;
xxix. C₁-C₆ alkylsulfonyloxy;
xxxi. —(C₁-C₆ alkyl)-NR_{f}—(C₁-C₆ alkyl)-NR_{b}R_{c};
wherein R_{f} is hydrogen, C₁-C₆ alkyl, substituted C₁-C₆ alkyl with substitutents selected from amino or C₁-C₆ alkylamido;
or
W is heteroaryloxy group optionally substituted with one or more substitutents such as C₁-C₆ alkyl, C₂-C₆ alkenyl, C₁-C₆ alkylcarbonyl, C₁-C₆ alkoxycarbonyl, formyl, carboxy, carboxamide, cyano, amino, hydroxyiminoalkyl, alkoxyiminoalkyl, aralkyloxyiminoalkyl, hydroxyiminoaminoalkyl, aryl, substituted aryl, heteroaryl, substituted heteroaryl, heterocyclyl, substituted heterocyclyl;
OR
when Q is 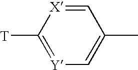
X′ and Y′ are as defined above;
T is hydrogen or 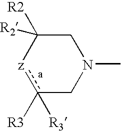
wherein
R₂ and R_{2′} are as defined above;
“a” is optional double bond;
R₃ and R_{3′} may be same or different, represent, hydrogen, methyl, hydroxyl, C₁-C₆ alkoxy or halogen;
with the proviso that when T is hydrogen, R₄ is not acetamido;
Z represents group selected from CH, NH, O, S, CH₂, C(R₆)R₆′, C═O, NR₇, C═C(R₈)R_{8′}, SO, SO₂, S—NH, S═NC(O)CH₃, S═NC(O)NHCH₃, S(O)═NH, S(O)═NCH₃, S(O)NC(O)CH₃, S(O)═NC(O)NHCH₃, S(O)═NC(O)NHCH₂CH₂Cl,
with proviso that when X is CH, Y is CF, one of X′ or Y′ or both X′, Y′ are N, then Z is not NH.
wherein R₆ and R₆′ may be same or different, represent, hydrogen, hydroxyl, amino, azido, C₁-C₆ alkoxy, C₁-C₆ alkylcarbonyloxy, C₁-C₆ alkylsulfonyloxy, heterocyclyl, substituted heterocyclyl, aryl, substituted aryl, heteroaryl, substituted heteroaryl, C₁-C₆ alkyl optionally substituted with one or more substituent selected from the group comprising azido, cyano, carboxamido, hydroxyl, C₁-C₆ alkyloxy, C₁-C₆ alkylcarbonyloxy, C₁-C₆ alkylcarbonyl or
C₂-C₆ alkenyl optionally substituted with one or more azido, cyano, carboxamido or hydroxyl;
or
R₆ and R₆′ are combined together to provide 3-7 member heterocyclyl bearing one or more heteroatom selected from N, O, S optionally substituted with substituent selected from group comprising C₁-C₆ alkyl, C₂-C₆ alkenyl, C₁-C₆ alkylcarbonyl, C₁-C₆ alkoxycarbonyl, formyl, carboxy, carboxamide, cyano, amino, hydroxyiminoalkyl, alkoxyiminoalkyl, aralkyloxyiminoalkyl, hydroxyiminoaminoalkyl, aryl, substituted aryl, heteroaryl, substituted heteroaryl, heterocyclyl, substituted heterocyclyl;
R₇ represents hydrogen, cyano, aralkyl, CO—OCH₂Ph, C₁-C₆-alkylcarbonyl, C₁-C₆ alkyl optionally substituted with one or more azido, cyano, heteroaryl, substituted heteroaryl, carboxamido or hydroxyl;
R₈, R_{8′} may be same or different, represent, hydrogen, C₁-C₆ alkyl, C₂-C₆ alkenyl, cyano, amino, C₁-C₆ alkylcarbonyl, C₁-C₆ alkoxycarbonyl, cyano, aryl, substituted aryl, heteroaryl, substituted heteroaryl, heterocyclyl, substituted heterocyclyl;
and isomers, polymorphs, N-oxides thereof or pharmaceutical acceptable salts thereof.

2. The compound of claim 1, wherein, X, Y, R₄ are as defined in claim 1 and Q is 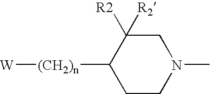
wherein n, R₂, R_{2′}, W, have the definitions as provided in claim 1.

3. The compound of claim 2, wherein n is 0 or 1.

4. The compound of claim 2, wherein R₂ and R_{2′} are each independently hydrogen, methyl, hydroxyl, halogen.

5. The compound of claim 4, wherein halogen is F.

6. The compound of claim 2, wherein W is heteroaryl or substituted heteroaryl, wherein substituents are as defined in claim 1.

7. The compound of claim 6, wherein heteroaryl is [1,2,3]-triazol, [1,2,4]-triazol, pyrrol, pyrazol, tetrazol, imidazol, [1,3,4]-oxadiazol, [1,2,4]-thiadiazol, [1,3,4]-thiadiazol, oxazol, isoxazol, thiazol, benzotriazol.

8. The compound of claim 6, wherein heteroaryl is [1,2,3]-triazol, [1,2,4]-triazol, pyrazol, tetrazol, [1,2,4]-thiadiazol, [1,3,4]-thiadiazol.

9. The compound of claim 6, wherein heteroaryl is [1,2,3]-triazol, tetrazol.

10. The compound of claim 2, wherein n is 0 or 1;
R₂, R_{2′} each and independently is hydrogen, hydroxyl, fluorine;
W is [1,2,3]-triazol or tetrazol; R₄ is as defined in claim 1.

11. The compound of claim 6, wherein the substituents of the heteroaryl are selected from the group comprising methyl, cyano, amino, ethoxycarbonyl, fluoro, difluoromethyl, formyl, [1,3]-dioxolan, hydroxymethyl, hydroxyethyl, carboxamido, carboxy, acetyl, methyl-oxiranyl, (E/Z)-2-cyano-vinyl, hydroxyimino-methyl, 1-methoxyimino-ethyl, 1-methoxyimino-methyl, 1-benzyloxyimino-methyl, hydroxyiminoamino-methyl, 4-N,N-dimethylaminoethylaminomethyl, phenyl, 4-nitrophenyl, 4-pyridinyl, 3-pyridinyl, morpholin-4-yl, diethylamino, piperazin-1-yl, methylsulfanylmethyl, methylsulfonylmethyl, benzylsulfanyl, thiophen-2-yl-methyl, morpholinocarbonylmethyl, 4-methylpiperazin-1-yl, 4-acetyl-piperazin-1-yl, 4-cyanopiperazin-1-yl, 4-benzylpiperazin-1-yl, ethoxycarbonylmethyl, acetylaminomethyl.

12. The compound of claim 6, wherein substituted heteroaryl is selected from the group comprising 3-ethoxycarbonyl-imidazol, fluoro-4-(1H-[1,2,3]-triazol, 4-difluoromethyl-1H-[1,2,3]-triazol, 4-formyl-1H-[1,2,3]-triazol, 4-[1,3]-dioxolan-2-yl-1H-[1,2,3]-triazol, 4-ethoxycarbonyl-1H-[1,2,3]-triazol, 4-hydroxymethyl-1H-[1,2,3]-triazol, 4-cyano-1H-[1,2,3]-triazol, 4-carboxamido-1H-[1,2,3]-triazol, 4-hydroxycarbonyl-1H-[1,2,3]-triazol, 4-acetyl-H-[1,2,3]-triazol, 4-(1-hydroxyethyl)-1H-[1,2,3]-triazol, 4-(methyl-oxiranyl)-1H-[1,2,3]-triazol, 4-(2-hydroxyethyl)-1H-[1,2,3]-triazol, 4-((E/Z)-2-cyano-vinyl)-1H-[1,2,3]-triazol, 4-(hydroxyimino-methyl)-1H-[1,2,3]-triazol, 4-(1-methoxyimino-ethyl)-1H-[1,2,3]-triazol, 4-(1-methoxyimino-methyl)-1H-[1,2,3]-triazol, 4-(1-benzyloxyimino-methyl)-1H-[1,2,3]-triazol, 4-(hydroxyiminoamino-methyl)-[1,2,3]-triazol, 4-benzotriazol, 4-N,N-dimethylaminoethylaminomethyl)-1H-[1,2,3]-triazol, 1-methyl-1H-tetrazol, 2-methyl-2H-tetrazol, 5-methyl-tetrazol, 5-phenyl-tetrazol, 5-(4-nitrophenyl)-tetrazol, 5-(4-pyridinyl)-tetrazol, 5-(3-pyridinyl)-tetrazol, 5-(morpholin-4-yl)-tetrazol, 5-amino-tetrazol, 5-(diethylamino)-tetrazol, 5-(piperazin-1-yl)-tetrazol, 5-methylsulfanylmethyl-tetrazol, 5-methylsulfonylmethyl-tetrazol, 5-benzylsulfanyl-tetrazol, 5-(thiophen-2-yl-methyl)-tetrazol, 5-(morpholinocarbonylmethyl)-tetrazol, 5-(4-methylpiperazin-1-yl)-tetrazol, 5-(4-acetyl-piperazin-1-yl)-tetrazol, 5-(4-cyanopiperazin-1-yl)-tetrazol, 5-(4-benzylpiperazin-1-yl)-tetrazol, 5-ethoxycarbonylmethyl-tetrazol, 5-hydroxyethyl-tetrazol, 5-acetylaminomethyl-tetrazol, 5-methyl-[1,2,4]-oxadiazol, 5-phenyl-[1,2,4]-oxadiazol, 5-pyridin-3-yl-[1,2,4]-oxadiazol, 5-methyl-[1,3,4]-oxadiazol, 5-phenyl-[1,3,4]-oxadiazol, 5-amino-2H-[1,3,4]-thiadiazol, 5-amino-2H-[1,2,4]-thiadiazol.

13. The compound of claim 6, wherein substituents of the heteroaryl are selected from the group comprising methyl, cyano, amino, fluoro, difluoromethyl, formyl, hydroxyl methyl, carboxamide, acetyl, 1-methoxyimino-methyl, 4pyridinyl, 3-pyridinyl, diethylamino, methylsulfonylmethyl.

14. The compound of claim 2, wherein X and Y are each independently CH, or CF.

15. The compound of claim 2, wherein at least one of X and Y is CF.

16. The compound of claim 2, wherein R4 is C₁-C₆ alkylamido, C₁-C₆ haloalkylamido, C₁-C₆ thioalkylamido, substituted C₁-C₆ thioalkylamido, wherein the alkyl group is substituted with halo; carbamate, thiocarbamate, urea, unsubstituted or substituted heteroaryl, wherein substituents of the heteroaryl are selected from the group comprising C₁-C₆ alkyl, nitro, cyano, hydroxyl, halo, amino, C₁-C₆ alkylcarbonyl, C₁-C₆ alkoxycarbonyl, carboxy.

17. The compound of claim 2, wherein R4 is acetamide, difluoroacetamide, thioacetamide, difluoroacetamide, carbamate, thiocarbamate, urea, [1,2,3]-triazol, [1,2,4]-triazol.

18. The compound of claim 2, wherein R₄ is acetamide, carbamate, [1,2,3]-triazol.

19. The compound of claim 10, wherein n, R₂, R_{2′}, W is as defined in claim 10, R₄ is acetamide, carbamate, [1,2,3]-triazol; X and Y are each independently CH, or CF.

20. The compound of claim 1, wherein X, Y, R₄ are as defined in claim 1; and Q is 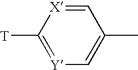
wherein X′, Y′ and T have the definition as provided in claim 1.

21. The compound of claim 20, wherein X′ and Y′ are each independently CH, CF, N, C—CH₃.

22. The compound of claim 20, wherein X′ and Y′ are each independently CH, CF, N, C—CH₃; X, Y, R₄ are as defined in claim 1;
T is hydrogen or 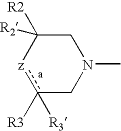
wherein R₂, R_{2′}, R₃, R_{3′}, Z, n, have definition as provided in claim 1, with proviso that when T is hydrogen, R₄ is not acetamido.

23. The compound of claim 22, wherein Z is selected from the group comprising CH, NH, O, S, CH₂, C(R₆)R₆′, C═O, NR₇, C═C(R₈)R_{8′}, SO, SO₂, S═NH, S═NC(O)CH₃, S═NC(O)NHCH₃, S(O)═NH, S(O)═NCH₃, S(O)NC(O)CH₃, S(O)═NC(O)NHCH₃, S(O)═NC(O)NHCH₂CH₂Cl,
with proviso that when X is CH, Y is CF, one of X′ or Y′ or both X′, Y′ are N, then Z is not NH.

24. The compound of claim 22, wherein Z is selected from the group S, SO, SO₂;
X′ and Y′ are each independently CH, CF, N, C—CH₃;
X, Y, are each independently CH or CF;
R₄ is as defined in claim 1;
R₂, R_{2′}, R₃, R_{3′}, are each independently selected from hydrogen, methyl, hydroxyl, fluorine.

25. The compound of claim 22, wherein R₄ is acetamido, [1,2,3]-triazol, methyl carbamate, t-butyl carbamate, OR₁ wherein R₁ is hydrogen, P(O)(OM)₂, wherein M is hydrogen, Na, methyl, ethyl, t-butyl, phenyl; or R₁ is an amino acid residue derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, attached to the oxygen via carbonyl of amino acid to form an ester linkage.

26. The compound of claim 25, wherein amino acid is L-alanine and L-valine.

27. The compound of claim 22, wherein at least one of the X, Y is CF and at least one of X′, Y′ is CF.

28. The compound of claim 22, wherein at least one of the X, Y is CF and at least one of X′, Y′ is N.

29. The compound of claim 22, wherein X, is CH, Y is CF, X′ is N and Y′ is C—CH₃.

30. The compound of claim 24, wherein Z, X, Y, X′, Y′, R₂, R_{2′}, R₃, R_{3′} are as defined in claim 24, R₄ is selected from the group comprising acetamido, [1,2,3]-triazol, methyl carbamate, t-butyl carbamate, OR₁, wherein R₁ is as defined in claim 25.

31. A compound or a pharmaceutically acceptable salt thereof which is
(S)—N-{3-[4-(4-(2-Cyanoaziridin-1-yl)-piperidin-1-yl)-3-fluorophenyl]-2-oxo-oxazolidin-5-ylmethyl}-acetamide;
(S)—N-{3-[4-(4-pyrrol-1-yl-piperidin-1-yl)-3-fluorophenyl]-2-oxo-oxazolidin-5-ylmethyl}-acetamide;
(S)—N-{3-[3,5-Difluoro-4-(4-pyrrol-1-yl-piperidin-1-yl)-phenyl]-2-oxo-oxazolidin-5-ylmethyl}-acetamide;
(S)—N-{3-[4-(4-(1H-imidazol-1-yl)-piperidin-1-yl)-3-fluorophenyl]-2-oxo-oxazolidin-5-ylmethyl}-acetamide;
(S)—N-{3-[4-(4-(1H-[1,2,3]-triazol-1-yl)-piperidin-1-yl)-3-fluorophenyl]-2-oxo-oxazolidin-5-ylmethyl}-acetamide;
(S)—N-{3-[4-(4 (2H-[1,2,3]-triazol-2-yl)-piperidin-1-yl)-3-fluorophenyl]-2-oxo-oxazolidin-5-ylmethyl}-acetamide;
(S)—N-{3-[4-(4-(1H-[1,2,3]-triazol-1-yl)-piperidin-1-yl)-3,5-difluorophenyl]-2-oxo-oxazolidin-5-ylmethyl}-acetamide;
(S)—N-{3-[4-(4-(2H-[1,2,3]-triazol-2-yl)-piperidin-1-yl)-3,5-difluorophenyl]-2-oxo-oxazolidin-5-ylmethyl}-acetamide;
(S)—N-{3-[4-(4-(1H-[1,2,3]-triazol-1-yl)-3-fluoropiperidin-1-yl)-3,5-difluorophenyl]-2-oxo-oxazolidin-5-ylmethyl}-acetamide;
(S)—N-{3-[4-(4-(2H-[1,2,3]-triazol-2-yl)-3-fluoropiperidin-1-yl)-3,5-difluorophenyl]-2-oxo-oxazolidin-5-ylmethyl}-acetamide;
(S)—N-{3-[4-(4-(1H-[1,2,3]-triazol-1-yl)-3-fluoropiperidin-1-yl)-3-fluorophenyl]-2-oxo-oxazolidin-5-ylmethyl}-acetamide;
(S)—N-{3-[4-(4-(2H-[1,2,3]-triazol-2-yl)-3-fluoropiperidin-1-yl)-3-fluorophenyl]-2-oxo-oxazolidin-5-ylmethyl}-acetamide;
(S)—N-{3-[4-(4-(1H-[1,2,4]-triazol-1-yl)-piperidin-1-yl)-3-fluorophenyl]-2-oxo-oxazolidin-5-ylmethyl}-acetamide;
(S)—N-{3-[4-(4-([1,2,3,4]-tetrazol-5-yl)-piperidin-1-yl)-3-fluorophenyl]-2-oxo-oxazolidin-5-ylmethyl}-acetamide;
(S)—N-{3-[4-(4-([1,2,3,4]-tetrazol-5-yl)-piperidin-1-yl)-3,5-difluorophenyl]-2-oxo-oxazolidin-5-ylmethyl}-acetamide;
(S)—N-{3-[4-(4-(1H-[1,2,3,4]-tetrazol-1-yl)-piperidin-1-yl)-3-fluorophenyl]-2-oxo-oxazolidin-5-ylmethyl}-acetamide;
(S)—N-{3-[4-(4-(2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(1H-[1,2,3,4]-tetrazol-1-yl)-piperidin-1-yl)-3,5-difluorophenyl]-2-oxo-oxazolidin-5-ylmethyl}-acetamide;
(S)—N-{3-[4-(4-(2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(1H-[1,2,3,4]-tetrazol-1-yl)-3-hydroxypiperidin-1-yl)-3-fluorophenyl]-2-oxo-oxazolidin-5-ylmethyl}-acetamide and isomer thereof;
(S)—N-{3-[4-(4-(1H-[1,2,3,4]-tetrazol-1-yl)-3-hydroxypiperidin-1-yl)-3,5-difluorophenyl]-2-oxo-oxazolidin-5-ylmethyl}-acetamide;
(S)—N-{3-[4-(4-(1H-[1,2,3,4]-tetrazol-1-yl)-3-fluoropiperidin-1-yl)-3,5-difluorophenyl]-2-oxo-oxazolidin-5-ylmethyl}-acetamide;
(S)—N-{3-[4-(4-(4-formyl-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-formyl-1H-[1,2,3]-triazol-1-yl)-piperidin-1-yl)-3,5-difluorophenyl]-2-oxo-oxazolidin-5-ylrmethyl}-acetamide;
(S)—N-{3-[4-(4-(4-formyl-1H-[1,2,3]-triazol-1-yl)-3-fluoropiperidin-1-yl)-3-fluorophenyl]-2-oxo-oxazolidin-5-ylmethyl}-acetamide;
(R)-{3-[4-(4-(4-formyl-1H-[1,2,3]-triazol-1-yl)-piperidin-1-yl)-3-fluorophenyl]-5-(1-1-[1,2,3]-triazol-1-ylmethyl)}-oxazolidin-2-one;
(S)—N-{3-[4-(4-(4-difluoromethyl-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-difluoromethyl-1H-[1,2,3]-triazol-1-yl)-piperidin-1-yl)-3,5-difluorophenyl]-2-oxo-oxazolidin-5-ylmethyl}-acetamide;
(S)—N-{3-[4-(4-(4-[1,3]-Dioxolan-2-yl-1H-[1,2,3]-triazol-1-yl)-piperidin-1-yl)-3-fluorophenyl]-5-(1H-[1,2,3]-triazol-1-ylmethyl)}-oxazolidin-2-one;
(S)—N-{3-[4-(4-(4-ethoxycarbonyl-1H-[1,2,3]-triazol-1-yl)-methyl-piperidin-1-yl)-3-fluoro-phenyl]-2-oxo-oxazolidin-5-ylmethyl}-acetamide;
(S)—N-{3-[4-(4-(5-ethoxycarbonyl-1H-[1,2,3]-triazol-1-yl)-methyl-piperidin-1-yl)-3-fluoro-phenyl]-2-oxo-oxazolidin-5-ylmethyl}-acetamide;
(S)—N-{3-[4-(4-(4-ethoxycarbonyl-1H-[1,2,3]-triazol-1-yl)-3-fluoropiperidin-1-yl)-3-fluorophenyl]-2-oxo-oxazolidin-5-ylmethyl}-acetamide;
(S)—N-{3-[4-(4-(4-ethoxycarbonyl-1H-[1,2,3]-triazol-1-yl)-piperidin-1-yl)-3,5-difluorophenyl]-2-oxo-oxazolidin-5-ylmethyl}-acetamide;
(R)-{3-[4-(4-(4-ethoxycarbonyl-1H-[1,2,3]-triazol-1-yl)-piperidin-1-yl)-3-fluorophenyl]-5-(1H-[1,2,3]-triazol-1-ylmethyl)}-oxazolidin-2-one;
(R)-{3-[4-(4-(4-ethoxycarbonyl-1H-[1,2,3]-triazol-1-yl)-piperidin-1-yl)-3,5-difluorophenyl]-5-(1H-[1,2,3]-triazol-1-ylmethyl)}-oxazolidin-2-one;
(S)—N-{3-[4-(4-(4-hydroxymethyl-1H-[1,2,3]-triazol-1-yl)-piperidin-1-yl)-3-fluorophenyl]-2-oxo-oxazolidin-5-ylmethyl}-acetamide;
(S)—N-{3-[4-(4-(5-hydroxymethyl-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-hydroxymethyl-1H-[1,2,3]-triazol-1-yl)-3-fluoropiperidin-1-yl)-3-fluorophenyl]-2-oxo-oxazolidin-5-ylmethyl}-acetamide;
(S)—N-{3-[4-(4-(4-hydroxymethyl-1H-[1,2,3]-triazol-1-yl)-piperidin-1-yl)-3,5-difluorophenyl]-2-oxo-oxazolidin-5-ylmethyl}-acetamide;
(R)-{3-[4-(4-(4-hydroxymethyl-1H-[1,2,3]-triazol-1-yl)-piperidin-1-yl)-3-fluorophenyl]-5-(1H-[1,2,3]-triazol-1-ylmethyl)}-oxazolidin-2-one;
(R)-{3-[4-(4-(4-hydroxymethyl-1H-[1,2,3]-triazol-1-yl)-piperidin-1-yl)-3,5-difluorophenyl]-5-(1H-[1,2,3]-triazol-1-ylmethyl)}-oxazolidin-2-one;
(S)—N-{3-[4-(4-((4-hydroxymethyl)-[1,2,3]-triazol-1-yl-methyl)-piperidin-1-yl)-3-fluoro-phenyl]-2-oxo-oxazolidin-5-ylmethyl}-acetamide;
(S)—N-{3-[4-(4-(4-cyano-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-carboxamido-1H-[1,2,3]-triazol-1-yl)-piperidin-1-yl)-3-fluorophenyl]-2-oxo-oxazolidin-5-ylmethyl}-acetamide;
(R)-{3-[4-(4-(4-carboxamido-1H-[1,2,3]-triazol-1-yl)-piperidin-1-yl)-3-fluorophenyl]-5-(1H-[1,2,3]-triazol-1-ylmethyl)}-oxazolidin-2-one;
(S)—N-{3-[4-(4-(4-carboxamido-1H-[1,2,3]-triazol-1-yl)-methyl-piperidin-1-yl)-3-fluoro-phenyl]-2-oxo-oxazolidin-5-ylmethyl}-acetamide;
(S)—N-{3-[4-(4-(5-carboxamido-1H-[1,2,3],-triazol-1-yl)-methyl-piperidin-1-yl)-3-fluoro-phenyl]-2-oxo-oxazolidin-5-ylmethyl}-acetamide;
(S)—N-{3-[4-(4-(4-hydroxycarbonyl-1H-[1,2,3]-triazol-1-yl)-methyl-piperidin-1-yl)-3-fluoro-phenyl]-2-oxo-oxazolidin-5-ylmethyl}-acetamide;
(S)—N-{3-[4-(4-(5-hydroxycarbonyl-1H-[1,2,3]-triazol-1-yl)-methyl-piperidin-1-yl)-3-fluoro-phenyl]-2-oxo-oxazolidin-5-ylmethyl}-acetamide;
(S)—N-{3-[4-(4-(4-acetyl-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-acetyl-1H-[1,2,3]-triazol-1-yl)-piperidin-1-yl)-3,5-difluorophenyl]-2-oxo-oxazolidin-5-ylmethyl}-acetamide;
(R)-{3-[4-(4-(4-acetyl-1H-[1,2,3]-triazol-1-yl)-piperidin-1-yl)-3-fluorophenyl]-5-(1H-[1,2,3]-triazol-1-ylmethyl)}-oxazolidin-2-one;
(S)—N-{3-[4-(4-(4-acetyl-1H-[1,2,3]-trizol-1-ylmethyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-(1-hydroxyethyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-(1-hydroxyethyl)-1H-[1,2,3]-triazol-1-yl)-piperidin-1-yl)-3,5-difluorophenyl]-2-oxo-oxazolidin-5-ylmethyl}-acetamide;
(R)-{3-[4-(4-(4-(2-hydroxyethyl)-1H-[1,2,3]-triazol-1-yl)-piperidin-1-yl)-3-fluorophenyl]-5-(1H-[1,2,3]-triazol-1-ylmethyl)}-oxazolidin-2-one;
(S)—N-{3-[4-(4-(4-(2-methyl-oxiranyl)-1H-[1,2,3]-triazol-1-yl)-piperidin-1-yl)-3-fluorophenyl]-2-oxo-oxazolidin-5-ylmethyl}-acetamide;
(R)-{3-[4-(4-(4-(2-dimethyl-oxiranyl)-1H-[1,2,3]-triazol-1-yl)-piperidin-1-yl)-3-fluorophenyl]-5-(1H-[1,2,3]-triazol-1-ylmethyl)}-oxazolidin-2-one;
(S)—N-{3-[4-(4-((4-((E/Z)-2-cyano-vinyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-hydroxyimino-methyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(1-methoxyimino-ethyl)-1H-[1,2,3]-triazol-1-yl)-piperidin-1-yl)-3,5-difluorophenyl]-2-oxo-oxazolidin-5-ylmethyl}-acetamide;
(S)—N-{3-[4-(4-(4-(1-methoxyimino-ethyl)-1H-[1,2,3]-triazol-1-yl)-piperidin-1-yl)-3-fluorophenyl]-2-oxo-oxazolidin-5-ylmethyl}-acetamide;
(R)-{3-[4-(4-(4-(1-methoxyimino-ethyl)-[1,2,3]-triazol-1-yl)-piperidin-1-yl)-3-fluorophenyl]-5-(1H-[1,2,3]-triazol-1-ylmethyl)}-oxazolidin-2-one;
(R)-{3-[4-(4-(4-(1-methoxyimino-ethyl)-[1,2,3]-triazol-1-yl)-piperidin-1-yl)-3,5-difluorophenyl]-5-(1H-[1,2,3]-triazol-1-ylmethyl)}-oxazolidin-2-one;
(S)—N-{3-[4-(4-(1-methoxyimino-methyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(1-methoxyimino-methyl)-1H-[1,2,3]-triazol-1-yl)-piperidin-1-yl)-3,5-difluorophenyl]-2-oxo-oxazolidin-5-ylmethyl}-acetamide;
(S)—N-{3-[4-(4-(1-benzyloxyimino-methyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(4-hydroxyiminoamino-methyl)-[1,2,3]-triazol-1-yl)-piperidin-1-yl)-3-fluorophenyl]-2-oxo-oxazolidin-5-ylmethyl}-acetamide;
(S)—N-{3-[4-(4-(4-N,N-dimethylaminoethylaminomethyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(2H-benzotriazol-2-yl)-piperidin-1-yl)-3,5-difluorophenyl]-2-oxo-oxazolidin-5-ylmethyl}-acetamide;
(S)—N-{3-[4-(4-(1H-benzotriazol-1-yl)-piperidin-1-yl)-3,5-difluorophenyl]-2-oxo-oxazolidin-5-ylmethyl}-acetamide;
(S)—N-{3-[4-(4-Benzotriazol-2-yl-piperidin-1-yl)-3-fluorophenyl]-2-oxo-oxazolidin-5-ylmethyl}-acetamide;
(S)—N-{3-[4-(4-Benzotriazol-1-yl-piperidin-1-yl)-3-fluorophenyl]-2-oxo-oxazolidin-5-ylmethyl}-acetamide;
(S)—N-{3-[4-(4-(4-(1H-[1,2,3,4]-tetrazol-1-ylmethyl)-1H-[1,2,3]-triazol-1-yl)-piperidin-1-yl)-3-fluorophenyl]-2-oxo-oxazolidin-5-ylmethyl}-acetamide;
(S)—N-{3-[4-(4-(2H-[1,2,3,4]-tetrazol-2-ylmethyl)-[1,2,3]-triazol-1-yl)-piperidin-1-yl)-3-fluorophenyl]-2-oxo-oxazolidin-5-ylmethyl}-acetamide;
(S)—N-{3-[4-(4-(1H-imidazol-1-ylmethyl)-[1,2,3]-triazol-1-yl)-piperidin-1-yl)-3-fluorophenyl]-2-oxo-oxazolidin-5-ylmethyl}-acetamide;
(R)-{3-[4-(4-(1H-[1,2,3]-triazol-1-yl)-3-fluoropiperidin-1-yl)-3-fluorophenyl]-5-(1H-[1,2,3]-triazol-1-ylmethyl)}-oxazolidin-2-one;
(R)-{3-[4-(4-(1H-[1,2,3]-triazol-1-yl)-piperidin-1-yl)-3-fluorophenyl]-5-(1H-[1,2,3]-triazol-1-ylmethyl)}-oxazolidin-2-one;
(R)-{3-[4-(4-(1H-[1,2,3]-triazol-1-yl)-piperidin-1-yl)-3,5-difluorophenyl]-5-(1H-[1,2,3]-triazol-1-ylmethyl)}-oxazolidin-2-one;
(R)-{3-[4-(4-(1H-[1,2,3]-triazol-1-yl)-3-fluoropiperidin-1-yl)-3,5-difluorophenyl]-5-(1H-[1,2,3]-triazol-1-ylmethyl)}-oxazolidin-2-one;
(S)—N-{3-[4-(4-([1,2,3]-triazol-1-yl)-piperidin-1-yl)-3-fluorophenyl]-2-oxo-oxazolidin-5-ylmethyl}-thioacetamide;
(S)—N-{3-[4-(4-(5-methyl-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-methyl-1H-[1,2,3,4]-tetrazol-1-yl)-piperidin-1-yl)-3,5-difluorophenyl]-2-oxo-oxazolidin-5-ylmethyl}-acetamide;
(S)—N-{3-[4-(4-(5-methyl-1H-[1,2,3,4]-tetrazol-1-yl)-piperidin-1-yl)-3,5-difluorophenyl]-2-oxo-oxazolidin-5-ylmethyl}-acetamide;
(S)—N-{3-[4-(4-(5-methyl-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(2-methyl-2H-tetrazol-5-yl)-piperidin-1-yl)-3-fluorophenyl]-2-oxo-oxazolidin-5-ylmethyl}-acetamide;
(S)—N-{3-[4-(4-(1-methyl-1H-tetrazol-5-yl)-piperidin-1-yl)-3-fluorophenyl]-2-oxo-oxazolidin-5-ylmethyl}-acetamide;
(S)—N-{3-[4-(4-(2-methyl-2H-tetrazol-5-yl)-piperidin-1-yl)-3,5-difluorophenyl]-2-oxo-oxazolidin-5-ylmethyl}-acetamide;
(S)—N-{3-[4-(4-(1-methyl-1H-tetrazol-5-yl)-piperidin-1-yl)-3,5-difluorophenyl]-2-oxo-oxazolidin-5-ylmethyl}-acetamide;
(R)-{3-[4-(4-(2-methyl-2H-tetrazol-5-yl)-piperidin-1-yl)-3-fluorophenyl]-5-(1H-[1,2,3]-triazol-1ylmethyl)}-oxazolidin-2-one;
(R)-{3-[4-(4-(1-methyl-1H-tetrazol-5-yl)-piperidin-1-yl)-3-fluorophenyl]-5-(1H-[1,2,3]-triazol-1-ylmethyl)}-oxazolidin-2-one;
(S)—N-{3-[4-(4-(1-methyl)-1H-tetrazol-5-ylmethyl-piperidin-1-yl)-3,5-difluoro-phenyl]-2-oxo-oxazolidin-5-ylmethyl}-acetamide;
(S)—N-{3-[4-(4-(2-methyl)-2H-tetrazol-5-ylmethyl-piperidin-1-yl)-3,5-difluoro-phenyl]-2-oxo-oxazolidin-5-ylmethyl}-acetamide;
(S)—N-{3-[4-(4-(1-methyl-1H-tetrazol-5-ylmethyl)-piperidin-1-yl)-3-fluoro-phenyl]-2-oxo-oxazolidin-5-ylmethyl}-acetamide;
(S)—N-{3-[4-(4-(2-methyl-2H-tetrazol-5-ylmethyl)-piperidin-1-yl)-3-fluoro-phenyl]-2-oxo-oxazolidin-5-ylmethyl}-acetamide;
(S)—N-{3-[4-(4-(5-methylsulfanylmethyl-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-methylsulfanylmethyl-1H-[1,2,3,4]-tetrazol-1-yl)-piperidin-1-yl)-3-fluorophenyl]-2-oxo-oxazolidin-5-ylmethyl}-acetamide;
(S)—N-{3-[4-(4-(5-methylsulfanylmethyl-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-methylsulfanylmethyl-1H-[1,2,3,4]-tetrazol-1-yl)-piperidin-1-yl)-3,5-fluorophenyl]-2-oxo-oxazolidin-5-ylmethyl}-acetamide;
(S)—N-{3-[4-(4-(5-benzylsulfanyl-1H-[1,2,3,4]-tetrazol-1-yl)-piperidin-1-yl)-3,5-difluorophenyl]-2-oxo-oxazolidin-5-ylmethyl}-acetamide;
(S)—N-{3-[4-(4-(5-(benzylsulfanyl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-(benzylsulfanyl)-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-(benzylsulfanyl)-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-methylsulfonylmethyl-1H-[1,2,3,4]-tetrazol-1-yl)-piperidin-1-yl)-3,5-difluorophenyl]-2-oxo-oxazolidin-5-ylmethyl}-acetamide;
(S)—N-{3-[4-(4-(5-methylsulfonylmethyl-1H-[1,2,3,4]-tetrazol-1-yl)-piperidin-1-yl)-3-fluorophenyl]-2-oxo-oxazolidin-5-ylmethyl}-acetamide;
(S)—N-{3-[4-(4-(5-(thiophen-2-yl-methyl)-1H-[1,2,3,4]-tetrazol-1-yl)-piperidin-1-yl)-3-fluorophenyl]-2-oxo-oxazolidin-5-ylmethyl}-acetamide;
(S)—N-{3-[4-(4-(5-(thiophen-2-yl-methyl)-1H-[1,2,3,4]-tetrazol-1-yl)-piperidin-1-yl)-3-fluorophenyl]-2-oxo-oxazolidin-5-ylmethyl}-acetamide;
(S)—N-{3-[4-(4-(5-(thiophen-2-yl-methyl)-1H-[1,2,3,4]-tetrazol-1-yl)-piperidin-1-yl)-3,5-difluorophenyl]-2-oxo-oxazolidin-5-ylmethyl}-acetamide;
(S)—N-{3-[4-(4-(5-(thiophen-2-yl-methyl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-(morpholinocarbonylmethyl)-1H-[1,2,3,4]-tetrazol-1-yl)-piperidin-1-yl)-3-fluorophenyl]-2-oxo-oxazolidin-5-ylmethyl}-acetamide;
(S)—N-{3-[4-(4-(5-(morpholinocarbonylmethyl)-1H-[1,2,3,4]-tetrazol-1-yl)-piperidin-1-yl)-3,5-difluorophenyl]-2-oxo-oxazolidin-5-ylmethyl}-acetamide;
(S)—N-{3-[4-(4-(5-(morpholin-4-yl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-(morpholin-4-yl)-2H-[1,2,3,4]-tetrazol-2-yl}-piperidin-1-yl)-3-fluorophenyl]-2-oxo-oxazolidin-5-ylmethyl)-acetamide;
(S)—N-{3-[4-(4-(5-phenyl-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-phenyl-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-(4-nitrophenyl)-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-(4-nitrophenyl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-(4-pyridinyl)-1H-[1,2,3,4]-tetrazol-1-yl)-piperidin-1-yl)-3-fluorophenyl]-2-oxo-oxazolidin-5-ylmethyl}-acetamide;
(S)—N-{3-[4-(4-(5-(3-pyridinyl)-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-(4-pyridinyl)-1H-[1,2,3,4]-tetrazol-1-yl)-piperidin-1-yl)-3,5-difluorophenyl]-2-oxo-oxazolidin-5-ylmethyl}-acetamide;
(S)—N-{3-[4-(4-(5-(3-pyridinyl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-amino-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-amino-2H-[1,2,3,4]-tetrazol-1-yl)-piperidin-1-yl)-3,5-difluorophenyl]-2-oxo-oxazolidin-5-ylmethyl}-acetamide;
(S)—N-{3-[4-(4-(5-(diethylamino)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-(piperazin-1-yl)-2H-[1,2,3,4],-tetrazol-2-yl)-piperidin-1-yl)-3-fluorophenyl]-2-oxo-oxazolidin-5-ylmethyl}-acetamide;
(S)—N-{3-[4-(4-(5-(piperazin-1-yl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(5-(4-methylpiperazin-1-yl)-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-(4-methylpiperazin-1-yl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-(4-acetyl-piperazin-1-yl)-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-(4-acetyl-piperazin-1-yl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-(4-cyanopiperazin-1-yl)-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-(4-cyanopiperazin-1-yl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-(4-benzylpiperazin-1-yl)-2H-[1,2,3,4]-tetrazol-2-yl)-piperidin-1-yl)-3-fluorophenyl]-2-oxo-oxazolidin-5-ylmethyl}-acetamide;
(S)—N-{3-[4-(4-(5-(4-benzylpiperazin-1-yl)-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-1H-tetrazol-5-ylmethyl-piperidin-1-yl)-3-fluoro-phenyl]-2-oxo-oxazolidin-5-ylmethyl}-acetamide;
(S)—N-{3-[4-(4-1H-tetrazol-5-ylmethyl-piperidin-1-yl)-3,5-difluorophenyl]-2-oxo-oxazolidin-5-ylmethyl}-acetamide;
(S)—N-{3-[4-(4-([1,2,3]-triazol-1-yl-methyl)-piperidin-1-yl)-3-fluoro-phenyl]-2-oxo-oxazolidin-5-ylmethyl}-acetamide;
(R)-{3-[4-(4-(1H-[1,2,3,4]-tetrazol-1-yl)-piperidin-1-yl)-3-fluorophenyl]-5-(1H-[1,2,3]-triazol-1-ylmethyl)}-oxazolidin-2-one;
(R)-{3-[4-(4-(2H-[1,2,3,4]-tetrazol-2-yl)-piperidin-1-yl)-3-fluorophenyl]-5-(1H-[1,2,3]-triazol-1-ylmethyl)}-oxazolidin-2-one;
(S)-{3-[4-(4-([1,2,3,4]-tetrazol-5-yl)-piperidin-1-yl)-3-fluorophenyl]-5-(1H-[1,2,3]-triazol-1-ylmethyl)}-oxazolidin-2-one;
(S)—N-{3-[4-(4-(5-methyl-[1,2,4]-oxadiazol-3-yl)-piperidin-1-yl)-3-fluorophenyl]-2-oxo-oxazolidin-5-ylmethyl}-acetamide;
(S)—N-{3-[4-(4-(5-methyl-[1,2,4]-oxadiazol-3-ylmethyl)-piperidin-1-yl)-3-fluoro-phenyl]-2-oxo-oxazolidin-5-ylmethyl}-acetamide;
(S)—N-{3-[4-(4-(5-methyl-[1,2,4]-oxadiazol-3-ylmethyl)-piperidin-1-yl)-3,5-difluoro-phenyl]-2-oxo-oxazolidin-5-ylmethyl}-acetamide;
(S)—N-{3-[4-(4-(5-phenyl-[1,2,4]-oxadiazol-3-ylmethyl)-piperidin-1-yl)-3-fluoro-phenyl]-2-oxo-oxazolidin-5-ylmethyl}-acetamide;
(S)—N-{3-[4-(4-(5-phenyl-[1,2,4]-oxadiazol-3-ylmethyl)-piperidin-1-yl)-3,5-difluoro-phenyl]-2-oxo-oxazolidin-5-ylmethyl}-acetamide;
(S)—N-{3-[4-(4-((5-pyridin-3-yl)-[1,2,4]-oxadiazol-3-ylmethyl)-piperidin-1-yl)-3-fluoro-phenyl]-2-oxo-oxazolidin-5-ylmethyl}-acetamide;
(S)—N-{3-[4-(4-(5-methyl-[1,3,4]-oxadiazol-2-ylmethyl)-piperidin-1-yl)-3-fluoro-phenyl]-2-oxo-oxazolidin-5-ylmethyl}-acetamide;
(S)—N-{3-[4-(4-(5-phenyl-[1,3,4]-oxadiazol-2-ylmethyl)-piperidin-1-yl)-3-fluoro-phenyl]-2-oxo-oxazolidin-5-ylmethyl}-acetamide;
(S)—N-{3-[4-(4-(5-Amino-2H-[1,3,4]-thiadiazol-2-yl)-piperidin-1-yl)-3-fluorophenyl]-2-oxo-oxazolidin-5-ylmethyl}-acetamide;
(S)—N-{3-[4-(4-(5-Amino-[1,3,4]-thiadiazol-2-yl)-piperidin-1-yl)-3,5-difluoro-phenyl]-2-oxo-oxazolidin-5-ylmethyl}-acetamide;
(S)—N-{3-[4-(4-(5-Amino-[1,3,4]-thiadiazol-2-ylmethyl)-piperidin-1-yl)-3,5-difluoro-phenyl]-2-oxo-oxazolidin-5-ylmethyl}-acetamide;
(S)—N-{3-[4-(4-(3-ethoxycarbonyl-imidazol-1-yl)-methyl-piperidin-1-yl)-3-fluoro-phenyl]-2-oxo-oxazolidin-5-ylmethyl}-acetamide;
(S)—N-{3-[3,5-Difluoro-4-(4-2H-tetrazol-2-yl-piperidin-1-yl)-phenyl]-2-oxo-oxazolidin-5-ylmethyl}-carbamic acid methyl ester;
(S)—N-{3-[3,5-Difluoro-4-(4-1H-tetrazol-1-yl-piperidin-1-yl)-phenyl]-2-oxo-oxazolidin-5-ylmethyl}-carbamic acid methyl ester;
(S)—N-{3-[3,5-Difluoro-4-(4-[1,2,3]-thiazol-1-yl-piperidin-1-yl)-phenyl]-2-oxo-oxazolidin-5-ylmethyl}-carbamic acid methyl ester;
(S)—N-{3-[3-fluoro-4-(4-2H-tetrazol-2-yl-piperidin-1-yl)-phenyl]-2-oxo-oxazolidin-5-ylmethyl}-carbamic acid methyl ester;
(S)—N-{3-[3-fluoro-4-(4-1H-tetrazol-1-yl-piperidin-1-yl)-phenyl]-2-oxo-oxazolidin-5-ylmethyl}-carbamic acid methyl ester;
(S)—N-{3-[4-(4-2H-[1,2,3]-triazol-2-yl-piperidin-1-yl)-3,5-difluorophenyl]-2-oxo-oxazolidin-5-ylmethyl}-carbamic acid methyl ester;
(S)—N-{3-[3,5-Difluoro-4-(4-[1,2,3]triazol-2-yl-piperidin-1-yl)-phenyl]-2-oxo-oxazolidin-5-ylmethyl}-2,2-difluoro-acetamide;
(S)—N-{3-[3,5-Difluoro-4-(4-[1,2,3]-triazol-1-yl-piperidin-1-yl)-phenyl]-2-oxo-oxazolidin-5-ylmethyl}-2,2-difluoro-acetamide;
(S)—N-{3-[3,5-Difluoro-4-(4-[1,2,3,4]-tetrazol-2-yl-piperidin-1-yl)-phenyl]-2-oxo-oxazolidin-5-ylmethyl}-2,2-difluoro-acetamide;
(S)—N-{3-[3,5-Difluoro-4-(4-[1,2,3,4]-triazol-1-yl-piperidin-1-yl)-phenyl]-2-oxo-oxazolidin-5-ylmethyl}-2,2-difluoro-acetamide;
(S)—N-{3-[3,5-Difluoro-4-(4-2H-tetrazole-2-yl-piperidin-1-yl)-phenyl]-2-oxo-oxazolidin-5-ylmethyl}-2,2-dichloro-acetamide;
(S)—N-{3-[3-fluoro-4-(4-2H-tetrazol-2-yl-piperidin-1-yl)-phenyl]-2-oxo-oxazolidin-5-ylmethyl}-carbamic acid ethyl ester;
(S)—N-{3-[3,5-Difluoro-4-(4-1H-tetrazole-1-yl-piperidin-1-yl)-phenyl]-2-oxo-oxazolidin-5-ylmethyl}-2,2-difluoro-thioacetamide;
(S)—N-{3-[3,5-Difluoro-4-(4-1H-tetrazole-1-yl-piperidin-1-yl)-phenyl]-2-oxo-oxazolidin-5-ylmethyl}-cyclopropane carboxamide;
(S)—N-{3-[3,5-Difluoro-4-(4-[1,2,3]-triazol-1-yl-piperidin-1-yl)-phenyl]-2-oxo-oxazolidin-5-ylmethyl}-propionamide;
(S)—N-{3-[3,5-Difluoro-4-(4-[1,2,3]-triazol-2-yl-piperidin-1-yl)-phenyl]-2-oxo-oxazolidin-5-ylmethyl}-propionamide;
(S)—N-{3-[3,5-Difluoro-4-(4-2H-tetrazol-2-yl-piperidin-1-yl)-phenyl]-2-oxo-oxazolidin-5-ylmethyl}-propionamide;
(S)—N-{3-[3,5-Difluoro-4-(4-1H-tetrazol-1-yl-piperidin-1-yl)-phenyl]-2-oxo-oxazolidin-5-ylmethyl}-propionamide;
(S)N-{3-[3-Fluoro-4-(4-2H-tetrazol-2-yl-piperidin-1-yl)-phenyl]-2-oxo-oxazolidin-5-ylmethyl}-propionamide;
(S)N-{3-[3-Fluoro-4-(4-1H-tetrazol-1-yl-piperidin-1-yl)-phenyl]-2-oxo-oxazolidin-5-ylmethyl}-propionamide;
(S)—N-{3-[3,5-Difluoro-4-(4-[1,2,3]-triazol-2-yl-piperidin-1-yl)-phenyl]-2-oxo-oxazolidin-5-ylmethyl}-propionamide;
(S)—N-{3-[3,5-Difluoro-4-(4-[1,2,3]-triazol-1-yl-piperidin-1-yl)-phenyl]-2-oxo-oxazolidin-5-ylmethyl}-propionamide;
(S)—N-{3-[3,5-Difluoro-4-(4-(5-methyl-tetrazol-2-yl)-piperidin-1-yl)-phenyl]-2-oxo-oxazolidin-5-ylmethyl}-propionamide;
(S)—N-{3-[3,5-Difluoro-4-(4-(5-methyl-tetrazol-1-yl)-piperidin-1-yl)-phenyl]-2-oxo-oxazolidin-5-ylmethyl}-propionamide;
(S)—N-{3-[3,5-Difluoro-4-(4-(4-methyl-5-thioxo-4,5-dihydro-tetrazol-1-yl)-piperidin-1-yl)-phenyl]-2-oxo-oxazolidin-5-ylmethyl}-acetamide;
(S)—N-{3-[3,5-Difluoro-4-(4-(4-methyl-5-thioxo-4,5-dihydro-[1,2,4]triazol-1-yl)-piperidin-1-yl)-phenyl]-2-oxo-oxazolidin-5-ylmethyl}-acetamide;
(S)—N-{3-[3-fluoro-4-(4-(4-methyl-5-thioxo-4,5-dihydro-[1,2,4]triazol-1-yl)-piperidin-1-yl)-phenyl]-2-oxo-oxazolidin-5-ylmethyl}-acetamide;
(S)—N-{3-[4-(4-(isoxazol-3-yloxymethyl)-piperidin-1-yl)-3-fluorophenyl]-2-oxo-oxazolidin-5-ylmethyl}-acetamide;
(S)—N-{3-[4-(4-([1,2,3]-triazol-1-yl)-methyl-piperidin-1-yl)-3-fluoro-phenyl]-5-(isoxazol-3-yloxymethyl)}-oxazolidin-2-one;
(R)-{3-[4-(4-((4-hydroxymethyl)-[1,2,3]-triazol-1-yl)-methyl-piperidin-1-yl)-3-fluoro-phenyl]-5-(isoxazol-3-yloxymethyl)}-oxazolidin-2-one;
cis-(R)-{3-[4-(4-(5-ethoxycarbonyl-1H-[1,2,3]-triazol-1-yl)-piperidin-1-yl)-3-fluorophenyl]-5-(5-ethoxycarbonyl-1H-[1,2,3]-triazol-1-ylmethyl)}-oxazolidin-2-one;
cis-(R)-{3-[4-(4-(5-ethoxycarbonyl-1H-[1,2,3]-triazol-1-yl)-piperidin-1-yl)-3-fluoro-phenyl]-5-(4-ethoxycarbonyl-1H-[1,2,3]-triazol-1-ylmethyl)}-oxazolidin-2-one;
trans-(R)-{3-[4-(4-(5-ethoxycarbonyl-1H-[1,2,3]-triazol-1-yl)-piperidin-1-yl)-3-fluoro-phenyl]-5-(5-ethoxycarbonyl-1H-[1,2,3]-triazol-1-ylmethyl)}-oxazolidin-2-one;
trans-(R)-{3-[4-(4-(5-ethoxycarbonyl-1H-[1,2,3]-triazol-1-yl)-piperidin-1-yl)-3-fluoro-phenyl]-5-(4-ethoxycarbonyl-1H-[1,2,3]-triazol-1-ylmethyl)}-oxazolidin-2-one;
trans-(R)-{3-[4-(4-(5-hydroxymethyl-1H-[1,2,3]-triazol-1-yl)-piperidin-1-yl)-3-fluoro-phenyl]-5-(4-hydroxymethyl-1H-[1,2,3]-triazol-1-ylmethyl)}-oxazolidin-2-one;
trans-(R)-{3-[4-(4-(5-hydroxymethyl-1H-[1,2,3]-triazol-1-yl)-piperidin-1-yl)-3-fluoro-phenyl]-5-(5-hydroxymethyl-1H-[1,2,3]-triazol-1-ylmethyl)}-oxazolidin-2-one;
trans-(R)-{3-[4-(4-(5-cyano-1H-[1,2,3]-triazol-1-yl)-piperidin-1-yl)-3-fluoro-phenyl]-5-(5-cyano-1H-[1,2,3]-triazol-1-ylmethyl)}-oxazolidin-2-one;
trans-(R)-{3-[4-(4-(5-cyano-1H-[1,2,3]-triazol-1-yl)-piperidin-1-yl)-3-fluoro-phenyl]-5-(4-cyano-1H-[1,2,3]-triazol-1-ylmethyl)}-oxazolidin-2-one;
(3R,5S)—N-{3-[4-(Fluoro-4-(1H-[1,2,3]-triazol-1-yl)-piperidin-1-yl)-3-fluoro-phenyl]-2-oxo-oxazolidin-5-ylmethyl}-acetamide;
(R)-{3-[4-(4-(5-ethoxycarbonylmethyl-2H-[1,2,3,4]-tetrazol-2-yl)-piperidin-1-yl)-3,5-difluorophenyl]-5-(1H-[1,2,3]-triazol-1-ylmethyl)}-oxazolidin-2-one;
(S)—N-{3-[4-(4-(5-hydroxyethyl-2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4-(5-acetylaminomethyl-2H-[1,2,3,4]-tetrazol-2-yl)-piperidin-1-yl)-3,5-difluorophenyl]-2-oxo-oxazolidin-5-ylmethyl}-acetamide;
(R)-{3-(4-phenyl-3-fluorophenyl)-5-1H-1,2,3-triazol-1-ylmethyl}-oxazolidin-2-one;
(R)-{3-(4-phenyl-3-fluorophenyl)-5-1H-1,2,4-triazol-1-ylmethyl}-oxazolidin-2-one;
(R)-{3-(4-phenyl-3-fluorophenyl)-5-1H-1,2,3-triazol-2-ylmethyl}-oxazolidin-2-one;
(R)-{3-[4-(4-(piperidin-1-yl)-3-fluorophenyl)-3-fluorophenyl]-2-oxo-oxazolidin-5-ylmethyl}-methanesulfonate;
(S)—N-{3-[4-(4-(piperidin-1-yl)-3-fluorophenyl)-3-fluorophenyl]-2-oxo-oxazolidin-5-ylmethyl}-acetamide;
(S)-{3-[4-(4-(piperidin-1-yl)-3-fluorophenyl)-3-fluorophenyl]-5-imidazol-1-ylmethyl}-oxazolidin-2-one;
(R)-{3-[4-(4-(piperidin-1-yl)-3-fluorophenyl)-3-fluorophenyl]-5-1H-1,2,3-triazol-1-ylmethyl}-oxazolidin-2-one;
(R)-{3-[4-(4-(piperidin-1-yl)-3-fluorophenyl)-3-fluorophenyl]-5-1H-1,2,3-triazol-2-ylmethyl}-oxazolidin-2-one;
(R)-{3-[4-(4-(piperidin-1-yl)-3-fluorophenyl)-3-fluorophenyl]-5-1H-1,2,3,4-tetrazol-1-ylmethyl}-oxazolidin-2-one;
(S)—N-{3-[4-(4-(4,4-dimethoxypiperidin-1-yl)-3-fluorophenyl)-3-fluorophenyl]-2-oxo-oxazolidin-5-ylmethyl}-acetamide;
(R)-{3-[4-(4-(1,4-dioxa-8-azaspiro[4.5]dec-8-yl)-3-fluorophenyl)-3-fluorophenyl]-2-oxo-oxazolidin-5-ylmethyl}-methanesulfonate;
(S)—N-{3-[4-(4-(1,4-dioxa-8-azaspiro[4.5]dec-8-yl)-3-fluorophenyl)-3-fluorophenyl]-2-oxo-oxazolidin-5-ylmethyl}-acetamide;
(S)—N-{3-[4-(6-(1,4-dioxa-8-azaspiro[4.5]dec-8-yl)-pyridin-3-yl)-3-fluorophenyl]-2-oxo-oxazolidin-5-ylmethyl}-acetamide;
(R)-{3-[4-(4-(4-oxo-piperidin-1-yl)-3-fluorophenyl)-3-fluorophenyl]-2-oxo-oxazolidin-5-ylmethyl}-methanesulfonate;
(S)—N-{3-[4-(4-(4-oxo-piperidin-1-yl)-3-fluorophenyl)-3-fluorophenyl]-2-oxo-oxazolidin-5-ylmethyl}-acetamide;
(R)-{3-[4-(4-(4-hydroxy-piperidin-1-yl)-3-fluorophenyl)-3-fluorophenyl]-2-oxo-oxazolidin-5-ylmethyl}-methanesulfonate;
(S)—N-{3-[4-(4-(4-hydroxy-piperidin-1-yl)-3-fluorophenyl)-3-fluorophenyl]-2-oxo-oxazolidin-5-ylmethyl}-acetamide;
(S)—N-{3-[4-(6-(4-hydroxy-piperidin-1-yl)-pyridin-3-yl)-3-fluorophenyl]-2-oxo-oxazolidin-5-ylmethyl}-acetamide;
(R)-{3-[4-(4-(4-methanesulfonyloxy-piperidin-1-yl)-3-fluorophenyl)-3-fluorophenyl]-2-oxo-oxazolidin-5-ylmethyl}-methanesulfonate;
(S)—N-{3-[4-(4-(4-methanesulfonyloxy-piperidin-1-yl)-3-fluorophenyl)-3-fluorophenyl]-2-oxo-oxazolidin-5-ylmethyl}-acetamide;
(S)—N-{3-[4-(6-(4-methanesulfonyloxy-piperidin-1-yl)-pyridin-3-yl)-3-fluorophenyl]-2-oxo-oxazolidin-5-ylmethyl}-acetamide;
(S)—N-{3-[4-(4-(4-cyanomethylidene-piperidin-1-yl)-3-fluorophenyl)-3-fluorophenyl]-2-oxo-oxazolidin-5-ylmethyl}-acetamide;
(R)-{3-[4-(4-(4-cyanomethylidene-piperidin-1-yl)-3-fluorophenyl)-3-fluorophenyl]-5-(1H-1,2,3-triazol-1-yl)-methyl}-oxazolidin-2-one;
(R)-{3-[4-(4-(4-cyanomethylidene-piperidin-1-yl)-3-fluorophenyl)-3-fluorophenyl]-5-(1H-tetrazol-1-yl)methyl}-oxazolidin-2-one;
(S)—N-{3-[4-(4-(4-cyanomethyl-3,4-dehydropiperidin-1-yl)-3-fluorophenyl)-3-fluorophenyl]-2-oxo-oxazolidin-5-ylmethyl}-acetamide;
(S)—N-{3-[4-(4-(4-cyanomethyl-piperidin-1-yl)-3-fluorophenyl)-3-fluorophenyl]-2-oxo-oxazolidin-5-ylmethyl}-acetamide;
(S)—N-{3-[4-(4-(4-hydroxy-4-hydroxymethyl-piperidin-1-yl)-3-fluorophenyl)-3-fluorophenyl]-2-oxo-oxazolidin-5-ylmethyl}-acetamide;
(S)—N-{3-[4-(4-(4-azidomethyl-4-hydroxy-piperidin-1-yl)-3-fluorophenyl)-3-fluorophenyl]-2-oxo-oxazolidin-5-ylmethyl}-acetamide;
(S)—N-{3-[4-(4-(4-(1H-1,2,3-triazol-1-yl)-piperidin-1-yl)-3-fluorophenyl)-3-fluorophenyl]-2-oxo-oxazolidin-5-ylmethyl}-acetamide and isomer thereof;
(S)—N-{3-[4-(4-(4-(1H-1,2,3,4-tetrazol-1-yl)-piperidin-1-yl)-3-fluorophenyl)-3-fluorophenyl]-2-oxo-oxazolidin-5-ylmethyl}-acetamide and isomer thereof;
(S)—N-{3-[4-(6-(4-Oxopiperidin-1-yl)-pyridin-3-yl)-3-fluorophenyl]-2-oxo-oxazolidin-5-ylmethyl}-acetamide;
(S)—N-{3-[4-(6-(4-Hydroxy-4-methoxymethylpiperidin-1-yl)-pyridin-3-yl)-3-fluorophenyl]-2-oxo-oxazolidin-5-ylmethyl}-acetamide;
(S)—N-{3-[4-(6-(4-Hydroxy-4-acetyloxymethylpiperidin-1-yl)-pyridin-3-yl)-3-fluorophenyl]-2-oxo-oxazolidin-5-ylmethyl}-acetamide;
(R)-{3-[4-(4-(piperazin-1-yl)-3-fluorophenyl)-3-fluorophenyl]-5-1H-1,2,3-triazol-1-ylmethyl}-oxazolidin-2-one;
(R)-{3-[4-(4-(piperazin-1-yl)-3-fluorophenyl)-3-fluorophenyl]-5-1H-1,2,3,4-tetrazol-1-ylmethyl}-oxazolidin-2-one;
(R)-{3-[4-(4-(piperazin-1-yl)-3-fluorophenyl)-3-fluorophenyl]-2-oxo-oxazolidin-5-ylmethyl}-methanesulfonate hydrochloride;
(R)-{3-[4-(4-(piperazin-1-yl)-3-fluorophenyl)-3-fluorophenyl]-5-(1,2,3-triazol-1-yl)methyl}-oxazolidin-2-one hydrochloride;
(S)-{3-[4-(4-(4-hydroxymethylcarbonyl-piperazin-1-yl)-3-fluorophenyl)-3-fluorophenyl]-2-oxo-oxazolidin-5-ylmethyl}-acetamide;
(R)-{3-[4-(4-(4-(2-nitrofuran-5-yl-methyl)-piperazin-1-yl)-3-fluorophenyl)-3-fluorophenyl]-2-oxo-oxazolidin-5-ylmethyl}-methanesulfonate;
(S)—N-{3-[4-(4-(4-(2-nitrofuran-5-yl-methyl)-piperazin-1-yl)-3-fluorophenyl)-3-fluorophenyl]-2-oxo-oxazolidin-5-ylmethyl}-acetamide;
(R)-{3-[4-(4-(4-benzyloxycarbonyl-piperazin-1-yl)-3-fluorophenyl)-3-fluorophenyl]-2-oxo-oxazolidin-5-ylmethyl}-methanesulfonate;
(S)-{3-[4-(4-benzyloxycarbonyl-piperazin-1-yl-3-fluorophenyl)-3-fluorophenyl]-5-1H-1,2,3-triazol-1-ylmethyl}-oxazolidin-2-one;
(S)—N-{3-[4-(4-benzyloxycarbonyl-piperazin-1-yl-3-fluorophenyl)-3-fluorophenyl]-2-oxo-oxazolidin-5-ylmethyl}-acetamide;
(S)—N-{3-[4-(4-(1-oxa-6-azaspiro[2.5]oct-6-yl)-3-fluorophenyl)-3-fluorophenyl]-2-oxo-oxazolidin-5-ylmethyl}-acetamide;
(S)—N-{3-[4-(4-(morpholin-1-yl)-phenyl)-3-fluorophenyl]-2-oxo-oxazolidin-5-ylmethyl}-acetamide;
(S)—N-{3-[4-(4-(thiomorpholin-4-yl)-3-fluorophenyl)-3-fluorophenyl]-2-oxo-oxazolidin-5-ylmethyl}-acetamide;
(S)—N-{3-[4-(6-(thiomorpholin-4-yl)-pyridin-3-yl)-3-fluorophenyl]-2-oxo-oxazolidin-5-ylmethyl}-methanesulfonate;
(S)—N-{3-[4-(6-(thiomorpholin-4-yl)-pyridin-3-yl)-3-fluorophenyl]-2-oxo-oxazolidin-5-ylmethyl}-acetamide;
(R)-{3-[4-(6-(Thiomorpholin-4-yl)-pyridin-3-yl)-3-fluorophenyl]-2-oxo-oxazolidin-5-ylmethyl}-methansulphonate;
(S)—N-{3-[4-(6-(Thiomorpholin-4-yl)-pyridin-3-yl)-3-fluorophenyl]-2-oxo-oxazolidin-5-ylmethyl}-formamide;
(S)—N-{3-[4-(6-(Thiomorpholin-4-yl)-pyridin-3-yl)-3-fluorophenyl]-2-oxo-oxazolidin-5-ylmethyl}-propionamide;
(S)-{3-[4-(6-(thiomorpholin-4-yl)-pyridin-3-yl)-3-fluorophenyl]-2-oxo-oxazolidin-5-ylmethyl}-carbamic acid methyl ester;
(S)-{3-[4-(6-thiomorpholin-4-yl-pyridin-3-yl)-3-fluorophenyl]-5-1,2,3-triazol-1-ylmethyl}-oxazolidin-2-one;
(S)—N-{3-[6-(6-(Thiomorpholin-4-yl)-pyridin-3-yl)-pyridin-3-yl]-2-oxo-oxazolidin-5-ylmethyl}-acetamide;
(S)—N-{3-[6-(4-(Thiomorpholin-4-yl)-3-fluorophenyl)-pyridin-3-yl]-2-oxo-oxazolidin-5-ylmethyl}-acetamide;
(S)—N-{3-[4-(6-(Thiomorpholin-4-yl)-pyridin-3-yl)-phenyl]-2-oxo-oxazolidin-5-yl methyl}-acetamide;
(S)—N-{3-[4-(2-(Thiomorpholin-4-yl)-pyrimidin-5-yl)-3-fluorophenyl]-2-oxo-oxazolidin-5-ylmethyl}-carbamic acid methyl ester;
(S)—N-{3-[4-(2-(Thiomorpholin-4-yl)-pyrimidin-5-yl)-3-fluorophenyl]-5-hydroxymethyl}-oxazolidin-2-one;
(S)—N-{3-[4-(2-(Thiomorpholin-4-yl)-pyrimidin-5-yl)-3-fluorophenyl]-2-oxo-oxazolidin-5-ylmethyl}-acetamide;
(S)—N-{3-[4-(2-(Thiomorpholin-4-yl)-pyrimidin-5-yl)-3-fluorophenyl]-5-1,2,3-triazol-1-ylmethyl}-oxazolidin-2-one;
(S)—N-{3-[4-(6-(Thiomorpholin-4-yl)-3-methylpyridin-3-yl)-3-fluorophenyl]-2-oxo-oxazolidin-5-ylmethyl}-carbamic acid methyl ester;
(S)—N-{3-[4-(6-(Thiomorpholin-4-yl)-3-methylpyridin-3-yl)-3-fluorophenyl]-2-oxo-oxazolidin-5-ylmethyl}-acetamide;
(S)—N-{3-[4-(6-(Thiomorpholin-4-yl)-3-methylpyridin-3-yl)-3-fluorophenyl]-5-hydroxymethyl}-oxazolidin-2-one;
(S)—N-{3-[4-(6-(Thiomorpholin-4-yl)-3-methylpyridin-3-yl)-3-fluorophenyl]-5-1,2,3-triazol-1-ylmethyl}-oxazolidin-2-one;
(S)—N-{3-[4-(2-(S-Oxo-thiomorpholin-4-yl)-pyridin-5-yl)-3-fluorophenyl]-5-hydroxymethyl}-oxazolidin-2-one;
(S)—N-{3-[4-(4-(S-oxo-thiomorpholin-4-yl)-3-fluorophenyl)-3-fluorophenyl]-2-oxo-oxazolidin-5-ylmethyl}-acetamide;
(S)—N-{3-[6-[6-(S-oxo-thiomorpholin-4-yl)-pyridin-3-yl]-pyridin-3-yl]-2-oxo-oxazolidin-5-ylmethyl}-acetamide;
(S)—N-{3-[4-(6-(S-oxo-thiomorpholin-4-yl)-pyridin-3-yl)-3-fluorophenyl]-2-oxo-oxazolidin-5-ylmethyl}-acetamide;
(R)-{3-[4-(6-(S-Oxo-thiomorpholin-4-yl)-pyridin-3-yl)-3-fluorophenyl]-2-oxo-oxazolidin-5-ylmethyl}-methansulphonate;
(S)—N-{3-[4-(6-(S-Oxo-thiomorpholin-4-yl)-pyridin-3-yl)-phenyl]-2-oxo-oxazolidin-5-ylmethyl}-acetamide;
(S)—N-{3-[4-(2-(S-Oxo-thiomorpholin-4-yl)-pyrimidin-5-yl)-3-fluorophenyl]-2-oxo-oxazolidin-5-ylmethyl}-carbamic acid methyl ester;
(S)—N-{3-[4-(2-(S-Oxo-thiomorpholin-4-yl)-pyrimidin-5-yl)-3-fluorophenyl]-2-oxo-oxazolidin-5-ylmethyl}-acetamide;
(S)—N-{3-[4-(2-(S-Oxo-thiomorpholin-4-yl)-pyrimidin-5-yl)-3-fluorophenyl]-5-1,2,3-triazol-1-ylmethyl}-oxazolidin-2-one;
(S)—N-{3-[4-(6-(S-Oxo-thiomorpholin-4-yl)-pyrimidin-5-yl)-3-fluorophenyl]-5-hydroxymethyl}-oxazolidin-2-one;
(S)—N-{3-[4-(6-(S-Oxo-thiomorpholin-4-yl)-3-methylpyridin-3-yl)-3-fluorophenyl]-2-oxo-oxazolidin-5-ylmethyl}-carbamic acid methyl ester;
(S)—N-{3-[4-(6-(S-Oxo-thiomorpholin-4-yl)-3-methylpyridin-3-yl)-3-fluorophenyl]-5-1,2,3-triazol-1-ylmethyl}-oxazolidin-2-one;
(S)—N-{3-[4-(6-(S-Oxo-thiomorpholin-4-yl)-3-methylpyridin-3-yl)-3-fluorophenyl]-5-hydroxymethyl}-oxazolidin-2-one;
(S)—N-{3-[4-(6-(S-Oxo-thiomorpholin-4-yl)-3-methylpyridin-3-yl)-3-fluorophenyl]-2-oxo-oxazolidin-5-ylmethyl}-acetamide;
(S)—N-{3-[4-(6-(S-oxo-3-fluoro-thiomorpholin-4-yl)-pyridin-3-yl)-3-fluorophenyl]-2-oxo-oxazolidin-5-ylmethyl}-acetamide;
(S)—N-{3-[4-(6-(S,S-dioxo-thiomorpholin-4-yl)-pyridin-3-yl)-3-fluorophenyl]-2-oxo-oxazolidin-5-ylmethyl}-acetamide;
(S)—N-{3-[4-(6-(S,S-dioxo-thiomorpholin-4-yl)-pyridin-3-yl)-3,5-difluorophenyl]-2-oxo-oxazolidin-5-ylmethyl}-acetamide;
(S)—N-{3-[4-(6-(S,S-dioxo-3-methyl-thiomorpholin-4-yl)-pyridin-3-yl)-3-fluorophenyl)]-2-oxo-oxazolidin-5-ylmethyl}-acetamide;
(S)—N-{3-[4-(4-(S,S-dioxo-thiomorpholin-4-yl)-3-fluorophenyl)-3-fluorophenyl]-2-oxo-oxazolidin-5-ylmethyl}-acetamide;
(S)—N-{3-[6-[6-(S,S-dioxo-thiomorpholin-4-yl)-pyridin-3-yl]-pyridin-3-yl]-2-oxo-oxazolidin-5-ylmethyl}-acetamide;
(S)—N-{3-[6-(4-(S,S-dioxo-thiomorpholin-4-yl)-3-fluorophenyl)-pyridin-3-yl]-2-oxo-oxazolidin-5-ylmethyl}-acetamide;
(R)-3-{4-[6-(S,S-dioxo-thiomorpholin-4-yl)-pyridin-3-yl]-3-fluorophenyl}-5-hydroxymethyl-oxazolidin-2-one;
(2S,5R)-2-Amino-propionic acid 3-{4-[6-(S,S-dioxo-thiomorpholin-4-yl)-pyridin-3-yl]-3-fluoro-phenyl}-2-oxo-oxazolidin-5-ylmethyl ester;
(2S,5R)-2-Amino-propionic acid 3-{4-[6-(S,S-dioxo-thiomorpholin-4-yl)-pyridin-3-yl]-3-fluoro-phenyl}-2-oxo-oxazolidin-5-ylmethyl ester methanesulfonic acid salt;
(2S,5R)-2-Amino-3-methyl-butyric acid 3-{4-[6-(S,S-dioxo-thiomorpholin-4-yl)-pyridin-3-yl]-3-fluoro-phenyl}-2-oxo-oxazolidin-5-ylmethyl ester;
(2S,5R)-2-Amino-3-methyl-butyric acid 3-{4-[6-(S,S-dioxo-thiomorpholin-4-yl)-pyridin-3-yl]-3-fluoro-phenyl}-2-oxo-oxazolidin-5-ylmethyl ester methane sulfonic acid salt;
(R)-Acetic acid 3-{4-[6-(S,S-dioxo-thiomorpholin-4-yl)-pyridin-3-yl]-3-fluoro-phenyl}-2-oxo-oxazolidin-5-ylmethyl ester;
(S)—N-{3-[4-(6-(S,S-Dioxo-thiomorpholin-4-yl)-pyridin-3-yl)-3-fluorophenyl]-2-oxo-oxazolidin-5-ylmethyl}-formamide;
(R)-{3-[4-(6-(S,S-Dioxo-thiomorpholin-4-yl)-pyridin-3-yl)-3-fluorophenyl]-2-oxo-oxazolidin-5-ylmethyl}-methansulphonate;
(S)—N-{3-[4-(6-(S,S-Dioxo-thiomorpholin-4-yl)-pyridin-3-yl)-3-fluorophenyl]-2-oxo-oxazolidin-5-ylmethyl}-propionamide;
(S)—N-{3-[4-(6-(S,S-Dioxo-thiomorpholin-4-yl)-pyridin-3-yl)-3-fluorophenyl]-2-oxo-oxazolidin-5-ylmethyl}-carbamic acid methyl ester;
N-Acetyl-(R)—N-{3-4-{2-(S,S-Dioxo-thiomorpholin-4-yl)-pyridyl-5-yl-3-fluorophenyl-1-yl-]}-2-oxo-oxazolidin-5-ylmethyl}-aminocarbonyl-oxymethyl acetate;
(S)—N-{3-[4-(6-(S,S-Dioxo-thiomorpholin-4-yl)-pyridin-3-yl)-3-fluorophenyl]-5-1,2,3-triazol-1-ylmethyl}-oxazolidin-2-one;
(S)—N-{3-[4-(6-(S,S-Dioxo-thiomorpholin-4-yl)-pyridin-3-yl)-3-fluorophenyl]-2-oxo-oxazolidin-5-ylmethyl}-urea;
(S)—N-{3-[4-(6-(S,S-Dioxo-thiomorpholin-4-yl)-pyridin-3-yl)-phenyl]-2-oxo-oxazolidin-5-ylmethyl}-acetamide;
(S)—N-{3-[6-(6-(S,S-Dioxo-thiomorpholin-4-yl)-pyridin-3-yl)-pyridin-3-yl]-2-oxo-oxazolidin-5-ylmethyl}-acetamide;
(S)—N-{3-[4-(2-(S,S-Dioxo-thiomorpholin-4-yl)-pyrimidin-5-yl)-3-fluorophenyl]-2-oxo-oxazolidin-5-ylmethyl}-carbamic acid methyl ester;
(S)—N-{3-[4-(2-(S,S-Dioxo-thiomorpholin-4-yl)-pyrimidin-5-yl)-3-fluorophenyl]-5-hydroxymethyl}-oxazolidin-2-one;
(S)—N-{3-[4-(2-(S,S-Dioxo-thiomorpholin-4-yl)-pyrimidin-5-yl)-3-fluorophenyl]-2-oxo-oxazolidin-5-ylmethyl}-acetamide;
(S)—N-{3-[4-(2-(S,S-Dioxo-thiomorpholin-4-yl)-pyrimidin-5-yl)-3-fluorophenyl]-5-1,2,3-triazol-1-ylmethyl}-oxazolidin-2-one;
(S)—N-{3-[4-(6-(S,S-Dioxo-thiomorpholin-4-yl)-3-methylpyridin-3-yl)-3-fluorophenyl]-2-oxo-oxazolidin-5-ylmethyl}-carbamic acid methyl ester;
(S)—N-{3-[4-(6-(S,S-Dioxo-thiomorpholin-4-yl)-3-methylpyridin-3-yl)-3-fluorophenyl]-5-hydroxymethyl}-oxazolidin-2-one;
(S)—N-{3-[4-(6-(S,S-Dioxo-thiomorpholin-4-yl)-3-methylpyridin-3-yl)-3-fluorophenyl]-2-oxo-oxazolidin-5-ylmethyl}-acetamide;
(S)—N-{3-[4-(6-(S,S-Dioxo-thiomorpholin-4-yl)-3-methylpyridin-3-yl)-3-fluorophenyl]-5-1,2,3-triazol-1-ylmethyl}-oxazolidin-2-one;
(S)—N-{3-[4-(6-(S,S-Dioxo-3-methyl-thiomorpholin-4-yl)-pyridin-3-yl)-3-fluorophenyl]-2-oxo-oxazolidin-5-ylmethyl}-difluoroacetamide;
(S)—N-{3-[4-(6-(S-Oxo-S—(N-methylimino)-thiomorpholin-4-yl)-pyridyl-3-yl)-3-fluorophenyl-1-yl]-2-oxo-oxazolidin-5-ylmethyl}-acetamide;
(S)—N-{3-[4-(6-(S-Oxo-S—(N-methylimino)-thiomorpholin-4-yl)-pyridin-3-yl)-phenyl]-2-oxo-oxazolidin-5-ylmethyl}-acetamide;
(S)—N-{3-[4-(6-(S-Oxo-S—(N-acetylimino)-thiomorpholin-4-yl)-pyridyl-3-yl)-3-fluorophenyl-1-yl]-2-oxo-oxazolidin-5-ylmethyl}-acetamide;
(S)—N-{3-[4-(6-(S-Oxo-S-(1-chloroethylurido)-thiomorpholin-4-yl)-pyridyl-3-yl)-3-fluorophenyl-1-yl]-2-oxo-oxazolidin-5-ylmethyl}-acetamide;
(S)—N-{3-[4-(6-(S-Oxo-S-imino-thiomorpholin-4-yl)-pyridyl-3-yl)-3-fluorophenyl-1-yl]-2-oxo-oxazolidin-5-ylmethyl}-acetamide;
(S)—N-{3-[4-(6-(1,4-Dioxa-8-azaspiro[4.5]dec-8-yl)-pyridin-3-yl)-3-fluorophenyl]-2-oxo-oxazolidin-5-ylmethyl}-acetamide;
(S)—N-{3-[4-(6-(S-Oxo-3,3-dichloro-thiomorpholin-4-yl)-pyridin-3-yl)-3-fluorophenyl]-2-oxo-oxazolidin-5-ylmethyl}-acetamide;
(R)-Phosphoric acid {3-[4-(6-(S,S-dioxo-thiomorpholin-4-yl)-pyridin-3-yl)-3-fluorophenyl]-2-oxo-oxazolidin-5-ylmethyl}ester disodium salt;
Phosphoric acid mono (R)—N-{3-{4-{2-(thiomorpholin-4-yl)-pyrimidin-5-yl}-3-fluorophenyl]}-2-oxo-oxazolidin-5-ylmethyl}ester;
Phosphoric acid mono (R)—N-{3-{4-(2-(S-oxo-thiomorpholin-4-yl)-pyrimidin-5-yl}-3-fluorophenyl]}-2-oxo-oxazolidin-5-ylmethyl}ester;
Phosphoric acid mono (R)—N-{3-{4-{2-(S,S-dioxo-thiomorpholin-4-yl)-pyrimidin-5-yl}-3-fluorophenyl]}-2-oxo-oxazolidin-5-ylmethyl}-ester;
(R)-Phosphoric acid mono {3-[4-(6-(S,S-dioxo-thiomorpholin-4-yl)-pyridin-3-yl)-3-fluorophenyl]-2-oxo-oxazolidin-5-ylmethyl}ester;
Phosphoric acid mono (R)—N-{3-{4-{2-(S,S-Dioxo thiomorpholin-4-yl)-pyrimidin-5-yl}-3-fluorophenyl]}-2-oxo-oxazolidin-5-ylmethyl}-ester disodium salt;
(R)-Phosphoric acid mono {3-[4-(6-(thiomorpholin-4-yl)-3-methylpyridin-3-yl)-3-fluorophenyl]-2-oxo-oxazolidin-5-ylmethyl}ester;
(R)-Phosphoric acid mono {3-[4-(6-(S-oxo-thiomorpholin-4-yl)-3-methylpyridin-3-yl)-3-fluorophenyl]-2-oxo-oxazolidin-5-ylmethyl}ester;
(R)-Phosphoric acid mono {3-[4-(6-(S,S-dioxo-thiomorpholin-4-yl)-3-methylpyridin-3-yl)-3-fluorophenyl]-2-oxo-oxazolidin-5-ylmethyl}ester;
(R)-Phosphoric acid mono {3-[4-(6-(S-oxo-thiomorpholin-4-yl)-3-methylpyridin-3-yl)-3-fluorophenyl]-2-oxo-oxazolidin-5-ylmethyl}ester disodium salt;
(5S)—N-[3-(3′-Chloromethyl-2-fluoro-biphenyl-4-yl)-2-oxo-oxazolidin-5-ylmethyl]-acetamide;
(5S)—N-[3-(2-Fluoro-3′-fluoromethyl-biphenyl-4-yl)-2-oxo-oxazolidin-5-ylmethyl]-acetamide;
(5S)—N-[3-(2-Fluoro-3′-azidomethyl-biphenyl-4-yl)-2-oxo-oxazolidin-5-ylmethyl]-acetamide;
(5S)—N-[3-(3′-Aminomethyl-2-fluoro-biphenyl-4-yl)-2-oxo-oxazolidin-5-ylmethyl]-acetamide;
(5S)-2-({4′-[5-(Acetylamino-methyl)-2-oxo-oxazolidin-3-yl]-2′-fluoro-biphenyl-3-ylmethyl}-amino)-acetamide;
(5S)-2-({4′-[5-(Acetylamino-methyl)-2-oxo-oxazolidin-3-yl]-2′-fluoro-biphenyl-3-ylmethyl}-carbamoylmethyl-amino)-acetamide;
(5S)—N-{3-[4-(4-Azido-piperidin-1-yl)-3,5-difluoro-phenyl]-2-oxo-oxazolidin-5-ylmethyl}-acetamide;
(5S)-{3-[2-Fluoro-1,1′-biphenyl-3′-methoxy-4-yl]-2-oxo-oxazolidin-5yl-methyl}-acetamide;
(5S)-{3-[(2-Fluoro-1,1′-biphenyl-3′-yl)methanol]-2-oxo-oxazolidin-5yl-methyl}-acetamide;
(5S)-{3-[(3,5-Difluoro-4-[6-(4-thiomorpholin-1,1-dioxide-4-yl)-3-pyridinyl]phenyl]-2-oxo-oxazolidin-5yl-methyl}-acetamide;

32. A compound or a pharmaceutically acceptable salt thereof which is
(S)—N-{3-[4-(4-(1H-[1,2,3,4]-tetrazol-1-yl)-piperidin-1-yl)-3-fluorophenyl]-2-oxo-oxazolidin-5-ylmethyl}-acetamide;
(S)—N-{3-[4-(4-(2H-[1,2,3,4]-tetrazol-2-yl)-piperidin-1-yl)-3,5-difluorophenyl]-2-oxo-oxazolidin-5-ylmethyl}-acetamide;
(S)—N-{3-[4-(4 (1H-[1,2,3,4]-tetrazol-1-yl)-piperidin-1-yl)-3,5-difluorophenyl]-2-oxo-oxazolidin-5-ylmethyl}-acetamide
(S)—N-{3-[4-(4-(1H-[1,2,3]-triazol-1-yl)-piperidin-1-yl)-3,5-difluorophenyl]-2-oxo-oxazolidin-5-ylmethyl}-acetamide;
(S)—N-{3-[3,5-difluoro-4-(4-2H-tetrazol-2-yl-piperidin-1-yl)-phenyl]-2-oxo-oxazolidin-5-ylmethyl}-propionamide;
(S)—N-{3-[3,5-difluoro-4-(4-1H-tetrazol-1-yl-piperidin-1-yl)-phenyl]-2-oxo-oxazolidin-5-ylmethyl}-propionamide;
(S)—N-{3-[4-(4-(2H-benzotriazol-2-yl)-piperidin-1-yl)-3,5-difluorophenyl]-2-oxo-oxazolidin-5-ylmethyl}-acetamide;
(S)—N-{3-[3,5-difluoro-4-(4-[1,2,3]-triazol-1-yl-piperidin-1-yl)-phenyl]-2-oxo-oxazolidin-5-ylmethyl}-carbamic acid methyl ester;
(S)—N-{3-[3-fluoro-4-(4-2H-tetrazol-2-yl-piperidin-1-yl)-phenyl]-2-oxo-oxazolidin-5-ylrmethyl}-carbamic acid methyl ester;
(S)—N-{3-[3-fluoro-4-(4-1H-tetrazol-1-yl-piperidin-1-yl)-phenyl]-2-oxo-oxazolidin-5-ylmethyl}-carbamic acid methyl ester;
(S)—N-{3-[3,5-difluoro-4-(4-(4-methyl-5-thioxo-4,5-dihydro-tetrazol-1-yl)-piperidin-1-yl)-phenyl]-2-oxo-oxazolidin-5-ylmethyl}-acetamide;
(S)—N-{3-[3,5-difluoro-4-(4-[1,2,3]-triazol-1-yl-piperidin-1-yl)-phenyl]-2-oxo-oxazolidin-5-ylmethyl}-2,2-difluoro-acetamide;

33. A compound or a pharmaceutically acceptable salt thereof which is
(S)—N-{3-[4-(4-(thiomorpholin-4-yl)-3-fluorophenyl)-3-fluorophenyl]-2-oxo-oxazolidin-5-ylmethyl}-acetamide;
(S)—N-{3-[4-(4-(S-oxo-thiomorpholin-4-yl)-3-fluorophenyl)-3-fluorophenyl]-2-oxo-oxazolidin-5-ylmethyl}-acetamide;
(S)—N-{3-[4-(4-(S,S-dioxo-thiomorpholin-4-yl)-3-fluorophenyl)-3-fluorophenyl]-2-oxo-oxazobidin-5-ylmethyl}-acetamide;
(S)—N-{3-[4-(6-(thiomorpholin-4-yl)-pyridin-3-yl)-3-fluorophenyl]-2-oxo-oxazolidin-5-ylmethyl}-acetamide;
(S)—N-{3-[4-(6-(S-oxo-thiomorpholin-4-yl)-pyridin-3-yl)-3-fluorophenyl]-2-oxo-oxazolidin-5-ylmethyl}-acetamide;
(S)—N-{3-[4-(6-(S,S-dioxo-thiomorpholin-4-yl)-pyridin-3-yl)-3-fluorophenyl]-2-oxo-oxazolidin-5-ylmethyl}-acetamide;
(S)—N-{3-[4-(6-(S,S-dioxo-3-methyl-thiomorpholin-4-yl)-pyridin-3-yl)-3-fluorophenyl]-2-oxo-oxazolidin-5-ylmethyl}-acetamide;
(R)-3-{4-[6-(S,S-Dioxo-thiomorpholin-4-yl)-pyridin-3-yl]-3-fluorophenyl}-5-hydroxymethyl-oxazolidin-2-one;
(R)-Phosphoric acid mono {3-[4-(6-(S,S-dioxo-thiomorpholin-4-yl)-pyridin-3-yl)-3-fluorophenyl]-2-oxo-oxazolidin-5-ylmethyl}ester;
(R)-Phosphoric acid {3-[4-(6-(S,S-dioxo-thiomorpholin-4-yl)-pyridin-3-yl)-3-fluorophenyl]-2-oxo-oxazolidin-5-ylmethyl}ester disodium salt;
(2S,5R)-2-Amino-propionic acid 3-{4-[6-(S,S-dioxo-thiomorpholin-4-yl)-pyridin-3-yl]-3-fluoro-phenyl}-2-oxo-oxazolidin-5-ylmethyl ester;
(2S,5R)-2-Amino-propionic acid 3-{4-[6-(S,S-dioxo-thiomorpholin-4-yl)-pyridin-3-yl]-3-fluoro-phenyl}-2-oxo-oxazolidin-5-ylmethyl ester methanesulphonic acid salt;
(2S,5R)-2-Amino-3-methyl-butyric acid 3-{4-[6-(S,S-dioxo-thiomorpholin-4-yl)-pyridin-3-yl]-3-fluoro-phenyl}-2-oxo-oxazolidin-5-ylmethyl ester;
(2S,5R)-2-Amino-3-methyl-butyric acid 3-{4-[6-(S,S-dioxo-thiomorpholin-4-yl)-pyridin-3-yl]-3-fluoro-phenyl}-2-oxo-oxazolidin-5-ylmethyl ester methanesulphonic acid salt;
(2S,5R)-Acetic acid 3-{4-[6-(S,S-dioxo-thiomorpholin-4-yl)-pyridin-3-yl]-3-fluoro-phenyl}-2-oxo-oxazolidin-5-ylmethyl ester;
(S)—N-{3-[4-(6-(Thiomorpholin-4-yl)-pyridin-3-yl)-3-fluorophenyl]-2-oxo-oxazolidin-5-ylmethyl}-formamide,
(S)—N-{3-[4-(6-(S,S-Dioxo-thiomorpholin-4-yl)-pyridin-3-yl)-3-fluorophenyl]-2-oxo-oxazolidin-5-ylmethyl}-formamide;
(S)-{3-[4-(6-(thiomorpholin-4-yl)-pyridin-3-yl)-3-fluorophenyl]-2-oxo-oxazolidin-5-ylmethyl}-carbamic acid methyl ester;
(S)—N-{3-[4-(2-(S,S-Dioxo-thiomorpholin-4-yl)-pyridin-5-yl)-3-fluorophenyl]-2-oxo-oxazolidin-5-ylmethyl}-carbamic acid methyl ester;
(S)—N-{3-[4-(2-(S,S-Dioxo-thiomorpholin-4-yl)-3-methylpyridin-5-yl)-3-fluorophenyl]-2-oxo-oxazolidin-5-ylmethyl}-carbamic acid methyl ester;
(S)—N-{3-[4-(2-(S,S-Dioxo-thiomorpholin-4-yl)-3-methylpyridin-5-yl)-3-fluorophenyl]-5-hydroxymethyl}-oxazolidin-2-one;
(R)-Phosphoric acid mono {3-[4-(2-(S,S-dioxo-thiomorpholin-4-yl)-3-methylpyridin-5-yl)-3-fluorophenyl]-2-oxo-oxazolidin-5-ylmethyl}ester;
(R)-Phosphoric acid mono {3-[4-(2-(S-oxo-thiomorpholin-4-yl)-3-methylpyridin-5-yl)-3-fluorophenyl]-2-oxo-oxazolidin-5-ylmethyl}ester disodium salt;
(S)—N-{3-[4-(2-(S,S-Dioxo-thiomorpholin-4-yl)-3-methylpyridin-5-yl)-3-fluorophenyl]-2-oxo-oxazolidin-5-ylmethyl}-acetamide;
(S)—N-{3-[4-(2-(S,S-Dioxo-thiomorpholin-4-yl)-3-methylpyridin-5-yl)-3-fluorophenyl]-5-1,2,3-triazol-1-ylmethyl}-oxazolidin-2-one;

34. Pharmaceutical composition comprising a compound according to claim 1 and an excipient, diluent, solvent or a carrier.

35. Pharmaceutical composition comprising a compound according to claim 31 and an excipient, diluent, solvent or a carrier.

36. Pharmaceutical composition comprising a compound according to claim 32 and an excipient, diluent, solvent or a carrier.

37. Pharmaceutical composition comprising a compound according to claim 33 and an excipient, diluent, solvent or a carrier.

38. A method for treating a disease caused by a microbial infection in a human or animal comprising administering an effective amount of a compound according to claim 1, to the human or animal in need thereof.

39. A method for treating a disease caused by a microbial infection in a human or animal comprising administering an effective amount of a compound according to claim 31, to the human or animal in need thereof.

40. A method for treating a disease caused by a microbial infection in a human or animal comprising administering a pharmaceutical composition according to claim 32, to the human or animal in need thereof.

41. A method for treating a disease caused by a microbial infection in a human or animal comprising administering a pharmaceutical composition according to claim 33, to the human or animal in need thereof.

42. A method for preventing a disease caused by a microbial infection in a human or animal comprising administering an effective amount of a compound according to claim 1, to the human or animal at risk of being infected.

43. A method for preventing a disease caused by a microbial infection in a human or animal comprising administering an effective amount of a compound according to claim 31, to the human or animal at risk of being infected.

44. A method for preventing a disease caused by a microbial infection in a human or animal comprising administering a pharmaceutical composition according to claim 32, to the human or animal at risk of being infected.

45. A method for preventing a disease caused by a microbial infection in a human or animal comprising administering a pharmaceutical composition according to claim 33, to the human or animal at risk of being infected.

46. The animal in claim 38-45 is selected from mammal, fish, bird.

47. The microbial infection in claims 38-45 are central nervous system infections, external ear infections, infections of the middle ear, such as acute otitis media, infections of the cranial sinuses, eye infections, infections of the oral cavity, such as infections of the teeth, guns and mucosa, upper respiratory tract infections, lower respiratory tract infections, genitourinary infections, gastrointestinal infections, gynecological infections, mastitis, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients, osteomyelitis, endocarditis and diabetic foot.

48. The method of preparation of a compound according to claim 1.
